(19) United States
(12) Patent Application Publication
(10) Pub. No.: US 2004/0091941 A1
(43) Pub. Date: May 13, 2004

(54) Spotting pattern for placement of compounds in an array

(76) Inventors: William R. Ewing, Encinitas, CA (US); Robert K. Neeper, Ramona, CA (US); Rhett L. Affleck, Poway, CA (US); John E. Lillig, Poway, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/638,275

(22) Filed: Aug. 8, 2003

Related U.S. Application Data
(60) Provisional application No. 60/403,729, filed on Aug. 13, 2002.

(51) Int. Cl. G01N 33/53; G01N 33/543; B01J 33/00

(52) U.S. Cl. 435/7.1; 436/518; 502/1

(57) ABSTRACT

A plurality of samples of different substances are tested for their ability to enhance or inhibit a biological process. The samples are deposited on a supporting surface, such as a ChemCard, in an array comprising at least two dots of each sample, wherein at least one of the dots of each sample is at least a predetermined distance from at least one of the dots of each of the plurality of samples. One or more assay reagents in a gel sheet, for example, are then brought in contact with the compounds and reactions with certain of the compounds are evident as the active compounds diffuse into the one or more assay reagents. The concept of having unique neighbors for each occurrence of a dot allows the definitive correlation of active compounds with the dot (and the compound) that caused the biological activity.

### PRIORITY DATA

This application claims priority to U.S. Provisional Patent Application Serial No. 60/403,729, filed Aug. 13, 2002, which is incorporated herein by reference in its entirety.

### BACKGROUND OF THE INVENTION

1. Field of the Invention

The present invention relates to placement of compounds in an array such that absolute identification of specific compounds that exhibit biological activity is possible.

2. Description of the Related Art

High Throughput Screening (HTS) is the process by which a very large number of substances can be simultaneously tested for biological reaction with an assay reagent. For example, one widely used HTS technique utilizes 96 well test plates that are approximately 8 cm×12 cm. Various compounds are placed in the wells and simultaneously tested for biological activity as an assay reagent is placed in each of the wells.

While the use of 96 well plates greatly improves the testing efficiency of large numbers of substances, there is a need for increased efficiency. As such, many firms in the industry are working towards decreasing the size of the wells on the plates so that an increased number of compounds may be simultaneously tested. For example, many assays now use 384 well plates. However, as the size of the wells further decreases, additional complexities are introduced in to the HTS process. For example, the manufacture of the wells in the plates becomes increasingly complex and expensive. In addition, the accurate dispensing of compounds into smaller wells and other fluid handling steps becomes more difficult and error prone.

Other researchers have increased the number of compounds on a plate by eliminating the use of wells altogether. For example, U.S. Pat. No. 5,976,813, entitled “CONTINUOUS FORMAT HIGH THROUGHPUT SCREENING,” discloses an assay format in which multiple spots of candidate materials (such as chemical compounds) are spotted onto a supporting layer, preferably in dry form, and are then transferred into a preferably porous assay matrix, such as a gel, a filter, a fibrous material, or the like, where an assay is performed. In the context of this type of assay, the supporting layer carrying an array of (preferably dried) assay materials is referred to by the name “ChemCard,” which is proprietary to Discovery Partners International, Inc. Such usage in this disclosure is simply for purposes of convenience, and is neither an indication that ChemCard is considered generic or descriptive, nor an indication that the invention is limited to any particular type of ChemCard available from Discovery Partners International, Inc. or similar cards from any other vendor. The use of ChemCards carrying large numbers of dried candidate compounds (for example), that are to be assayed for a particular desired activity, provides a convenient, rapid, and powerful format for high throughput assays.

Assays of this type, which occur in a preferably porous matrix or other material in which reactants can diffuse, can sometimes produce initially ambiguous results which will require some interpretation or translation to eliminate the ambiguity. Because the reactants are not held in discrete locations, a positive result can be in the form of a “spot” that has diffused out to a diameter greater than that of the original dot on the ChemCard. This spot can be of a diameter that encompasses the corresponding locations of multiple dots.

During the course of some assays, the compound travels from the original ChemCard into one or more preferably porous assay matrix layers (e.g. gel layers) or onto another surface. Although the compounds generally keep their relative x, y centers, they may diffuse radially, even non-symmetrically, becoming more dilute. The eventual spot created by the differential signal in the assay response to an “active” compound (hereafter referred to simply as “spot”) is on an image derived from a medium that is not the original ChemCard. Therefore, there can be a discrepancy between the relative position of the center of the active spot and the relative position in which the compound was originally placed (hereafter referred to as the compound “dot”). Unlike assays performed in wells, there is not necessarily a visual outline to indicate where each compound is centered. Thus, if no errors were introduced in the x and y coordinates during the assay process then identification of each compound responsible for a spot could be made. However, error is introduced at each step of the assay process making definitive identification of the compound that produced the spot increasingly difficult. For example, error may be introduced by the liquid handler that places the compounds on the ChemCards (whether robotic or human), the diffusion of the compound between ChemCards, gels and membranes, the flexibility (distortion) of gels, and the nonlinear aspects of image collection. Each of these factors contributes to an error that is equal to the distance between the center of an imaged spot and the intended center of the compound dot on the original ChemCard, herein referred to as the dot-spot error, or DSE. The intended center of the compound dot on the ChemCard (the “intended location” of the compound dot) is the location that the compound dot is to be placed on the ChemCard, which may be different than the center of the location that the compound dot is actually placed on the ChemCard (e.g. the “actual location” of the compound dot). In other words, due to errors introduced in the process of placing the compound dot on the ChemCard, such as those introduced by the liquid handler, for example, the intended location of the dot may be different than the actual location of the dot. In application, if the DSE is less than half of the pitch (e.g. distance) between compound dots then the active spots may be absolutely correlated with their respective dots and a ChemCard carrying only a single instance of each compound may allow accurate identification of the compounds corresponding to the active spots. However, if the DSE is greater than one half the pitch between compound dots, ambiguity in the determination of the active spot is present. As such, a more sophisticated method of placing compounds in an array so that the active spots may be accurately correlated with their respective dot is desired.

### SUMMARY OF THE INVENTION

In one embodiment, the present invention comprises an array of n unique materials, each of which appears in the array at least two times and which has a plurality of neighboring materials, wherein for each of the n materials in the array, the neighboring materials in one occurrence of the material are different from the neighboring materials of all other occurrences of the material, wherein n is greater than 25. The materials may be considered neighboring materials of a particular material if they are within a predetermined radius around the particular material. The predetermined radius may be in the range of about 1 mm to 1 cm. The predetermined radius may be about 3mm. Alternatively, the materials may be considered neighboring materials of a particular material if they may be within a predetermined dot-pitch distance from the particular material. The predetermined dot-pitch distance may be 1 dot pitch. The predetermined dot-pitch may be between 1 and 5 dot pitch distances. The predetermined dot-pitch distance may be greater than 5 dot pitch distances. The value of n may be greater than 300, greater than 4000, or greater than 9000. The array may further be combined with a planar porous assay matrix, such that a surface of the porous assay matrix is in contact with each of the n materials of the array in such a manner that the materials can diffuse into the porous assay matrix. The porous assay matrix may contain at least one assay reagent capable of interacting with any of the materials in the array that is active in a test assay, wherein the test assay displays positive results (if any) within a time period t, during which time t the materials diffuse within the porous assay matrix to form a spot of diameter d, and wherein “neighboring materials” may be within a predetermined distance of the center of the spot. The array may further comprise a second porous assay matrix in contact with the porous assay matrix and containing at least one assay reagent capable of interacting with any of the materials in the array that is active in a test assay, wherein the test assay displays positive results (if any) within a time period t, during which time t the materials diffuse within the porous assay matrix and the second porous assay matrix to form a spot of diameter d on the second porous assay matrix and wherein “neighboring materials” may be within a predetermined distance of the center of the spot. The “neighboring materials” may include materials within a radius of about 3.5 mm.

In another embodiment, any number of porous assay matrices may be used in an assay process. For example, a porous gel matrix may initially be applied to the ChemCard. After sufficient diffusion of the compound dots, one or more porous, or non-porous, assay matrices may be temporarily contacted with the initial porous gel matrix.

In another embodiment, the present invention includes a method for creating an array of a plurality of materials, each material occurring at least twice within the array and having different neighboring materials in each occurrence. This method comprises (a) creating a candidate arrangement of materials containing n pairs of the materials in a spatial relationship, (b) testing whether a first occurrence and a second occurrence of each of the materials have different neighbors, (c) if the criteria of (b) is not satisfied, changing the spatial relationship of at least some of the n materials, and (d) repeating steps (b) and (c) until the criteria of (b) is satisfied. The candidate arrangement created in step (a) may comprise each of the plurality of materials such that when the criteria of (b) is satisfied, the array is complete. The candidate arrangement created in step (a) may comprise a portion of the plurality of materials, the method further comprising (e) expanding the candidate arrangement of materials by increasing n by a predetermined number, and (f) repeating steps (b)-(e) until a final candidate arrangement is created containing each of the plurality of materials. Changing the spatial relationship of at least some of the n the materials may comprise determining whether a placement of a nth material in every open location within the candidate arrangement fails to satisfy the criteria of (b), decrementing n if the nth material fails to satisfy the criteria of (b) in every open location within the candidate arrangement, and changing the spatial relationship of at least some of n the materials.

In another embodiment, a method of testing a plurality of samples of different substances for their ability to enhance or inhibit a biological process comprises depositing in an array at least two dots of each of the samples onto a planar matrix such that each of the at least two dots is centered at its own distinct site, wherein at least one of the at least two dots of each of the samples is at least a predetermined distance from at least one of the dots of each of the plurality of samples, transferring the array from the planar matrix into a uniformly dispersed assay reagent, and observing the interaction of each of the substances with the assay reagent and correlating the interaction with an ability of each of the substances to enhance or inhibit the biological process. The method may further comprise depositing in a second array at least two dots of each of the samples onto a second planar matrix such that each of the at least two dots is centered at its own distinct site, wherein at least one of the at least two dots of each of the samples is at least a predetermined distance from at least one of the dots of each of the plurality of samples, and a pattern of placement of the dots on the second array is different than a pattern of placement of the dots on the first array, placing the uniformly dispersed assay reagent on the second planar matrix, and observing the interaction of each of the substances with the assay reagent and correlating the interaction with an ability of each of the substances to enhance or inhibit the biological process. The method may further comprise placing a second uniformly dispersed assay reagent on the uniformly dispersed assay reagent, observing a second interaction of each of the substances with the second uniformly dispersed assay reagent, and correlating the second interaction with an ability of each of the substances to enhance or inhibit the biological process. The method may further comprise placing a third uniformly dispersed assay reagent on the second uniformly dispersed assay reagent, observing a third interaction of each of the substances with the third uniformly dispersed assay reagent, and correlating the third interaction with an ability of each of the substances to enhance or inhibit the biological process. The planar matrix may comprise a plurality of alignment dots configured to diffuse into the uniformly dispersed assay reagent, the second uniformly dispersed assay reagent, and the third uniformly dispersed assay reagent; the alignment dots on the third uniformly dispersed assay reagent providing reference points for the orientation of the third uniformly dispersed assay reagent with respect to the planar matrix.

In another embodiment, a method of testing a plurality of samples of different substances for their ability to enhance or inhibit a biological process comprises depositing in an array at least one dot of each of the samples onto a plurality of planar matrixes such that each of the at least one dot is centered at its own distinct site, wherein at least one of the dots of each of the samples is at least a predetermined distance from at least one of the dots of each of the plurality of samples, transferring the array of samples from the plurality of planar matrixes into a uniformly dispersed assay reagent, and observing the interaction of each of the substances with the assay reagent and correlating the interaction with an ability of each of the substances to enhance or inhibit the biological process.

In another embodiment, a system for testing the interaction of a plurality of samples of different compounds with an assay reagent comprises a relatively flat carrier configured to support the plurality of samples and a dispensing mechanism configured to dispense at least two dots of each of the plurality of samples of different compounds on the flat carrier such that each of the at least two dots of each of the plurality of samples has a different set of neighboring compounds within a predetermined distance. The system may further comprise a detection mechanism configured to detect interactions between each of the plurality of samples dispensed on the flat carrier and the assay reagent. The dispensing mechanism, may simultaneously dispense a portion of the plurality of samples, where the pipettes in the dispense mechanism are arranged in a particular relative orientation, on at least two locations on the flat container. None of the samples in the portion of the plurality of samples may be neighboring compounds. The portion of the plurality of samples may comprise twelve samples. The assay reagent may be uniformly dispersed in a gel sheet. The flat carrier may comprise a plurality of alignment dots configured to diffuse into the assay reagent and provide reference points for the orientation of the gel sheet with respect to the flat carrier.

Another embodiment comprises an array of samples in which each sample appears at least twice and there may be at least 768 dispenses of groups of 12 samples.

Another embodiment comprises an array of samples in which each sample appears at least twice and there may be at least 4608 samples, wherein the array comprises 144 columns and 64 rows and the samples may be arranged substantially according to the relative coordinates shown in Table 1.

In another embodiment, a method for performing an assay comprises providing a substantially planar substrate having an array of at least two dots of each of a plurality of test materials, wherein each of the dots has a plurality of neighboring test materials and is placed such that the neighboring materials in one occurrence of the material are different from the neighboring materials of all other occurrences of the material, and transferring the array of test materials into a uniformly-dispersed assay reagent that participates in indicating which of the test materials may be active in the assay while maintaining the relative positioning of the test materials vis-a-vis each other. Transferring the array of test materials may comprise contacting the array with the assay reagent and allowing the materials to diffuse into the assay reagent. Transferring the array may comprise transferring the materials into a first matrix, and then contacting the first matrix with the assay reagent. Transferring the array may comprise contacting the array with a gel into which the materials can diffuse.

These and other objects and features of the present invention will become more fully apparent from the following description and appended claims taken in conjunction with the following drawings, where like reference numbers indicate identical or functionally similar elements.

### BRIEF DESCRIPTION OF THE DRAWINGS

FIG. 1 is a perspective view of a ChemCard and a gel sheet.

FIG. 2 is a perspective view of a gel sheet placed on a ChemCard.

FIG. 3 is a top view of an image of spots transposed on a ChemCard having a plurality of compound dots placed thereon.

FIG. 4 is a flow chart illustrating a method of placing pairs of compounds in an array so that the active dot may be accurately correlating with a resultant active spot.

FIG. 5 is a flow chart illustrating one method of performing block 440 of FIG. 4.

FIG. 6a, 6b, and 6c are diagrams representing a portion of an exemplary process of placing compounds in an array so that each occurrence of a particular compound has different neighbors.

FIG. 7 is a flow chart illustrating a method of determining an array S_{A} such that each of the compounds in each of the A dispense positions satisfy the neighbor constraints.

FIG. 8 is a flowchart illustrating one method of performing the neighbor constraint checks for an exemplary compound M.

FIG. 9 illustrates a portion of a placement array geographically arranged as the compounds would be placed on a ChemCard.

### DETAILED DESCRIPTION

In the following description, reference is made to the accompanying drawings, which form a part hereof, and which show, by way of illustration, specific examples or processes in which the invention may be practiced. Where possible, the same reference numbers are used throughout the drawings to refer to the same or like components. In some instances, numerous specific details are set forth in order to provide a thorough understanding of the invention. The invention, however, may be practiced without the specific details or with certain alternative equivalent devices and/or components and methods to those described herein. In other instances, well-known methods and devices and/or components have not been described in detail so as not to unnecessarily obscure aspects of the invention.

In one embodiment, two compound dots are microarrayed for each compound to be tested in order to create a pair of active spots for each active compound. For each compound, the two dots are placed in positions on the same ChemCard such that each has a different set of neighbors within a known distance (a “neighbor distance”) that is greater than the DSE. Similarly, “neighbors” of a particular compound, as used herein, is defined as those compound within a “neighbor distance” of the center of the particular compound dot. In an advantageous embodiment, the neighbor distance is maximized by an algorithm such that the distance between one pair of every combination of compounds is maximized. In order to allow positive identification of the dot corresponding to an active spot the neighbor distance should be greater than the determined DSE (representative of the errors introduced in the assay process). In various embodiments, the neighbor distance may vary depending on the complexity of an assay process. For example, in a simple assay process, with a corresponding low DSE, the algorithm may quickly provide a pattern for placing the compound pairs on the ChemCard (e.g. the neighbor distance may be only slightly larger than the DSE). Likewise, in a multi-step assay, or otherwise error prone assay process, the DSE may be much larger and require an increasingly complex algorithm, or multiple iterations of an algorithm, in order to provide a pattern for placing the compound pairs on the ChemCard with a neighbor distance that is greater than the DSE. Thus, no compounds within the DSE of a particular dot will be within the DSE of the particular dot's counterpart dot. This concept of having unique neighbors for each occurrence of a dot allows the correlation of active spots with the dots (and the compounds) that caused the biological activity. While a system and method for placing pairs of compounds on a ChemCard is described in detail herein, one of skill in the art will recognize that the concepts described herein may be applied when additional occurrences of each compound are used. For example, in an embodiment that places four occurrences of each compound on a single ChemCard, the systems and methods described herein may be applied to ensure that each of the four occurrences of a specific compound have unique neighbors. In addition, one of skill in the art will recognize that a plurality of patterns that produce unique neighbors for each occurrence of a particular compound dot may be determined.

In one embodiment, ChemCards having the same or similar dimensions as typical welled microplates used for HTS are used in order to allow the use of existing detectors and plate handlers.

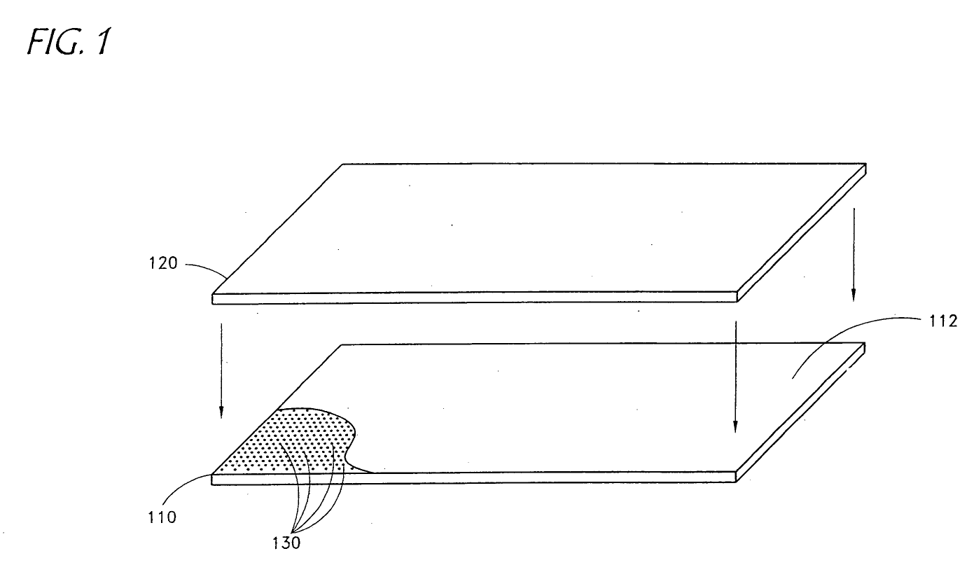

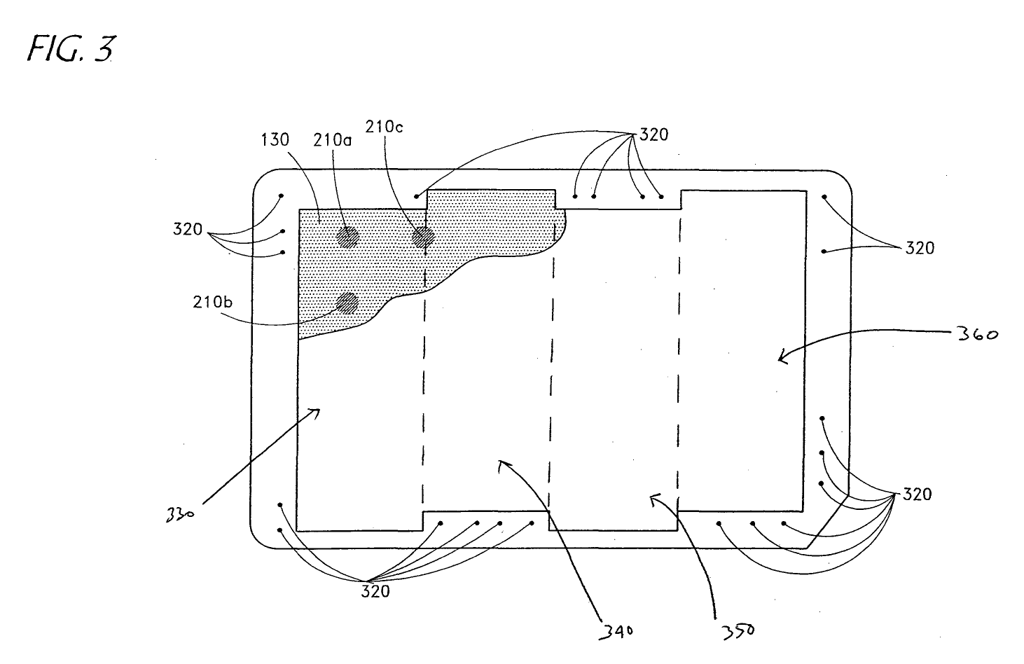

FIG. 1 is a perspective view of a ChemCard 110 and a preferably porous assay matrix (e.g. a gel sheet 120). Throughout this description the term gel and gel sheet are used as one example of a porous assay matrix layer. It is contemplated that any other porous assay matrix layer may be used in place of any gel or gel sheet described herein, and a reference to a gel in this detailed description does not exclude the substitution of other types of assay matrices, which substitution is expressly contemplated. For example, a non-porous matrix, such as a non-porous hydrophilic sheet, may be used to carry reagents. These reagents may be attached, such as by a covalent bond, for example, or may be free to diffuse from the surface of the porous or non-porous sheet. A plurality of compound dots 130 are placed on a top surface 112 of the ChemCard 110 in an array. In one embodiment, rows and columns of dots 130 are placed on the ChemCard 110 in a honeycomb pattern, rather than a square array, to maximize the number of the compounds that may be placed on the ChemCard 110. In the illustration of FIG. 1, only a portion of the dots 130 are shown on the ChemCard 110. In operation, some or substantially all of the surface 112 of the ChemCard 110 may have dots 130 placed thereon. In one embodiment, dots 130 are not placed on certain portions of the ChemCard 110 in order to allow the placement of alignment dots (discussed below with reference to FIG. 3). In addition, dots 130 may not be placed on designated regions of the ChemCard so that the regions can be used for positive or negative assay controls.

In an advantageous embodiment, the top surface 112 of the ChemCard 110 is flat in order to allow the gel sheet 120 to come in complete contact with the compound dots 130 on the ChemCard. The direct and complete contact of each of the compound dots 130 is necessary for dissolution and transfer of the compounds from the top surface 112 into the gel sheet 120.

In one embodiment, the gel sheet 120 comprises a uniformly dispersed assay reagent in a substrate. The composition and method of manufacturing the gel sheet 120 is described in more detail in the co-pending application titled, “SYSTEMS AND METHODS FOR CASTING AND HANDLING ASSAY MATRICES,” application Ser. No. 10/219081, which is hereby incorporated by reference for all purposes.

In operation, after the compound dots 130 are placed on the ChemCard 110, the gel sheet 120 may be placed in direct contact with the ChemCard 110, or in direct contact with a medium into which the compound dots 130 have been transferred in registry from the ChemCard 110. As such, each of the compounds in the dots 130 is in direct contact with the assay reagent in the gel sheet 120. Those particular compounds that biologically react with the assay reagent in the gel sheet 120 typically diffuse, directly or indirectly, into the gel sheet 120 and create an identifiable diffusion spot on the gel sheet. In addition, cards or gel sheets comprising other assay reagents may be applied to the gel sheet 120 with the diffusion spots. In this way, a multi step assay may be performed on a set of compound dots 130 on a single ChemCard 110. However, as the number of steps in the assay process increases (e.g. additional assay reagents in gel sheets are applied), the correlation of the eventual active spots with the original dots becomes increasingly difficult.

In one embodiment, the compounds are transferred to the ChemCard 110 as solutions of compound in a solvent, such as dimethyl sulfoxide (DMSO). The amount of compound in each dot is determined based on the known concentration at which assays are typically run and the thickness of the gel sheet or sheets 120. In an advantageous embodiment, each of the dots comprises about 20 ng of compound. However, the amount of compound varies with the particular ChemCard and its intended assay system, and may be any suitable value.

The minimum volume of the DMSO solution may be determined by the maximum concentration of the compound that will dissolve in the DMSO solvent. If the concentration of a compound is too high, precipitation of the compound may occur. On the other hand, if the concentration of a compound is too low (and the volume of solution is not increased), the interaction between the compound and the assay reagent in the gel sheet may not be identifiable. In an advantageous embodiment, the concentration of compound in DMSO solvent is about 1 mg/mL. Thus, if each dot comprises about 20 ng of compound, the volume of the DMSO solution placed on the ChemCard in forming each dot 130 is about 20 nL (i.e. 20 ng/1 mg/mL=20 nL). In laboratory tests, 20 nL dots of solution occupy a diameter of less than about 1 mm, and typically fall within the range from about 0.6 to 0.7 mm. In addition, the radius of the dots 130 depends on the contact angle between the DMSO solution and the surface of the ChemCard, such that as the contact angle decreases, the dot 130 radius increases. One of skill in the art will recognize that dots of different sizes, having different concentrations of compounds, and using different solvents, may work equally as well in the present system. The placement of the compound dots 130 on the ChemCard 110 may also be performed in other manners, other than by creating a solution.

In one advantageous embodiment, the number of compound dots 130 on a single ChemCard 110 is 9216. Thus, if compounds are placed in duplicate on the ChemCard 110, 4608 different compound dot 130 pairs may be placed on each ChemCard.

The compounds awaiting placement on the ChemCard are typically stored in and robotically transferred from standard microwell plates, or microplates. In a system using 4608 pairs of compounds per ChemCard 110, multiples of standard sized microplates may be used to supply the compound solutions for spotting. For example, forty eight 96 well microplates, twelve 384 well microplates, or three 1,536 well microplates may store the necessary 4608 compounds. In addition, the efficiency of using a ChemCard carrying 9,216 compound dots may be improved by using a number of rows and columns that are multiples of standard microplate rows and columns. For example, in one embodiment, the ChemCard comprises 64 rows and 144 columns such that a number of standard microplate rows and columns may be used to fill the 64 rows and 144 columns. For example, the 64 rows may be evenly divided and filled by microplates with 8, 16, or 32 rows. Likewise, the 144 columns may be evenly divided and filled by microplates with 12, 24, or 48 columns.

In one embodiment, a dispensing mechanism dispenses a plurality of compounds simultaneously on the ChemCard 110. For example, the dispensing mechanism may comprise a plurality of pipettes for distribution of the compounds. The number of pipettes may vary greatly depending on the particular objectives of the dispensing (microarraying) process, and typically range from 1 to 96 pipettes. In one embodiment, the position of each of the plurality of pipettes are at fixed locations relative to one another, such that all of the plurality of pipettes move in parallel. In another embodiment, each of the pipettes moves independent of the others. In one embodiment, the range of movement of each pipette is exclusive, such that only a single pipette may dispense at each location on the ChemCard 110. The number of parallel pipettes used in a particular microarraying system may largely affect the neighbor distance that may be obtained. Specifically, in a system using a 96 pipette dispensing mechanism, the number of possible dispense locations on a ChemCard is less than the number of possible dispense locations a 12 pipette dispensing mechanism would have on the same ChemCard. Thus, the number of pipettes in the dispensing mechanism is directly related to the maximum neighbor distance that may be obtained.

In a typical compound dispensing system, each of the pipettes in the dispensing mechanism aspirates a compound from a microwell in a microplate, the dispensing mechanism is positioned over a predetermined location of the ChemCard, and the pipettes dispense the compound in a predetermined location. The pipettes may store enough of each compound so that multiple dispenses of the compound may occur without refilling the pipette from the microplate. Before loading the pipettes with different compounds, the pipettes should be thoroughly washed in order to ensure that each of the compounds placed by the pipettes are pure. The washing process typically requires a significant amount of time to perform. For example, in certain embodiment, the washing process requires more time to perform than the process of loading and aspirating each of the pipettes.

In one embodiment, all of the pipettes in a particular dispensing mechanism dispenses simultaneously. For example, in a 12 pipette dispensing mechanism, each of the pipettes may dispense (fire) simultaneously, depositing compounds from each of the 12 pipettes on the ChemCard. Likewise, a 96 pipette dispensing mechanism may simultaneously dispense each of the compounds in the 96 pipettes. In another embodiment, only a selected portion of the pipettes may be simultaneously dispensed. For example, in a system comprising a 96 pipette dispensing mechanism, the dispensing mechanism may simultaneously dispense only 12 of the 96 total pipettes. The 96 pipette dispensing mechanism may then move to another location over the ChemCard and dispense another 12 pipettes. In this particular example, the process may be repeated 8 times, aspirating 12 pipettes at each location, in order to dispense each of the 96 pipettes. Thus, the dispensing mechanism may dispense 8 different sets of 12 compounds, at multiple locations, without washing the pipettes. By reducing the number of wash cycles required (by having more pipettes operate in parallel, for example) to place a specific number of compounds on a ChemCard the time required to place the compounds on a ChemCard may be reduced. In addition, the dispensing of only a portion of the total pipettes in a dispense mechanism may allow a larger neighbor distance in the array. For example, if only 12 pipettes of a 96 pipette dispensing mechanism are dispensed at each location above the ChemCard, the neighbor distance may be equivalent to the neighbor distance in a system using a simultaneous aspirating 12 pipette dispensing mechanism.

If the dispensing mechanism simultaneously fires more than one pipette, the pitch between the pipettes may well need to be greater than the predetermined distance between neighbors for the particular assay process. Otherwise, each occurrence of the compound in a particular dispensing mechanism would have the same neighbors. In one exemplary embodiment, the dispensing mechanism comprises 12 pipettes, such that 12 compounds are simultaneously picked up from a microplate and simultaneously dispensed on the ChemCard. Each set of 12 compounds is dispensed on at least two different locations on the ChemCard before washing the pipettes and retrieving the next 12 compounds for placement. Subsequent dispenses of different compounds may be placed in the areas between the compounds placed previously. As such, compounds from different groups of dispenses may be neighbors, e.g. within a neighbor distance of one another. In one embodiment, an ink-jet type dispensing mechanism may be used to print the desired dots on the ChemCard 110. In another embodiment, a pin spotter may be used to apply the desired dots to the ChemCard 110.

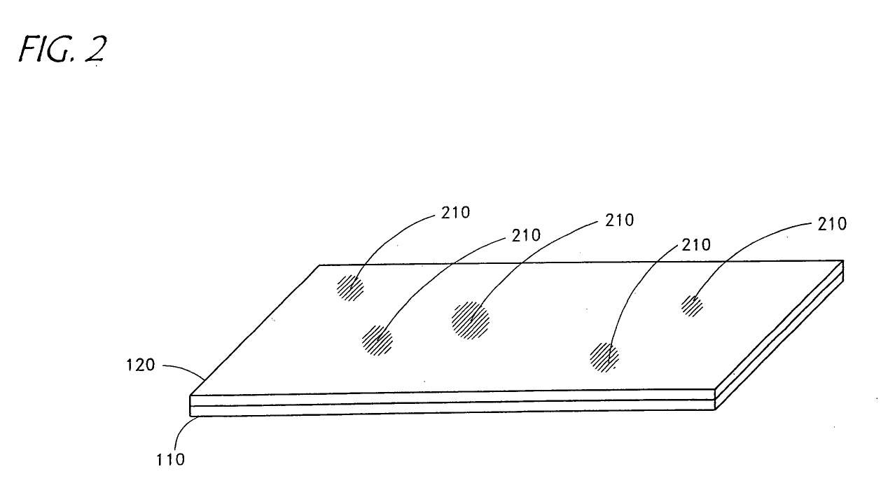

FIG. 2 is a perspective view of a gel sheet 120 placed on a ChemCard 110. As discussed above, the ChemCard 110 includes a plurality of compound dots to be assayed for a particular biological activity. For example, in one embodiment, the ChemCard 110 is covered with 9216 dots comprising pairs of 4608 different compounds. The dots can be transferred directly or indirectly into a substrate (such as a gel) in which assay results are read. As the compounds diffuse into the gel sheet 120, those compounds that biologically react with the assay reagent(s) in the gel sheet 120 form detectable spots 210. The spots 210 may then be identified by a detection mechanism, such as a human or (preferably) machine vision system which determines the dots 130 on the ChemCard 110 that produced the spots 210 (the “active dots”). However, as indicated in FIG. 2, the size of the spots 210 on the gel sheet 120 is substantially larger than the size of the dots 130 on the ChemCard (FIG. 1). As such, the definitive identification of the active dots becomes increasingly complex. Furthermore, during the course of an assay the compound may travel from the original ChemCard 110 into multiple gel layers 120 or onto other surfaces. In fact, the compounds may be transferred to any number of gel layers 120 in an assay process and any number of gel layers 120 carrying reagents may be applied to layers carrying the compounds. Also, any number of non-porous surfaces carrying reagents may be applied to a gel layer 120 in the assay process. The attachment of reagents to a non-porous surfaces, such as a solid surface, may be accomplished by dipping, or otherwise covering, the surface of the non-porous surface with a reagent solution. Alternatively, reagents may be attached to a non-porous surface chemically (such as by a covalent bond). Thus, the eventual spot 210 created by the differential signal in the assay response to an active compound dot 130a ( active dots 130a are those dots 130 that correspond to an active spot 210) is on a medium that is not the original ChemCard 110. Therefore, the identification of the absolute center of the spots 210 may not accurately identify the active dot 130a as there may be a discrepancy between the relative position of the center of the active spot 210 and the intended center of the compound dot 130 (FIG. 1) on the ChemCard 110.

FIG. 3 is a top view of an image of spots (e.g. a photo of the final gel layer 120 used in the assay process) transposed on a ChemCard 110 having a plurality of compound dots 130 placed thereon. The image of spots 210a, 210b, and 210c may be captured from the most recently applied gel layer 120 in a multiple step assay process. For example, a first gel layer 120 may be placed on the ChemCard 110. The first gel layer 120 may have a first assay, or alternatively, may not have any assay reagents. The first gel layer 120 may be removed from the ChemCard 110 and placed on a second gel layer 120 (having different assay reagents than the first gel layer 120). The first and second gel layers may then be separated and a third gel layer placed in contact with the second gel layer. The image of spots 210 on the third gel layer may then be captured. The spots 210 are thus representative of the biological reactions between the compounds from dots 130 through the first gel layer, the second gel layer, and the third gel layer. It should be understood that in some instances, the actual compound may not itself be present in the spot that is imaged, but instead, the compound has acted as a catalyst or trigger for the creation of an imageable spot from other reagents.

Throughout each stage of the above described multiple stage assay process, a degree of error is introduced in the absolute locations of the spots 210 with respect to the intended and actual locations of their corresponding dots 130 on the ChemCard (e.g. cumulatively the DSE). As such, the center of the active dot 130a (e.g. the actual location) associated with each of the spots 210 may not lie in the absolute center of the spot 210. For example, in FIG. 3, the active dot 130a reacts with each of the gel layers in the assay process to create active spot 210a. However, through the errors introduced in the various steps of the assay process, the center of the active dot 130a is not in the absolute center of the spot 210a. As such, the identification of active dots (e.g. dot 130a) may not be positively identified by comparing the locations of the image including spots 210 to the original dot 130 locations on the ChemCard.

In one embodiment, alignment dots 320 are placed on the periphery of the ChemCard in order to ensure that the orientation and alignment of the final image containing spots 210 may be correlated with the original placement of the compound dots 130 for determination of the active dots 130a. In the embodiment of FIG. 3, the twenty-two alignment dots provide an asymmetric pattern in both mirror planes, even with the absence, obscuration, or addition of some spots developing in the alignment dot region. This asymmetry is desirable since during the assay process porous assay matrixes (such as gel sheets), membranes, and images can be flipped relative to the original ChemCard. In one embodiment, the alignment dots 320 are outside of the array where the compounds are placed to reduce interference with the assay of the compounds and, conversely, to reduce the effect of active spots on the alignment dots 320, as well as to make dot dispensing more efficient. The placement of the alignment dots outside the array of compounds may also advantageously increase the neighbor distance by providing a larger area for a multi-pipette dispensing mechanism to simultaneously dispense multiple compounds. In contrast, if the alignment dots are place in the middle of the array of compounds, the possible dispense locations for a simultaneous multi-pipette dispense is decreased.

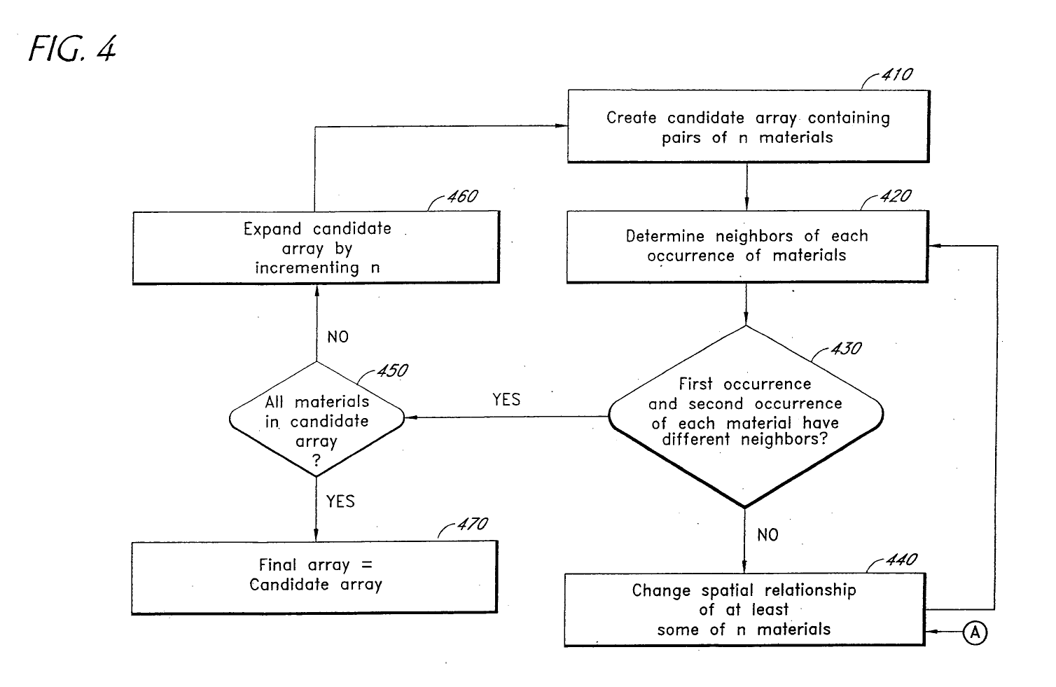

FIG. 4 is a flow chart illustrating a method of placing pairs of compounds in an array so that the active dot 130a may be accurately correlating with a resultant, active spot 210. FIG. 4 illustrates the concept of placing the pairs of compounds in an array such that each occurrence of a particular compound has different neighbors. The neighbor distance should be selected to include at least all of the compounds within the DSE for the particular assay. Accordingly, because the DSE depends on several aspects of the assay process, the number of neighbors for each compound may vary greatly depending on the particular assay method being used. In one embodiment, neighbors of a particular compound dot 130 include all dots 130 within a 3 mm radius. In other embodiments, the neighbors of a particular compound dot may include those dots within a smaller radius, e.g. 1 mm, 1.5 mm, 2 mm, or a larger radius, e.g. 4 mm, 6 mm, 1 cm. Those of skill in the art will recognize that the techniques disclosed herein are equally applicable to each of the above examples. Alternatively, the neighbors can include all those compounds within a given dot-pitch distance, such as those within a radius of 1 dot pitch, or 1.5, 2, 3, 4, 5, 8, or 10 dot pitch distances. Likewise, the neighbors may include an absolute number of neighboring dots. For example, in a system using a honeycomb pattern dot placement, the number of neighbors for any particular dot (excluding those dots on the edges of the card) may include the 6 immediately adjacent neighbors. In like manner, in a system using a honeycomb pattern dot placement, the number of neighbors may include two rings of surrounding dots, or 18 dots. One of skill in the art will recognize that an absolute number of neighboring dots may be extended to include any desired number of surrounding dots. Furthermore, in a system using any other pattern for dot placement (such as a grid pattern, for example) each of the above described methods for determining neighbors is equally applicable.

In block 410, a candidate array of pairs of n compounds is created. As mentioned above, in one embodiment, 4608 different compounds are placed on the ChemCard 110. Thus, in an embodiment using pairs of each compound, a total of 9216 dots will be placed on the ChemCard 110. In one embodiment, the candidate array comprises a matrix of X rows and Y columns, where X * Y=the total number of dots (e.g. 9216), where each coordinate of the assay contains a representation of one of the compounds (e.g. numbers 1-4608). As such, the array would contain two occurrences of each number 1-4608. In one embodiment, the candidate array begins with n=1, such that only a single pair of compounds are positioned in the array. However, it is contemplated that any number of compounds may be positioned in the array in block 410. In fact, in one embodiment all of the compounds that are to be placed in the array are assigned a random location in the array in block 410 such that the array is initially filled with two complete sets of the numbers from 1-4608.

In block 420, the neighbors of each occurrence of a compound are determined. In one embodiment, the neighbors of a particular compound include a predetermined number of compounds surrounding the particular compound in the X and Y directions in the candidate array. In another embodiment, neighbors of a particular compound include those compounds within a predetermined radius of the particular compound.

In decision block 430, the method determines whether a first occurrence and second occurrence of each compound have different neighbors (i.e. exclusively unique neighbors). This process is referred to herein as a constraint check, wherein the constraints are satisfied when the first occurrence and second occurrence of a compound pair have none of the same neighbors. For example, each occurrence of a particular compound may have 40 neighbors. Block 430 determines whether any of the 40 neighbors to the first occurrence are the same as any of the 40 neighbors to the second occurrence. If decision block 430 determines that there are occurrences of compounds in the array that have the same neighbors, the candidate array is changed in block 440. If the candidate array including pairs of compounds having the same neighbors is used in the assay process the active spots will not be definitively matched to a compound dot on the ChemCard.

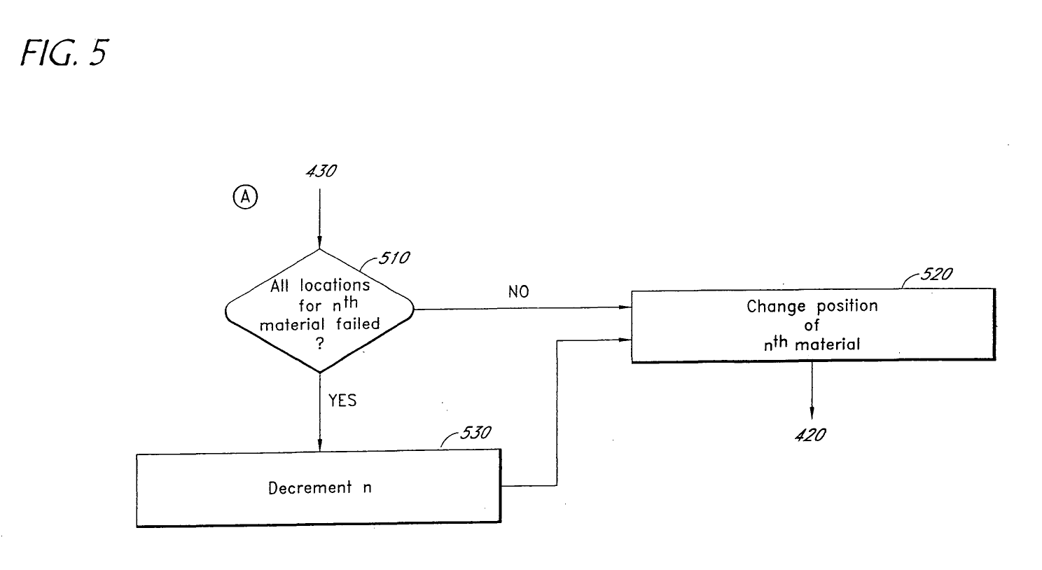

In block 440, the spatial relationship (e.g. coordinates in the candidate array) of at least some of the materials in the array are changed. Many different methods of changing the spatial relationship of the array are possible in this block 440. For example, in one embodiment, the entire candidate array may be refilled in random order. In another embodiment, the locations of those pairs of compounds that are both neighbors to a particular compound may be changed. For example, if 100 pairs (200 dots) of neighbors are common to pairs of compounds, the locations of the 200 dots may be adjusted at random, shifted, or adjusted by an algorithm, leaving the remaining dots in their respective positions. In another embodiment, a genetic algorithm may be implemented to adjust the spatial arrangement of the compounds in the candidate array. See the discussion with reference to FIG. 5 for further detail on changing the spatial relationship of dots in the candidate array.

After the spatial relationship of the compounds in the candidate array has been adjusted (block 440), the method returns to block 420 which determines the neighbors of each occurrence of the compounds, and to block 430 which repeats the constraint check on the changed candidate array. The process of blocks 420, 430 and 440 continues until every pair of compounds passes the constraint check of block 430.

After the constraints of block 430 have been satisfied, block 450 determines whether all compounds that are to be placed in the candidate array have already been placed in the array. For example, in an embodiment using 4,608 different compounds, when n=4,608 all of the compounds have been placed in the array. Thus, in block 430, while n is less than the total number of compounds to be placed in the array (4,608 in one exemplary embodiment), the method moves to block 460. On the other hand, if all compounds have been placed in the array (e.g. n=the total number of compounds to be placed in the array), the method moves to block 470. Thus, in an embodiment that places all compounds in the candidate array prior to performing any constraint check (e.g. all compounds are placed in block 410), blocks 450 and 460 are not necessary because all n pairs of materials are always placed in the candidate array. Therefore, in such an embodiment, when the constraint check of block 430 is satisfied, the method continues directly to block 470.

In block 460, the candidate array is expanded by incrementing n, i.e. the number of pairs of compounds in the array. In one embodiment, the position of the additional compound pair(s) may be determined at random. In another embodiment, the additional compound pair(s) may be inserted in to those remaining locations in the candidate array that are furthest away from one another.

In block 470, the candidate array becomes the final array that will be used for placement of the compound dots 130 on the ChemCard 110 (See FIGS. 1 and 3, for example).

FIG. 5 is a flow chart illustrating one method of performing block 440 of FIG. 4. When the process of FIG. 5 is implemented in the placement method of FIG. 4, the position of the compound pair that was most recently added to the candidate array is changed if there is a constraint violation in block 430. If the position of the compound pair that was most recently added to the candidate array cannot be changed to conform to satisfy the constraints in block 430, the position of the previously added compound pair (e.g. n−1) will be adjusted and location of acceptable positions for the most recently added pair of compounds (e.g. n) will again be attempted.

In decision block 510, if all possible locations in the candidate array for the nth pair of compounds, e.g. the most recently added pair of compounds, have been unsuccessfully tested against the constraints requirements of block 430, then block 530 decrements the value of n. Thus, block 530 performs a step back (or more than one step back) in the addition of new pairs of compounds to the candidate array in order to work around a constraint violation that could not be avoided through movement of the most recently added pair of compounds.

In block 520, the position of at least one of the nth pair of compounds in the candidate array is changed. If n has just been decremented in block 530, the nth pair of compounds has previously satisfied the constraints of block 430. However, the position of the nth pair in the candidate array is adjusted to another location that satisfies the constraints of block 430 in an attempt to change the spatial arrangement so that the now n+1 pair of compounds (i.e. the pair that failed the constraints test for every location in block 510) may be moved to a location that satisfies the constraints of block 430. One of skill in the art will recognize that the step back function of block 530 may step back multiple levels in the placement of compound pairs (e.g. n may be decremented multiple times) in order to change the spatial arrangement of the candidate array so that the previously constraint violating pair of compounds may satisfy the constraints of block 430.

After block 520 has changed the position of at least one of the nth pair of compounds, the method returns to block 420 which determines the neighbors of each occurrence of compounds, and to block 430 which performs the constraint check. The process of blocks 520, 420, 430, and 510 will continue until either (a) a position for the nth pair of compounds in the candidate arrangement that satisfies the constraints of block 430 is identified or (b) n is decremented by block 530 as a result of the inability to satisfy the constraints of block 430 by adjusting the position of the nth pair of compounds.

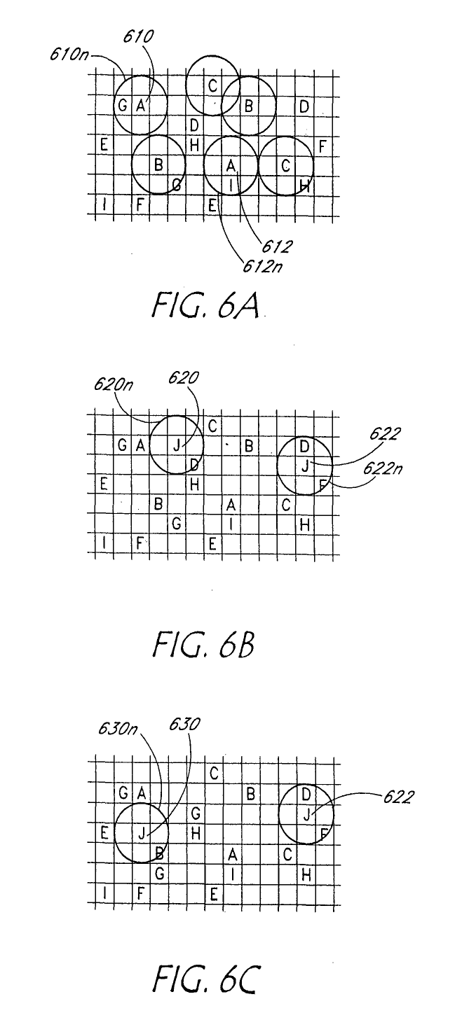

FIG. 6a, 6b, and 6c are diagrams representing a portion of an exemplary process of placing compounds in an array so that each occurrence of a particular compound has different neighbors. FIG. 6a illustrates the position of compound pairs represented by the letters A, B, C, D, E, F, G, H, and I, in a portion of an array. As shown in FIG. 6, two occurrences of each compound are positioned in the array. For example, compound A is placed at locations 610 and 612. FIG. 6a also illustrates the range of neighbors that will be considered for constraints checks. In particular, the range of neighbors in FIG. 6 covers a radius of approximately 1.5 times the pitch of the compounds. However, as discussed above, the neighbor distance may vary depending on the particular assay process. For example, in one embodiment with compound dots placed in a honeycomb pattern on the ChemCard with a vertical pitch of about 1.125 mm, a horizontal pitch of about 1.5 mm, and a diagonal pitch of about 0.95 mm, an algorithm has determined an array of 4608 compound pairs that have unique neighbors within a neighbor distance of about 3.5 mm. Alternatively, the range of neighbors may include a certain number of surrounding compounds in each direction rather than being limited by an absolute distance.

In FIG. 6a, the range of neighbors for the occurrences of compound A at location 610 and 612 are indicated by circles 610n and 612n, respectively. Thus, neighbors of the occurrence of compound A at location 610 include compound G and neighbors of the occurrence of compound A at location 612 include compound I. Because each of the occurrences of compound A (i.e. at location 610 and 612) have different neighbors, the position of the pair of compound A dots satisfies the constraints, as described with reference to block 430 of FIG. 5.

FIG. 6b illustrates the position of compound pairs represented by the letters A-I and, additionally, a candidate placement of compound J. As indicated in FIG. 6b, the pair of compound J dots are preliminarily placed at locations 620 and 622 within the candidate array, and the neighbors of the two occurrences include those compounds that are at least partially within the circles 620n and 622n, respectively. In particular, neighbors of the occurrence of compound J at location 620 include compound D and neighbors of the occurrence of compound J at location 622 include compounds D and F. Because each of the occurrences of compound J have a common neighbor, D, the position of the pair of compound J dots does not satisfy the placement constraints. If compound J were left in the position indicated in FIG. 6b, the reaction by either compound D or J in the assay process may create spots on the final gel sheet that are not definitively attributable to either compound D or J. Thus, the candidate arrangement of compounds shown in FIG. 6b should be adjusted.

FIG. 6c illustrates the position of compound pairs represented by the letters A-J, wherein the position of compound J previously at location 620 (FIG. 6b) has been changed to a new location 630. The neighbors of compound J at location 630 include compound B and the neighbors of compound J at location 622 include compounds D and F. Thus, the movement of compound J from location 620 to 630 removes the duplicate neighbors from the occurrences of compound J. As such, the current candidate array satisfies the placement constraints and additional compounds, if any, may be added using a similar process.

In one embodiment, the ChemCard comprises columns on the leftmost and rightmost areas of the field of view-that are not microarrayed with either alignment dots or compounds. These regions are left for the blank during the microarraying process such that they can be used for assay controls by the end user. There are many different HTS assays that can be run on the same compounds, and many copies of each ChemCard can be made for the various assays that will be run on them. Each assay can have its own control compounds to verify and quantitate an active response to the assay (positive controls). One feature of this embodiment is that no array locations need to be sacrificed for negative controls, since background, defined as the lack of influence of compounds, exists throughout the image. In contrast, in a typical microplate assay, wells must be used for negative controls.

In one embodiment, an assay process uses a robotic dispensing mechanism to place the compounds on the ChemCard. For example, in an exemplary embodiment, the dispensing mechanism comprises 12 pipettes that dispense each compound twice on the ChemCard (creating two dots of each compound) so that each of the two dots has a different set of neighboring compounds. As discussed above, the number of simultaneous pipette dispenses at each location over the ChemCard may affect both the efficiency of the microarraying process (e.g. the number of washings may be decreased by firing only a portion of the pipettes at each location) and the neighbor distance (e.g. as the number of simultaneous pipette dispenses increases, the neighbor distance decreases).

In an assay using a ChemCard having 4608 unique compounds (and thus 9216 dots), the dispensing mechanism makes 768 (9216/12=768) dispenses on the ChemCard in order to place each of the 9216 dots. As such, the algorithm that determines the locations of the dots on the ChemCard must allow for the dispensing of all 12 pipettes at each location. Table 1 contains the relative positions of the 9216 dots, in X and Y coordinates ranging from (0,0) to (64,144), according to one advantageous embodiment. Specifically, the column labeled “DOT#” assigns a number from 1-9216 to each specific dot, “REP” is either 1 or 2 indicating the 1^{st} and 2^{nd} placement of a compound, “COL” identifies the horizontal position (X), and “ROW” identifies the vertical position (Y). Each pair of sequential odd and even numbers, beginning with DOT# 1, represent the two dots of a particular compound (e.g. REP 1 and REP 2). For example, DOT# 1 and 2 are the same compound, DOT# 75 and 76 are the same compound, and DOT# 4227 and 4228 are the same compound.

The dot positions indicated in Table 1 were formulated for use with compounds having a diameter of about 0.7 mm, a minimum dot-pitch of about 0.95 mm and using a neighbor distance of about 3.5 mm. As such, each of the compounds has a plurality of neighbors. The arrangement of the compounds in Table 1 was determined so that each occurrence of a particular compound (two occurrences of each compound are used in this example) has exclusively different neighbors. The arrangement of compounds in Table 1 is exemplary and represents only one of a plurality of possible patterns for compounds in a (64,144) array according to the present invention.

While the position of the dots shown in Table 1 was formulated so that a dispensing mechanism comprising 12 pipettes may dispense all 12 compounds at each dispensing mechanism location, one of skill in the art will recognize the dots may be placed in other manners. For example, the dots may be placed by a single pipette that makes 9216 separate dispenses on the ChemCard (e.g. 2 dispenses of each compounds). Alternatively, the agorithm may be modified to generate a pattern that may be used in a system using a sing mechanism comprising any number of pipettes, such as 2, 4, 6, 8, 10, 14, 20, 24, 96, for example.

In addition, if a particular assay process is a multi step process or is otherwise more prone to errors in the locations of the dots, the range of neighbors may be expanded to include additional dots. For example, neighbors may be defined as those dots within 5 mm of one another.

TABLE 1

| DOT# | REP | COL | ROW |
|---|---|---|---|
| 1 | 1 | 37 | 5 |
| 2 | 2 | 73 | 9 |
| 3 | 1 | 115 | 6 |
| 4 | 2 | 4 | 4 |
| 5 | 1 | 49 | 5 |
| 6 | 2 | 85 | 9 |
| 7 | 1 | 127 | 6 |
| 8 | 2 | 16 | 4 |
| 9 | 1 | 61 | 5 |
| 10 | 2 | 97 | 9 |
| 11 | 1 | 139 | 6 |
| 12 | 2 | 28 | 4 |
| 13 | 1 | 73 | 6 |
| 14 | 2 | 1 | 4 |
| 15 | 1 | 11 | 42 |
| 16 | 2 | 120 | 35 |
| 17 | 1 | 85 | 6 |
| 18 | 2 | 13 | 4 |
| 19 | 1 | 23 | 42 |
| 20 | 2 | 132 | 35 |
| 21 | 1 | 97 | 6 |
| 22 | 2 | 25 | 4 |
| 23 | 1 | 35 | 42 |
| 24 | 2 | 144 | 35 |
| 25 | 1 | 46 | 40 |
| 26 | 2 | 45 | 35 |
| 27 | 1 | 113 | 33 |
| 28 | 2 | 11 | 41 |
| 29 | 1 | 58 | 40 |
| 30 | 2 | 57 | 35 |
| 31 | 1 | 125 | 33 |
| 32 | 2 | 23 | 41 |
| 33 | 1 | 70 | 40 |
| 34 | 2 | 69 | 35 |
| 35 | 1 | 137 | 33 |
| 36 | 2 | 35 | 41 |
| 37 | 1 | 11 | 4 |
| 38 | 2 | 6 | 4 |
| 39 | 1 | 12 | 10 |
| 40 | 2 | 39 | 40 |
| 41 | 1 | 23 | 4 |
| 42 | 2 | 18 | 4 |
| 43 | 1 | 24 | 10 |
| 44 | 2 | 51 | 40 |
| 45 | 1 | 35 | 4 |
| 46 | 2 | 30 | 4 |
| 47 | 1 | 36 | 10 |
| 48 | 2 | 63 | 40 |
| 49 | 1 | 1 | 42 |
| 50 | 2 | 117 | 5 |
| 51 | 1 | 79 | 43 |
| 52 | 2 | 47 | 8 |
| 53 | 1 | 13 | 42 |
| 54 | 2 | 129 | 5 |
| 55 | 1 | 91 | 43 |
| 56 | 2 | 59 | 8 |
| 57 | 1 | 25 | 42 |
| 58 | 2 | 141 | 5 |
| 59 | 1 | 103 | 43 |
| 60 | 2 | 71 | 8 |
| 61 | 1 | 1 | 40 |
| 62 | 2 | 10 | 38 |
| 63 | 1 | 3 | 38 |
| 64 | 2 | 44 | 3 |
| 65 | 1 | 13 | 40 |
| 66 | 2 | 22 | 38 |
| 67 | 1 | 15 | 38 |
| 68 | 2 | 56 | 3 |
| 69 | 1 | 25 | 40 |
| 70 | 2 | 34 | 38 |
| 71 | 1 | 27 | 38 |
| 72 | 2 | 68 | 3 |
| 73 | 1 | 11 | 6 |
| 74 | 2 | 113 | 34 |
| 75 | 1 | 77 | 7 |
| 76 | 2 | 45 | 39 |
| 77 | 1 | 23 | 6 |
| 78 | 2 | 125 | 34 |
| 79 | 1 | 89 | 7 |
| 80 | 2 | 57 | 39 |
| 81 | 1 | 35 | 6 |
| 82 | 2 | 137 | 34 |
| 83 | 1 | 101 | 7 |
| 84 | 2 | 69 | 39 |
| 85 | 1 | 119 | 3 |
| 86 | 2 | 73 | 11 |
| 87 | 1 | 111 | 3 |
| 88 | 2 | 74 | 36 |
| 89 | 1 | 131 | 3 |
| 90 | 2 | 85 | 11 |
| 91 | 1 | 123 | 3 |
| 92 | 2 | 86 | 36 |
| 93 | 1 | 143 | 3 |
| 94 | 2 | 97 | 11 |
| 95 | 1 | 135 | 3 |
| 96 | 2 | 98 | 36 |
| 97 | 1 | 37 | 13 |
| 98 | 2 | 73 | 17 |
| 99 | 1 | 115 | 14 |
| 100 | 2 | 4 | 12 |
| 101 | 1 | 49 | 13 |
| 102 | 2 | 85 | 17 |
| 103 | 1 | 127 | 14 |
| 104 | 2 | 16 | 12 |
| 105 | 1 | 61 | 13 |
| 106 | 2 | 97 | 17 |
| 107 | 1 | 139 | 14 |
| 108 | 2 | 28 | 12 |
| 109 | 1 | 73 | 14 |
| 110 | 2 | 1 | 12 |
| 111 | 1 | 11 | 50 |
| 112 | 2 | 120 | 43 |
| 113 | 1 | 85 | 14 |
| 114 | 2 | 13 | 12 |
| 115 | 1 | 23 | 50 |
| 116 | 2 | 132 | 43 |
| 117 | 1 | 97 | 14 |
| 118 | 2 | 25 | 12 |
| 119 | 1 | 35 | 50 |
| 120 | 2 | 144 | 43 |
| 121 | 1 | 46 | 48 |
| 122 | 2 | 45 | 43 |
| 123 | 1 | 113 | 41 |
| 124 | 2 | 11 | 49 |
| 125 | 1 | 58 | 48 |
| 126 | 2 | 57 | 43 |
| 127 | 1 | 125 | 41 |
| 128 | 2 | 23 | 49 |
| 129 | 1 | 70 | 48 |
| 130 | 2 | 69 | 43 |
| 131 | 1 | 137 | 41 |
| 132 | 2 | 35 | 49 |
| 133 | 1 | 11 | 12 |
| 134 | 2 | 6 | 12 |
| 135 | 1 | 12 | 18 |
| 136 | 2 | 39 | 48 |
| 137 | 1 | 23 | 12 |
| 138 | 2 | 18 | 12 |
| 139 | 1 | 24 | 18 |
| 140 | 2 | 51 | 48 |
| 141 | 1 | 35 | 12 |
| 142 | 2 | 30 | 12 |
| 143 | 1 | 36 | 18 |
| 144 | 2 | 63 | 48 |
| 145 | 1 | 1 | 50 |
| 146 | 2 | 117 | 13 |
| 147 | 1 | 79 | 51 |
| 148 | 2 | 47 | 16 |
| 149 | 1 | 13 | 50 |
| 150 | 2 | 129 | 13 |
| 151 | 1 | 91 | 51 |
| 152 | 2 | 59 | 16 |
| 153 | 1 | 25 | 50 |
| 154 | 2 | 141 | 13 |
| 155 | 1 | 103 | 51 |
| 156 | 2 | 71 | 16 |
| 157 | 1 | 1 | 48 |
| 158 | 2 | 10 | 46 |
| 159 | 1 | 3 | 46 |
| 160 | 2 | 44 | 11 |
| 161 | 1 | 13 | 48 |
| 162 | 2 | 22 | 46 |
| 163 | 1 | 15 | 46 |
| 164 | 2 | 56 | 11 |
| 165 | 1 | 25 | 48 |
| 166 | 2 | 34 | 46 |
| 167 | 1 | 27 | 46 |
| 168 | 2 | 68 | 11 |
| 169 | 1 | 11 | 14 |
| 170 | 2 | 113 | 42 |
| 171 | 1 | 77 | 15 |
| 172 | 2 | 45 | 47 |
| 173 | 1 | 23 | 14 |
| 174 | 2 | 125 | 42 |
| 175 | 1 | 89 | 15 |
| 176 | 2 | 57 | 47 |
| 177 | 1 | 35 | 14 |
| 178 | 2 | 137 | 42 |
| 179 | 1 | 101 | 15 |
| 180 | 2 | 69 | 47 |
| 181 | 1 | 119 | 11 |
| 182 | 2 | 73 | 19 |
| 183 | 1 | 111 | 11 |
| 184 | 2 | 74 | 44 |
| 185 | 1 | 131 | 11 |
| 186 | 2 | 85 | 19 |
| 187 | 1 | 123 | 11 |
| 188 | 2 | 86 | 44 |
| 189 | 1 | 143 | 11 |
| 190 | 2 | 97 | 19 |
| 191 | 1 | 135 | 11 |
| 192 | 2 | 98 | 44 |
| 193 | 1 | 37 | 21 |
| 194 | 2 | 73 | 25 |
| 195 | 1 | 115 | 22 |
| 196 | 2 | 4 | 20 |
| 197 | 1 | 49 | 21 |
| 198 | 2 | 85 | 25 |
| 199 | 1 | 127 | 22 |
| 200 | 2 | 16 | 20 |
| 201 | 1 | 61 | 21 |
| 202 | 2 | 97 | 25 |
| 203 | 1 | 139 | 22 |
| 204 | 2 | 28 | 20 |
| 205 | 1 | 73 | 22 |
| 206 | 2 | 1 | 20 |
| 207 | 1 | 11 | 58 |
| 208 | 2 | 120 | 51 |
| 209 | 1 | 85 | 22 |
| 210 | 2 | 13 | 20 |
| 211 | 1 | 23 | 58 |
| 212 | 2 | 132 | 51 |
| 213 | 1 | 97 | 22 |
| 214 | 2 | 25 | 20 |
| 215 | 1 | 35 | 58 |
| 216 | 2 | 144 | 51 |
| 217 | 1 | 46 | 56 |
| 218 | 2 | 45 | 51 |
| 219 | 1 | 113 | 49 |
| 220 | 2 | 11 | 57 |
| 221 | 1 | 58 | 56 |
| 222 | 2 | 57 | 51 |
| 223 | 1 | 125 | 49 |
| 224 | 2 | 23 | 57 |
| 225 | 1 | 70 | 56 |
| 226 | 2 | 69 | 51 |
| 227 | 1 | 137 | 49 |
| 228 | 2 | 35 | 57 |
| 229 | 1 | 11 | 20 |
| 230 | 2 | 6 | 20 |
| 231 | 1 | 12 | 26 |
| 232 | 2 | 39 | 56 |
| 233 | 1 | 23 | 20 |
| 234 | 2 | 18 | 20 |
| 235 | 1 | 24 | 26 |
| 236 | 2 | 51 | 56 |
| 237 | 1 | 35 | 20 |
| 238 | 2 | 30 | 20 |
| 239 | 1 | 36 | 26 |
| 240 | 2 | 63 | 56 |
| 241 | 1 | 1 | 58 |
| 242 | 2 | 117 | 21 |
| 243 | 1 | 79 | 59 |
| 244 | 2 | 47 | 24 |
| 245 | 1 | 13 | 58 |
| 246 | 2 | 129 | 21 |
| 247 | 1 | 91 | 59 |
| 248 | 2 | 59 | 24 |
| 249 | 1 | 25 | 58 |
| 250 | 2 | 141 | 21 |
| 251 | 1 | 103 | 59 |
| 252 | 2 | 71 | 24 |
| 253 | 1 | 1 | 56 |
| 254 | 2 | 10 | 54 |
| 255 | 1 | 3 | 54 |
| 256 | 2 | 44 | 19 |
| 257 | 1 | 13 | 56 |
| 258 | 2 | 22 | 54 |
| 259 | 1 | 15 | 54 |
| 260 | 2 | 56 | 19 |
| 261 | 1 | 25 | 56 |
| 262 | 2 | 34 | 54 |
| 263 | 1 | 27 | 54 |
| 264 | 2 | 68 | 19 |
| 265 | 1 | 11 | 22 |
| 266 | 2 | 113 | 50 |
| 267 | 1 | 77 | 23 |
| 268 | 2 | 45 | 55 |
| 269 | 1 | 23 | 22 |
| 270 | 2 | 125 | 50 |
| 271 | 1 | 89 | 23 |
| 272 | 2 | 57 | 55 |
| 273 | 1 | 35 | 22 |
| 274 | 2 | 137 | 50 |
| 275 | 1 | 101 | 23 |
| 276 | 2 | 69 | 55 |
| 277 | 1 | 119 | 19 |
| 278 | 2 | 73 | 27 |
| 279 | 1 | 111 | 19 |
| 280 | 2 | 74 | 52 |
| 281 | 1 | 131 | 19 |
| 282 | 2 | 85 | 27 |
| 283 | 1 | 123 | 19 |
| 284 | 2 | 86 | 52 |
| 285 | 1 | 143 | 19 |
| 286 | 2 | 97 | 27 |
| 287 | 1 | 135 | 19 |
| 288 | 2 | 98 | 52 |
| 289 | 1 | 37 | 29 |
| 290 | 2 | 73 | 33 |
| 291 | 1 | 115 | 30 |
| 292 | 2 | 4 | 28 |
| 293 | 1 | 49 | 29 |
| 294 | 2 | 85 | 33 |
| 295 | 1 | 127 | 30 |
| 296 | 2 | 16 | 28 |
| 297 | 1 | 61 | 29 |
| 298 | 2 | 97 | 33 |
| 299 | 1 | 139 | 30 |
| 300 | 2 | 28 | 28 |
| 301 | 1 | 73 | 30 |
| 302 | 2 | 1 | 28 |
| 303 | 1 | 11 | 66 |
| 304 | 2 | 120 | 59 |
| 305 | 1 | 85 | 30 |
| 306 | 2 | 13 | 28 |
| 307 | 1 | 23 | 66 |
| 308 | 2 | 132 | 59 |
| 309 | 1 | 97 | 30 |
| 310 | 2 | 25 | 28 |
| 311 | 1 | 35 | 66 |
| 312 | 2 | 144 | 59 |
| 313 | 1 | 46 | 64 |
| 314 | 2 | 45 | 59 |
| 315 | 1 | 113 | 57 |
| 316 | 2 | 11 | 65 |
| 317 | 1 | 58 | 64 |
| 318 | 2 | 57 | 59 |
| 319 | 1 | 125 | 57 |
| 320 | 2 | 23 | 65 |
| 321 | 1 | 70 | 64 |
| 322 | 2 | 69 | 59 |
| 323 | 1 | 137 | 57 |
| 324 | 2 | 35 | 65 |
| 325 | 1 | 11 | 28 |
| 326 | 2 | 6 | 28 |
| 327 | 1 | 12 | 34 |
| 328 | 2 | 39 | 64 |
| 329 | 1 | 23 | 28 |
| 330 | 2 | 18 | 28 |
| 331 | 1 | 24 | 34 |
| 332 | 2 | 51 | 64 |
| 333 | 1 | 35 | 28 |
| 334 | 2 | 30 | 28 |
| 335 | 1 | 36 | 34 |
| 336 | 2 | 63 | 64 |
| 337 | 1 | 1 | 66 |
| 338 | 2 | 117 | 29 |
| 339 | 1 | 79 | 67 |
| 340 | 2 | 47 | 32 |
| 341 | 1 | 13 | 66 |
| 342 | 2 | 129 | 29 |
| 343 | 1 | 91 | 67 |
| 344 | 2 | 59 | 32 |
| 345 | 1 | 25 | 66 |
| 346 | 2 | 141 | 29 |
| 347 | 1 | 103 | 67 |
| 348 | 2 | 71 | 32 |
| 349 | 1 | 1 | 64 |
| 350 | 2 | 10 | 62 |
| 351 | 1 | 3 | 62 |
| 352 | 2 | 44 | 27 |
| 353 | 1 | 13 | 64 |
| 354 | 2 | 22 | 62 |
| 355 | 1 | 15 | 62 |
| 356 | 2 | 56 | 27 |
| 357 | 1 | 25 | 64 |
| 358 | 2 | 34 | 62 |
| 359 | 1 | 27 | 62 |
| 360 | 2 | 68 | 27 |
| 361 | 1 | 11 | 30 |
| 362 | 2 | 113 | 58 |
| 363 | 1 | 77 | 31 |
| 364 | 2 | 45 | 63 |
| 365 | 1 | 23 | 30 |
| 366 | 2 | 125 | 58 |
| 367 | 1 | 89 | 31 |
| 368 | 2 | 57 | 63 |
| 369 | 1 | 35 | 30 |
| 370 | 2 | 137 | 58 |
| 371 | 1 | 101 | 31 |
| 372 | 2 | 69 | 63 |
| 373 | 1 | 119 | 27 |
| 374 | 2 | 73 | 35 |
| 375 | 1 | 111 | 27 |
| 376 | 2 | 74 | 60 |
| 377 | 1 | 131 | 27 |
| 378 | 2 | 85 | 35 |
| 379 | 1 | 123 | 27 |
| 380 | 2 | 86 | 60 |
| 381 | 1 | 143 | 27 |
| 382 | 2 | 97 | 35 |
| 383 | 1 | 135 | 27 |
| 384 | 2 | 98 | 60 |
| 385 | 1 | 9 | 4 |
| 386 | 2 | 8 | 42 |
| 387 | 1 | 78 | 38 |
| 388 | 2 | 119 | 2 |
| 389 | 1 | 21 | 4 |
| 390 | 2 | 20 | 42 |
| 391 | 1 | 90 | 38 |
| 392 | 2 | 131 | 2 |
| 393 | 1 | 33 | 4 |
| 394 | 2 | 32 | 42 |
| 395 | 1 | 102 | 38 |
| 396 | 2 | 143 | 2 |
| 397 | 1 | 84 | 8 |
| 398 | 2 | 37 | 4 |
| 399 | 1 | 74 | 6 |
| 400 | 2 | 47 | 35 |
| 401 | 1 | 96 | 8 |
| 402 | 2 | 49 | 4 |
| 403 | 1 | 86 | 6 |
| 404 | 2 | 59 | 35 |
| 405 | 1 | 108 | 8 |
| 406 | 2 | 61 | 4 |
| 407 | 1 | 98 | 6 |
| 408 | 2 | 71 | 35 |
| 409 | 1 | 74 | 4 |
| 410 | 2 | 120 | 33 |
| 411 | 1 | 116 | 2 |
| 412 | 2 | 38 | 36 |
| 413 | 1 | 86 | 4 |
| 414 | 2 | 132 | 33 |
| 415 | 1 | 128 | 2 |
| 416 | 2 | 50 | 36 |
| 417 | 1 | 98 | 4 |
| 418 | 2 | 144 | 33 |
| 419 | 1 | 140 | 2 |
| 420 | 2 | 62 | 36 |
| 421 | 1 | 5 | 11 |
| 422 | 2 | 9 | 7 |
| 423 | 1 | 7 | 9 |
| 424 | 2 | 43 | 35 |
| 425 | 1 | 17 | 11 |
| 426 | 2 | 21 | 7 |
| 427 | 1 | 19 | 9 |
| 428 | 2 | 55 | 35 |
| 429 | 1 | 29 | 11 |
| 430 | 2 | 33 | 7 |
| 431 | 1 | 31 | 9 |
| 432 | 2 | 67 | 35 |
| 433 | 1 | 120 | 36 |
| 434 | 2 | 76 | 41 |
| 435 | 1 | 44 | 8 |
| 436 | 2 | 9 | 38 |
| 437 | 1 | 132 | 36 |
| 438 | 2 | 88 | 41 |
| 439 | 1 | 56 | 8 |
| 440 | 2 | 21 | 38 |
| 441 | 1 | 144 | 36 |
| 442 | 2 | 100 | 41 |
| 443 | 1 | 68 | 8 |
| 444 | 2 | 33 | 38 |
| 445 | 1 | 76 | 4 |
| 446 | 2 | 81 | 6 |
| 447 | 1 | 40 | 39 |
| 448 | 2 | 117 | 36 |
| 449 | 1 | 88 | 4 |
| 450 | 2 | 93 | 6 |
| 451 | 1 | 52 | 39 |
| 452 | 2 | 129 | 36 |
| 453 | 1 | 100 | 4 |
| 454 | 2 | 105 | 6 |
| 455 | 1 | 64 | 39 |
| 456 | 2 | 141 | 36 |
| 457 | 1 | 38 | 35 |
| 458 | 2 | 8 | 4 |
| 459 | 1 | 118 | 38 |
| 460 | 2 | 12 | 9 |
| 461 | 1 | 50 | 35 |
| 462 | 2 | 20 | 4 |
| 463 | 1 | 130 | 38 |
| 464 | 2 | 24 | 9 |
| 465 | 1 | 62 | 35 |
| 466 | 2 | 32 | 4 |
| 467 | 1 | 142 | 38 |
| 468 | 2 | 36 | 9 |
| 469 | 1 | 114 | 2 |
| 470 | 2 | 77 | 6 |
| 471 | 1 | 10 | 37 |
| 472 | 2 | 37 | 1 |
| 473 | 1 | 126 | 2 |
| 474 | 2 | 89 | 6 |
| 475 | 1 | 22 | 37 |
| 476 | 2 | 49 | 1 |
| 477 | 1 | 138 | 2 |
| 478 | 2 | 101 | 6 |
| 479 | 1 | 34 | 37 |
| 480 | 2 | 61 | 1 |
| 481 | 1 | 9 | 12 |
| 482 | 2 | 8 | 50 |
| 483 | 1 | 78 | 46 |
| 484 | 2 | 119 | 10 |
| 485 | 1 | 21 | 12 |
| 486 | 2 | 20 | 50 |
| 487 | 1 | 90 | 46 |
| 488 | 2 | 131 | 10 |
| 489 | 1 | 33 | 12 |
| 490 | 2 | 32 | 50 |
| 491 | 1 | 102 | 46 |
| 492 | 2 | 143 | 10 |
| 493 | 1 | 84 | 16 |
| 494 | 2 | 37 | 12 |
| 495 | 1 | 74 | 14 |
| 496 | 2 | 47 | 43 |
| 497 | 1 | 96 | 16 |
| 498 | 2 | 49 | 12 |
| 499 | 1 | 86 | 14 |
| 500 | 2 | 59 | 43 |
| 501 | 1 | 108 | 16 |
| 502 | 2 | 61 | 12 |
| 503 | 1 | 98 | 14 |
| 504 | 2 | 71 | 43 |
| 505 | 1 | 74 | 12 |
| 506 | 2 | 120 | 41 |
| 507 | 1 | 116 | 10 |
| 508 | 2 | 38 | 44 |
| 509 | 1 | 86 | 12 |
| 510 | 2 | 132 | 41 |
| 511 | 1 | 128 | 10 |
| 512 | 2 | 50 | 44 |
| 513 | 1 | 98 | 12 |
| 514 | 2 | 144 | 41 |
| 515 | 1 | 140 | 10 |
| 516 | 2 | 62 | 44 |
| 517 | 1 | 5 | 19 |
| 518 | 2 | 9 | 15 |
| 519 | 1 | 7 | 17 |
| 520 | 2 | 43 | 43 |
| 521 | 1 | 17 | 19 |
| 522 | 2 | 21 | 15 |
| 523 | 1 | 19 | 17 |
| 524 | 2 | 55 | 43 |
| 525 | 1 | 29 | 19 |
| 526 | 2 | 33 | 15 |
| 527 | 1 | 31 | 17 |
| 528 | 2 | 67 | 43 |
| 529 | 1 | 120 | 44 |
| 530 | 2 | 76 | 49 |
| 531 | 1 | 44 | 16 |
| 532 | 2 | 9 | 46 |
| 533 | 1 | 132 | 44 |
| 534 | 2 | 88 | 49 |
| 535 | 1 | 56 | 16 |
| 536 | 2 | 21 | 46 |
| 537 | 1 | 144 | 44 |
| 538 | 2 | 100 | 49 |
| 539 | 1 | 68 | 16 |
| 540 | 2 | 33 | 46 |
| 541 | 1 | 76 | 12 |
| 542 | 2 | 81 | 14 |
| 543 | 1 | 40 | 47 |
| 544 | 2 | 117 | 44 |
| 545 | 1 | 88 | 12 |
| 546 | 2 | 93 | 14 |
| 547 | 1 | 52 | 47 |
| 548 | 2 | 129 | 44 |
| 549 | 1 | 100 | 12 |
| 550 | 2 | 105 | 14 |
| 551 | 1 | 64 | 47 |
| 552 | 2 | 141 | 44 |
| 553 | 1 | 38 | 43 |
| 554 | 2 | 8 | 12 |
| 555 | 1 | 118 | 46 |
| 556 | 2 | 12 | 17 |
| 557 | 1 | 50 | 43 |
| 558 | 2 | 20 | 12 |
| 559 | 1 | 130 | 46 |
| 560 | 2 | 24 | 17 |
| 561 | 1 | 62 | 43 |
| 562 | 2 | 32 | 12 |
| 563 | 1 | 142 | 46 |
| 564 | 2 | 36 | 17 |
| 565 | 1 | 114 | 10 |
| 566 | 2 | 77 | 14 |
| 567 | 1 | 10 | 45 |
| 568 | 2 | 37 | 9 |
| 569 | 1 | 126 | 10 |
| 570 | 2 | 89 | 14 |
| 571 | 1 | 22 | 45 |
| 572 | 2 | 49 | 9 |
| 573 | 1 | 138 | 10 |
| 574 | 2 | 101 | 14 |
| 575 | 1 | 34 | 45 |
| 576 | 2 | 61 | 9 |
| 577 | 1 | 9 | 20 |
| 578 | 2 | 8 | 58 |
| 579 | 1 | 78 | 54 |
| 580 | 2 | 119 | 18 |
| 581 | 1 | 21 | 20 |
| 582 | 2 | 20 | 58 |
| 583 | 1 | 90 | 54 |
| 584 | 2 | 131 | 18 |
| 585 | 1 | 33 | 20 |
| 586 | 2 | 32 | 58 |
| 587 | 1 | 102 | 54 |
| 588 | 2 | 143 | 18 |
| 589 | 1 | 84 | 24 |
| 590 | 2 | 37 | 20 |
| 591 | 1 | 74 | 22 |
| 592 | 2 | 47 | 51 |
| 593 | 1 | 96 | 24 |
| 594 | 2 | 49 | 20 |
| 595 | 1 | 86 | 22 |
| 596 | 2 | 59 | 51 |
| 597 | 1 | 108 | 24 |
| 598 | 2 | 61 | 20 |
| 599 | 1 | 98 | 22 |
| 600 | 2 | 71 | 51 |
| 601 | 1 | 74 | 20 |
| 602 | 2 | 120 | 49 |
| 603 | 1 | 116 | 18 |
| 604 | 2 | 38 | 52 |
| 605 | 1 | 86 | 20 |
| 606 | 2 | 132 | 49 |
| 607 | 1 | 128 | 18 |
| 608 | 2 | 50 | 52 |
| 609 | 1 | 98 | 20 |
| 610 | 2 | 144 | 49 |
| 611 | 1 | 140 | 18 |
| 612 | 2 | 62 | 52 |
| 613 | 1 | 5 | 27 |
| 614 | 2 | 9 | 23 |
| 615 | 1 | 7 | 25 |
| 616 | 2 | 43 | 51 |
| 617 | 1 | 17 | 27 |
| 618 | 2 | 21 | 23 |
| 619 | 1 | 19 | 25 |
| 620 | 2 | 55 | 51 |
| 621 | 1 | 29 | 27 |
| 622 | 2 | 33 | 23 |
| 623 | 1 | 31 | 25 |
| 624 | 2 | 67 | 51 |
| 625 | 1 | 120 | 52 |
| 626 | 2 | 76 | 57 |
| 627 | 1 | 44 | 24 |
| 628 | 2 | 9 | 54 |
| 629 | 1 | 132 | 52 |
| 630 | 2 | 88 | 57 |
| 631 | 1 | 56 | 24 |
| 632 | 2 | 21 | 54 |
| 633 | 1 | 144 | 52 |
| 634 | 2 | 100 | 57 |
| 635 | 1 | 68 | 24 |
| 636 | 2 | 33 | 54 |
| 637 | 1 | 76 | 20 |
| 638 | 2 | 81 | 22 |
| 639 | 1 | 40 | 55 |
| 640 | 2 | 117 | 52 |
| 641 | 1 | 88 | 20 |
| 642 | 2 | 93 | 22 |
| 643 | 1 | 52 | 55 |
| 644 | 2 | 129 | 52 |
| 645 | 1 | 100 | 20 |
| 646 | 2 | 105 | 22 |
| 647 | 1 | 64 | 55 |
| 648 | 2 | 141 | 52 |
| 649 | 1 | 38 | 51 |
| 650 | 2 | 8 | 20 |
| 651 | 1 | 118 | 54 |
| 652 | 2 | 12 | 25 |
| 653 | 1 | 50 | 51 |
| 654 | 2 | 20 | 20 |
| 655 | 1 | 130 | 54 |
| 656 | 2 | 24 | 25 |
| 657 | 1 | 62 | 51 |
| 658 | 2 | 32 | 20 |
| 659 | 1 | 142 | 54 |
| 660 | 2 | 36 | 25 |
| 661 | 1 | 114 | 18 |
| 662 | 2 | 77 | 22 |
| 663 | 1 | 10 | 53 |
| 664 | 2 | 37 | 17 |
| 665 | 1 | 126 | 18 |
| 666 | 2 | 89 | 22 |
| 667 | 1 | 22 | 53 |
| 668 | 2 | 49 | 17 |
| 669 | 1 | 138 | 18 |
| 670 | 2 | 101 | 22 |
| 671 | 1 | 34 | 53 |
| 672 | 2 | 61 | 17 |
| 673 | 1 | 9 | 28 |
| 674 | 2 | 8 | 66 |
| 675 | 1 | 78 | 62 |
| 676 | 2 | 119 | 26 |
| 677 | 1 | 21 | 28 |
| 678 | 2 | 20 | 66 |
| 679 | 1 | 90 | 62 |
| 680 | 2 | 131 | 26 |
| 681 | 1 | 33 | 28 |
| 682 | 2 | 32 | 66 |
| 683 | 1 | 102 | 62 |
| 684 | 2 | 143 | 26 |
| 685 | 1 | 84 | 32 |
| 686 | 2 | 37 | 28 |
| 687 | 1 | 74 | 30 |
| 688 | 2 | 47 | 59 |
| 689 | 1 | 96 | 32 |
| 690 | 2 | 49 | 28 |
| 691 | 1 | 86 | 30 |
| 692 | 2 | 59 | 59 |
| 693 | 1 | 108 | 32 |
| 694 | 2 | 61 | 28 |
| 695 | 1 | 98 | 30 |
| 696 | 2 | 71 | 59 |
| 697 | 1 | 74 | 28 |
| 698 | 2 | 120 | 57 |
| 699 | 1 | 116 | 26 |
| 700 | 2 | 38 | 60 |
| 701 | 1 | 86 | 28 |
| 702 | 2 | 132 | 57 |
| 703 | 1 | 128 | 26 |
| 704 | 2 | 50 | 60 |
| 705 | 1 | 98 | 28 |
| 706 | 2 | 144 | 57 |
| 707 | 1 | 140 | 26 |
| 708 | 2 | 62 | 60 |
| 709 | 1 | 5 | 35 |
| 710 | 2 | 9 | 31 |
| 711 | 1 | 7 | 33 |
| 712 | 2 | 43 | 59 |
| 713 | 1 | 17 | 35 |
| 714 | 2 | 21 | 31 |
| 715 | 1 | 19 | 33 |
| 716 | 2 | 55 | 59 |
| 717 | 1 | 29 | 35 |
| 718 | 2 | 33 | 31 |
| 719 | 1 | 31 | 33 |
| 720 | 2 | 67 | 59 |
| 721 | 1 | 120 | 60 |
| 722 | 2 | 76 | 65 |
| 723 | 1 | 44 | 32 |
| 724 | 2 | 9 | 62 |
| 725 | 1 | 132 | 60 |
| 726 | 2 | 88 | 65 |
| 727 | 1 | 56 | 32 |
| 728 | 2 | 21 | 62 |
| 729 | 1 | 144 | 60 |
| 730 | 2 | 100 | 65 |
| 731 | 1 | 68 | 32 |
| 732 | 2 | 33 | 62 |
| 733 | 1 | 76 | 28 |
| 734 | 2 | 81 | 30 |
| 735 | 1 | 40 | 63 |
| 736 | 2 | 117 | 60 |
| 737 | 1 | 88 | 28 |
| 738 | 2 | 93 | 30 |
| 739 | 1 | 52 | 63 |
| 740 | 2 | 129 | 60 |
| 741 | 1 | 100 | 28 |
| 742 | 2 | 105 | 30 |
| 743 | 1 | 64 | 63 |
| 744 | 2 | 141 | 60 |
| 745 | 1 | 38 | 59 |
| 746 | 2 | 8 | 28 |
| 747 | 1 | 118 | 62 |
| 748 | 2 | 12 | 33 |
| 749 | 1 | 50 | 59 |
| 750 | 2 | 20 | 28 |
| 751 | 1 | 130 | 62 |
| 752 | 2 | 24 | 33 |
| 753 | 1 | 62 | 59 |
| 754 | 2 | 32 | 28 |
| 755 | 1 | 142 | 62 |
| 756 | 2 | 36 | 33 |
| 757 | 1 | 114 | 26 |
| 758 | 2 | 77 | 30 |
| 759 | 1 | 10 | 61 |
| 760 | 2 | 37 | 25 |
| 761 | 1 | 126 | 26 |
| 762 | 2 | 89 | 30 |
| 763 | 1 | 22 | 61 |
| 764 | 2 | 49 | 25 |
| 765 | 1 | 138 | 26 |
| 766 | 2 | 101 | 30 |
| 767 | 1 | 34 | 61 |
| 768 | 2 | 61 | 25 |
| 769 | 1 | 115 | 5 |
| 770 | 2 | 7 | 41 |
| 771 | 1 | 37 | 38 |
| 772 | 2 | 82 | 4 |
| 773 | 1 | 127 | 5 |
| 774 | 2 | 19 | 41 |
| 775 | 1 | 49 | 38 |
| 776 | 2 | 94 | 4 |
| 777 | 1 | 139 | 5 |
| 778 | 2 | 31 | 41 |
| 779 | 1 | 61 | 38 |
| 780 | 2 | 106 | 4 |
| 781 | 1 | 115 | 3 |
| 782 | 2 | 44 | 36 |
| 783 | 1 | 113 | 39 |
| 784 | 2 | 78 | 7 |
| 785 | 1 | 127 | 3 |
| 786 | 2 | 56 | 36 |
| 787 | 1 | 125 | 39 |
| 788 | 2 | 90 | 7 |
| 789 | 1 | 139 | 3 |
| 790 | 2 | 68 | 36 |
| 791 | 1 | 137 | 39 |
| 792 | 2 | 102 | 7 |
| 793 | 1 | 109 | 7 |
| 794 | 2 | 3 | 7 |
| 795 | 1 | 47 | 2 |
| 796 | 2 | 113 | 38 |
| 797 | 1 | 121 | 7 |
| 798 | 2 | 15 | 7 |
| 799 | 1 | 59 | 2 |
| 800 | 2 | 125 | 38 |
| 801 | 1 | 133 | 7 |
| 802 | 2 | 27 | 7 |
| 803 | 1 | 71 | 2 |
| 804 | 2 | 137 | 38 |
| 805 | 1 | 113 | 1 |
| 806 | 2 | 120 | 1 |
| 807 | 1 | 114 | 7 |
| 808 | 2 | 109 | 1 |
| 809 | 1 | 125 | 1 |
| 810 | 2 | 132 | 1 |
| 811 | 1 | 126 | 7 |
| 812 | 2 | 121 | 1 |
| 813 | 1 | 137 | 1 |
| 814 | 2 | 144 | 1 |
| 815 | 1 | 138 | 7 |
| 816 | 2 | 133 | 1 |
| 817 | 1 | 7 | 11 |
| 818 | 2 | 1 | 36 |
| 819 | 1 | 84 | 7 |
| 820 | 2 | 110 | 2 |
| 821 | 1 | 19 | 11 |
| 822 | 2 | 13 | 36 |
| 823 | 1 | 96 | 7 |
| 824 | 2 | 122 | 2 |
| 825 | 1 | 31 | 11 |
| 826 | 2 | 25 | 36 |
| 827 | 1 | 108 | 7 |
| 828 | 2 | 134 | 2 |
| 829 | 1 | 115 | 37 |
| 830 | 2 | 73 | 5 |
| 831 | 1 | 38 | 5 |
| 832 | 2 | 4 | 41 |
| 833 | 1 | 127 | 37 |
| 834 | 2 | 85 | 5 |
| 835 | 1 | 50 | 5 |
| 836 | 2 | 16 | 41 |
| 837 | 1 | 139 | 37 |
| 838 | 2 | 97 | 5 |
| 839 | 1 | 62 | 5 |
| 840 | 2 | 28 | 41 |
| 841 | 1 | 113 | 3 |
| 842 | 2 | 47 | 3 |
| 843 | 1 | 82 | 5 |
| 844 | 2 | 44 | 40 |
| 845 | 1 | 125 | 3 |
| 846 | 2 | 59 | 3 |
| 847 | 1 | 94 | 5 |
| 848 | 2 | 56 | 40 |
| 849 | 1 | 137 | 3 |
| 850 | 2 | 71 | 3 |
| 851 | 1 | 106 | 5 |
| 852 | 2 | 68 | 40 |
| 853 | 1 | 77 | 38 |
| 854 | 2 | 7 | 43 |
| 855 | 1 | 3 | 10 |
| 856 | 2 | 112 | 2 |
| 857 | 1 | 89 | 38 |
| 858 | 2 | 19 | 43 |
| 859 | 1 | 15 | 10 |
| 860 | 2 | 124 | 2 |
| 861 | 1 | 101 | 38 |
| 862 | 2 | 31 | 43 |
| 863 | 1 | 27 | 10 |
| 864 | 2 | 136 | 2 |
| 865 | 1 | 115 | 13 |
| 866 | 2 | 7 | 49 |
| 867 | 1 | 37 | 46 |
| 868 | 2 | 82 | 12 |
| 869 | 1 | 127 | 13 |
| 870 | 2 | 19 | 49 |
| 871 | 1 | 49 | 46 |
| 872 | 2 | 94 | 12 |
| 873 | 1 | 139 | 13 |
| 874 | 2 | 31 | 49 |
| 875 | 1 | 61 | 46 |
| 876 | 2 | 106 | 12 |
| 877 | 1 | 115 | 11 |
| 878 | 2 | 44 | 44 |
| 879 | 1 | 113 | 47 |
| 880 | 2 | 78 | 15 |
| 881 | 1 | 127 | 11 |
| 882 | 2 | 56 | 44 |
| 883 | 1 | 125 | 47 |
| 884 | 2 | 90 | 15 |
| 885 | 1 | 139 | 11 |
| 886 | 2 | 68 | 44 |
| 887 | 1 | 137 | 47 |
| 888 | 2 | 102 | 15 |
| 889 | 1 | 109 | 15 |
| 890 | 2 | 3 | 15 |
| 891 | 1 | 47 | 10 |
| 892 | 2 | 113 | 46 |
| 893 | 1 | 121 | 15 |
| 894 | 2 | 15 | 15 |
| 895 | 1 | 59 | 10 |
| 896 | 2 | 125 | 46 |
| 897 | 1 | 133 | 15 |
| 898 | 2 | 27 | 15 |
| 899 | 1 | 71 | 10 |
| 900 | 2 | 137 | 46 |
| 901 | 1 | 113 | 9 |
| 902 | 2 | 120 | 9 |
| 903 | 1 | 114 | 15 |
| 904 | 2 | 109 | 9 |
| 905 | 1 | 125 | 9 |
| 906 | 2 | 132 | 9 |
| 907 | 1 | 126 | 15 |
| 908 | 2 | 121 | 9 |
| 909 | 1 | 137 | 9 |
| 910 | 2 | 144 | 9 |
| 911 | 1 | 138 | 15 |
| 912 | 2 | 133 | 9 |
| 913 | 1 | 7 | 19 |
| 914 | 2 | 1 | 44 |
| 915 | 1 | 84 | 15 |
| 916 | 2 | 110 | 10 |
| 917 | 1 | 19 | 19 |
| 918 | 2 | 13 | 44 |
| 919 | 1 | 96 | 15 |
| 920 | 2 | 122 | 10 |
| 921 | 1 | 31 | 19 |
| 922 | 2 | 25 | 44 |
| 923 | 1 | 108 | 15 |
| 924 | 2 | 134 | 10 |
| 925 | 1 | 115 | 45 |
| 926 | 2 | 73 | 13 |
| 927 | 1 | 38 | 13 |
| 928 | 2 | 4 | 49 |
| 929 | 1 | 127 | 45 |
| 930 | 2 | 85 | 13 |
| 931 | 1 | 50 | 13 |
| 932 | 2 | 16 | 49 |
| 933 | 1 | 139 | 45 |
| 934 | 2 | 97 | 13 |
| 935 | 1 | 62 | 13 |
| 936 | 2 | 28 | 49 |
| 937 | 1 | 113 | 11 |
| 938 | 2 | 47 | 11 |
| 939 | 1 | 82 | 13 |
| 940 | 2 | 44 | 48 |
| 941 | 1 | 125 | 11 |
| 942 | 2 | 59 | 11 |
| 943 | 1 | 94 | 13 |
| 944 | 2 | 56 | 48 |
| 945 | 1 | 137 | 11 |
| 946 | 2 | 71 | 11 |
| 947 | 1 | 106 | 13 |
| 948 | 2 | 68 | 48 |
| 949 | 1 | 77 | 46 |
| 950 | 2 | 7 | 51 |
| 951 | 1 | 3 | 18 |
| 952 | 2 | 112 | 10 |
| 953 | 1 | 89 | 46 |
| 954 | 2 | 19 | 51 |
| 955 | 1 | 15 | 18 |
| 956 | 2 | 124 | 10 |
| 957 | 1 | 101 | 46 |
| 958 | 2 | 31 | 51 |
| 959 | 1 | 27 | 18 |
| 960 | 2 | 136 | 10 |
| 961 | 1 | 115 | 21 |
| 962 | 2 | 7 | 57 |
| 963 | 1 | 37 | 54 |
| 964 | 2 | 82 | 20 |
| 965 | 1 | 127 | 21 |
| 966 | 2 | 19 | 57 |
| 967 | 1 | 49 | 54 |
| 968 | 2 | 94 | 20 |
| 969 | 1 | 139 | 21 |
| 970 | 2 | 31 | 57 |
| 971 | 1 | 61 | 54 |
| 972 | 2 | 106 | 20 |
| 973 | 1 | 115 | 19 |
| 974 | 2 | 44 | 52 |
| 975 | 1 | 113 | 55 |
| 976 | 2 | 78 | 23 |
| 977 | 1 | 127 | 19 |
| 978 | 2 | 56 | 52 |
| 979 | 1 | 125 | 55 |
| 980 | 2 | 90 | 23 |
| 981 | 1 | 139 | 19 |
| 982 | 2 | 68 | 52 |
| 983 | 1 | 137 | 55 |
| 984 | 2 | 102 | 23 |
| 985 | 1 | 109 | 23 |
| 986 | 2 | 3 | 23 |
| 987 | 1 | 47 | 18 |
| 988 | 2 | 113 | 54 |
| 989 | 1 | 121 | 23 |
| 990 | 2 | 15 | 23 |
| 991 | 1 | 59 | 18 |
| 992 | 2 | 125 | 54 |
| 993 | 1 | 133 | 23 |
| 994 | 2 | 27 | 23 |
| 995 | 1 | 71 | 18 |
| 996 | 2 | 137 | 54 |
| 997 | 1 | 113 | 17 |
| 998 | 2 | 120 | 17 |
| 999 | 1 | 114 | 23 |
| 1000 | 2 | 109 | 17 |
| 1001 | 1 | 125 | 17 |
| 1002 | 2 | 132 | 17 |
| 1003 | 1 | 126 | 23 |
| 1004 | 2 | 121 | 17 |
| 1005 | 1 | 137 | 17 |
| 1006 | 2 | 144 | 17 |
| 1007 | 1 | 138 | 23 |
| 1008 | 2 | 133 | 17 |
| 1009 | 1 | 7 | 27 |
| 1010 | 2 | 1 | 52 |
| 1011 | 1 | 84 | 23 |
| 1012 | 2 | 110 | 18 |
| 1013 | 1 | 19 | 27 |
| 1014 | 2 | 13 | 52 |
| 1015 | 1 | 96 | 23 |
| 1016 | 2 | 122 | 18 |
| 1017 | 1 | 31 | 27 |
| 1018 | 2 | 25 | 52 |
| 1019 | 1 | 108 | 23 |
| 1020 | 2 | 134 | 18 |
| 1021 | 1 | 115 | 53 |
| 1022 | 2 | 73 | 21 |
| 1023 | 1 | 38 | 21 |
| 1024 | 2 | 4 | 57 |
| 1025 | 1 | 127 | 53 |
| 1026 | 2 | 85 | 21 |
| 1027 | 1 | 50 | 21 |
| 1028 | 2 | 16 | 57 |
| 1029 | 1 | 139 | 53 |
| 1030 | 2 | 97 | 21 |
| 1031 | 1 | 62 | 21 |
| 1032 | 2 | 28 | 57 |
| 1033 | 1 | 113 | 19 |
| 1034 | 2 | 47 | 19 |
| 1035 | 1 | 82 | 21 |
| 1036 | 2 | 44 | 56 |
| 1037 | 1 | 125 | 19 |
| 1038 | 2 | 59 | 19 |
| 1039 | 1 | 94 | 21 |
| 1040 | 2 | 56 | 56 |
| 1041 | 1 | 137 | 19 |
| 1042 | 2 | 71 | 19 |
| 1043 | 1 | 106 | 21 |
| 1044 | 2 | 68 | 56 |
| 1045 | 1 | 77 | 54 |
| 1046 | 2 | 7 | 59 |
| 1047 | 1 | 3 | 26 |
| 1048 | 2 | 112 | 18 |
| 1049 | 1 | 89 | 54 |
| 1050 | 2 | 19 | 59 |
| 1051 | 1 | 15 | 26 |
| 1052 | 2 | 124 | 18 |
| 1053 | 1 | 101 | 54 |
| 1054 | 2 | 31 | 59 |
| 1055 | 1 | 27 | 26 |
| 1056 | 2 | 136 | 18 |
| 1057 | 1 | 115 | 29 |
| 1058 | 2 | 7 | 65 |
| 1059 | 1 | 37 | 62 |
| 1060 | 2 | 82 | 28 |
| 1061 | 1 | 127 | 29 |
| 1062 | 2 | 19 | 65 |
| 1063 | 1 | 49 | 62 |
| 1064 | 2 | 94 | 28 |
| 1065 | 1 | 139 | 29 |
| 1066 | 2 | 31 | 65 |
| 1067 | 1 | 61 | 62 |
| 1068 | 2 | 106 | 28 |
| 1069 | 1 | 115 | 27 |
| 1070 | 2 | 44 | 60 |
| 1071 | 1 | 113 | 63 |
| 1072 | 2 | 78 | 31 |
| 1073 | 1 | 127 | 27 |
| 1074 | 2 | 56 | 60 |
| 1075 | 1 | 125 | 63 |
| 1076 | 2 | 90 | 31 |
| 1077 | 1 | 139 | 27 |
| 1078 | 2 | 68 | 60 |
| 1079 | 1 | 137 | 63 |
| 1080 | 2 | 102 | 31 |
| 1081 | 1 | 109 | 31 |
| 1082 | 2 | 3 | 31 |
| 1083 | 1 | 47 | 26 |
| 1084 | 2 | 113 | 62 |
| 1085 | 1 | 121 | 31 |
| 1086 | 2 | 15 | 31 |
| 1087 | 1 | 59 | 26 |
| 1088 | 2 | 125 | 62 |
| 1089 | 1 | 133 | 31 |
| 1090 | 2 | 27 | 31 |
| 1091 | 1 | 71 | 26 |
| 1092 | 2 | 137 | 62 |
| 1093 | 1 | 113 | 25 |
| 1094 | 2 | 120 | 25 |
| 1095 | 1 | 114 | 31 |
| 1096 | 2 | 109 | 25 |
| 1097 | 1 | 125 | 25 |
| 1098 | 2 | 132 | 25 |
| 1099 | 1 | 126 | 31 |
| 1100 | 2 | 121 | 25 |
| 1101 | 1 | 137 | 25 |
| 1102 | 2 | 144 | 25 |
| 1103 | 1 | 138 | 31 |
| 1104 | 2 | 133 | 25 |
| 1105 | 1 | 7 | 35 |
| 1106 | 2 | 1 | 60 |
| 1107 | 1 | 84 | 31 |
| 1108 | 2 | 110 | 26 |
| 1109 | 1 | 19 | 35 |
| 1110 | 2 | 13 | 60 |
| 1111 | 1 | 96 | 31 |
| 1112 | 2 | 122 | 26 |
| 1113 | 1 | 31 | 35 |
| 1114 | 2 | 25 | 60 |
| 1115 | 1 | 108 | 31 |
| 1116 | 2 | 134 | 26 |
| 1117 | 1 | 115 | 61 |
| 1118 | 2 | 73 | 29 |
| 1119 | 1 | 38 | 29 |
| 1120 | 2 | 4 | 65 |
| 1121 | 1 | 127 | 61 |
| 1122 | 2 | 85 | 29 |
| 1123 | 1 | 50 | 29 |
| 1124 | 2 | 16 | 65 |
| 1125 | 1 | 139 | 61 |
| 1126 | 2 | 97 | 29 |
| 1127 | 1 | 62 | 29 |
| 1128 | 2 | 28 | 65 |
| 1129 | 1 | 113 | 27 |
| 1130 | 2 | 47 | 27 |
| 1131 | 1 | 82 | 29 |
| 1132 | 2 | 44 | 64 |
| 1133 | 1 | 125 | 27 |
| 1134 | 2 | 59 | 27 |
| 1135 | 1 | 94 | 29 |
| 1136 | 2 | 56 | 64 |
| 1137 | 1 | 137 | 27 |
| 1138 | 2 | 71 | 27 |
| 1139 | 1 | 106 | 29 |
| 1140 | 2 | 68 | 64 |
| 1141 | 1 | 77 | 62 |
| 1142 | 2 | 7 | 67 |
| 1143 | 1 | 3 | 34 |
| 1144 | 2 | 112 | 26 |
| 1145 | 1 | 89 | 62 |
| 1146 | 2 | 19 | 67 |
| 1147 | 1 | 15 | 34 |
| 1148 | 2 | 124 | 26 |
| 1149 | 1 | 101 | 62 |
| 1150 | 2 | 31 | 67 |
| 1151 | 1 | 27 | 34 |
| 1152 | 2 | 136 | 26 |
| 1153 | 1 | 75 | 4 |
| 1154 | 2 | 110 | 39 |
| 1155 | 1 | 9 | 37 |
| 1156 | 2 | 77 | 37 |
| 1157 | 1 | 87 | 4 |
| 1158 | 2 | 122 | 39 |
| 1159 | 1 | 21 | 37 |
| 1160 | 2 | 89 | 37 |
| 1161 | 1 | 99 | 4 |
| 1162 | 2 | 134 | 39 |
| 1163 | 1 | 33 | 37 |
| 1164 | 2 | 101 | 37 |
| 1165 | 1 | 78 | 40 |
| 1166 | 2 | 73 | 4 |
| 1167 | 1 | 44 | 35 |
| 1168 | 2 | 2 | 37 |
| 1169 | 1 | 90 | 40 |
| 1170 | 2 | 85 | 4 |
| 1171 | 1 | 56 | 35 |
| 1172 | 2 | 14 | 37 |
| 1173 | 1 | 102 | 40 |
| 1174 | 2 | 97 | 4 |
| 1175 | 1 | 68 | 35 |
| 1176 | 2 | 26 | 37 |
| 1177 | 1 | 8 | 36 |
| 1178 | 2 | 42 | 2 |
| 1179 | 1 | 74 | 37 |
| 1180 | 2 | 8 | 8 |
| 1181 | 1 | 20 | 36 |
| 1182 | 2 | 54 | 2 |
| 1183 | 1 | 86 | 37 |
| 1184 | 2 | 20 | 8 |
| 1185 | 1 | 32 | 36 |
| 1186 | 2 | 66 | 2 |
| 1187 | 1 | 98 | 37 |
| 1188 | 2 | 32 | 8 |
| 1189 | 1 | 83 | 11 |
| 1190 | 2 | 111 | 4 |
| 1191 | 1 | 82 | 39 |
| 1192 | 2 | 1 | 7 |
| 1193 | 1 | 95 | 11 |
| 1194 | 2 | 123 | 4 |
| 1195 | 1 | 94 | 39 |
| 1196 | 2 | 13 | 7 |
| 1197 | 1 | 107 | 11 |
| 1198 | 2 | 135 | 4 |
| 1199 | 1 | 106 | 39 |
| 1200 | 2 | 25 | 7 |
| 1201 | 1 | 78 | 8 |
| 1202 | 2 | 9 | 43 |
| 1203 | 1 | 37 | 2 |
| 1204 | 2 | 77 | 40 |
| 1205 | 1 | 90 | 8 |
| 1206 | 2 | 21 | 43 |
| 1207 | 1 | 49 | 2 |
| 1208 | 2 | 89 | 40 |
| 1209 | 1 | 102 | 8 |
| 1210 | 2 | 33 | 43 |
| 1211 | 1 | 61 | 2 |
| 1212 | 2 | 101 | 40 |
| 1213 | 1 | 39 | 34 |
| 1214 | 2 | 11 | 39 |
| 1215 | 1 | 43 | 36 |
| 1216 | 2 | 77 | 4 |
| 1217 | 1 | 51 | 34 |
| 1218 | 2 | 23 | 39 |
| 1219 | 1 | 55 | 36 |
| 1220 | 2 | 89 | 4 |
| 1221 | 1 | 63 | 34 |
| 1222 | 2 | 35 | 39 |
| 1223 | 1 | 67 | 36 |
| 1224 | 2 | 101 | 4 |
| 1225 | 1 | 109 | 8 |
| 1226 | 2 | 75 | 6 |
| 1227 | 1 | 48 | 35 |
| 1228 | 2 | 83 | 37 |
| 1229 | 1 | 121 | 8 |
| 1230 | 2 | 87 | 6 |
| 1231 | 1 | 60 | 35 |
| 1232 | 2 | 95 | 37 |
| 1233 | 1 | 133 | 8 |
| 1234 | 2 | 99 | 6 |
| 1235 | 1 | 72 | 35 |
| 1236 | 2 | 107 | 37 |
| 1237 | 1 | 2 | 7 |
| 1238 | 2 | 41 | 33 |
| 1239 | 1 | 119 | 35 |
| 1240 | 2 | 82 | 43 |
| 1241 | 1 | 14 | 7 |
| 1242 | 2 | 53 | 33 |
| 1243 | 1 | 131 | 35 |
| 1244 | 2 | 94 | 43 |
| 1245 | 1 | 26 | 7 |
| 1246 | 2 | 65 | 33 |
| 1247 | 1 | 143 | 35 |
| 1248 | 2 | 106 | 43 |
| 1249 | 1 | 75 | 12 |
| 1250 | 2 | 110 | 47 |
| 1251 | 1 | 9 | 45 |
| 1252 | 2 | 77 | 45 |
| 1253 | 1 | 87 | 12 |
| 1254 | 2 | 122 | 47 |
| 1255 | 1 | 21 | 45 |
| 1256 | 2 | 89 | 45 |
| 1257 | 1 | 99 | 12 |
| 1258 | 2 | 134 | 47 |
| 1259 | 1 | 33 | 45 |
| 1260 | 2 | 101 | 45 |
| 1261 | 1 | 78 | 48 |
| 1262 | 2 | 73 | 12 |
| 1263 | 1 | 44 | 43 |
| 1264 | 2 | 2 | 45 |
| 1265 | 1 | 90 | 48 |
| 1266 | 2 | 85 | 12 |
| 1267 | 1 | 56 | 43 |
| 1268 | 2 | 14 | 45 |
| 1269 | 1 | 102 | 48 |
| 1270 | 2 | 97 | 12 |
| 1271 | 1 | 68 | 43 |
| 1272 | 2 | 26 | 45 |
| 1273 | 1 | 8 | 44 |
| 1274 | 2 | 42 | 10 |
| 1275 | 1 | 74 | 45 |
| 1276 | 2 | 8 | 16 |
| 1277 | 1 | 20 | 44 |
| 1278 | 2 | 54 | 10 |
| 1279 | 1 | 86 | 45 |
| 1280 | 2 | 20 | 16 |
| 1281 | 1 | 32 | 44 |
| 1282 | 2 | 66 | 10 |
| 1283 | 1 | 98 | 45 |
| 1284 | 2 | 32 | 16 |
| 1285 | 1 | 83 | 19 |
| 1286 | 2 | 111 | 12 |
| 1287 | 1 | 82 | 47 |
| 1288 | 2 | 1 | 15 |
| 1289 | 1 | 95 | 19 |
| 1290 | 2 | 123 | 12 |
| 1291 | 1 | 94 | 47 |
| 1292 | 2 | 13 | 15 |
| 1293 | 1 | 107 | 19 |
| 1294 | 2 | 135 | 12 |
| 1295 | 1 | 106 | 47 |
| 1296 | 2 | 25 | 15 |
| 1297 | 1 | 78 | 16 |
| 1298 | 2 | 9 | 51 |
| 1299 | 1 | 37 | 10 |
| 1300 | 2 | 77 | 48 |
| 1301 | 1 | 90 | 16 |
| 1302 | 2 | 21 | 51 |
| 1303 | 1 | 49 | 10 |
| 1304 | 2 | 89 | 48 |
| 1305 | 1 | 102 | 16 |
| 1306 | 2 | 33 | 51 |
| 1307 | 1 | 61 | 10 |
| 1308 | 2 | 101 | 48 |
| 1309 | 1 | 39 | 42 |
| 1310 | 2 | 11 | 47 |
| 1311 | 1 | 43 | 44 |
| 1312 | 2 | 77 | 12 |
| 1313 | 1 | 51 | 42 |
| 1314 | 2 | 23 | 47 |
| 1315 | 1 | 55 | 44 |
| 1316 | 2 | 89 | 12 |
| 1317 | 1 | 63 | 42 |
| 1318 | 2 | 35 | 47 |
| 1319 | 1 | 67 | 44 |
| 1320 | 2 | 101 | 12 |
| 1321 | 1 | 109 | 16 |
| 1322 | 2 | 75 | 14 |
| 1323 | 1 | 48 | 43 |
| 1324 | 2 | 83 | 45 |
| 1325 | 1 | 121 | 16 |
| 1326 | 2 | 87 | 14 |
| 1327 | 1 | 60 | 43 |
| 1328 | 2 | 95 | 45 |
| 1329 | 1 | 133 | 16 |
| 1330 | 2 | 99 | 14 |
| 1331 | 1 | 72 | 43 |
| 1332 | 2 | 107 | 45 |
| 1333 | 1 | 2 | 15 |
| 1334 | 2 | 41 | 41 |
| 1335 | 1 | 119 | 43 |
| 1336 | 2 | 82 | 51 |
| 1337 | 1 | 14 | 15 |
| 1338 | 2 | 53 | 41 |
| 1339 | 1 | 131 | 43 |
| 1340 | 2 | 94 | 51 |
| 1341 | 1 | 26 | 15 |
| 1342 | 2 | 65 | 41 |
| 1343 | 1 | 143 | 43 |
| 1344 | 2 | 106 | 51 |
| 1345 | 1 | 75 | 20 |
| 1346 | 2 | 110 | 55 |
| 1347 | 1 | 9 | 53 |
| 1348 | 2 | 77 | 53 |
| 1349 | 1 | 87 | 20 |
| 1350 | 2 | 122 | 55 |
| 1351 | 1 | 21 | 53 |
| 1352 | 2 | 89 | 53 |
| 1353 | 1 | 99 | 20 |
| 1354 | 2 | 134 | 55 |
| 1355 | 1 | 33 | 53 |
| 1356 | 2 | 101 | 53 |
| 1357 | 1 | 78 | 56 |
| 1358 | 2 | 73 | 20 |
| 1359 | 1 | 44 | 51 |
| 1360 | 2 | 2 | 53 |
| 1361 | 1 | 90 | 56 |
| 1362 | 2 | 85 | 20 |
| 1363 | 1 | 56 | 51 |
| 1364 | 2 | 14 | 53 |
| 1365 | 1 | 102 | 56 |
| 1366 | 2 | 97 | 20 |
| 1367 | 1 | 68 | 51 |
| 1368 | 2 | 26 | 53 |
| 1369 | 1 | 8 | 52 |
| 1370 | 2 | 42 | 18 |
| 1371 | 1 | 74 | 53 |
| 1372 | 2 | 8 | 24 |
| 1373 | 1 | 20 | 52 |
| 1374 | 2 | 54 | 18 |
| 1375 | 1 | 86 | 53 |
| 1376 | 2 | 20 | 24 |
| 1377 | 1 | 32 | 52 |
| 1378 | 2 | 66 | 18 |
| 1379 | 1 | 98 | 53 |
| 1380 | 2 | 32 | 24 |
| 1381 | 1 | 83 | 27 |
| 1382 | 2 | 111 | 20 |
| 1383 | 1 | 82 | 55 |
| 1384 | 2 | 1 | 23 |
| 1385 | 1 | 95 | 27 |
| 1386 | 2 | 123 | 20 |
| 1387 | 1 | 94 | 55 |
| 1388 | 2 | 13 | 23 |
| 1389 | 1 | 107 | 27 |
| 1390 | 2 | 135 | 20 |
| 1391 | 1 | 106 | 55 |
| 1392 | 2 | 25 | 23 |
| 1393 | 1 | 78 | 24 |
| 1394 | 2 | 9 | 59 |
| 1395 | 1 | 37 | 18 |
| 1396 | 2 | 77 | 56 |
| 1397 | 1 | 90 | 24 |
| 1398 | 2 | 21 | 59 |
| 1399 | 1 | 49 | 18 |
| 1400 | 2 | 89 | 56 |
| 1401 | 1 | 102 | 24 |
| 1402 | 2 | 33 | 59 |
| 1403 | 1 | 61 | 18 |
| 1404 | 2 | 101 | 56 |
| 1405 | 1 | 39 | 50 |
| 1406 | 2 | 11 | 55 |
| 1407 | 1 | 43 | 52 |
| 1408 | 2 | 77 | 20 |
| 1409 | 1 | 51 | 50 |
| 1410 | 2 | 23 | 55 |
| 1411 | 1 | 55 | 52 |
| 1412 | 2 | 89 | 20 |
| 1413 | 1 | 63 | 50 |
| 1414 | 2 | 35 | 55 |
| 1415 | 1 | 67 | 52 |
| 1416 | 2 | 101 | 20 |
| 1417 | 1 | 109 | 24 |
| 1418 | 2 | 75 | 22 |
| 1419 | 1 | 48 | 51 |
| 1420 | 2 | 83 | 53 |
| 1421 | 1 | 121 | 24 |
| 1422 | 2 | 87 | 22 |
| 1423 | 1 | 60 | 51 |
| 1424 | 2 | 95 | 53 |
| 1425 | 1 | 133 | 24 |
| 1426 | 2 | 99 | 22 |
| 1427 | 1 | 72 | 51 |
| 1428 | 2 | 107 | 53 |
| 1429 | 1 | 2 | 23 |
| 1430 | 2 | 41 | 49 |
| 1431 | 1 | 119 | 51 |
| 1432 | 2 | 82 | 59 |
| 1433 | 1 | 14 | 23 |
| 1434 | 2 | 53 | 49 |
| 1435 | 1 | 131 | 51 |
| 1436 | 2 | 94 | 59 |
| 1437 | 1 | 26 | 23 |
| 1438 | 2 | 65 | 49 |
| 1439 | 1 | 143 | 51 |
| 1440 | 2 | 106 | 59 |
| 1441 | 1 | 75 | 28 |
| 1442 | 2 | 110 | 63 |
| 1443 | 1 | 9 | 61 |
| 1444 | 2 | 77 | 61 |
| 1445 | 1 | 87 | 28 |
| 1446 | 2 | 122 | 63 |
| 1447 | 1 | 21 | 61 |
| 1448 | 2 | 89 | 61 |
| 1449 | 1 | 99 | 28 |
| 1450 | 2 | 134 | 63 |
| 1451 | 1 | 33 | 61 |
| 1452 | 2 | 101 | 61 |
| 1453 | 1 | 78 | 64 |
| 1454 | 2 | 73 | 28 |
| 1455 | 1 | 44 | 59 |
| 1456 | 2 | 2 | 61 |
| 1457 | 1 | 90 | 64 |
| 1458 | 2 | 85 | 28 |
| 1459 | 1 | 56 | 59 |
| 1460 | 2 | 14 | 61 |
| 1461 | 1 | 102 | 64 |
| 1462 | 2 | 97 | 28 |
| 1463 | 1 | 68 | 59 |
| 1464 | 2 | 26 | 61 |
| 1465 | 1 | 8 | 60 |
| 1466 | 2 | 42 | 26 |
| 1467 | 1 | 74 | 61 |
| 1468 | 2 | 8 | 32 |
| 1469 | 1 | 20 | 60 |
| 1470 | 2 | 54 | 26 |
| 1471 | 1 | 86 | 61 |
| 1472 | 2 | 20 | 32 |
| 1473 | 1 | 32 | 60 |
| 1474 | 2 | 66 | 26 |
| 1475 | 1 | 98 | 61 |
| 1476 | 2 | 32 | 32 |
| 1477 | 1 | 83 | 35 |
| 1478 | 2 | 111 | 28 |
| 1479 | 1 | 82 | 63 |
| 1480 | 2 | 1 | 31 |
| 1481 | 1 | 95 | 35 |
| 1482 | 2 | 123 | 28 |
| 1483 | 1 | 94 | 63 |
| 1484 | 2 | 13 | 31 |
| 1485 | 1 | 107 | 35 |
| 1486 | 2 | 135 | 28 |
| 1487 | 1 | 106 | 63 |
| 1488 | 2 | 25 | 31 |
| 1489 | 1 | 78 | 32 |
| 1490 | 2 | 9 | 67 |
| 1491 | 1 | 37 | 26 |
| 1492 | 2 | 77 | 64 |
| 1493 | 1 | 90 | 32 |
| 1494 | 2 | 21 | 67 |
| 1495 | 1 | 49 | 26 |
| 1496 | 2 | 89 | 64 |
| 1497 | 1 | 102 | 32 |
| 1498 | 2 | 33 | 67 |
| 1499 | 1 | 61 | 26 |
| 1500 | 2 | 101 | 64 |
| 1501 | 1 | 39 | 58 |
| 1502 | 2 | 11 | 63 |
| 1503 | 1 | 43 | 60 |
| 1504 | 2 | 77 | 28 |
| 1505 | 1 | 51 | 58 |
| 1506 | 2 | 23 | 63 |
| 1507 | 1 | 55 | 60 |
| 1508 | 2 | 89 | 28 |
| 1509 | 1 | 63 | 58 |
| 1510 | 2 | 35 | 63 |
| 1511 | 1 | 67 | 60 |
| 1512 | 2 | 101 | 28 |
| 1513 | 1 | 109 | 32 |
| 1514 | 2 | 75 | 30 |
| 1515 | 1 | 48 | 59 |
| 1516 | 2 | 83 | 61 |
| 1517 | 1 | 121 | 32 |
| 1518 | 2 | 87 | 30 |
| 1519 | 1 | 60 | 59 |
| 1520 | 2 | 95 | 61 |
| 1521 | 1 | 133 | 32 |
| 1522 | 2 | 99 | 30 |
| 1523 | 1 | 72 | 59 |
| 1524 | 2 | 107 | 61 |
| 1525 | 1 | 2 | 31 |
| 1526 | 2 | 41 | 57 |
| 1527 | 1 | 119 | 59 |
| 1528 | 2 | 82 | 67 |
| 1529 | 1 | 14 | 31 |
| 1530 | 2 | 53 | 57 |
| 1531 | 1 | 131 | 59 |
| 1532 | 2 | 94 | 67 |
| 1533 | 1 | 26 | 31 |
| 1534 | 2 | 65 | 57 |
| 1535 | 1 | 143 | 59 |
| 1536 | 2 | 106 | 67 |
| 1537 | 1 | 12 | 5 |
| 1538 | 2 | 80 | 9 |
| 1539 | 1 | 2 | 42 |
| 1540 | 2 | 84 | 40 |
| 1541 | 1 | 24 | 5 |
| 1542 | 2 | 92 | 9 |
| 1543 | 1 | 14 | 42 |
| 1544 | 2 | 96 | 40 |
| 1545 | 1 | 36 | 5 |
| 1546 | 2 | 104 | 9 |
| 1547 | 1 | 26 | 42 |
| 1548 | 2 | 108 | 40 |
| 1549 | 1 | 46 | 3 |
| 1550 | 2 | 37 | 33 |
| 1551 | 1 | 46 | 36 |
| 1552 | 2 | 109 | 6 |
| 1553 | 1 | 58 | 3 |
| 1554 | 2 | 49 | 33 |
| 1555 | 1 | 58 | 36 |
| 1556 | 2 | 121 | 6 |
| 1557 | 1 | 70 | 3 |
| 1558 | 2 | 61 | 33 |
| 1559 | 1 | 70 | 36 |
| 1560 | 2 | 133 | 6 |
| 1561 | 1 | 81 | 11 |
| 1562 | 2 | 73 | 36 |
| 1563 | 1 | 112 | 1 |
| 1564 | 2 | 48 | 33 |
| 1565 | 1 | 93 | 11 |
| 1566 | 2 | 85 | 36 |
| 1567 | 1 | 124 | 1 |
| 1568 | 2 | 60 | 33 |
| 1569 | 1 | 105 | 11 |
| 1570 | 2 | 97 | 36 |
| 1571 | 1 | 136 | 1 |
| 1572 | 2 | 72 | 33 |
| 1573 | 1 | 1 | 39 |
| 1574 | 2 | 83 | 36 |
| 1575 | 1 | 12 | 4 |
| 1576 | 2 | 117 | 1 |
| 1577 | 1 | 13 | 39 |
| 1578 | 2 | 95 | 36 |
| 1579 | 1 | 24 | 4 |
| 1580 | 2 | 129 | 1 |
| 1581 | 1 | 25 | 39 |
| 1582 | 2 | 107 | 36 |
| 1583 | 1 | 36 | 4 |
| 1584 | 2 | 141 | 1 |
| 1585 | 1 | 113 | 37 |
| 1586 | 2 | 109 | 2 |
| 1587 | 1 | 116 | 8 |
| 1588 | 2 | 10 | 7 |
| 1589 | 1 | 125 | 37 |
| 1590 | 2 | 121 | 2 |
| 1591 | 1 | 128 | 8 |
| 1592 | 2 | 22 | 7 |
| 1593 | 1 | 137 | 37 |
| 1594 | 2 | 133 | 2 |
| 1595 | 1 | 140 | 8 |
| 1596 | 2 | 34 | 7 |
| 1597 | 1 | 77 | 9 |
| 1598 | 2 | 1 | 37 |
| 1599 | 1 | 2 | 6 |
| 1600 | 2 | 9 | 40 |
| 1601 | 1 | 89 | 9 |
| 1602 | 2 | 13 | 37 |
| 1603 | 1 | 14 | 6 |
| 1604 | 2 | 21 | 40 |
| 1605 | 1 | 101 | 9 |
| 1606 | 2 | 25 | 37 |
| 1607 | 1 | 26 | 6 |
| 1608 | 2 | 33 | 40 |
| 1609 | 1 | 7 | 37 |
| 1610 | 2 | 37 | 34 |
| 1611 | 1 | 44 | 37 |
| 1612 | 2 | 80 | 37 |
| 1613 | 1 | 19 | 37 |
| 1614 | 2 | 49 | 34 |
| 1615 | 1 | 56 | 37 |
| 1616 | 2 | 92 | 37 |
| 1617 | 1 | 31 | 37 |
| 1618 | 2 | 61 | 34 |
| 1619 | 1 | 68 | 37 |
| 1620 | 2 | 104 | 37 |
| 1621 | 1 | 48 | 5 |
| 1622 | 2 | 73 | 37 |
| 1623 | 1 | 81 | 38 |
| 1624 | 2 | 41 | 8 |
| 1625 | 1 | 60 | 5 |
| 1626 | 2 | 85 | 37 |
| 1627 | 1 | 93 | 38 |
| 1628 | 2 | 53 | 8 |
| 1629 | 1 | 72 | 5 |
| 1630 | 2 | 97 | 37 |
| 1631 | 1 | 105 | 38 |
| 1632 | 2 | 65 | 8 |
| 1633 | 1 | 12 | 13 |
| 1634 | 2 | 80 | 17 |
| 1635 | 1 | 2 | 50 |
| 1636 | 2 | 84 | 48 |
| 1637 | 1 | 24 | 13 |
| 1638 | 2 | 92 | 17 |
| 1639 | 1 | 14 | 50 |
| 1640 | 2 | 96 | 48 |
| 1641 | 1 | 36 | 13 |
| 1642 | 2 | 104 | 17 |
| 1643 | 1 | 26 | 50 |
| 1644 | 2 | 108 | 48 |
| 1645 | 1 | 46 | 11 |
| 1646 | 2 | 37 | 41 |
| 1647 | 1 | 46 | 44 |
| 1648 | 2 | 109 | 14 |
| 1649 | 1 | 58 | 11 |
| 1650 | 2 | 49 | 41 |
| 1651 | 1 | 58 | 44 |
| 1652 | 2 | 121 | 14 |
| 1653 | 1 | 70 | 11 |
| 1654 | 2 | 61 | 41 |
| 1655 | 1 | 70 | 44 |
| 1656 | 2 | 133 | 14 |
| 1657 | 1 | 81 | 19 |
| 1658 | 2 | 73 | 44 |
| 1659 | 1 | 112 | 9 |
| 1660 | 2 | 48 | 41 |
| 1661 | 1 | 93 | 19 |
| 1662 | 2 | 85 | 44 |
| 1663 | 1 | 124 | 9 |
| 1664 | 2 | 60 | 41 |
| 1665 | 1 | 105 | 19 |
| 1666 | 2 | 97 | 44 |
| 1667 | 1 | 136 | 9 |
| 1668 | 2 | 72 | 41 |
| 1669 | 1 | 1 | 47 |
| 1670 | 2 | 83 | 44 |
| 1671 | 1 | 12 | 12 |
| 1672 | 2 | 117 | 9 |
| 1673 | 1 | 13 | 47 |
| 1674 | 2 | 95 | 44 |
| 1675 | 1 | 24 | 12 |
| 1676 | 2 | 129 | 9 |
| 1677 | 1 | 25 | 47 |
| 1678 | 2 | 107 | 44 |
| 1679 | 1 | 36 | 12 |
| 1680 | 2 | 141 | 9 |
| 1681 | 1 | 113 | 45 |
| 1682 | 2 | 109 | 10 |
| 1683 | 1 | 116 | 16 |
| 1684 | 2 | 10 | 15 |
| 1685 | 1 | 125 | 45 |
| 1686 | 2 | 121 | 10 |
| 1687 | 1 | 128 | 16 |
| 1688 | 2 | 22 | 15 |
| 1689 | 1 | 137 | 45 |
| 1690 | 2 | 133 | 10 |
| 1691 | 1 | 140 | 16 |
| 1692 | 2 | 34 | 15 |
| 1693 | 1 | 77 | 17 |
| 1694 | 2 | 1 | 45 |
| 1695 | 1 | 2 | 14 |
| 1696 | 2 | 9 | 48 |
| 1697 | 1 | 89 | 17 |
| 1698 | 2 | 13 | 45 |
| 1699 | 1 | 14 | 14 |
| 1700 | 2 | 21 | 48 |
| 1701 | 1 | 101 | 17 |
| 1702 | 2 | 25 | 45 |
| 1703 | 1 | 26 | 14 |
| 1704 | 2 | 33 | 48 |
| 1705 | 1 | 7 | 45 |
| 1706 | 2 | 37 | 42 |
| 1707 | 1 | 44 | 45 |
| 1708 | 2 | 80 | 45 |
| 1709 | 1 | 19 | 45 |
| 1710 | 2 | 49 | 42 |
| 1711 | 1 | 56 | 45 |
| 1712 | 2 | 92 | 45 |
| 1713 | 1 | 31 | 45 |
| 1714 | 2 | 61 | 42 |
| 1715 | 1 | 68 | 45 |
| 1716 | 2 | 104 | 45 |
| 1717 | 1 | 48 | 13 |
| 1718 | 2 | 73 | 45 |
| 1719 | 1 | 81 | 46 |
| 1720 | 2 | 41 | 16 |
| 1721 | 1 | 60 | 13 |
| 1722 | 2 | 85 | 45 |
| 1723 | 1 | 93 | 46 |
| 1724 | 2 | 53 | 16 |
| 1725 | 1 | 72 | 13 |
| 1726 | 2 | 97 | 45 |
| 1727 | 1 | 105 | 46 |
| 1728 | 2 | 65 | 16 |
| 1729 | 1 | 12 | 21 |
| 1730 | 2 | 80 | 25 |
| 1731 | 1 | 2 | 58 |
| 1732 | 2 | 84 | 56 |
| 1733 | 1 | 24 | 21 |
| 1734 | 2 | 92 | 25 |
| 1735 | 1 | 14 | 58 |
| 1736 | 2 | 96 | 56 |
| 1737 | 1 | 36 | 21 |
| 1738 | 2 | 104 | 25 |
| 1739 | 1 | 26 | 58 |
| 1740 | 2 | 108 | 56 |
| 1741 | 1 | 46 | 19 |
| 1742 | 2 | 37 | 49 |
| 1743 | 1 | 46 | 52 |
| 1744 | 2 | 109 | 22 |
| 1745 | 1 | 58 | 19 |
| 1746 | 2 | 49 | 49 |
| 1747 | 1 | 58 | 52 |
| 1748 | 2 | 121 | 22 |
| 1749 | 1 | 70 | 19 |
| 1750 | 2 | 61 | 49 |
| 1751 | 1 | 70 | 52 |
| 1752 | 2 | 133 | 22 |
| 1753 | 1 | 81 | 27 |
| 1754 | 2 | 73 | 52 |
| 1755 | 1 | 112 | 17 |
| 1756 | 2 | 48 | 49 |
| 1757 | 1 | 93 | 27 |
| 1758 | 2 | 85 | 52 |
| 1759 | 1 | 124 | 17 |
| 1760 | 2 | 60 | 49 |
| 1761 | 1 | 105 | 27 |
| 1762 | 2 | 97 | 52 |
| 1763 | 1 | 136 | 17 |
| 1764 | 2 | 72 | 49 |
| 1765 | 1 | 1 | 55 |
| 1766 | 2 | 83 | 52 |
| 1767 | 1 | 12 | 20 |
| 1768 | 2 | 117 | 17 |
| 1769 | 1 | 13 | 55 |
| 1770 | 2 | 95 | 52 |
| 1771 | 1 | 24 | 20 |
| 1772 | 2 | 129 | 17 |
| 1773 | 1 | 25 | 55 |
| 1774 | 2 | 107 | 52 |
| 1775 | 1 | 36 | 20 |
| 1776 | 2 | 141 | 17 |
| 1777 | 1 | 113 | 53 |
| 1778 | 2 | 109 | 18 |
| 1779 | 1 | 116 | 24 |
| 1780 | 2 | 10 | 23 |
| 1781 | 1 | 125 | 53 |
| 1782 | 2 | 121 | 18 |
| 1783 | 1 | 128 | 24 |
| 1784 | 2 | 22 | 23 |
| 1785 | 1 | 137 | 53 |
| 1786 | 2 | 133 | 18 |
| 1787 | 1 | 140 | 24 |
| 1788 | 2 | 34 | 23 |
| 1789 | 1 | 77 | 25 |
| 1790 | 2 | 1 | 53 |
| 1791 | 1 | 2 | 22 |
| 1792 | 2 | 9 | 56 |
| 1793 | 1 | 89 | 25 |
| 1794 | 2 | 13 | 53 |
| 1795 | 1 | 14 | 22 |
| 1796 | 2 | 21 | 56 |
| 1797 | 1 | 101 | 25 |
| 1798 | 2 | 25 | 53 |
| 1799 | 1 | 26 | 22 |
| 1800 | 2 | 33 | 56 |
| 1801 | 1 | 7 | 53 |
| 1802 | 2 | 37 | 50 |
| 1803 | 1 | 44 | 53 |
| 1804 | 2 | 80 | 53 |
| 1805 | 1 | 19 | 53 |
| 1806 | 2 | 49 | 50 |
| 1807 | 1 | 56 | 53 |
| 1808 | 2 | 92 | 53 |
| 1809 | 1 | 31 | 53 |
| 1810 | 2 | 61 | 50 |
| 1811 | 1 | 68 | 53 |
| 1812 | 2 | 104 | 53 |
| 1813 | 1 | 48 | 21 |
| 1814 | 2 | 73 | 53 |
| 1815 | 1 | 81 | 54 |
| 1816 | 2 | 41 | 24 |
| 1817 | 1 | 60 | 21 |
| 1818 | 2 | 85 | 53 |
| 1819 | 1 | 93 | 54 |
| 1820 | 2 | 53 | 24 |
| 1821 | 1 | 72 | 21 |
| 1822 | 2 | 97 | 53 |
| 1823 | 1 | 105 | 54 |
| 1824 | 2 | 65 | 24 |
| 1825 | 1 | 12 | 29 |
| 1826 | 2 | 80 | 33 |
| 1827 | 1 | 2 | 66 |
| 1828 | 2 | 84 | 64 |
| 1829 | 1 | 24 | 29 |
| 1830 | 2 | 92 | 33 |
| 1831 | 1 | 14 | 66 |
| 1832 | 2 | 96 | 64 |
| 1833 | 1 | 36 | 29 |
| 1834 | 2 | 104 | 33 |
| 1835 | 1 | 26 | 66 |
| 1836 | 2 | 108 | 64 |
| 1837 | 1 | 46 | 27 |
| 1838 | 2 | 37 | 57 |
| 1839 | 1 | 46 | 60 |
| 1840 | 2 | 109 | 30 |
| 1841 | 1 | 58 | 27 |
| 1842 | 2 | 49 | 57 |
| 1843 | 1 | 58 | 60 |
| 1844 | 2 | 121 | 30 |
| 1845 | 1 | 70 | 27 |
| 1846 | 2 | 61 | 57 |
| 1847 | 1 | 70 | 60 |
| 1848 | 2 | 133 | 30 |
| 1849 | 1 | 81 | 35 |
| 1850 | 2 | 73 | 60 |
| 1851 | 1 | 112 | 25 |
| 1852 | 2 | 48 | 57 |
| 1853 | 1 | 93 | 35 |
| 1854 | 2 | 85 | 60 |
| 1855 | 1 | 124 | 25 |
| 1856 | 2 | 60 | 57 |
| 1857 | 1 | 105 | 35 |
| 1858 | 2 | 97 | 60 |
| 1859 | 1 | 136 | 25 |
| 1860 | 2 | 72 | 57 |
| 1861 | 1 | 1 | 63 |
| 1862 | 2 | 83 | 60 |
| 1863 | 1 | 12 | 28 |
| 1864 | 2 | 117 | 25 |
| 1865 | 1 | 13 | 63 |
| 1866 | 2 | 95 | 60 |
| 1867 | 1 | 24 | 28 |
| 1868 | 2 | 129 | 25 |
| 1869 | 1 | 25 | 63 |
| 1870 | 2 | 107 | 60 |
| 1871 | 1 | 36 | 28 |
| 1872 | 2 | 141 | 25 |
| 1873 | 1 | 113 | 61 |
| 1874 | 2 | 109 | 26 |
| 1875 | 1 | 116 | 32 |
| 1876 | 2 | 10 | 31 |
| 1877 | 1 | 125 | 61 |
| 1878 | 2 | 121 | 26 |
| 1879 | 1 | 128 | 32 |
| 1880 | 2 | 22 | 31 |
| 1881 | 1 | 137 | 61 |
| 1882 | 2 | 133 | 26 |
| 1883 | 1 | 140 | 32 |
| 1884 | 2 | 34 | 31 |
| 1885 | 1 | 77 | 33 |
| 1886 | 2 | 1 | 61 |
| 1887 | 1 | 2 | 30 |
| 1888 | 2 | 9 | 64 |
| 1889 | 1 | 89 | 33 |
| 1890 | 2 | 13 | 61 |
| 1891 | 1 | 14 | 30 |
| 1892 | 2 | 21 | 64 |
| 1893 | 1 | 101 | 33 |
| 1894 | 2 | 25 | 61 |
| 1895 | 1 | 26 | 30 |
| 1896 | 2 | 33 | 64 |
| 1897 | 1 | 7 | 61 |
| 1898 | 2 | 37 | 58 |
| 1899 | 1 | 44 | 61 |
| 1900 | 2 | 80 | 61 |
| 1901 | 1 | 19 | 61 |
| 1902 | 2 | 49 | 58 |
| 1903 | 1 | 56 | 61 |
| 1904 | 2 | 92 | 61 |
| 1905 | 1 | 31 | 61 |
| 1906 | 2 | 61 | 58 |
| 1907 | 1 | 68 | 61 |
| 1908 | 2 | 104 | 61 |
| 1909 | 1 | 48 | 29 |
| 1910 | 2 | 73 | 61 |
| 1911 | 1 | 81 | 62 |
| 1912 | 2 | 41 | 32 |
| 1913 | 1 | 60 | 29 |
| 1914 | 2 | 85 | 61 |
| 1915 | 1 | 93 | 62 |
| 1916 | 2 | 53 | 32 |
| 1917 | 1 | 72 | 29 |
| 1918 | 2 | 97 | 61 |
| 1919 | 1 | 105 | 62 |
| 1920 | 2 | 65 | 32 |
| 1921 | 1 | 11 | 10 |
| 1922 | 2 | 109 | 33 |
| 1923 | 1 | 45 | 33 |
| 1924 | 2 | 111 | 35 |
| 1925 | 1 | 23 | 10 |
| 1926 | 2 | 121 | 33 |
| 1927 | 1 | 57 | 33 |
| 1928 | 2 | 123 | 35 |
| 1929 | 1 | 35 | 10 |
| 1930 | 2 | 133 | 33 |
| 1931 | 1 | 69 | 33 |
| 1932 | 2 | 135 | 35 |
| 1933 | 1 | 75 | 40 |
| 1934 | 2 | 1 | 5 |
| 1935 | 1 | 41 | 35 |
| 1936 | 2 | 116 | 34 |
| 1937 | 1 | 87 | 40 |
| 1938 | 2 | 13 | 5 |
| 1939 | 1 | 53 | 35 |
| 1940 | 2 | 128 | 34 |
| 1941 | 1 | 99 | 40 |
| 1942 | 2 | 25 | 5 |
| 1943 | 1 | 65 | 35 |
| 1944 | 2 | 140 | 34 |
| 1945 | 1 | 5 | 7 |
| 1946 | 2 | 40 | 8 |
| 1947 | 1 | 74 | 41 |
| 1948 | 2 | 12 | 41 |
| 1949 | 1 | 17 | 7 |
| 1950 | 2 | 52 | 8 |
| 1951 | 1 | 86 | 41 |
| 1952 | 2 | 24 | 41 |
| 1953 | 1 | 29 | 7 |
| 1954 | 2 | 64 | 8 |
| 1955 | 1 | 98 | 41 |
| 1956 | 2 | 36 | 41 |
| 1957 | 1 | 84 | 41 |
| 1958 | 2 | 46 | 33 |
| 1959 | 1 | 43 | 8 |
| 1960 | 2 | 37 | 36 |
| 1961 | 1 | 96 | 41 |
| 1962 | 2 | 58 | 33 |
| 1963 | 1 | 55 | 8 |
| 1964 | 2 | 49 | 36 |
| 1965 | 1 | 108 | 41 |
| 1966 | 2 | 70 | 33 |
| 1967 | 1 | 67 | 8 |
| 1968 | 2 | 61 | 36 |
| 1969 | 1 | 2 | 38 |
| 1970 | 2 | 109 | 34 |
| 1971 | 1 | 112 | 3 |
| 1972 | 2 | 1 | 6 |
| 1973 | 1 | 14 | 38 |
| 1974 | 2 | 121 | 34 |
| 1975 | 1 | 124 | 3 |
| 1976 | 2 | 13 | 6 |
| 1977 | 1 | 26 | 38 |
| 1978 | 2 | 133 | 34 |
| 1979 | 1 | 136 | 3 |
| 1980 | 2 | 25 | 6 |
| 1981 | 1 | 42 | 36 |
| 1982 | 2 | 83 | 7 |
| 1983 | 1 | 48 | 40 |
| 1984 | 2 | 37 | 3 |
| 1985 | 1 | 54 | 36 |
| 1986 | 2 | 95 | 7 |
| 1987 | 1 | 60 | 40 |
| 1988 | 2 | 49 | 3 |
| 1989 | 1 | 66 | 36 |
| 1990 | 2 | 107 | 7 |
| 1991 | 1 | 72 | 40 |
| 1992 | 2 | 61 | 3 |
| 1993 | 1 | 7 | 7 |
| 1994 | 2 | 110 | 40 |
| 1995 | 1 | 75 | 10 |
| 1996 | 2 | 109 | 3 |
| 1997 | 1 | 19 | 7 |
| 1998 | 2 | 122 | 40 |
| 1999 | 1 | 87 | 10 |
| 2000 | 2 | 121 | 3 |
| 2001 | 1 | 31 | 7 |
| 2002 | 2 | 134 | 40 |
| 2003 | 1 | 99 | 10 |
| 2004 | 2 | 133 | 3 |
| 2005 | 1 | 81 | 10 |
| 2006 | 2 | 73 | 7 |
| 2007 | 1 | 80 | 43 |
| 2008 | 2 | 115 | 1 |
| 2009 | 1 | 93 | 10 |
| 2010 | 2 | 85 | 7 |
| 2011 | 1 | 92 | 43 |
| 2012 | 2 | 127 | 1 |
| 2013 | 1 | 105 | 10 |
| 2014 | 2 | 97 | 7 |
| 2015 | 1 | 104 | 43 |
| 2016 | 2 | 139 | 1 |
| 2017 | 1 | 11 | 18 |
| 2018 | 2 | 109 | 41 |
| 2019 | 1 | 45 | 41 |
| 2020 | 2 | 111 | 43 |
| 2021 | 1 | 23 | 18 |
| 2022 | 2 | 121 | 41 |
| 2023 | 1 | 57 | 41 |
| 2024 | 2 | 123 | 43 |
| 2025 | 1 | 35 | 18 |
| 2026 | 2 | 133 | 41 |
| 2027 | 1 | 69 | 41 |
| 2028 | 2 | 135 | 43 |
| 2029 | 1 | 75 | 48 |
| 2030 | 2 | 1 | 13 |
| 2031 | 1 | 41 | 43 |
| 2032 | 2 | 116 | 42 |
| 2033 | 1 | 87 | 48 |
| 2034 | 2 | 13 | 13 |
| 2035 | 1 | 53 | 43 |
| 2036 | 2 | 128 | 42 |
| 2037 | 1 | 99 | 48 |
| 2038 | 2 | 25 | 13 |
| 2039 | 1 | 65 | 43 |
| 2040 | 2 | 140 | 42 |
| 2041 | 1 | 5 | 15 |
| 2042 | 2 | 40 | 16 |
| 2043 | 1 | 74 | 49 |
| 2044 | 2 | 12 | 49 |
| 2045 | 1 | 17 | 15 |
| 2046 | 2 | 52 | 16 |
| 2047 | 1 | 86 | 49 |
| 2048 | 2 | 24 | 49 |
| 2049 | 1 | 29 | 15 |
| 2050 | 2 | 64 | 16 |
| 2051 | 1 | 98 | 49 |
| 2052 | 2 | 36 | 49 |
| 2053 | 1 | 84 | 49 |
| 2054 | 2 | 46 | 41 |
| 2055 | 1 | 43 | 16 |
| 2056 | 2 | 37 | 44 |
| 2057 | 1 | 96 | 49 |
| 2058 | 2 | 58 | 41 |
| 2059 | 1 | 55 | 16 |
| 2060 | 2 | 49 | 44 |
| 2061 | 1 | 108 | 49 |
| 2062 | 2 | 70 | 41 |
| 2063 | 1 | 67 | 16 |
| 2064 | 2 | 61 | 44 |
| 2065 | 1 | 2 | 46 |
| 2066 | 2 | 109 | 42 |
| 2067 | 1 | 112 | 11 |
| 2068 | 2 | 1 | 14 |
| 2069 | 1 | 14 | 46 |
| 2070 | 2 | 121 | 42 |
| 2071 | 1 | 124 | 11 |
| 2072 | 2 | 13 | 14 |
| 2073 | 1 | 26 | 46 |
| 2074 | 2 | 133 | 42 |
| 2075 | 1 | 136 | 11 |
| 2076 | 2 | 25 | 14 |
| 2077 | 1 | 42 | 44 |
| 2078 | 2 | 83 | 15 |
| 2079 | 1 | 48 | 48 |
| 2080 | 2 | 37 | 11 |
| 2081 | 1 | 54 | 44 |
| 2082 | 2 | 95 | 15 |
| 2083 | 1 | 60 | 48 |
| 2084 | 2 | 49 | 11 |
| 2085 | 1 | 66 | 44 |
| 2086 | 2 | 107 | 15 |
| 2087 | 1 | 72 | 48 |
| 2088 | 2 | 61 | 11 |
| 2089 | 1 | 7 | 15 |
| 2090 | 2 | 110 | 48 |
| 2091 | 1 | 75 | 18 |
| 2092 | 2 | 109 | 11 |
| 2093 | 1 | 19 | 15 |
| 2094 | 2 | 122 | 48 |
| 2095 | 1 | 87 | 18 |
| 2096 | 2 | 121 | 11 |
| 2097 | 1 | 31 | 15 |
| 2098 | 2 | 134 | 48 |
| 2099 | 1 | 99 | 18 |
| 2100 | 2 | 133 | 11 |
| 2101 | 1 | 81 | 18 |
| 2102 | 2 | 73 | 15 |
| 2103 | 1 | 80 | 51 |
| 2104 | 2 | 115 | 9 |
| 2105 | 1 | 93 | 18 |
| 2106 | 2 | 85 | 15 |
| 2107 | 1 | 92 | 51 |
| 2108 | 2 | 127 | 9 |
| 2109 | 1 | 105 | 18 |
| 2110 | 2 | 97 | 15 |
| 2111 | 1 | 104 | 51 |
| 2112 | 2 | 139 | 9 |
| 2113 | 1 | 11 | 26 |
| 2114 | 2 | 109 | 49 |
| 2115 | 1 | 45 | 49 |
| 2116 | 2 | 111 | 51 |
| 2117 | 1 | 23 | 26 |
| 2118 | 2 | 121 | 49 |
| 2119 | 1 | 57 | 49 |
| 2120 | 2 | 123 | 51 |
| 2121 | 1 | 35 | 26 |
| 2122 | 2 | 133 | 49 |
| 2123 | 1 | 69 | 49 |
| 2124 | 2 | 135 | 51 |
| 2125 | 1 | 75 | 56 |
| 2126 | 2 | 1 | 21 |
| 2127 | 1 | 41 | 51 |
| 2128 | 2 | 116 | 50 |
| 2129 | 1 | 87 | 56 |
| 2130 | 2 | 13 | 21 |
| 2131 | 1 | 53 | 51 |
| 2132 | 2 | 128 | 50 |
| 2133 | 1 | 99 | 56 |
| 2134 | 2 | 25 | 21 |
| 2135 | 1 | 65 | 51 |
| 2136 | 2 | 140 | 50 |
| 2137 | 1 | 5 | 23 |
| 2138 | 2 | 40 | 24 |
| 2139 | 1 | 74 | 57 |
| 2140 | 2 | 12 | 57 |
| 2141 | 1 | 17 | 23 |
| 2142 | 2 | 52 | 24 |
| 2143 | 1 | 86 | 57 |
| 2144 | 2 | 24 | 57 |
| 2145 | 1 | 29 | 23 |
| 2146 | 2 | 64 | 24 |
| 2147 | 1 | 98 | 57 |
| 2148 | 2 | 36 | 57 |
| 2149 | 1 | 84 | 57 |
| 2150 | 2 | 46 | 49 |
| 2151 | 1 | 43 | 24 |
| 2152 | 2 | 37 | 52 |
| 2153 | 1 | 96 | 57 |
| 2154 | 2 | 58 | 49 |
| 2155 | 1 | 55 | 24 |
| 2156 | 2 | 49 | 52 |
| 2157 | 1 | 108 | 57 |
| 2158 | 2 | 70 | 49 |
| 2159 | 1 | 67 | 24 |
| 2160 | 2 | 61 | 52 |
| 2161 | 1 | 2 | 54 |
| 2162 | 2 | 109 | 50 |
| 2163 | 1 | 112 | 19 |
| 2164 | 2 | 1 | 22 |
| 2165 | 1 | 14 | 54 |
| 2166 | 2 | 121 | 50 |
| 2167 | 1 | 124 | 19 |
| 2168 | 2 | 13 | 22 |
| 2169 | 1 | 26 | 54 |
| 2170 | 2 | 133 | 50 |
| 2171 | 1 | 136 | 19 |
| 2172 | 2 | 25 | 22 |
| 2173 | 1 | 42 | 52 |
| 2174 | 2 | 83 | 23 |
| 2175 | 1 | 48 | 56 |
| 2176 | 2 | 37 | 19 |
| 2177 | 1 | 54 | 52 |
| 2178 | 2 | 95 | 23 |
| 2179 | 1 | 60 | 56 |
| 2180 | 2 | 49 | 19 |
| 2181 | 1 | 66 | 52 |
| 2182 | 2 | 107 | 23 |
| 2183 | 1 | 72 | 56 |
| 2184 | 2 | 61 | 19 |
| 2185 | 1 | 7 | 23 |
| 2186 | 2 | 110 | 56 |
| 2187 | 1 | 75 | 26 |
| 2188 | 2 | 109 | 19 |
| 2189 | 1 | 19 | 23 |
| 2190 | 2 | 122 | 56 |
| 2191 | 1 | 87 | 26 |
| 2192 | 2 | 121 | 19 |
| 2193 | 1 | 31 | 23 |
| 2194 | 2 | 134 | 56 |
| 2195 | 1 | 99 | 26 |
| 2196 | 2 | 133 | 19 |
| 2197 | 1 | 81 | 26 |
| 2198 | 2 | 73 | 23 |
| 2199 | 1 | 80 | 59 |
| 2200 | 2 | 115 | 17 |
| 2201 | 1 | 93 | 26 |
| 2202 | 2 | 85 | 23 |
| 2203 | 1 | 92 | 59 |
| 2204 | 2 | 127 | 17 |
| 2205 | 1 | 105 | 26 |
| 2206 | 2 | 97 | 23 |
| 2207 | 1 | 104 | 59 |
| 2208 | 2 | 139 | 17 |
| 2209 | 1 | 11 | 34 |
| 2210 | 2 | 109 | 57 |
| 2211 | 1 | 45 | 57 |
| 2212 | 2 | 111 | 59 |
| 2213 | 1 | 23 | 34 |
| 2214 | 2 | 121 | 57 |
| 2215 | 1 | 57 | 57 |
| 2216 | 2 | 123 | 59 |
| 2217 | 1 | 35 | 34 |
| 2218 | 2 | 133 | 57 |
| 2219 | 1 | 69 | 57 |
| 2220 | 2 | 135 | 59 |
| 2221 | 1 | 75 | 64 |
| 2222 | 2 | 1 | 29 |
| 2223 | 1 | 41 | 59 |
| 2224 | 2 | 116 | 58 |
| 2225 | 1 | 87 | 64 |
| 2226 | 2 | 13 | 29 |
| 2227 | 1 | 53 | 59 |
| 2228 | 2 | 128 | 58 |
| 2229 | 1 | 99 | 64 |
| 2230 | 2 | 25 | 29 |
| 2231 | 1 | 65 | 59 |
| 2232 | 2 | 140 | 58 |
| 2233 | 1 | 5 | 31 |
| 2234 | 2 | 40 | 32 |
| 2235 | 1 | 74 | 65 |
| 2236 | 2 | 12 | 65 |
| 2237 | 1 | 17 | 31 |
| 2238 | 2 | 52 | 32 |
| 2239 | 1 | 86 | 65 |
| 2240 | 2 | 24 | 65 |
| 2241 | 1 | 29 | 31 |
| 2242 | 2 | 64 | 32 |
| 2243 | 1 | 98 | 65 |
| 2244 | 2 | 36 | 65 |
| 2245 | 1 | 84 | 65 |
| 2246 | 2 | 46 | 57 |
| 2247 | 1 | 43 | 32 |
| 2248 | 2 | 37 | 60 |
| 2249 | 1 | 96 | 65 |
| 2250 | 2 | 58 | 57 |
| 2251 | 1 | 55 | 32 |
| 2252 | 2 | 49 | 60 |
| 2253 | 1 | 108 | 65 |
| 2254 | 2 | 70 | 57 |
| 2255 | 1 | 67 | 32 |
| 2256 | 2 | 61 | 60 |
| 2257 | 1 | 2 | 62 |
| 2258 | 2 | 109 | 58 |
| 2259 | 1 | 112 | 27 |
| 2260 | 2 | 1 | 30 |
| 2261 | 1 | 14 | 62 |
| 2262 | 2 | 121 | 58 |
| 2263 | 1 | 124 | 27 |
| 2264 | 2 | 13 | 30 |
| 2265 | 1 | 26 | 62 |
| 2266 | 2 | 133 | 58 |
| 2267 | 1 | 136 | 27 |
| 2268 | 2 | 25 | 30 |
| 2269 | 1 | 42 | 60 |
| 2270 | 2 | 83 | 31 |
| 2271 | 1 | 48 | 64 |
| 2272 | 2 | 37 | 27 |
| 2273 | 1 | 54 | 60 |
| 2274 | 2 | 95 | 31 |
| 2275 | 1 | 60 | 64 |
| 2276 | 2 | 49 | 27 |
| 2277 | 1 | 66 | 60 |
| 2278 | 2 | 107 | 31 |
| 2279 | 1 | 72 | 64 |
| 2280 | 2 | 61 | 27 |
| 2281 | 1 | 7 | 31 |
| 2282 | 2 | 110 | 64 |
| 2283 | 1 | 75 | 34 |
| 2284 | 2 | 109 | 27 |
| 2285 | 1 | 19 | 31 |
| 2286 | 2 | 122 | 64 |
| 2287 | 1 | 87 | 34 |
| 2288 | 2 | 121 | 27 |
| 2289 | 1 | 31 | 31 |
| 2290 | 2 | 134 | 64 |
| 2291 | 1 | 99 | 34 |
| 2292 | 2 | 133 | 27 |
| 2293 | 1 | 81 | 34 |
| 2294 | 2 | 73 | 31 |
| 2295 | 1 | 80 | 67 |
| 2296 | 2 | 115 | 25 |
| 2297 | 1 | 93 | 34 |
| 2298 | 2 | 85 | 31 |
| 2299 | 1 | 92 | 67 |
| 2300 | 2 | 127 | 25 |
| 2301 | 1 | 105 | 34 |
| 2302 | 2 | 97 | 31 |
| 2303 | 1 | 104 | 67 |
| 2304 | 2 | 139 | 25 |
| 2305 | 1 | 12 | 42 |
| 2306 | 2 | 7 | 8 |
| 2307 | 1 | 119 | 40 |
| 2308 | 2 | 73 | 38 |
| 2309 | 1 | 24 | 42 |
| 2310 | 2 | 19 | 8 |
| 2311 | 1 | 131 | 40 |
| 2312 | 2 | 85 | 38 |
| 2313 | 1 | 36 | 42 |
| 2314 | 2 | 31 | 8 |
| 2315 | 1 | 143 | 40 |
| 2316 | 2 | 97 | 38 |
| 2317 | 1 | 110 | 37 |
| 2318 | 2 | 10 | 43 |
| 2319 | 1 | 44 | 6 |
| 2320 | 2 | 115 | 34 |
| 2321 | 1 | 122 | 37 |
| 2322 | 2 | 22 | 43 |
| 2323 | 1 | 56 | 6 |
| 2324 | 2 | 127 | 34 |
| 2325 | 1 | 134 | 37 |
| 2326 | 2 | 34 | 43 |
| 2327 | 1 | 68 | 6 |
| 2328 | 2 | 139 | 34 |
| 2329 | 1 | 46 | 8 |
| 2330 | 2 | 1 | 38 |
| 2331 | 1 | 83 | 39 |
| 2332 | 2 | 46 | 7 |
| 2333 | 1 | 58 | 8 |
| 2334 | 2 | 13 | 38 |
| 2335 | 1 | 95 | 39 |
| 2336 | 2 | 58 | 7 |
| 2337 | 1 | 70 | 8 |
| 2338 | 2 | 25 | 38 |
| 2339 | 1 | 107 | 39 |
| 2340 | 2 | 70 | 7 |
| 2341 | 1 | 45 | 36 |
| 2342 | 2 | 37 | 35 |
| 2343 | 1 | 2 | 10 |
| 2344 | 2 | 112 | 33 |
| 2345 | 1 | 57 | 36 |
| 2346 | 2 | 49 | 35 |
| 2347 | 1 | 14 | 10 |
| 2348 | 2 | 124 | 33 |
| 2349 | 1 | 69 | 36 |
| 2350 | 2 | 61 | 35 |
| 2351 | 1 | 26 | 10 |
| 2352 | 2 | 136 | 33 |
| 2353 | 1 | 5 | 4 |
| 2354 | 2 | 73 | 43 |
| 2355 | 1 | 46 | 6 |
| 2356 | 2 | 76 | 7 |
| 2357 | 1 | 17 | 4 |
| 2358 | 2 | 85 | 43 |
| 2359 | 1 | 58 | 6 |
| 2360 | 2 | 88 | 7 |
| 2361 | 1 | 29 | 4 |
| 2362 | 2 | 97 | 43 |
| 2363 | 1 | 70 | 6 |
| 2364 | 2 | 100 | 7 |
| 2365 | 1 | 77 | 36 |
| 2366 | 2 | 110 | 6 |
| 2367 | 1 | 115 | 8 |
| 2368 | 2 | 42 | 34 |
| 2369 | 1 | 89 | 36 |
| 2370 | 2 | 122 | 6 |
| 2371 | 1 | 127 | 8 |
| 2372 | 2 | 54 | 34 |
| 2373 | 1 | 101 | 36 |
| 2374 | 2 | 134 | 6 |
| 2375 | 1 | 139 | 8 |
| 2376 | 2 | 66 | 34 |
| 2377 | 1 | 81 | 43 |
| 2378 | 2 | 12 | 37 |
| 2379 | 1 | 74 | 8 |
| 2380 | 2 | 118 | 35 |
| 2381 | 1 | 93 | 43 |
| 2382 | 2 | 24 | 37 |
| 2383 | 1 | 86 | 8 |
| 2384 | 2 | 130 | 35 |
| 2385 | 1 | 105 | 43 |
| 2386 | 2 | 36 | 37 |
| 2387 | 1 | 98 | 8 |
| 2388 | 2 | 142 | 35 |
| 2389 | 1 | 119 | 7 |
| 2390 | 2 | 84 | 43 |
| 2391 | 1 | 82 | 11 |
| 2392 | 2 | 39 | 6 |
| 2393 | 1 | 131 | 7 |
| 2394 | 2 | 96 | 43 |
| 2395 | 1 | 94 | 11 |
| 2396 | 2 | 51 | 6 |
| 2397 | 1 | 143 | 7 |
| 2398 | 2 | 108 | 43 |
| 2399 | 1 | 106 | 11 |
| 2400 | 2 | 63 | 6 |
| 2401 | 1 | 12 | 50 |
| 2402 | 2 | 7 | 16 |
| 2403 | 1 | 119 | 48 |
| 2404 | 2 | 73 | 46 |
| 2405 | 1 | 24 | 50 |
| 2406 | 2 | 19 | 16 |
| 2407 | 1 | 131 | 48 |
| 2408 | 2 | 85 | 46 |
| 2409 | 1 | 36 | 50 |
| 2410 | 2 | 31 | 16 |
| 2411 | 1 | 143 | 48 |
| 2412 | 2 | 97 | 46 |
| 2413 | 1 | 110 | 45 |
| 2414 | 2 | 10 | 51 |
| 2415 | 1 | 44 | 14 |
| 2416 | 2 | 115 | 42 |
| 2417 | 1 | 122 | 45 |
| 2418 | 2 | 22 | 51 |
| 2419 | 1 | 56 | 14 |
| 2420 | 2 | 127 | 42 |
| 2421 | 1 | 134 | 45 |
| 2422 | 2 | 34 | 51 |
| 2423 | 1 | 68 | 14 |
| 2424 | 2 | 139 | 42 |
| 2425 | 1 | 46 | 16 |
| 2426 | 2 | 1 | 46 |
| 2427 | 1 | 83 | 47 |
| 2428 | 2 | 46 | 15 |
| 2429 | 1 | 58 | 16 |
| 2430 | 2 | 13 | 46 |
| 2431 | 1 | 95 | 47 |
| 2432 | 2 | 58 | 15 |
| 2433 | 1 | 70 | 16 |
| 2434 | 2 | 25 | 46 |
| 2435 | 1 | 107 | 47 |
| 2436 | 2 | 70 | 15 |
| 2437 | 1 | 45 | 44 |
| 2438 | 2 | 37 | 43 |
| 2439 | 1 | 2 | 18 |
| 2440 | 2 | 112 | 41 |
| 2441 | 1 | 57 | 44 |
| 2442 | 2 | 49 | 43 |
| 2443 | 1 | 14 | 18 |
| 2444 | 2 | 124 | 41 |
| 2445 | 1 | 69 | 44 |
| 2446 | 2 | 61 | 43 |
| 2447 | 1 | 26 | 18 |
| 2448 | 2 | 136 | 41 |
| 2449 | 1 | 5 | 12 |
| 2450 | 2 | 73 | 51 |
| 2451 | 1 | 46 | 14 |
| 2452 | 2 | 76 | 15 |
| 2453 | 1 | 17 | 12 |
| 2454 | 2 | 85 | 51 |
| 2455 | 1 | 58 | 14 |
| 2456 | 2 | 88 | 15 |
| 2457 | 1 | 29 | 12 |
| 2458 | 2 | 97 | 51 |
| 2459 | 1 | 70 | 14 |
| 2460 | 2 | 100 | 15 |
| 2461 | 1 | 77 | 44 |
| 2462 | 2 | 110 | 14 |
| 2463 | 1 | 115 | 16 |
| 2464 | 2 | 42 | 42 |
| 2465 | 1 | 89 | 44 |
| 2466 | 2 | 122 | 14 |
| 2467 | 1 | 127 | 16 |
| 2468 | 2 | 54 | 42 |
| 2469 | 1 | 101 | 44 |
| 2470 | 2 | 134 | 14 |
| 2471 | 1 | 139 | 16 |
| 2472 | 2 | 66 | 42 |
| 2473 | 1 | 81 | 51 |
| 2474 | 2 | 12 | 45 |
| 2475 | 1 | 74 | 16 |
| 2476 | 2 | 118 | 43 |
| 2477 | 1 | 93 | 51 |
| 2478 | 2 | 24 | 45 |
| 2479 | 1 | 86 | 16 |
| 2480 | 2 | 130 | 43 |
| 2481 | 1 | 105 | 51 |
| 2482 | 2 | 36 | 45 |
| 2483 | 1 | 98 | 16 |
| 2484 | 2 | 142 | 43 |
| 2485 | 1 | 119 | 15 |
| 2486 | 2 | 84 | 51 |
| 2487 | 1 | 82 | 19 |
| 2488 | 2 | 39 | 14 |
| 2489 | 1 | 131 | 15 |
| 2490 | 2 | 96 | 51 |
| 2491 | 1 | 94 | 19 |
| 2492 | 2 | 51 | 14 |
| 2493 | 1 | 143 | 15 |
| 2494 | 2 | 108 | 51 |
| 2495 | 1 | 106 | 19 |
| 2496 | 2 | 63 | 14 |
| 2497 | 1 | 12 | 58 |
| 2498 | 2 | 7 | 24 |
| 2499 | 1 | 119 | 56 |
| 2500 | 2 | 73 | 54 |
| 2501 | 1 | 24 | 58 |
| 2502 | 2 | 19 | 24 |
| 2503 | 1 | 131 | 56 |
| 2504 | 2 | 85 | 54 |
| 2505 | 1 | 36 | 58 |
| 2506 | 2 | 31 | 24 |
| 2507 | 1 | 143 | 56 |
| 2508 | 2 | 97 | 54 |
| 2509 | 1 | 110 | 53 |
| 2510 | 2 | 10 | 59 |
| 2511 | 1 | 44 | 22 |
| 2512 | 2 | 115 | 50 |
| 2513 | 1 | 122 | 53 |
| 2514 | 2 | 22 | 59 |
| 2515 | 1 | 56 | 22 |
| 2516 | 2 | 127 | 50 |
| 2517 | 1 | 134 | 53 |
| 2518 | 2 | 34 | 59 |
| 2519 | 1 | 68 | 22 |
| 2520 | 2 | 139 | 50 |
| 2521 | 1 | 46 | 24 |
| 2522 | 2 | 1 | 54 |
| 2523 | 1 | 83 | 55 |
| 2524 | 2 | 46 | 23 |
| 2525 | 1 | 58 | 24 |
| 2526 | 2 | 13 | 54 |
| 2527 | 1 | 95 | 55 |
| 2528 | 2 | 58 | 23 |
| 2529 | 1 | 70 | 24 |
| 2530 | 2 | 25 | 54 |
| 2531 | 1 | 107 | 55 |
| 2532 | 2 | 70 | 23 |
| 2533 | 1 | 45 | 52 |
| 2534 | 2 | 37 | 51 |
| 2535 | 1 | 2 | 26 |
| 2536 | 2 | 112 | 49 |
| 2537 | 1 | 57 | 52 |
| 2538 | 2 | 49 | 51 |
| 2539 | 1 | 14 | 26 |
| 2540 | 2 | 124 | 49 |
| 2541 | 1 | 69 | 52 |
| 2542 | 2 | 61 | 51 |
| 2543 | 1 | 26 | 26 |
| 2544 | 2 | 136 | 49 |
| 2545 | 1 | 5 | 20 |
| 2546 | 2 | 73 | 59 |
| 2547 | 1 | 46 | 22 |
| 2548 | 2 | 76 | 23 |
| 2549 | 1 | 17 | 20 |
| 2550 | 2 | 85 | 59 |
| 2551 | 1 | 58 | 22 |
| 2552 | 2 | 88 | 23 |
| 2553 | 1 | 29 | 20 |
| 2554 | 2 | 97 | 59 |
| 2555 | 1 | 70 | 22 |
| 2556 | 2 | 100 | 23 |
| 2557 | 1 | 77 | 52 |
| 2558 | 2 | 110 | 22 |
| 2559 | 1 | 115 | 24 |
| 2560 | 2 | 42 | 50 |
| 2561 | 1 | 89 | 52 |
| 2562 | 2 | 122 | 22 |
| 2563 | 1 | 127 | 24 |
| 2564 | 2 | 54 | 50 |
| 2565 | 1 | 101 | 52 |
| 2566 | 2 | 134 | 22 |
| 2567 | 1 | 139 | 24 |
| 2568 | 2 | 66 | 50 |
| 2569 | 1 | 81 | 59 |
| 2570 | 2 | 12 | 53 |
| 2571 | 1 | 74 | 24 |
| 2572 | 2 | 118 | 51 |
| 2573 | 1 | 93 | 59 |
| 2574 | 2 | 24 | 53 |
| 2575 | 1 | 86 | 24 |
| 2576 | 2 | 130 | 51 |
| 2577 | 1 | 105 | 59 |
| 2578 | 2 | 36 | 53 |
| 2579 | 1 | 98 | 24 |
| 2580 | 2 | 142 | 51 |
| 2581 | 1 | 119 | 23 |
| 2582 | 2 | 84 | 59 |
| 2583 | 1 | 82 | 27 |
| 2584 | 2 | 39 | 22 |
| 2585 | 1 | 131 | 23 |
| 2586 | 2 | 96 | 59 |
| 2587 | 1 | 94 | 27 |
| 2588 | 2 | 51 | 22 |
| 2589 | 1 | 143 | 23 |
| 2590 | 2 | 108 | 59 |
| 2591 | 1 | 106 | 27 |
| 2592 | 2 | 63 | 22 |
| 2593 | 1 | 12 | 66 |
| 2594 | 2 | 7 | 32 |
| 2595 | 1 | 119 | 64 |
| 2596 | 2 | 73 | 62 |
| 2597 | 1 | 24 | 66 |
| 2598 | 2 | 19 | 32 |
| 2599 | 1 | 131 | 64 |
| 2600 | 2 | 85 | 62 |
| 2601 | 1 | 36 | 66 |
| 2602 | 2 | 31 | 32 |
| 2603 | 1 | 143 | 64 |
| 2604 | 2 | 97 | 62 |
| 2605 | 1 | 110 | 61 |
| 2606 | 2 | 10 | 67 |
| 2607 | 1 | 44 | 30 |
| 2608 | 2 | 115 | 58 |
| 2609 | 1 | 122 | 61 |
| 2610 | 2 | 22 | 67 |
| 2611 | 1 | 56 | 30 |
| 2612 | 2 | 127 | 58 |
| 2613 | 1 | 134 | 61 |
| 2614 | 2 | 34 | 67 |
| 2615 | 1 | 68 | 30 |
| 2616 | 2 | 139 | 58 |
| 2617 | 1 | 46 | 32 |
| 2618 | 2 | 1 | 62 |
| 2619 | 1 | 83 | 63 |
| 2620 | 2 | 46 | 31 |
| 2621 | 1 | 58 | 32 |
| 2622 | 2 | 13 | 62 |
| 2623 | 1 | 95 | 63 |
| 2624 | 2 | 58 | 31 |
| 2625 | 1 | 70 | 32 |
| 2626 | 2 | 25 | 62 |
| 2627 | 1 | 107 | 63 |
| 2628 | 2 | 70 | 31 |
| 2629 | 1 | 45 | 60 |
| 2630 | 2 | 37 | 59 |
| 2631 | 1 | 2 | 34 |
| 2632 | 2 | 112 | 57 |
| 2633 | 1 | 57 | 60 |
| 2634 | 2 | 49 | 59 |
| 2635 | 1 | 14 | 34 |
| 2636 | 2 | 124 | 57 |
| 2637 | 1 | 69 | 60 |
| 2638 | 2 | 61 | 59 |
| 2639 | 1 | 26 | 34 |
| 2640 | 2 | 136 | 57 |
| 2641 | 1 | 5 | 28 |
| 2642 | 2 | 73 | 67 |
| 2643 | 1 | 46 | 30 |
| 2644 | 2 | 76 | 31 |
| 2645 | 1 | 17 | 28 |
| 2646 | 2 | 85 | 67 |
| 2647 | 1 | 58 | 30 |
| 2648 | 2 | 88 | 31 |
| 2649 | 1 | 29 | 28 |
| 2650 | 2 | 97 | 67 |
| 2651 | 1 | 70 | 30 |
| 2652 | 2 | 100 | 31 |
| 2653 | 1 | 77 | 60 |
| 2654 | 2 | 110 | 30 |
| 2655 | 1 | 115 | 32 |
| 2656 | 2 | 42 | 58 |
| 2657 | 1 | 89 | 60 |
| 2658 | 2 | 122 | 30 |
| 2659 | 1 | 127 | 32 |
| 2660 | 2 | 54 | 58 |
| 2661 | 1 | 101 | 60 |
| 2662 | 2 | 134 | 30 |
| 2663 | 1 | 139 | 32 |
| 2664 | 2 | 66 | 58 |
| 2665 | 1 | 81 | 67 |
| 2666 | 2 | 12 | 61 |
| 2667 | 1 | 74 | 32 |
| 2668 | 2 | 118 | 59 |
| 2669 | 1 | 93 | 67 |
| 2670 | 2 | 24 | 61 |
| 2671 | 1 | 86 | 32 |
| 2672 | 2 | 130 | 59 |
| 2673 | 1 | 105 | 67 |
| 2674 | 2 | 36 | 61 |
| 2675 | 1 | 98 | 32 |
| 2676 | 2 | 142 | 59 |
| 2677 | 1 | 119 | 31 |
| 2678 | 2 | 84 | 67 |
| 2679 | 1 | 82 | 35 |
| 2680 | 2 | 39 | 30 |
| 2681 | 1 | 131 | 31 |
| 2682 | 2 | 96 | 67 |
| 2683 | 1 | 94 | 35 |
| 2684 | 2 | 51 | 30 |
| 2685 | 1 | 143 | 31 |
| 2686 | 2 | 108 | 67 |
| 2687 | 1 | 106 | 35 |
| 2688 | 2 | 63 | 30 |
| 2689 | 1 | 78 | 6 |
| 2690 | 2 | 3 | 5 |
| 2691 | 1 | 77 | 11 |
| 2692 | 2 | 42 | 6 |
| 2693 | 1 | 90 | 6 |
| 2694 | 2 | 15 | 5 |
| 2695 | 1 | 89 | 11 |
| 2696 | 2 | 54 | 6 |
| 2697 | 1 | 102 | 6 |
| 2698 | 2 | 27 | 5 |
| 2699 | 1 | 101 | 11 |
| 2700 | 2 | 66 | 6 |
| 2701 | 1 | 81 | 7 |
| 2702 | 2 | 2 | 40 |
| 2703 | 1 | 118 | 1 |
| 2704 | 2 | 3 | 4 |
| 2705 | 1 | 93 | 7 |
| 2706 | 2 | 14 | 40 |
| 2707 | 1 | 130 | 1 |
| 2708 | 2 | 15 | 4 |
| 2709 | 1 | 105 | 7 |
| 2710 | 2 | 26 | 40 |
| 2711 | 1 | 142 | 1 |
| 2712 | 2 | 27 | 4 |
| 2713 | 1 | 77 | 39 |
| 2714 | 2 | 109 | 35 |
| 2715 | 1 | 75 | 38 |
| 2716 | 2 | 41 | 5 |
| 2717 | 1 | 89 | 39 |
| 2718 | 2 | 121 | 35 |
| 2719 | 1 | 87 | 38 |
| 2720 | 2 | 53 | 5 |
| 2721 | 1 | 101 | 39 |
| 2722 | 2 | 133 | 35 |
| 2723 | 1 | 99 | 38 |
| 2724 | 2 | 65 | 5 |
| 2725 | 1 | 112 | 36 |
| 2726 | 2 | 8 | 38 |
| 2727 | 1 | 115 | 7 |
| 2728 | 2 | 114 | 37 |
| 2729 | 1 | 124 | 36 |
| 2730 | 2 | 20 | 38 |
| 2731 | 1 | 127 | 7 |
| 2732 | 2 | 126 | 37 |
| 2733 | 1 | 136 | 36 |
| 2734 | 2 | 32 | 38 |
| 2735 | 1 | 139 | 7 |
| 2736 | 2 | 138 | 37 |
| 2737 | 1 | 79 | 11 |
| 2738 | 2 | 81 | 8 |
| 2739 | 1 | 83 | 6 |
| 2740 | 2 | 110 | 36 |
| 2741 | 1 | 91 | 11 |
| 2742 | 2 | 93 | 8 |
| 2743 | 1 | 95 | 6 |
| 2744 | 2 | 122 | 36 |
| 2745 | 1 | 103 | 11 |
| 2746 | 2 | 105 | 8 |
| 2747 | 1 | 107 | 6 |
| 2748 | 2 | 134 | 36 |
| 2749 | 1 | 112 | 5 |
| 2750 | 2 | 79 | 8 |
| 2751 | 1 | 4 | 11 |
| 2752 | 2 | 48 | 39 |
| 2753 | 1 | 124 | 5 |
| 2754 | 2 | 91 | 8 |
| 2755 | 1 | 16 | 11 |
| 2756 | 2 | 60 | 39 |
| 2757 | 1 | 136 | 5 |
| 2758 | 2 | 103 | 8 |
| 2759 | 1 | 28 | 11 |
| 2760 | 2 | 72 | 39 |
| 2761 | 1 | 115 | 4 |
| 2762 | 2 | 109 | 4 |
| 2763 | 1 | 45 | 8 |
| 2764 | 2 | 73 | 39 |
| 2765 | 1 | 127 | 4 |
| 2766 | 2 | 121 | 4 |
| 2767 | 1 | 57 | 8 |
| 2768 | 2 | 85 | 39 |
| 2769 | 1 | 139 | 4 |
| 2770 | 2 | 133 | 4 |
| 2771 | 1 | 69 | 8 |
| 2772 | 2 | 97 | 39 |
| 2773 | 1 | 119 | 8 |
| 2774 | 2 | 12 | 40 |
| 2775 | 1 | 109 | 36 |
| 2776 | 2 | 78 | 5 |
| 2777 | 1 | 131 | 8 |
| 2778 | 2 | 24 | 40 |
| 2779 | 1 | 121 | 36 |
| 2780 | 2 | 90 | 5 |
| 2781 | 1 | 143 | 8 |
| 2782 | 2 | 36 | 40 |
| 2783 | 1 | 133 | 36 |
| 2784 | 2 | 102 | 5 |
| 2785 | 1 | 78 | 14 |
| 2786 | 2 | 3 | 13 |
| 2787 | 1 | 77 | 19 |
| 2788 | 2 | 42 | 14 |
| 2789 | 1 | 90 | 14 |
| 2790 | 2 | 15 | 13 |
| 2791 | 1 | 89 | 19 |
| 2792 | 2 | 54 | 14 |
| 2793 | 1 | 102 | 14 |
| 2794 | 2 | 27 | 13 |
| 2795 | 1 | 101 | 19 |
| 2796 | 2 | 66 | 14 |
| 2797 | 1 | 81 | 15 |
| 2798 | 2 | 2 | 48 |
| 2799 | 1 | 118 | 9 |
| 2800 | 2 | 3 | 12 |
| 2801 | 1 | 93 | 15 |
| 2802 | 2 | 14 | 48 |
| 2803 | 1 | 130 | 9 |
| 2804 | 2 | 15 | 12 |
| 2805 | 1 | 105 | 15 |
| 2806 | 2 | 26 | 48 |
| 2807 | 1 | 142 | 9 |
| 2808 | 2 | 27 | 12 |
| 2809 | 1 | 77 | 47 |
| 2810 | 2 | 109 | 43 |
| 2811 | 1 | 75 | 46 |
| 2812 | 2 | 41 | 13 |
| 2813 | 1 | 89 | 47 |
| 2814 | 2 | 121 | 43 |
| 2815 | 1 | 87 | 46 |
| 2816 | 2 | 53 | 13 |
| 2817 | 1 | 101 | 47 |
| 2818 | 2 | 133 | 43 |
| 2819 | 1 | 99 | 46 |
| 2820 | 2 | 65 | 13 |
| 2821 | 1 | 112 | 44 |
| 2822 | 2 | 8 | 46 |
| 2823 | 1 | 115 | 15 |
| 2824 | 2 | 114 | 45 |
| 2825 | 1 | 124 | 44 |
| 2826 | 2 | 20 | 46 |
| 2827 | 1 | 127 | 15 |
| 2828 | 2 | 126 | 45 |
| 2829 | 1 | 136 | 44 |
| 2830 | 2 | 32 | 46 |
| 2831 | 1 | 139 | 15 |
| 2832 | 2 | 138 | 45 |
| 2833 | 1 | 79 | 19 |
| 2834 | 2 | 81 | 16 |
| 2835 | 1 | 83 | 14 |
| 2836 | 2 | 110 | 44 |
| 2837 | 1 | 91 | 19 |
| 2838 | 2 | 93 | 16 |
| 2839 | 1 | 95 | 14 |
| 2840 | 2 | 122 | 44 |
| 2841 | 1 | 103 | 19 |
| 2842 | 2 | 105 | 16 |
| 2843 | 1 | 107 | 14 |
| 2844 | 2 | 134 | 44 |
| 2845 | 1 | 112 | 13 |
| 2846 | 2 | 79 | 16 |
| 2847 | 1 | 4 | 19 |
| 2848 | 2 | 48 | 47 |
| 2849 | 1 | 124 | 13 |
| 2850 | 2 | 91 | 16 |
| 2851 | 1 | 16 | 19 |
| 2852 | 2 | 60 | 47 |
| 2853 | 1 | 136 | 13 |
| 2854 | 2 | 103 | 16 |
| 2855 | 1 | 28 | 19 |
| 2856 | 2 | 72 | 47 |
| 2857 | 1 | 115 | 12 |
| 2858 | 2 | 109 | 12 |
| 2859 | 1 | 45 | 16 |
| 2860 | 2 | 73 | 47 |
| 2861 | 1 | 127 | 12 |
| 2862 | 2 | 121 | 12 |
| 2863 | 1 | 57 | 16 |
| 2864 | 2 | 85 | 47 |
| 2865 | 1 | 139 | 12 |
| 2866 | 2 | 133 | 12 |
| 2867 | 1 | 69 | 16 |
| 2868 | 2 | 97 | 47 |
| 2869 | 1 | 119 | 16 |
| 2870 | 2 | 12 | 48 |
| 2871 | 1 | 109 | 44 |
| 2872 | 2 | 78 | 13 |
| 2873 | 1 | 131 | 16 |
| 2874 | 2 | 24 | 48 |
| 2875 | 1 | 121 | 44 |
| 2876 | 2 | 90 | 13 |
| 2877 | 1 | 143 | 16 |
| 2878 | 2 | 36 | 48 |
| 2879 | 1 | 133 | 44 |
| 2880 | 2 | 102 | 13 |
| 2881 | 1 | 78 | 22 |
| 2882 | 2 | 3 | 21 |
| 2883 | 1 | 77 | 27 |
| 2884 | 2 | 42 | 22 |
| 2885 | 1 | 90 | 22 |
| 2886 | 2 | 15 | 21 |
| 2887 | 1 | 89 | 27 |
| 2888 | 2 | 54 | 22 |
| 2889 | 1 | 102 | 22 |
| 2890 | 2 | 27 | 21 |
| 2891 | 1 | 101 | 27 |
| 2892 | 2 | 66 | 22 |
| 2893 | 1 | 81 | 23 |
| 2894 | 2 | 2 | 56 |
| 2895 | 1 | 118 | 17 |
| 2896 | 2 | 3 | 20 |
| 2897 | 1 | 93 | 23 |
| 2898 | 2 | 14 | 56 |
| 2899 | 1 | 130 | 17 |
| 2900 | 2 | 15 | 20 |
| 2901 | 1 | 105 | 23 |
| 2902 | 2 | 26 | 56 |
| 2903 | 1 | 142 | 17 |
| 2904 | 2 | 27 | 20 |
| 2905 | 1 | 77 | 55 |
| 2906 | 2 | 109 | 51 |
| 2907 | 1 | 75 | 54 |
| 2908 | 2 | 41 | 21 |
| 2909 | 1 | 89 | 55 |
| 2910 | 2 | 121 | 51 |
| 2911 | 1 | 87 | 54 |
| 2912 | 2 | 53 | 21 |
| 2913 | 1 | 101 | 55 |
| 2914 | 2 | 133 | 51 |
| 2915 | 1 | 99 | 54 |
| 2916 | 2 | 65 | 21 |
| 2917 | 1 | 112 | 52 |
| 2918 | 2 | 8 | 54 |
| 2919 | 1 | 115 | 23 |
| 2920 | 2 | 114 | 53 |
| 2921 | 1 | 124 | 52 |
| 2922 | 2 | 20 | 54 |
| 2923 | 1 | 127 | 23 |
| 2924 | 2 | 126 | 53 |
| 2925 | 1 | 136 | 52 |
| 2926 | 2 | 32 | 54 |
| 2927 | 1 | 139 | 23 |
| 2928 | 2 | 138 | 53 |
| 2929 | 1 | 79 | 27 |
| 2930 | 2 | 81 | 24 |
| 2931 | 1 | 83 | 22 |
| 2932 | 2 | 110 | 52 |
| 2933 | 1 | 91 | 27 |
| 2934 | 2 | 93 | 24 |
| 2935 | 1 | 95 | 22 |
| 2936 | 2 | 122 | 52 |
| 2937 | 1 | 103 | 27 |
| 2938 | 2 | 105 | 24 |
| 2939 | 1 | 107 | 22 |
| 2940 | 2 | 134 | 52 |
| 2941 | 1 | 112 | 21 |
| 2942 | 2 | 79 | 24 |
| 2943 | 1 | 4 | 27 |
| 2944 | 2 | 48 | 55 |
| 2945 | 1 | 124 | 21 |
| 2946 | 2 | 91 | 24 |
| 2947 | 1 | 16 | 27 |
| 2948 | 2 | 60 | 55 |
| 2949 | 1 | 136 | 21 |
| 2950 | 2 | 103 | 24 |
| 2951 | 1 | 28 | 27 |
| 2952 | 2 | 72 | 55 |
| 2953 | 1 | 115 | 20 |
| 2954 | 2 | 109 | 20 |
| 2955 | 1 | 45 | 24 |
| 2956 | 2 | 73 | 55 |
| 2957 | 1 | 127 | 20 |
| 2958 | 2 | 121 | 20 |
| 2959 | 1 | 57 | 24 |
| 2960 | 2 | 85 | 55 |
| 2961 | 1 | 139 | 20 |
| 2962 | 2 | 133 | 20 |
| 2963 | 1 | 69 | 24 |
| 2964 | 2 | 97 | 55 |
| 2965 | 1 | 119 | 24 |
| 2966 | 2 | 12 | 56 |
| 2967 | 1 | 109 | 52 |
| 2968 | 2 | 78 | 21 |
| 2969 | 1 | 131 | 24 |
| 2970 | 2 | 24 | 56 |
| 2971 | 1 | 121 | 52 |
| 2972 | 2 | 90 | 21 |
| 2973 | 1 | 143 | 24 |
| 2974 | 2 | 36 | 56 |
| 2975 | 1 | 133 | 52 |
| 2976 | 2 | 102 | 21 |
| 2977 | 1 | 78 | 30 |
| 2978 | 2 | 3 | 29 |
| 2979 | 1 | 77 | 35 |
| 2980 | 2 | 42 | 30 |
| 2981 | 1 | 90 | 30 |
| 2982 | 2 | 15 | 29 |
| 2983 | 1 | 89 | 35 |
| 2984 | 2 | 54 | 30 |
| 2985 | 1 | 102 | 30 |
| 2986 | 2 | 27 | 29 |
| 2987 | 1 | 101 | 35 |
| 2988 | 2 | 66 | 30 |
| 2989 | 1 | 81 | 31 |
| 2990 | 2 | 2 | 64 |
| 2991 | 1 | 118 | 25 |
| 2992 | 2 | 3 | 28 |
| 2993 | 1 | 93 | 31 |
| 2994 | 2 | 14 | 64 |
| 2995 | 1 | 130 | 25 |
| 2996 | 2 | 15 | 28 |
| 2997 | 1 | 105 | 31 |
| 2998 | 2 | 26 | 64 |
| 2999 | 1 | 142 | 25 |
| 3000 | 2 | 27 | 28 |
| 3001 | 1 | 77 | 63 |
| 3002 | 2 | 109 | 59 |
| 3003 | 1 | 75 | 62 |
| 3004 | 2 | 41 | 29 |
| 3005 | 1 | 89 | 63 |
| 3006 | 2 | 121 | 59 |
| 3007 | 1 | 87 | 62 |
| 3008 | 2 | 53 | 29 |
| 3009 | 1 | 101 | 63 |
| 3010 | 2 | 133 | 59 |
| 3011 | 1 | 99 | 62 |
| 3012 | 2 | 65 | 29 |
| 3013 | 1 | 112 | 60 |
| 3014 | 2 | 8 | 62 |
| 3015 | 1 | 115 | 31 |
| 3016 | 2 | 114 | 61 |
| 3017 | 1 | 124 | 60 |
| 3018 | 2 | 20 | 62 |
| 3019 | 1 | 127 | 31 |
| 3020 | 2 | 126 | 61 |
| 3021 | 1 | 136 | 60 |
| 3022 | 2 | 32 | 62 |
| 3023 | 1 | 139 | 31 |
| 3024 | 2 | 138 | 61 |
| 3025 | 1 | 79 | 35 |
| 3026 | 2 | 81 | 32 |
| 3027 | 1 | 83 | 30 |
| 3028 | 2 | 110 | 60 |
| 3029 | 1 | 91 | 35 |
| 3030 | 2 | 93 | 32 |
| 3031 | 1 | 95 | 30 |
| 3032 | 2 | 122 | 60 |
| 3033 | 1 | 103 | 35 |
| 3034 | 2 | 105 | 32 |
| 3035 | 1 | 107 | 30 |
| 3036 | 2 | 134 | 60 |
| 3037 | 1 | 112 | 29 |
| 3038 | 2 | 79 | 32 |
| 3039 | 1 | 4 | 35 |
| 3040 | 2 | 48 | 63 |
| 3041 | 1 | 124 | 29 |
| 3042 | 2 | 91 | 32 |
| 3043 | 1 | 16 | 35 |
| 3044 | 2 | 60 | 63 |
| 3045 | 1 | 136 | 29 |
| 3046 | 2 | 103 | 32 |
| 3047 | 1 | 28 | 35 |
| 3048 | 2 | 72 | 63 |
| 3049 | 1 | 115 | 28 |
| 3050 | 2 | 109 | 28 |
| 3051 | 1 | 45 | 32 |
| 3052 | 2 | 73 | 63 |
| 3053 | 1 | 127 | 28 |
| 3054 | 2 | 121 | 28 |
| 3055 | 1 | 57 | 32 |
| 3056 | 2 | 85 | 63 |
| 3057 | 1 | 139 | 28 |
| 3058 | 2 | 133 | 28 |
| 3059 | 1 | 69 | 32 |
| 3060 | 2 | 97 | 63 |
| 3061 | 1 | 119 | 32 |
| 3062 | 2 | 12 | 64 |
| 3063 | 1 | 109 | 60 |
| 3064 | 2 | 78 | 29 |
| 3065 | 1 | 131 | 32 |
| 3066 | 2 | 24 | 64 |
| 3067 | 1 | 121 | 60 |
| 3068 | 2 | 90 | 29 |
| 3069 | 1 | 143 | 32 |
| 3070 | 2 | 36 | 64 |
| 3071 | 1 | 133 | 60 |
| 3072 | 2 | 102 | 29 |
| 3073 | 1 | 5 | 42 |
| 3074 | 2 | 1 | 8 |
| 3075 | 1 | 3 | 42 |
| 3076 | 2 | 118 | 37 |
| 3077 | 1 | 17 | 42 |
| 3078 | 2 | 13 | 8 |
| 3079 | 1 | 15 | 42 |
| 3080 | 2 | 130 | 37 |
| 3081 | 1 | 29 | 42 |
| 3082 | 2 | 25 | 8 |
| 3083 | 1 | 27 | 42 |
| 3084 | 2 | 142 | 37 |
| 3085 | 1 | 75 | 37 |
| 3086 | 2 | 45 | 34 |
| 3087 | 1 | 119 | 1 |
| 3088 | 2 | 6 | 40 |
| 3089 | 1 | 87 | 37 |
| 3090 | 2 | 57 | 34 |
| 3091 | 1 | 131 | 1 |
| 3092 | 2 | 18 | 40 |
| 3093 | 1 | 99 | 37 |
| 3094 | 2 | 69 | 34 |
| 3095 | 1 | 143 | 1 |
| 3096 | 2 | 30 | 40 |
| 3097 | 1 | 40 | 36 |
| 3098 | 2 | 8 | 41 |
| 3099 | 1 | 11 | 38 |
| 3100 | 2 | 73 | 40 |
| 3101 | 1 | 52 | 36 |
| 3102 | 2 | 20 | 41 |
| 3103 | 1 | 23 | 38 |
| 3104 | 2 | 85 | 40 |
| 3105 | 1 | 64 | 36 |
| 3106 | 2 | 32 | 41 |
| 3107 | 1 | 35 | 38 |
| 3108 | 2 | 97 | 40 |
| 3109 | 1 | 73 | 8 |
| 3110 | 2 | 114 | 3 |
| 3111 | 1 | 7 | 6 |
| 3112 | 2 | 109 | 37 |
| 3113 | 1 | 85 | 8 |
| 3114 | 2 | 126 | 3 |
| 3115 | 1 | 19 | 6 |
| 3116 | 2 | 121 | 37 |
| 3117 | 1 | 97 | 8 |
| 3118 | 2 | 138 | 3 |
| 3119 | 1 | 31 | 6 |
| 3120 | 2 | 133 | 37 |
| 3121 | 1 | 77 | 5 |
| 3122 | 2 | 73 | 41 |
| 3123 | 1 | 43 | 1 |
| 3124 | 2 | 73 | 42 |
| 3125 | 1 | 89 | 5 |
| 3126 | 2 | 85 | 41 |
| 3127 | 1 | 55 | 1 |
| 3128 | 2 | 85 | 42 |
| 3129 | 1 | 101 | 5 |
| 3130 | 2 | 97 | 41 |
| 3131 | 1 | 67 | 1 |
| 3132 | 2 | 97 | 42 |
| 3133 | 1 | 47 | 7 |
| 3134 | 2 | 43 | 37 |
| 3135 | 1 | 115 | 33 |
| 3136 | 2 | 109 | 39 |
| 3137 | 1 | 59 | 7 |
| 3138 | 2 | 55 | 37 |
| 3139 | 1 | 127 | 33 |
| 3140 | 2 | 121 | 39 |
| 3141 | 1 | 71 | 7 |
| 3142 | 2 | 67 | 37 |
| 3143 | 1 | 139 | 33 |
| 3144 | 2 | 133 | 39 |
| 3145 | 1 | 8 | 5 |
| 3146 | 2 | 12 | 11 |
| 3147 | 1 | 116 | 6 |
| 3148 | 2 | 1 | 11 |
| 3149 | 1 | 20 | 5 |
| 3150 | 2 | 24 | 11 |
| 3151 | 1 | 128 | 6 |
| 3152 | 2 | 13 | 11 |
| 3153 | 1 | 32 | 5 |
| 3154 | 2 | 36 | 11 |
| 3155 | 1 | 140 | 6 |
| 3156 | 2 | 25 | 11 |
| 3157 | 1 | 116 | 5 |
| 3158 | 2 | 109 | 38 |
| 3159 | 1 | 10 | 5 |
| 3160 | 2 | 37 | 8 |
| 3161 | 1 | 128 | 5 |
| 3162 | 2 | 121 | 38 |
| 3163 | 1 | 22 | 5 |
| 3164 | 2 | 49 | 8 |
| 3165 | 1 | 140 | 5 |
| 3166 | 2 | 133 | 38 |
| 3167 | 1 | 34 | 5 |
| 3168 | 2 | 61 | 8 |
| 3169 | 1 | 5 | 50 |
| 3170 | 2 | 1 | 16 |
| 3171 | 1 | 3 | 50 |
| 3172 | 2 | 118 | 45 |
| 3173 | 1 | 17 | 50 |
| 3174 | 2 | 13 | 16 |
| 3175 | 1 | 15 | 50 |
| 3176 | 2 | 130 | 45 |
| 3177 | 1 | 29 | 50 |
| 3178 | 2 | 25 | 16 |
| 3179 | 1 | 27 | 50 |
| 3180 | 2 | 142 | 45 |
| 3181 | 1 | 75 | 45 |
| 3182 | 2 | 45 | 42 |
| 3183 | 1 | 119 | 9 |
| 3184 | 2 | 6 | 48 |
| 3185 | 1 | 87 | 45 |
| 3186 | 2 | 57 | 42 |
| 3187 | 1 | 131 | 9 |
| 3188 | 2 | 18 | 48 |
| 3189 | 1 | 99 | 45 |
| 3190 | 2 | 69 | 42 |
| 3191 | 1 | 143 | 9 |
| 3192 | 2 | 30 | 48 |
| 3193 | 1 | 40 | 44 |
| 3194 | 2 | 8 | 49 |
| 3195 | 1 | 11 | 46 |
| 3196 | 2 | 73 | 48 |
| 3197 | 1 | 52 | 44 |
| 3198 | 2 | 20 | 49 |
| 3199 | 1 | 23 | 46 |
| 3200 | 2 | 85 | 48 |
| 3201 | 1 | 64 | 44 |
| 3202 | 2 | 32 | 49 |
| 3203 | 1 | 35 | 46 |
| 3204 | 2 | 97 | 48 |
| 3205 | 1 | 73 | 16 |
| 3206 | 2 | 114 | 11 |
| 3207 | 1 | 7 | 14 |
| 3208 | 2 | 109 | 45 |
| 3209 | 1 | 85 | 16 |
| 3210 | 2 | 126 | 11 |
| 3211 | 1 | 19 | 14 |
| 3212 | 2 | 121 | 45 |
| 3213 | 1 | 97 | 16 |
| 3214 | 2 | 138 | 11 |
| 3215 | 1 | 31 | 14 |
| 3216 | 2 | 133 | 45 |
| 3217 | 1 | 77 | 13 |
| 3218 | 2 | 73 | 49 |
| 3219 | 1 | 43 | 9 |
| 3220 | 2 | 73 | 50 |
| 3221 | 1 | 89 | 13 |
| 3222 | 2 | 85 | 49 |
| 3223 | 1 | 55 | 9 |
| 3224 | 2 | 85 | 50 |
| 3225 | 1 | 101 | 13 |
| 3226 | 2 | 97 | 49 |
| 3227 | 1 | 67 | 9 |
| 3228 | 2 | 97 | 50 |
| 3229 | 1 | 47 | 15 |
| 3230 | 2 | 43 | 45 |
| 3231 | 1 | 115 | 41 |
| 3232 | 2 | 109 | 47 |
| 3233 | 1 | 59 | 15 |
| 3234 | 2 | 55 | 45 |
| 3235 | 1 | 127 | 41 |
| 3236 | 2 | 121 | 47 |
| 3237 | 1 | 71 | 15 |
| 3238 | 2 | 67 | 45 |
| 3239 | 1 | 139 | 41 |
| 3240 | 2 | 133 | 47 |
| 3241 | 1 | 8 | 13 |
| 3242 | 2 | 12 | 19 |
| 3243 | 1 | 116 | 14 |
| 3244 | 2 | 1 | 19 |
| 3245 | 1 | 20 | 13 |
| 3246 | 2 | 24 | 19 |
| 3247 | 1 | 128 | 14 |
| 3248 | 2 | 13 | 19 |
| 3249 | 1 | 32 | 13 |
| 3250 | 2 | 36 | 19 |
| 3251 | 1 | 140 | 14 |
| 3252 | 2 | 25 | 19 |
| 3253 | 1 | 116 | 13 |
| 3254 | 2 | 109 | 46 |
| 3255 | 1 | 10 | 13 |
| 3256 | 2 | 37 | 16 |
| 3257 | 1 | 128 | 13 |
| 3258 | 2 | 121 | 46 |
| 3259 | 1 | 22 | 13 |
| 3260 | 2 | 49 | 16 |
| 3261 | 1 | 140 | 13 |
| 3262 | 2 | 133 | 46 |
| 3263 | 1 | 34 | 13 |
| 3264 | 2 | 61 | 16 |
| 3265 | 1 | 5 | 58 |
| 3266 | 2 | 1 | 24 |
| 3267 | 1 | 3 | 58 |
| 3268 | 2 | 118 | 53 |
| 3269 | 1 | 17 | 58 |
| 3270 | 2 | 13 | 24 |
| 3271 | 1 | 15 | 58 |
| 3272 | 2 | 130 | 53 |
| 3273 | 1 | 29 | 58 |
| 3274 | 2 | 25 | 24 |
| 3275 | 1 | 27 | 58 |
| 3276 | 2 | 142 | 53 |
| 3277 | 1 | 75 | 53 |
| 3278 | 2 | 45 | 50 |
| 3279 | 1 | 119 | 17 |
| 3280 | 2 | 6 | 56 |
| 3281 | 1 | 87 | 53 |
| 3282 | 2 | 57 | 50 |
| 3283 | 1 | 131 | 17 |
| 3284 | 2 | 18 | 56 |
| 3285 | 1 | 99 | 53 |
| 3286 | 2 | 69 | 50 |
| 3287 | 1 | 143 | 17 |
| 3288 | 2 | 30 | 56 |
| 3289 | 1 | 40 | 52 |
| 3290 | 2 | 8 | 57 |
| 3291 | 1 | 11 | 54 |
| 3292 | 2 | 73 | 56 |
| 3293 | 1 | 52 | 52 |
| 3294 | 2 | 20 | 57 |
| 3295 | 1 | 23 | 54 |
| 3296 | 2 | 85 | 56 |
| 3297 | 1 | 64 | 52 |
| 3298 | 2 | 32 | 57 |
| 3299 | 1 | 35 | 54 |
| 3300 | 2 | 97 | 56 |
| 3301 | 1 | 73 | 24 |
| 3302 | 2 | 114 | 19 |
| 3303 | 1 | 7 | 22 |
| 3304 | 2 | 109 | 53 |
| 3305 | 1 | 85 | 24 |
| 3306 | 2 | 126 | 19 |
| 3307 | 1 | 19 | 22 |
| 3308 | 2 | 121 | 53 |
| 3309 | 1 | 97 | 24 |
| 3310 | 2 | 138 | 19 |
| 3311 | 1 | 31 | 22 |
| 3312 | 2 | 133 | 53 |
| 3313 | 1 | 77 | 21 |
| 3314 | 2 | 73 | 57 |
| 3315 | 1 | 43 | 17 |
| 3316 | 2 | 73 | 58 |
| 3317 | 1 | 89 | 21 |
| 3318 | 2 | 85 | 57 |
| 3319 | 1 | 55 | 17 |
| 3320 | 2 | 85 | 58 |
| 3321 | 1 | 101 | 21 |
| 3322 | 2 | 97 | 57 |
| 3323 | 1 | 67 | 17 |
| 3324 | 2 | 97 | 58 |
| 3325 | 1 | 47 | 23 |
| 3326 | 2 | 43 | 53 |
| 3327 | 1 | 115 | 49 |
| 3328 | 2 | 109 | 55 |
| 3329 | 1 | 59 | 23 |
| 3330 | 2 | 55 | 53 |
| 3331 | 1 | 127 | 49 |
| 3332 | 2 | 121 | 55 |
| 3333 | 1 | 71 | 23 |
| 3334 | 2 | 67 | 53 |
| 3335 | 1 | 139 | 49 |
| 3336 | 2 | 133 | 55 |
| 3337 | 1 | 8 | 21 |
| 3338 | 2 | 12 | 27 |
| 3339 | 1 | 116 | 22 |
| 3340 | 2 | 1 | 27 |
| 3341 | 1 | 20 | 21 |
| 3342 | 2 | 24 | 27 |
| 3343 | 1 | 128 | 22 |
| 3344 | 2 | 13 | 27 |
| 3345 | 1 | 32 | 21 |
| 3346 | 2 | 36 | 27 |
| 3347 | 1 | 140 | 22 |
| 3348 | 2 | 25 | 27 |
| 3349 | 1 | 116 | 21 |
| 3350 | 2 | 109 | 54 |
| 3351 | 1 | 10 | 21 |
| 3352 | 2 | 37 | 24 |
| 3353 | 1 | 128 | 21 |
| 3354 | 2 | 121 | 54 |
| 3355 | 1 | 22 | 21 |
| 3356 | 2 | 49 | 24 |
| 3357 | 1 | 140 | 21 |
| 3358 | 2 | 133 | 54 |
| 3359 | 1 | 34 | 21 |
| 3360 | 2 | 61 | 24 |
| 3361 | 1 | 5 | 66 |
| 3362 | 2 | 1 | 32 |
| 3363 | 1 | 3 | 66 |
| 3364 | 2 | 118 | 61 |
| 3365 | 1 | 17 | 66 |
| 3366 | 2 | 13 | 32 |
| 3367 | 1 | 15 | 66 |
| 3368 | 2 | 130 | 61 |
| 3369 | 1 | 29 | 66 |
| 3370 | 2 | 25 | 32 |
| 3371 | 1 | 27 | 66 |
| 3372 | 2 | 142 | 61 |
| 3373 | 1 | 75 | 61 |
| 3374 | 2 | 45 | 58 |
| 3375 | 1 | 119 | 25 |
| 3376 | 2 | 6 | 64 |
| 3377 | 1 | 87 | 61 |
| 3378 | 2 | 57 | 58 |
| 3379 | 1 | 131 | 25 |
| 3380 | 2 | 18 | 64 |
| 3381 | 1 | 99 | 61 |
| 3382 | 2 | 69 | 58 |
| 3383 | 1 | 143 | 25 |
| 3384 | 2 | 30 | 64 |
| 3385 | 1 | 40 | 60 |
| 3386 | 2 | 8 | 65 |
| 3387 | 1 | 11 | 62 |
| 3388 | 2 | 73 | 64 |
| 3389 | 1 | 52 | 60 |
| 3390 | 2 | 20 | 65 |
| 3391 | 1 | 23 | 62 |
| 3392 | 2 | 85 | 64 |
| 3393 | 1 | 64 | 60 |
| 3394 | 2 | 32 | 65 |
| 3395 | 1 | 35 | 62 |
| 3396 | 2 | 97 | 64 |
| 3397 | 1 | 73 | 32 |
| 3398 | 2 | 114 | 27 |
| 3399 | 1 | 7 | 30 |
| 3400 | 2 | 109 | 61 |
| 3401 | 1 | 85 | 32 |
| 3402 | 2 | 126 | 27 |
| 3403 | 1 | 19 | 30 |
| 3404 | 2 | 121 | 61 |
| 3405 | 1 | 97 | 32 |
| 3406 | 2 | 138 | 27 |
| 3407 | 1 | 31 | 30 |
| 3408 | 2 | 133 | 61 |
| 3409 | 1 | 77 | 29 |
| 3410 | 2 | 73 | 65 |
| 3411 | 1 | 43 | 25 |
| 3412 | 2 | 73 | 66 |
| 3413 | 1 | 89 | 29 |
| 3414 | 2 | 85 | 65 |
| 3415 | 1 | 55 | 25 |
| 3416 | 2 | 85 | 66 |
| 3417 | 1 | 101 | 29 |
| 3418 | 2 | 97 | 65 |
| 3419 | 1 | 67 | 25 |
| 3420 | 2 | 97 | 66 |
| 3421 | 1 | 47 | 31 |
| 3422 | 2 | 43 | 61 |
| 3423 | 1 | 115 | 57 |
| 3424 | 2 | 109 | 63 |
| 3425 | 1 | 59 | 31 |
| 3426 | 2 | 55 | 61 |
| 3427 | 1 | 127 | 57 |
| 3428 | 2 | 121 | 63 |
| 3429 | 1 | 71 | 31 |
| 3430 | 2 | 67 | 61 |
| 3431 | 1 | 139 | 57 |
| 3432 | 2 | 133 | 63 |
| 3433 | 1 | 8 | 29 |
| 3434 | 2 | 12 | 35 |
| 3435 | 1 | 116 | 30 |
| 3436 | 2 | 1 | 35 |
| 3437 | 1 | 20 | 29 |
| 3438 | 2 | 24 | 35 |
| 3439 | 1 | 128 | 30 |
| 3440 | 2 | 13 | 35 |
| 3441 | 1 | 32 | 29 |
| 3442 | 2 | 36 | 35 |
| 3443 | 1 | 140 | 30 |
| 3444 | 2 | 25 | 35 |
| 3445 | 1 | 116 | 29 |
| 3446 | 2 | 109 | 62 |
| 3447 | 1 | 10 | 29 |
| 3448 | 2 | 37 | 32 |
| 3449 | 1 | 128 | 29 |
| 3450 | 2 | 121 | 62 |
| 3451 | 1 | 22 | 29 |
| 3452 | 2 | 49 | 32 |
| 3453 | 1 | 140 | 29 |
| 3454 | 2 | 133 | 62 |
| 3455 | 1 | 34 | 29 |
| 3456 | 2 | 61 | 32 |
| 3457 | 1 | 46 | 2 |
| 3458 | 2 | 9 | 6 |
| 3459 | 1 | 39 | 37 |
| 3460 | 2 | 1 | 9 |
| 3461 | 1 | 58 | 2 |
| 3462 | 2 | 21 | 6 |
| 3463 | 1 | 51 | 37 |
| 3464 | 2 | 13 | 9 |
| 3465 | 1 | 70 | 2 |
| 3466 | 2 | 33 | 6 |
| 3467 | 1 | 63 | 37 |
| 3468 | 2 | 25 | 9 |
| 3469 | 1 | 47 | 40 |
| 3470 | 2 | 109 | 5 |
| 3471 | 1 | 37 | 39 |
| 3472 | 2 | 37 | 6 |
| 3473 | 1 | 59 | 40 |
| 3474 | 2 | 121 | 5 |
| 3475 | 1 | 49 | 39 |
| 3476 | 2 | 49 | 6 |
| 3477 | 1 | 71 | 40 |
| 3478 | 2 | 133 | 5 |
| 3479 | 1 | 61 | 39 |
| 3480 | 2 | 61 | 6 |
| 3481 | 1 | 79 | 6 |
| 3482 | 2 | 120 | 34 |
| 3483 | 1 | 112 | 37 |
| 3484 | 2 | 1 | 41 |
| 3485 | 1 | 91 | 6 |
| 3486 | 2 | 132 | 34 |
| 3487 | 1 | 124 | 37 |
| 3488 | 2 | 13 | 41 |
| 3489 | 1 | 103 | 6 |
| 3490 | 2 | 144 | 34 |
| 3491 | 1 | 136 | 37 |
| 3492 | 2 | 25 | 41 |
| 3493 | 1 | 83 | 40 |
| 3494 | 2 | 37 | 37 |
| 3495 | 1 | 81 | 5 |
| 3496 | 2 | 117 | 7 |
| 3497 | 1 | 95 | 40 |
| 3498 | 2 | 49 | 37 |
| 3499 | 1 | 93 | 5 |
| 3500 | 2 | 129 | 7 |
| 3501 | 1 | 107 | 40 |
| 3502 | 2 | 61 | 37 |
| 3503 | 1 | 105 | 5 |
| 3504 | 2 | 141 | 7 |
| 3505 | 1 | 118 | 8 |
| 3506 | 2 | 6 | 43 |
| 3507 | 1 | 37 | 40 |
| 3508 | 2 | 1 | 43 |
| 3509 | 1 | 130 | 8 |
| 3510 | 2 | 18 | 43 |
| 3511 | 1 | 49 | 40 |
| 3512 | 2 | 13 | 43 |
| 3513 | 1 | 142 | 8 |
| 3514 | 2 | 30 | 43 |
| 3515 | 1 | 61 | 40 |
| 3516 | 2 | 25 | 43 |
| 3517 | 1 | 75 | 43 |
| 3518 | 2 | 1 | 10 |
| 3519 | 1 | 109 | 40 |
| 3520 | 2 | 44 | 39 |
| 3521 | 1 | 87 | 43 |
| 3522 | 2 | 13 | 10 |
| 3523 | 1 | 121 | 40 |
| 3524 | 2 | 56 | 39 |
| 3525 | 1 | 99 | 43 |
| 3526 | 2 | 25 | 10 |
| 3527 | 1 | 133 | 40 |
| 3528 | 2 | 68 | 39 |
| 3529 | 1 | 111 | 33 |
| 3530 | 2 | 37 | 7 |
| 3531 | 1 | 2 | 39 |
| 3532 | 2 | 2 | 4 |
| 3533 | 1 | 123 | 33 |
| 3534 | 2 | 49 | 7 |
| 3535 | 1 | 14 | 39 |
| 3536 | 2 | 14 | 4 |
| 3537 | 1 | 135 | 33 |
| 3538 | 2 | 61 | 7 |
| 3539 | 1 | 26 | 39 |
| 3540 | 2 | 26 | 4 |
| 3541 | 1 | 74 | 5 |
| 3542 | 2 | 73 | 10 |
| 3543 | 1 | 39 | 38 |
| 3544 | 2 | 47 | 1 |
| 3545 | 1 | 86 | 5 |
| 3546 | 2 | 85 | 10 |
| 3547 | 1 | 51 | 38 |
| 3548 | 2 | 59 | 1 |
| 3549 | 1 | 98 | 5 |
| 3550 | 2 | 97 | 10 |
| 3551 | 1 | 63 | 38 |
| 3552 | 2 | 71 | 1 |
| 3553 | 1 | 46 | 10 |
| 3554 | 2 | 9 | 14 |
| 3555 | 1 | 39 | 45 |
| 3556 | 2 | 1 | 17 |
| 3557 | 1 | 58 | 10 |
| 3558 | 2 | 21 | 14 |
| 3559 | 1 | 51 | 45 |
| 3560 | 2 | 13 | 17 |
| 3561 | 1 | 70 | 10 |
| 3562 | 2 | 33 | 14 |
| 3563 | 1 | 63 | 45 |
| 3564 | 2 | 25 | 17 |
| 3565 | 1 | 47 | 48 |
| 3566 | 2 | 109 | 13 |
| 3567 | 1 | 37 | 47 |
| 3568 | 2 | 37 | 14 |
| 3569 | 1 | 59 | 48 |
| 3570 | 2 | 121 | 13 |
| 3571 | 1 | 49 | 47 |
| 3572 | 2 | 49 | 14 |
| 3573 | 1 | 71 | 48 |
| 3574 | 2 | 133 | 13 |
| 3575 | 1 | 61 | 47 |
| 3576 | 2 | 61 | 14 |
| 3577 | 1 | 79 | 14 |
| 3578 | 2 | 120 | 42 |
| 3579 | 1 | 112 | 45 |
| 3580 | 2 | 1 | 49 |
| 3581 | 1 | 91 | 14 |
| 3582 | 2 | 132 | 42 |
| 3583 | 1 | 124 | 45 |
| 3584 | 2 | 13 | 49 |
| 3585 | 1 | 103 | 14 |
| 3586 | 2 | 144 | 42 |
| 3587 | 1 | 136 | 45 |
| 3588 | 2 | 25 | 49 |
| 3589 | 1 | 83 | 48 |
| 3590 | 2 | 37 | 45 |
| 3591 | 1 | 81 | 13 |
| 3592 | 2 | 117 | 15 |
| 3593 | 1 | 95 | 48 |
| 3594 | 2 | 49 | 45 |
| 3595 | 1 | 93 | 13 |
| 3596 | 2 | 129 | 15 |
| 3597 | 1 | 107 | 48 |
| 3598 | 2 | 61 | 45 |
| 3599 | 1 | 105 | 13 |
| 3600 | 2 | 141 | 15 |
| 3601 | 1 | 118 | 16 |
| 3602 | 2 | 6 | 51 |
| 3603 | 1 | 37 | 48 |
| 3604 | 2 | 1 | 51 |
| 3605 | 1 | 130 | 16 |
| 3606 | 2 | 18 | 51 |
| 3607 | 1 | 49 | 48 |
| 3608 | 2 | 13 | 51 |
| 3609 | 1 | 142 | 16 |
| 3610 | 2 | 30 | 51 |
| 3611 | 1 | 61 | 48 |
| 3612 | 2 | 25 | 51 |
| 3613 | 1 | 75 | 51 |
| 3614 | 2 | 1 | 18 |
| 3615 | 1 | 109 | 48 |
| 3616 | 2 | 44 | 47 |
| 3617 | 1 | 87 | 51 |
| 3618 | 2 | 13 | 18 |
| 3619 | 1 | 121 | 48 |
| 3620 | 2 | 56 | 47 |
| 3621 | 1 | 99 | 51 |
| 3622 | 2 | 25 | 18 |
| 3623 | 1 | 133 | 48 |
| 3624 | 2 | 68 | 47 |
| 3625 | 1 | 111 | 41 |
| 3626 | 2 | 37 | 15 |
| 3627 | 1 | 2 | 47 |
| 3628 | 2 | 2 | 12 |
| 3629 | 1 | 123 | 41 |
| 3630 | 2 | 49 | 15 |
| 3631 | 1 | 14 | 47 |
| 3632 | 2 | 14 | 12 |
| 3633 | 1 | 135 | 41 |
| 3634 | 2 | 61 | 15 |
| 3635 | 1 | 26 | 47 |
| 3636 | 2 | 26 | 12 |
| 3637 | 1 | 74 | 13 |
| 3638 | 2 | 73 | 18 |
| 3639 | 1 | 39 | 46 |
| 3640 | 2 | 47 | 9 |
| 3641 | 1 | 86 | 13 |
| 3642 | 2 | 85 | 18 |
| 3643 | 1 | 51 | 46 |
| 3644 | 2 | 59 | 9 |
| 3645 | 1 | 98 | 13 |
| 3646 | 2 | 97 | 18 |
| 3647 | 1 | 63 | 46 |
| 3648 | 2 | 71 | 9 |
| 3649 | 1 | 46 | 18 |
| 3650 | 2 | 9 | 22 |
| 3651 | 1 | 39 | 53 |
| 3652 | 2 | 1 | 25 |
| 3653 | 1 | 58 | 18 |
| 3654 | 2 | 21 | 22 |
| 3655 | 1 | 51 | 53 |
| 3656 | 2 | 13 | 25 |
| 3657 | 1 | 70 | 18 |
| 3658 | 2 | 33 | 22 |
| 3659 | 1 | 63 | 53 |
| 3660 | 2 | 25 | 25 |
| 3661 | 1 | 47 | 56 |
| 3662 | 2 | 109 | 21 |
| 3663 | 1 | 37 | 55 |
| 3664 | 2 | 37 | 22 |
| 3665 | 1 | 59 | 56 |
| 3666 | 2 | 121 | 21 |
| 3667 | 1 | 49 | 55 |
| 3668 | 2 | 49 | 22 |
| 3669 | 1 | 71 | 56 |
| 3670 | 2 | 133 | 21 |
| 3671 | 1 | 61 | 55 |
| 3672 | 2 | 61 | 22 |
| 3673 | 1 | 79 | 22 |
| 3674 | 2 | 120 | 50 |
| 3675 | 1 | 112 | 53 |
| 3676 | 2 | 1 | 57 |
| 3677 | 1 | 91 | 22 |
| 3678 | 2 | 132 | 50 |
| 3679 | 1 | 124 | 53 |
| 3680 | 2 | 13 | 57 |
| 3681 | 1 | 103 | 22 |
| 3682 | 2 | 144 | 50 |
| 3683 | 1 | 136 | 53 |
| 3684 | 2 | 25 | 57 |
| 3685 | 1 | 83 | 56 |
| 3686 | 2 | 37 | 53 |
| 3687 | 1 | 81 | 21 |
| 3688 | 2 | 117 | 23 |
| 3689 | 1 | 95 | 56 |
| 3690 | 2 | 49 | 53 |
| 3691 | 1 | 93 | 21 |
| 3692 | 2 | 129 | 23 |
| 3693 | 1 | 107 | 56 |
| 3694 | 2 | 61 | 53 |
| 3695 | 1 | 105 | 21 |
| 3696 | 2 | 141 | 23 |
| 3697 | 1 | 118 | 24 |
| 3698 | 2 | 6 | 59 |
| 3699 | 1 | 37 | 56 |
| 3700 | 2 | 1 | 59 |
| 3701 | 1 | 130 | 24 |
| 3702 | 2 | 18 | 59 |
| 3703 | 1 | 49 | 56 |
| 3704 | 2 | 13 | 59 |
| 3705 | 1 | 142 | 24 |
| 3706 | 2 | 30 | 59 |
| 3707 | 1 | 61 | 56 |
| 3708 | 2 | 25 | 59 |
| 3709 | 1 | 75 | 59 |
| 3710 | 2 | 1 | 26 |
| 3711 | 1 | 109 | 56 |
| 3712 | 2 | 44 | 55 |
| 3713 | 1 | 87 | 59 |
| 3714 | 2 | 13 | 26 |
| 3715 | 1 | 121 | 56 |
| 3716 | 2 | 56 | 55 |
| 3717 | 1 | 99 | 59 |
| 3718 | 2 | 25 | 26 |
| 3719 | 1 | 133 | 56 |
| 3720 | 2 | 68 | 55 |
| 3721 | 1 | 111 | 49 |
| 3722 | 2 | 37 | 23 |
| 3723 | 1 | 2 | 55 |
| 3724 | 2 | 2 | 20 |
| 3725 | 1 | 123 | 49 |
| 3726 | 2 | 49 | 23 |
| 3727 | 1 | 14 | 55 |
| 3728 | 2 | 14 | 20 |
| 3729 | 1 | 135 | 49 |
| 3730 | 2 | 61 | 23 |
| 3731 | 1 | 26 | 55 |
| 3732 | 2 | 26 | 20 |
| 3733 | 1 | 74 | 21 |
| 3734 | 2 | 73 | 26 |
| 3735 | 1 | 39 | 54 |
| 3736 | 2 | 47 | 17 |
| 3737 | 1 | 86 | 21 |
| 3738 | 2 | 85 | 26 |
| 3739 | 1 | 51 | 54 |
| 3740 | 2 | 59 | 17 |
| 3741 | 1 | 98 | 21 |
| 3742 | 2 | 97 | 26 |
| 3743 | 1 | 63 | 54 |
| 3744 | 2 | 71 | 17 |
| 3745 | 1 | 46 | 26 |
| 3746 | 2 | 9 | 30 |
| 3747 | 1 | 39 | 61 |
| 3748 | 2 | 1 | 33 |
| 3749 | 1 | 58 | 26 |
| 3750 | 2 | 21 | 30 |
| 3751 | 1 | 51 | 61 |
| 3752 | 2 | 13 | 33 |
| 3753 | 1 | 70 | 26 |
| 3754 | 2 | 33 | 30 |
| 3755 | 1 | 63 | 61 |
| 3756 | 2 | 25 | 33 |
| 3757 | 1 | 47 | 64 |
| 3758 | 2 | 109 | 29 |
| 3759 | 1 | 37 | 63 |
| 3760 | 2 | 37 | 30 |
| 3761 | 1 | 59 | 64 |
| 3762 | 2 | 121 | 29 |
| 3763 | 1 | 49 | 63 |
| 3764 | 2 | 49 | 30 |
| 3765 | 1 | 71 | 64 |
| 3766 | 2 | 133 | 29 |
| 3767 | 1 | 61 | 63 |
| 3768 | 2 | 61 | 30 |
| 3769 | 1 | 79 | 30 |
| 3770 | 2 | 120 | 58 |
| 3771 | 1 | 112 | 61 |
| 3772 | 2 | 1 | 65 |
| 3773 | 1 | 91 | 30 |
| 3774 | 2 | 132 | 58 |
| 3775 | 1 | 124 | 61 |
| 3776 | 2 | 13 | 65 |
| 3777 | 1 | 103 | 30 |
| 3778 | 2 | 144 | 58 |
| 3779 | 1 | 136 | 61 |
| 3780 | 2 | 25 | 65 |
| 3781 | 1 | 83 | 64 |
| 3782 | 2 | 37 | 61 |
| 3783 | 1 | 81 | 29 |
| 3784 | 2 | 117 | 31 |
| 3785 | 1 | 95 | 64 |
| 3786 | 2 | 49 | 61 |
| 3787 | 1 | 93 | 29 |
| 3788 | 2 | 129 | 31 |
| 3789 | 1 | 107 | 64 |
| 3790 | 2 | 61 | 61 |
| 3791 | 1 | 105 | 29 |
| 3792 | 2 | 141 | 31 |
| 3793 | 1 | 118 | 32 |
| 3794 | 2 | 6 | 67 |
| 3795 | 1 | 37 | 64 |
| 3796 | 2 | 1 | 67 |
| 3797 | 1 | 130 | 32 |
| 3798 | 2 | 18 | 67 |
| 3799 | 1 | 49 | 64 |
| 3800 | 2 | 13 | 67 |
| 3801 | 1 | 142 | 32 |
| 3802 | 2 | 30 | 67 |
| 3803 | 1 | 61 | 64 |
| 3804 | 2 | 25 | 67 |
| 3805 | 1 | 75 | 67 |
| 3806 | 2 | 1 | 34 |
| 3807 | 1 | 109 | 64 |
| 3808 | 2 | 44 | 63 |
| 3809 | 1 | 87 | 67 |
| 3810 | 2 | 13 | 34 |
| 3811 | 1 | 121 | 64 |
| 3812 | 2 | 56 | 63 |
| 3813 | 1 | 99 | 67 |
| 3814 | 2 | 25 | 34 |
| 3815 | 1 | 133 | 64 |
| 3816 | 2 | 68 | 63 |
| 3817 | 1 | 111 | 57 |
| 3818 | 2 | 37 | 31 |
| 3819 | 1 | 2 | 63 |
| 3820 | 2 | 2 | 28 |
| 3821 | 1 | 123 | 57 |
| 3822 | 2 | 49 | 31 |
| 3823 | 1 | 14 | 63 |
| 3824 | 2 | 14 | 28 |
| 3825 | 1 | 135 | 57 |
| 3826 | 2 | 61 | 31 |
| 3827 | 1 | 26 | 63 |
| 3828 | 2 | 26 | 28 |
| 3829 | 1 | 74 | 29 |
| 3830 | 2 | 73 | 34 |
| 3831 | 1 | 39 | 62 |
| 3832 | 2 | 47 | 25 |
| 3833 | 1 | 86 | 29 |
| 3834 | 2 | 85 | 34 |
| 3835 | 1 | 51 | 62 |
| 3836 | 2 | 59 | 25 |
| 3837 | 1 | 98 | 29 |
| 3838 | 2 | 97 | 34 |
| 3839 | 1 | 63 | 62 |
| 3840 | 2 | 71 | 25 |
| 3841 | 1 | 117 | 8 |
| 3842 | 2 | 81 | 9 |
| 3843 | 1 | 82 | 10 |
| 3844 | 2 | 38 | 33 |
| 3845 | 1 | 129 | 8 |
| 3846 | 2 | 93 | 9 |
| 3847 | 1 | 94 | 10 |
| 3848 | 2 | 50 | 33 |
| 3849 | 1 | 141 | 8 |
| 3850 | 2 | 105 | 9 |
| 3851 | 1 | 106 | 10 |
| 3852 | 2 | 62 | 33 |
| 3853 | 1 | 118 | 7 |
| 3854 | 2 | 79 | 38 |
| 3855 | 1 | 115 | 38 |
| 3856 | 2 | 43 | 7 |
| 3857 | 1 | 130 | 7 |
| 3858 | 2 | 91 | 38 |
| 3859 | 1 | 127 | 38 |
| 3860 | 2 | 55 | 7 |
| 3861 | 1 | 142 | 7 |
| 3862 | 2 | 103 | 38 |
| 3863 | 1 | 139 | 38 |
| 3864 | 2 | 67 | 7 |
| 3865 | 1 | 80 | 11 |
| 3866 | 2 | 46 | 37 |
| 3867 | 1 | 40 | 1 |
| 3868 | 2 | 78 | 11 |
| 3869 | 1 | 92 | 11 |
| 3870 | 2 | 58 | 37 |
| 3871 | 1 | 52 | 1 |
| 3872 | 2 | 90 | 11 |
| 3873 | 1 | 104 | 11 |
| 3874 | 2 | 70 | 37 |
| 3875 | 1 | 64 | 1 |
| 3876 | 2 | 102 | 11 |
| 3877 | 1 | 74 | 7 |
| 3878 | 2 | 38 | 1 |
| 3879 | 1 | 42 | 38 |
| 3880 | 2 | 82 | 42 |
| 3881 | 1 | 86 | 7 |
| 3882 | 2 | 50 | 1 |
| 3883 | 1 | 54 | 38 |
| 3884 | 2 | 94 | 42 |
| 3885 | 1 | 98 | 7 |
| 3886 | 2 | 62 | 1 |
| 3887 | 1 | 66 | 38 |
| 3888 | 2 | 106 | 42 |
| 3889 | 1 | 38 | 4 |
| 3890 | 2 | 110 | 3 |
| 3891 | 1 | 41 | 40 |
| 3892 | 2 | 2 | 8 |
| 3893 | 1 | 50 | 4 |
| 3894 | 2 | 122 | 3 |
| 3895 | 1 | 53 | 40 |
| 3896 | 2 | 14 | 8 |
| 3897 | 1 | 62 | 4 |
| 3898 | 2 | 134 | 3 |
| 3899 | 1 | 65 | 40 |
| 3900 | 2 | 26 | 8 |
| 3901 | 1 | 5 | 5 |
| 3902 | 2 | 6 | 36 |
| 3903 | 1 | 7 | 4 |
| 3904 | 2 | 43 | 6 |
| 3905 | 1 | 17 | 5 |
| 3906 | 2 | 18 | 36 |
| 3907 | 1 | 19 | 4 |
| 3908 | 2 | 55 | 6 |
| 3909 | 1 | 29 | 5 |
| 3910 | 2 | 30 | 36 |
| 3911 | 1 | 31 | 4 |
| 3912 | 2 | 67 | 6 |
| 3913 | 1 | 110 | 5 |
| 3914 | 2 | 110 | 35 |
| 3915 | 1 | 77 | 10 |
| 3916 | 2 | 38 | 37 |
| 3917 | 1 | 122 | 5 |
| 3918 | 2 | 122 | 35 |
| 3919 | 1 | 89 | 10 |
| 3920 | 2 | 50 | 37 |
| 3921 | 1 | 134 | 5 |
| 3922 | 2 | 134 | 35 |
| 3923 | 1 | 101 | 10 |
| 3924 | 2 | 62 | 37 |
| 3925 | 1 | 79 | 40 |
| 3926 | 2 | 9 | 36 |
| 3927 | 1 | 6 | 37 |
| 3928 | 2 | 117 | 37 |
| 3929 | 1 | 91 | 40 |
| 3930 | 2 | 21 | 36 |
| 3931 | 1 | 18 | 37 |
| 3932 | 2 | 129 | 37 |
| 3933 | 1 | 103 | 40 |
| 3934 | 2 | 33 | 36 |
| 3935 | 1 | 30 | 37 |
| 3936 | 2 | 141 | 37 |
| 3937 | 1 | 117 | 16 |
| 3938 | 2 | 81 | 17 |
| 3939 | 1 | 82 | 18 |
| 3940 | 2 | 38 | 41 |
| 3941 | 1 | 129 | 16 |
| 3942 | 2 | 93 | 17 |
| 3943 | 1 | 94 | 18 |
| 3944 | 2 | 50 | 41 |
| 3945 | 1 | 141 | 16 |
| 3946 | 2 | 105 | 17 |
| 3947 | 1 | 106 | 18 |
| 3948 | 2 | 62 | 41 |
| 3949 | 1 | 118 | 15 |
| 3950 | 2 | 79 | 46 |
| 3951 | 1 | 115 | 46 |
| 3952 | 2 | 43 | 15 |
| 3953 | 1 | 130 | 15 |
| 3954 | 2 | 91 | 46 |
| 3955 | 1 | 127 | 46 |
| 3956 | 2 | 55 | 15 |
| 3957 | 1 | 142 | 15 |
| 3958 | 2 | 103 | 46 |
| 3959 | 1 | 139 | 46 |
| 3960 | 2 | 67 | 15 |
| 3961 | 1 | 80 | 19 |
| 3962 | 2 | 46 | 45 |
| 3963 | 1 | 40 | 9 |
| 3964 | 2 | 78 | 19 |
| 3965 | 1 | 92 | 19 |
| 3966 | 2 | 58 | 45 |
| 3967 | 1 | 52 | 9 |
| 3968 | 2 | 90 | 19 |
| 3969 | 1 | 104 | 19 |
| 3970 | 2 | 70 | 45 |
| 3971 | 1 | 64 | 9 |
| 3972 | 2 | 102 | 19 |
| 3973 | 1 | 74 | 15 |
| 3974 | 2 | 38 | 9 |
| 3975 | 1 | 42 | 46 |
| 3976 | 2 | 82 | 50 |
| 3977 | 1 | 86 | 15 |
| 3978 | 2 | 50 | 9 |
| 3979 | 1 | 54 | 46 |
| 3980 | 2 | 94 | 50 |
| 3981 | 1 | 98 | 15 |
| 3982 | 2 | 62 | 9 |
| 3983 | 1 | 66 | 46 |
| 3984 | 2 | 106 | 50 |
| 3985 | 1 | 38 | 12 |
| 3986 | 2 | 110 | 11 |
| 3987 | 1 | 41 | 48 |
| 3988 | 2 | 2 | 16 |
| 3989 | 1 | 50 | 12 |
| 3990 | 2 | 122 | 11 |
| 3991 | 1 | 53 | 48 |
| 3992 | 2 | 14 | 16 |
| 3993 | 1 | 62 | 12 |
| 3994 | 2 | 134 | 11 |
| 3995 | 1 | 65 | 48 |
| 3996 | 2 | 26 | 16 |
| 3997 | 1 | 5 | 13 |
| 3998 | 2 | 6 | 44 |
| 3999 | 1 | 7 | 12 |
| 4000 | 2 | 43 | 14 |
| 4001 | 1 | 17 | 13 |
| 4002 | 2 | 18 | 44 |
| 4003 | 1 | 19 | 12 |
| 4004 | 2 | 55 | 14 |
| 4005 | 1 | 29 | 13 |
| 4006 | 2 | 30 | 44 |
| 4007 | 1 | 31 | 12 |
| 4008 | 2 | 67 | 14 |
| 4009 | 1 | 110 | 13 |
| 4010 | 2 | 110 | 43 |
| 4011 | 1 | 77 | 18 |
| 4012 | 2 | 38 | 45 |
| 4013 | 1 | 122 | 13 |
| 4014 | 2 | 122 | 43 |
| 4015 | 1 | 89 | 18 |
| 4016 | 2 | 50 | 45 |
| 4017 | 1 | 134 | 13 |
| 4018 | 2 | 134 | 43 |
| 4019 | 1 | 101 | 18 |
| 4020 | 2 | 62 | 45 |
| 4021 | 1 | 79 | 48 |
| 4022 | 2 | 9 | 44 |
| 4023 | 1 | 6 | 45 |
| 4024 | 2 | 117 | 45 |
| 4025 | 1 | 91 | 48 |
| 4026 | 2 | 21 | 44 |
| 4027 | 1 | 18 | 45 |
| 4028 | 2 | 129 | 45 |
| 4029 | 1 | 103 | 48 |
| 4030 | 2 | 33 | 44 |
| 4031 | 1 | 30 | 45 |
| 4032 | 2 | 141 | 45 |
| 4033 | 1 | 117 | 24 |
| 4034 | 2 | 81 | 25 |
| 4035 | 1 | 82 | 26 |
| 4036 | 2 | 38 | 49 |
| 4037 | 1 | 129 | 24 |
| 4038 | 2 | 93 | 25 |
| 4039 | 1 | 94 | 26 |
| 4040 | 2 | 50 | 49 |
| 4041 | 1 | 141 | 24 |
| 4042 | 2 | 105 | 25 |
| 4043 | 1 | 106 | 26 |
| 4044 | 2 | 62 | 49 |
| 4045 | 1 | 118 | 23 |
| 4046 | 2 | 79 | 54 |
| 4047 | 1 | 115 | 54 |
| 4048 | 2 | 43 | 23 |
| 4049 | 1 | 130 | 23 |
| 4050 | 2 | 91 | 54 |
| 4051 | 1 | 127 | 54 |
| 4052 | 2 | 55 | 23 |
| 4053 | 1 | 142 | 23 |
| 4054 | 2 | 103 | 54 |
| 4055 | 1 | 139 | 54 |
| 4056 | 2 | 67 | 23 |
| 4057 | 1 | 80 | 27 |
| 4058 | 2 | 46 | 53 |
| 4059 | 1 | 40 | 17 |
| 4060 | 2 | 78 | 27 |
| 4061 | 1 | 92 | 27 |
| 4062 | 2 | 58 | 53 |
| 4063 | 1 | 52 | 17 |
| 4064 | 2 | 90 | 27 |
| 4065 | 1 | 104 | 27 |
| 4066 | 2 | 70 | 53 |
| 4067 | 1 | 64 | 17 |
| 4068 | 2 | 102 | 27 |
| 4069 | 1 | 74 | 23 |
| 4070 | 2 | 38 | 17 |
| 4071 | 1 | 42 | 54 |
| 4072 | 2 | 82 | 58 |
| 4073 | 1 | 86 | 23 |
| 4074 | 2 | 50 | 17 |
| 4075 | 1 | 54 | 54 |
| 4076 | 2 | 94 | 58 |
| 4077 | 1 | 98 | 23 |
| 4078 | 2 | 62 | 17 |
| 4079 | 1 | 66 | 54 |
| 4080 | 2 | 106 | 58 |
| 4081 | 1 | 38 | 20 |
| 4082 | 2 | 110 | 19 |
| 4083 | 1 | 41 | 56 |
| 4084 | 2 | 2 | 24 |
| 4085 | 1 | 50 | 20 |
| 4086 | 2 | 122 | 19 |
| 4087 | 1 | 53 | 56 |
| 4088 | 2 | 14 | 24 |
| 4089 | 1 | 62 | 20 |
| 4090 | 2 | 134 | 19 |
| 4091 | 1 | 65 | 56 |
| 4092 | 2 | 26 | 24 |
| 4093 | 1 | 5 | 21 |
| 4094 | 2 | 6 | 52 |
| 4095 | 1 | 7 | 20 |
| 4096 | 2 | 43 | 22 |
| 4097 | 1 | 17 | 21 |
| 4098 | 2 | 18 | 52 |
| 4099 | 1 | 19 | 20 |
| 4100 | 2 | 55 | 22 |
| 4101 | 1 | 29 | 21 |
| 4102 | 2 | 30 | 52 |
| 4103 | 1 | 31 | 20 |
| 4104 | 2 | 67 | 22 |
| 4105 | 1 | 110 | 21 |
| 4106 | 2 | 110 | 51 |
| 4107 | 1 | 77 | 26 |
| 4108 | 2 | 38 | 53 |
| 4109 | 1 | 122 | 21 |
| 4110 | 2 | 122 | 51 |
| 4111 | 1 | 89 | 26 |
| 4112 | 2 | 50 | 53 |
| 4113 | 1 | 134 | 21 |
| 4114 | 2 | 134 | 51 |
| 4115 | 1 | 101 | 26 |
| 4116 | 2 | 62 | 53 |
| 4117 | 1 | 79 | 56 |
| 4118 | 2 | 9 | 52 |
| 4119 | 1 | 6 | 53 |
| 4120 | 2 | 117 | 53 |
| 4121 | 1 | 91 | 56 |
| 4122 | 2 | 21 | 52 |
| 4123 | 1 | 18 | 53 |
| 4124 | 2 | 129 | 53 |
| 4125 | 1 | 103 | 56 |
| 4126 | 2 | 33 | 52 |
| 4127 | 1 | 30 | 53 |
| 4128 | 2 | 141 | 53 |
| 4129 | 1 | 117 | 32 |
| 4130 | 2 | 81 | 33 |
| 4131 | 1 | 82 | 34 |
| 4132 | 2 | 38 | 57 |
| 4133 | 1 | 129 | 32 |
| 4134 | 2 | 93 | 33 |
| 4135 | 1 | 94 | 34 |
| 4136 | 2 | 50 | 57 |
| 4137 | 1 | 141 | 32 |
| 4138 | 2 | 105 | 33 |
| 4139 | 1 | 106 | 34 |
| 4140 | 2 | 62 | 57 |
| 4141 | 1 | 118 | 31 |
| 4142 | 2 | 79 | 62 |
| 4143 | 1 | 115 | 62 |
| 4144 | 2 | 43 | 31 |
| 4145 | 1 | 130 | 31 |
| 4146 | 2 | 91 | 62 |
| 4147 | 1 | 127 | 62 |
| 4148 | 2 | 55 | 31 |
| 4149 | 1 | 142 | 31 |
| 4150 | 2 | 103 | 62 |
| 4151 | 1 | 139 | 62 |
| 4152 | 2 | 67 | 31 |
| 4153 | 1 | 80 | 35 |
| 4154 | 2 | 46 | 61 |
| 4155 | 1 | 40 | 25 |
| 4156 | 2 | 78 | 35 |
| 4157 | 1 | 92 | 35 |
| 4158 | 2 | 58 | 61 |
| 4159 | 1 | 52 | 25 |
| 4160 | 2 | 90 | 35 |
| 4161 | 1 | 104 | 35 |
| 4162 | 2 | 70 | 61 |
| 4163 | 1 | 64 | 25 |
| 4164 | 2 | 102 | 35 |
| 4165 | 1 | 74 | 31 |
| 4166 | 2 | 38 | 25 |
| 4167 | 1 | 42 | 62 |
| 4168 | 2 | 82 | 66 |
| 4169 | 1 | 86 | 31 |
| 4170 | 2 | 50 | 25 |
| 4171 | 1 | 54 | 62 |
| 4172 | 2 | 94 | 66 |
| 4173 | 1 | 98 | 31 |
| 4174 | 2 | 62 | 25 |
| 4175 | 1 | 66 | 62 |
| 4176 | 2 | 106 | 66 |
| 4177 | 1 | 38 | 28 |
| 4178 | 2 | 110 | 27 |
| 4179 | 1 | 41 | 64 |
| 4180 | 2 | 2 | 32 |
| 4181 | 1 | 50 | 28 |
| 4182 | 2 | 122 | 27 |
| 4183 | 1 | 53 | 64 |
| 4184 | 2 | 14 | 32 |
| 4185 | 1 | 62 | 28 |
| 4186 | 2 | 134 | 27 |
| 4187 | 1 | 65 | 64 |
| 4188 | 2 | 26 | 32 |
| 4189 | 1 | 5 | 29 |
| 4190 | 2 | 6 | 60 |
| 4191 | 1 | 7 | 28 |
| 4192 | 2 | 43 | 30 |
| 4193 | 1 | 17 | 29 |
| 4194 | 2 | 18 | 60 |
| 4195 | 1 | 19 | 28 |
| 4196 | 2 | 55 | 30 |
| 4197 | 1 | 29 | 29 |
| 4198 | 2 | 30 | 60 |
| 4199 | 1 | 31 | 28 |
| 4200 | 2 | 67 | 30 |
| 4201 | 1 | 110 | 29 |
| 4202 | 2 | 110 | 59 |
| 4203 | 1 | 77 | 34 |
| 4204 | 2 | 38 | 61 |
| 4205 | 1 | 122 | 29 |
| 4206 | 2 | 122 | 59 |
| 4207 | 1 | 89 | 34 |
| 4208 | 2 | 50 | 61 |
| 4209 | 1 | 134 | 29 |
| 4210 | 2 | 134 | 59 |
| 4211 | 1 | 101 | 34 |
| 4212 | 2 | 62 | 61 |
| 4213 | 1 | 79 | 64 |
| 4214 | 2 | 9 | 60 |
| 4215 | 1 | 6 | 61 |
| 4216 | 2 | 117 | 61 |
| 4217 | 1 | 91 | 64 |
| 4218 | 2 | 21 | 60 |
| 4219 | 1 | 18 | 61 |
| 4220 | 2 | 129 | 61 |
| 4221 | 1 | 103 | 64 |
| 4222 | 2 | 33 | 60 |
| 4223 | 1 | 30 | 61 |
| 4224 | 2 | 141 | 61 |
| 4225 | 1 | 118 | 4 |
| 4226 | 2 | 38 | 3 |
| 4227 | 1 | 2 | 5 |
| 4228 | 2 | 110 | 33 |
| 4229 | 1 | 130 | 4 |
| 4230 | 2 | 50 | 3 |
| 4231 | 1 | 14 | 5 |
| 4232 | 2 | 122 | 33 |
| 4233 | 1 | 142 | 4 |
| 4234 | 2 | 62 | 3 |
| 4235 | 1 | 26 | 5 |
| 4236 | 2 | 134 | 33 |
| 4237 | 1 | 40 | 33 |
| 4238 | 2 | 110 | 1 |
| 4239 | 1 | 118 | 3 |
| 4240 | 2 | 8 | 11 |
| 4241 | 1 | 52 | 33 |
| 4242 | 2 | 122 | 1 |
| 4243 | 1 | 130 | 3 |
| 4244 | 2 | 20 | 11 |
| 4245 | 1 | 64 | 33 |
| 4246 | 2 | 134 | 1 |
| 4247 | 1 | 142 | 3 |
| 4248 | 2 | 32 | 11 |
| 4249 | 1 | 38 | 2 |
| 4250 | 2 | 2 | 36 |
| 4251 | 1 | 110 | 34 |
| 4252 | 2 | 38 | 34 |
| 4253 | 1 | 50 | 2 |
| 4254 | 2 | 14 | 36 |
| 4255 | 1 | 122 | 34 |
| 4256 | 2 | 50 | 34 |
| 4257 | 1 | 62 | 2 |
| 4258 | 2 | 26 | 36 |
| 4259 | 1 | 134 | 34 |
| 4260 | 2 | 62 | 34 |
| 4261 | 1 | 84 | 38 |
| 4262 | 2 | 110 | 38 |
| 4263 | 1 | 4 | 39 |
| 4264 | 2 | 74 | 38 |
| 4265 | 1 | 96 | 38 |
| 4266 | 2 | 122 | 38 |
| 4267 | 1 | 16 | 39 |
| 4268 | 2 | 86 | 38 |
| 4269 | 1 | 108 | 38 |
| 4270 | 2 | 134 | 38 |
| 4271 | 1 | 28 | 39 |
| 4272 | 2 | 98 | 38 |
| 4273 | 1 | 7 | 38 |
| 4274 | 2 | 110 | 4 |
| 4275 | 1 | 113 | 40 |
| 4276 | 2 | 82 | 41 |
| 4277 | 1 | 19 | 38 |
| 4278 | 2 | 122 | 4 |
| 4279 | 1 | 125 | 40 |
| 4280 | 2 | 94 | 41 |
| 4281 | 1 | 31 | 38 |
| 4282 | 2 | 134 | 4 |
| 4283 | 1 | 137 | 40 |
| 4284 | 2 | 106 | 41 |
| 4285 | 1 | 6 | 11 |
| 4286 | 2 | 80 | 4 |
| 4287 | 1 | 114 | 35 |
| 4288 | 2 | 83 | 43 |
| 4289 | 1 | 18 | 11 |
| 4290 | 2 | 92 | 4 |
| 4291 | 1 | 126 | 35 |
| 4292 | 2 | 95 | 43 |
| 4293 | 1 | 30 | 11 |
| 4294 | 2 | 104 | 4 |
| 4295 | 1 | 138 | 35 |
| 4296 | 2 | 107 | 43 |
| 4297 | 1 | 4 | 40 |
| 4298 | 2 | 38 | 40 |
| 4299 | 1 | 115 | 40 |
| 4300 | 2 | 43 | 40 |
| 4301 | 1 | 16 | 40 |
| 4302 | 2 | 50 | 40 |
| 4303 | 1 | 127 | 40 |
| 4304 | 2 | 55 | 40 |
| 4305 | 1 | 28 | 40 |
| 4306 | 2 | 62 | 40 |
| 4307 | 1 | 139 | 40 |
| 4308 | 2 | 67 | 40 |
| 4309 | 1 | 76 | 9 |
| 4310 | 2 | 74 | 39 |
| 4311 | 1 | 40 | 40 |
| 4312 | 2 | 44 | 33 |
| 4313 | 1 | 88 | 9 |
| 4314 | 2 | 86 | 39 |
| 4315 | 1 | 52 | 40 |
| 4316 | 2 | 56 | 33 |
| 4317 | 1 | 100 | 9 |
| 4318 | 2 | 98 | 39 |
| 4319 | 1 | 64 | 40 |
| 4320 | 2 | 68 | 33 |
| 4321 | 1 | 118 | 12 |
| 4322 | 2 | 38 | 11 |
| 4323 | 1 | 2 | 13 |
| 4324 | 2 | 110 | 41 |
| 4325 | 1 | 130 | 12 |
| 4326 | 2 | 50 | 11 |
| 4327 | 1 | 14 | 13 |
| 4328 | 2 | 122 | 41 |
| 4329 | 1 | 142 | 12 |
| 4330 | 2 | 62 | 11 |
| 4331 | 1 | 26 | 13 |
| 4332 | 2 | 134 | 41 |
| 4333 | 1 | 40 | 41 |
| 4334 | 2 | 110 | 9 |
| 4335 | 1 | 118 | 11 |
| 4336 | 2 | 8 | 19 |
| 4337 | 1 | 52 | 41 |
| 4338 | 2 | 122 | 9 |
| 4339 | 1 | 130 | 11 |
| 4340 | 2 | 20 | 19 |
| 4341 | 1 | 64 | 41 |
| 4342 | 2 | 134 | 9 |
| 4343 | 1 | 142 | 11 |
| 4344 | 2 | 32 | 19 |
| 4345 | 1 | 38 | 10 |
| 4346 | 2 | 2 | 44 |
| 4347 | 1 | 110 | 42 |
| 4348 | 2 | 38 | 42 |
| 4349 | 1 | 50 | 10 |
| 4350 | 2 | 14 | 44 |
| 4351 | 1 | 122 | 42 |
| 4352 | 2 | 50 | 42 |
| 4353 | 1 | 62 | 10 |
| 4354 | 2 | 26 | 44 |
| 4355 | 1 | 134 | 42 |
| 4356 | 2 | 62 | 42 |
| 4357 | 1 | 84 | 46 |
| 4358 | 2 | 110 | 46 |
| 4359 | 1 | 4 | 47 |
| 4360 | 2 | 74 | 46 |
| 4361 | 1 | 96 | 46 |
| 4362 | 2 | 122 | 46 |
| 4363 | 1 | 16 | 47 |
| 4364 | 2 | 86 | 46 |
| 4365 | 1 | 108 | 46 |
| 4366 | 2 | 134 | 46 |
| 4367 | 1 | 28 | 47 |
| 4368 | 2 | 98 | 46 |
| 4369 | 1 | 7 | 46 |
| 4370 | 2 | 110 | 12 |
| 4371 | 1 | 113 | 48 |
| 4372 | 2 | 82 | 49 |
| 4373 | 1 | 19 | 46 |
| 4374 | 2 | 122 | 12 |
| 4375 | 1 | 125 | 48 |
| 4376 | 2 | 94 | 49 |
| 4377 | 1 | 31 | 46 |
| 4378 | 2 | 134 | 12 |
| 4379 | 1 | 137 | 48 |
| 4380 | 2 | 106 | 49 |
| 4381 | 1 | 6 | 19 |
| 4382 | 2 | 80 | 12 |
| 4383 | 1 | 114 | 43 |
| 4384 | 2 | 83 | 51 |
| 4385 | 1 | 18 | 19 |
| 4386 | 2 | 92 | 12 |
| 4387 | 1 | 126 | 43 |
| 4388 | 2 | 95 | 51 |
| 4389 | 1 | 30 | 19 |
| 4390 | 2 | 104 | 12 |
| 4391 | 1 | 138 | 43 |
| 4392 | 2 | 107 | 51 |
| 4393 | 1 | 4 | 48 |
| 4394 | 2 | 38 | 48 |
| 4395 | 1 | 115 | 48 |
| 4396 | 2 | 43 | 48 |
| 4397 | 1 | 16 | 48 |
| 4398 | 2 | 50 | 48 |
| 4399 | 1 | 127 | 48 |
| 4400 | 2 | 55 | 48 |
| 4401 | 1 | 28 | 48 |
| 4402 | 2 | 62 | 48 |
| 4403 | 1 | 139 | 48 |
| 4404 | 2 | 67 | 48 |
| 4405 | 1 | 76 | 17 |
| 4406 | 2 | 74 | 47 |
| 4407 | 1 | 40 | 48 |
| 4408 | 2 | 44 | 41 |
| 4409 | 1 | 88 | 17 |
| 4410 | 2 | 86 | 47 |
| 4411 | 1 | 52 | 48 |
| 4412 | 2 | 56 | 41 |
| 4413 | 1 | 100 | 17 |
| 4414 | 2 | 98 | 47 |
| 4415 | 1 | 64 | 48 |
| 4416 | 2 | 68 | 41 |
| 4417 | 1 | 118 | 20 |
| 4418 | 2 | 38 | 19 |
| 4419 | 1 | 2 | 21 |
| 4420 | 2 | 110 | 49 |
| 4421 | 1 | 130 | 20 |
| 4422 | 2 | 50 | 19 |
| 4423 | 1 | 14 | 21 |
| 4424 | 2 | 122 | 49 |
| 4425 | 1 | 142 | 20 |
| 4426 | 2 | 62 | 19 |
| 4427 | 1 | 26 | 21 |
| 4428 | 2 | 134 | 49 |
| 4429 | 1 | 40 | 49 |
| 4430 | 2 | 110 | 17 |
| 4431 | 1 | 118 | 19 |
| 4432 | 2 | 8 | 27 |
| 4433 | 1 | 52 | 49 |
| 4434 | 2 | 122 | 17 |
| 4435 | 1 | 130 | 19 |
| 4436 | 2 | 20 | 27 |
| 4437 | 1 | 64 | 49 |
| 4438 | 2 | 134 | 17 |
| 4439 | 1 | 142 | 19 |
| 4440 | 2 | 32 | 27 |
| 4441 | 1 | 38 | 18 |
| 4442 | 2 | 2 | 52 |
| 4443 | 1 | 110 | 50 |
| 4444 | 2 | 38 | 50 |
| 4445 | 1 | 50 | 18 |
| 4446 | 2 | 14 | 52 |
| 4447 | 1 | 122 | 50 |
| 4448 | 2 | 50 | 50 |
| 4449 | 1 | 62 | 18 |
| 4450 | 2 | 26 | 52 |
| 4451 | 1 | 134 | 50 |
| 4452 | 2 | 62 | 50 |
| 4453 | 1 | 84 | 54 |
| 4454 | 2 | 110 | 54 |
| 4455 | 1 | 4 | 55 |
| 4456 | 2 | 74 | 54 |
| 4457 | 1 | 96 | 54 |
| 4458 | 2 | 122 | 54 |
| 4459 | 1 | 16 | 55 |
| 4460 | 2 | 86 | 54 |
| 4461 | 1 | 108 | 54 |
| 4462 | 2 | 134 | 54 |
| 4463 | 1 | 28 | 55 |
| 4464 | 2 | 98 | 54 |
| 4465 | 1 | 7 | 54 |
| 4466 | 2 | 110 | 20 |
| 4467 | 1 | 113 | 56 |
| 4468 | 2 | 82 | 57 |
| 4469 | 1 | 19 | 54 |
| 4470 | 2 | 122 | 20 |
| 4471 | 1 | 125 | 56 |
| 4472 | 2 | 94 | 57 |
| 4473 | 1 | 31 | 54 |
| 4474 | 2 | 134 | 20 |
| 4475 | 1 | 137 | 56 |
| 4476 | 2 | 106 | 57 |
| 4477 | 1 | 6 | 27 |
| 4478 | 2 | 80 | 20 |
| 4479 | 1 | 114 | 51 |
| 4480 | 2 | 83 | 59 |
| 4481 | 1 | 18 | 27 |
| 4482 | 2 | 92 | 20 |
| 4483 | 1 | 126 | 51 |
| 4484 | 2 | 95 | 59 |
| 4485 | 1 | 30 | 27 |
| 4486 | 2 | 104 | 20 |
| 4487 | 1 | 138 | 51 |
| 4488 | 2 | 107 | 59 |
| 4489 | 1 | 4 | 56 |
| 4490 | 2 | 38 | 56 |
| 4491 | 1 | 115 | 56 |
| 4492 | 2 | 43 | 56 |
| 4493 | 1 | 16 | 56 |
| 4494 | 2 | 50 | 56 |
| 4495 | 1 | 127 | 56 |
| 4496 | 2 | 55 | 56 |
| 4497 | 1 | 28 | 56 |
| 4498 | 2 | 62 | 56 |
| 4499 | 1 | 139 | 56 |
| 4500 | 2 | 67 | 56 |
| 4501 | 1 | 76 | 25 |
| 4502 | 2 | 74 | 55 |
| 4503 | 1 | 40 | 56 |
| 4504 | 2 | 44 | 49 |
| 4505 | 1 | 88 | 25 |
| 4506 | 2 | 86 | 55 |
| 4507 | 1 | 52 | 56 |
| 4508 | 2 | 56 | 49 |
| 4509 | 1 | 100 | 25 |
| 4510 | 2 | 98 | 55 |
| 4511 | 1 | 64 | 56 |
| 4512 | 2 | 68 | 49 |
| 4513 | 1 | 118 | 28 |
| 4514 | 2 | 38 | 27 |
| 4515 | 1 | 2 | 29 |
| 4516 | 2 | 110 | 57 |
| 4517 | 1 | 130 | 28 |
| 4518 | 2 | 50 | 27 |
| 4519 | 1 | 14 | 29 |
| 4520 | 2 | 122 | 57 |
| 4521 | 1 | 142 | 28 |
| 4522 | 2 | 62 | 27 |
| 4523 | 1 | 26 | 29 |
| 4524 | 2 | 134 | 57 |
| 4525 | 1 | 40 | 57 |
| 4526 | 2 | 110 | 25 |
| 4527 | 1 | 118 | 27 |
| 4528 | 2 | 8 | 35 |
| 4529 | 1 | 52 | 57 |
| 4530 | 2 | 122 | 25 |
| 4531 | 1 | 130 | 27 |
| 4532 | 2 | 20 | 35 |
| 4533 | 1 | 64 | 57 |
| 4534 | 2 | 134 | 25 |
| 4535 | 1 | 142 | 27 |
| 4536 | 2 | 32 | 35 |
| 4537 | 1 | 38 | 26 |
| 4538 | 2 | 2 | 60 |
| 4539 | 1 | 110 | 58 |
| 4540 | 2 | 38 | 58 |
| 4541 | 1 | 50 | 26 |
| 4542 | 2 | 14 | 60 |
| 4543 | 1 | 122 | 58 |
| 4544 | 2 | 50 | 58 |
| 4545 | 1 | 62 | 26 |
| 4546 | 2 | 26 | 60 |
| 4547 | 1 | 134 | 58 |
| 4548 | 2 | 62 | 58 |
| 4549 | 1 | 84 | 62 |
| 4550 | 2 | 110 | 62 |
| 4551 | 1 | 4 | 63 |
| 4552 | 2 | 74 | 62 |
| 4553 | 1 | 96 | 62 |
| 4554 | 2 | 122 | 62 |
| 4555 | 1 | 16 | 63 |
| 4556 | 2 | 86 | 62 |
| 4557 | 1 | 108 | 62 |
| 4558 | 2 | 134 | 62 |
| 4559 | 1 | 28 | 63 |
| 4560 | 2 | 98 | 62 |
| 4561 | 1 | 7 | 62 |
| 4562 | 2 | 110 | 28 |
| 4563 | 1 | 113 | 64 |
| 4564 | 2 | 82 | 65 |
| 4565 | 1 | 19 | 62 |
| 4566 | 2 | 122 | 28 |
| 4567 | 1 | 125 | 64 |
| 4568 | 2 | 94 | 65 |
| 4569 | 1 | 31 | 62 |
| 4570 | 2 | 134 | 28 |
| 4571 | 1 | 137 | 64 |
| 4572 | 2 | 106 | 65 |
| 4573 | 1 | 6 | 35 |
| 4574 | 2 | 80 | 28 |
| 4575 | 1 | 114 | 59 |
| 4576 | 2 | 83 | 67 |
| 4577 | 1 | 18 | 35 |
| 4578 | 2 | 92 | 28 |
| 4579 | 1 | 126 | 59 |
| 4580 | 2 | 95 | 67 |
| 4581 | 1 | 30 | 35 |
| 4582 | 2 | 104 | 28 |
| 4583 | 1 | 138 | 59 |
| 4584 | 2 | 107 | 67 |
| 4585 | 1 | 4 | 64 |
| 4586 | 2 | 38 | 64 |
| 4587 | 1 | 115 | 64 |
| 4588 | 2 | 43 | 64 |
| 4589 | 1 | 16 | 64 |
| 4590 | 2 | 50 | 64 |
| 4591 | 1 | 127 | 64 |
| 4592 | 2 | 55 | 64 |
| 4593 | 1 | 28 | 64 |
| 4594 | 2 | 62 | 64 |
| 4595 | 1 | 139 | 64 |
| 4596 | 2 | 67 | 64 |
| 4597 | 1 | 76 | 33 |
| 4598 | 2 | 74 | 63 |
| 4599 | 1 | 40 | 64 |
| 4600 | 2 | 44 | 57 |
| 4601 | 1 | 88 | 33 |
| 4602 | 2 | 86 | 63 |
| 4603 | 1 | 52 | 64 |
| 4604 | 2 | 56 | 57 |
| 4605 | 1 | 100 | 33 |
| 4606 | 2 | 98 | 63 |
| 4607 | 1 | 64 | 64 |
| 4608 | 2 | 68 | 57 |
| 4609 | 1 | 38 | 38 |
| 4610 | 2 | 74 | 40 |
| 4611 | 1 | 5 | 38 |
| 4612 | 2 | 2 | 41 |
| 4613 | 1 | 50 | 38 |
| 4614 | 2 | 86 | 40 |
| 4615 | 1 | 17 | 38 |
| 4616 | 2 | 14 | 41 |
| 4617 | 1 | 62 | 38 |
| 4618 | 2 | 98 | 40 |
| 4619 | 1 | 29 | 38 |
| 4620 | 2 | 26 | 41 |
| 4621 | 1 | 7 | 39 |
| 4622 | 2 | 75 | 5 |
| 4623 | 1 | 75 | 41 |
| 4624 | 2 | 38 | 7 |
| 4625 | 1 | 19 | 39 |
| 4626 | 2 | 87 | 5 |
| 4627 | 1 | 87 | 41 |
| 4628 | 2 | 50 | 7 |
| 4629 | 1 | 31 | 39 |
| 4630 | 2 | 99 | 5 |
| 4631 | 1 | 99 | 41 |
| 4632 | 2 | 62 | 7 |
| 4633 | 1 | 43 | 34 |
| 4634 | 2 | 118 | 39 |
| 4635 | 1 | 120 | 6 |
| 4636 | 2 | 74 | 10 |
| 4637 | 1 | 55 | 34 |
| 4638 | 2 | 130 | 39 |
| 4639 | 1 | 132 | 6 |
| 4640 | 2 | 86 | 10 |
| 4641 | 1 | 67 | 34 |
| 4642 | 2 | 142 | 39 |
| 4643 | 1 | 144 | 6 |
| 4644 | 2 | 98 | 10 |
| 4645 | 1 | 44 | 2 |
| 4646 | 2 | 2 | 9 |
| 4647 | 1 | 110 | 7 |
| 4648 | 2 | 10 | 11 |
| 4649 | 1 | 56 | 2 |
| 4650 | 2 | 14 | 9 |
| 4651 | 1 | 122 | 7 |
| 4652 | 2 | 22 | 11 |
| 4653 | 1 | 68 | 2 |
| 4654 | 2 | 26 | 9 |
| 4655 | 1 | 134 | 7 |
| 4656 | 2 | 34 | 11 |
| 4657 | 1 | 80 | 7 |
| 4658 | 2 | 38 | 8 |
| 4659 | 1 | 113 | 36 |
| 4660 | 2 | 39 | 1 |
| 4661 | 1 | 92 | 7 |
| 4662 | 2 | 50 | 8 |
| 4663 | 1 | 125 | 36 |
| 4664 | 2 | 51 | 1 |
| 4665 | 1 | 104 | 7 |
| 4666 | 2 | 62 | 8 |
| 4667 | 1 | 137 | 36 |
| 4668 | 2 | 63 | 1 |
| 4669 | 1 | 80 | 40 |
| 4670 | 2 | 74 | 11 |
| 4671 | 1 | 4 | 43 |
| 4672 | 2 | 111 | 1 |
| 4673 | 1 | 92 | 40 |
| 4674 | 2 | 86 | 11 |
| 4675 | 1 | 16 | 43 |
| 4676 | 2 | 123 | 1 |
| 4677 | 1 | 104 | 40 |
| 4678 | 2 | 98 | 11 |
| 4679 | 1 | 28 | 43 |
| 4680 | 2 | 135 | 1 |
| 4681 | 1 | 83 | 4 |
| 4682 | 2 | 10 | 42 |
| 4683 | 1 | 4 | 38 |
| 4684 | 2 | 41 | 38 |
| 4685 | 1 | 95 | 4 |
| 4686 | 2 | 22 | 42 |
| 4687 | 1 | 16 | 38 |
| 4688 | 2 | 53 | 38 |
| 4689 | 1 | 107 | 4 |
| 4690 | 2 | 34 | 42 |
| 4691 | 1 | 28 | 38 |
| 4692 | 2 | 65 | 38 |
| 4693 | 1 | 8 | 40 |
| 4694 | 2 | 110 | 8 |
| 4695 | 1 | 75 | 36 |
| 4696 | 2 | 3 | 36 |
| 4697 | 1 | 20 | 40 |
| 4698 | 2 | 122 | 8 |
| 4699 | 1 | 87 | 36 |
| 4700 | 2 | 15 | 36 |
| 4701 | 1 | 32 | 40 |
| 4702 | 2 | 134 | 8 |
| 4703 | 1 | 99 | 36 |
| 4704 | 2 | 27 | 36 |
| 4705 | 1 | 38 | 46 |
| 4706 | 2 | 74 | 48 |
| 4707 | 1 | 5 | 46 |
| 4708 | 2 | 2 | 49 |
| 4709 | 1 | 50 | 46 |
| 4710 | 2 | 86 | 48 |
| 4711 | 1 | 17 | 46 |
| 4712 | 2 | 14 | 49 |
| 4713 | 1 | 62 | 46 |
| 4714 | 2 | 98 | 48 |
| 4715 | 1 | 29 | 46 |
| 4716 | 2 | 26 | 49 |
| 4717 | 1 | 7 | 47 |
| 4718 | 2 | 75 | 13 |
| 4719 | 1 | 75 | 49 |
| 4720 | 2 | 38 | 15 |
| 4721 | 1 | 19 | 47 |
| 4722 | 2 | 87 | 13 |
| 4723 | 1 | 87 | 49 |
| 4724 | 2 | 50 | 15 |
| 4725 | 1 | 31 | 47 |
| 4726 | 2 | 99 | 13 |
| 4727 | 1 | 99 | 49 |
| 4728 | 2 | 62 | 15 |
| 4729 | 1 | 43 | 42 |
| 4730 | 2 | 118 | 47 |
| 4731 | 1 | 120 | 14 |
| 4732 | 2 | 74 | 18 |
| 4733 | 1 | 55 | 42 |
| 4734 | 2 | 130 | 47 |
| 4735 | 1 | 132 | 14 |
| 4736 | 2 | 86 | 18 |
| 4737 | 1 | 67 | 42 |
| 4738 | 2 | 142 | 47 |
| 4739 | 1 | 144 | 14 |
| 4740 | 2 | 98 | 18 |
| 4741 | 1 | 44 | 10 |
| 4742 | 2 | 2 | 17 |
| 4743 | 1 | 110 | 15 |
| 4744 | 2 | 10 | 19 |
| 4745 | 1 | 56 | 10 |
| 4746 | 2 | 14 | 17 |
| 4747 | 1 | 122 | 15 |
| 4748 | 2 | 22 | 19 |
| 4749 | 1 | 68 | 10 |
| 4750 | 2 | 26 | 17 |
| 4751 | 1 | 134 | 15 |
| 4752 | 2 | 34 | 19 |
| 4753 | 1 | 80 | 15 |
| 4754 | 2 | 38 | 16 |
| 4755 | 1 | 113 | 44 |
| 4756 | 2 | 39 | 9 |
| 4757 | 1 | 92 | 15 |
| 4758 | 2 | 50 | 16 |
| 4759 | 1 | 125 | 44 |
| 4760 | 2 | 51 | 9 |
| 4761 | 1 | 104 | 15 |
| 4762 | 2 | 62 | 16 |
| 4763 | 1 | 137 | 44 |
| 4764 | 2 | 63 | 9 |
| 4765 | 1 | 80 | 48 |
| 4766 | 2 | 74 | 19 |
| 4767 | 1 | 4 | 51 |
| 4768 | 2 | 111 | 9 |
| 4769 | 1 | 92 | 48 |
| 4770 | 2 | 86 | 19 |
| 4771 | 1 | 16 | 51 |
| 4772 | 2 | 123 | 9 |
| 4773 | 1 | 104 | 48 |
| 4774 | 2 | 98 | 19 |
| 4775 | 1 | 28 | 51 |
| 4776 | 2 | 135 | 9 |
| 4777 | 1 | 83 | 12 |
| 4778 | 2 | 10 | 50 |
| 4779 | 1 | 4 | 46 |
| 4780 | 2 | 41 | 46 |
| 4781 | 1 | 95 | 12 |
| 4782 | 2 | 22 | 50 |
| 4783 | 1 | 16 | 46 |
| 4784 | 2 | 53 | 46 |
| 4785 | 1 | 107 | 12 |
| 4786 | 2 | 34 | 50 |
| 4787 | 1 | 28 | 46 |
| 4788 | 2 | 65 | 46 |
| 4789 | 1 | 8 | 48 |
| 4790 | 2 | 110 | 16 |
| 4791 | 1 | 75 | 44 |
| 4792 | 2 | 3 | 44 |
| 4793 | 1 | 20 | 48 |
| 4794 | 2 | 122 | 16 |
| 4795 | 1 | 87 | 44 |
| 4796 | 2 | 15 | 44 |
| 4797 | 1 | 32 | 48 |
| 4798 | 2 | 134 | 16 |
| 4799 | 1 | 99 | 44 |
| 4800 | 2 | 27 | 44 |
| 4801 | 1 | 38 | 54 |
| 4802 | 2 | 74 | 56 |
| 4803 | 1 | 5 | 54 |
| 4804 | 2 | 2 | 57 |
| 4805 | 1 | 50 | 54 |
| 4806 | 2 | 86 | 56 |
| 4807 | 1 | 17 | 54 |
| 4808 | 2 | 14 | 57 |
| 4809 | 1 | 62 | 54 |
| 4810 | 2 | 98 | 56 |
| 4811 | 1 | 29 | 54 |
| 4812 | 2 | 26 | 57 |
| 4813 | 1 | 7 | 55 |
| 4814 | 2 | 75 | 21 |
| 4815 | 1 | 75 | 57 |
| 4816 | 2 | 38 | 23 |
| 4817 | 1 | 19 | 55 |
| 4818 | 2 | 87 | 21 |
| 4819 | 1 | 87 | 57 |
| 4820 | 2 | 50 | 23 |
| 4821 | 1 | 31 | 55 |
| 4822 | 2 | 99 | 21 |
| 4823 | 1 | 99 | 57 |
| 4824 | 2 | 62 | 23 |
| 4825 | 1 | 43 | 50 |
| 4826 | 2 | 118 | 55 |
| 4827 | 1 | 120 | 22 |
| 4828 | 2 | 74 | 26 |
| 4829 | 1 | 55 | 50 |
| 4830 | 2 | 130 | 55 |
| 4831 | 1 | 132 | 22 |
| 4832 | 2 | 86 | 26 |
| 4833 | 1 | 67 | 50 |
| 4834 | 2 | 142 | 55 |
| 4835 | 1 | 144 | 22 |
| 4836 | 2 | 98 | 26 |
| 4837 | 1 | 44 | 18 |
| 4838 | 2 | 2 | 25 |
| 4839 | 1 | 110 | 23 |
| 4840 | 2 | 10 | 27 |
| 4841 | 1 | 56 | 18 |
| 4842 | 2 | 14 | 25 |
| 4843 | 1 | 122 | 23 |
| 4844 | 2 | 22 | 27 |
| 4845 | 1 | 68 | 18 |
| 4846 | 2 | 26 | 25 |
| 4847 | 1 | 134 | 23 |
| 4848 | 2 | 34 | 27 |
| 4849 | 1 | 80 | 23 |
| 4850 | 2 | 38 | 24 |
| 4851 | 1 | 113 | 52 |
| 4852 | 2 | 39 | 17 |
| 4853 | 1 | 92 | 23 |
| 4854 | 2 | 50 | 24 |
| 4855 | 1 | 125 | 52 |
| 4856 | 2 | 51 | 17 |
| 4857 | 1 | 104 | 23 |
| 4858 | 2 | 62 | 24 |
| 4859 | 1 | 137 | 52 |
| 4860 | 2 | 63 | 17 |
| 4861 | 1 | 80 | 56 |
| 4862 | 2 | 74 | 27 |
| 4863 | 1 | 4 | 59 |
| 4864 | 2 | 111 | 17 |
| 4865 | 1 | 92 | 56 |
| 4866 | 2 | 86 | 27 |
| 4867 | 1 | 16 | 59 |
| 4868 | 2 | 123 | 17 |
| 4869 | 1 | 104 | 56 |
| 4870 | 2 | 98 | 27 |
| 4871 | 1 | 28 | 59 |
| 4872 | 2 | 135 | 17 |
| 4873 | 1 | 83 | 20 |
| 4874 | 2 | 10 | 58 |
| 4875 | 1 | 4 | 54 |
| 4876 | 2 | 41 | 54 |
| 4877 | 1 | 95 | 20 |
| 4878 | 2 | 22 | 58 |
| 4879 | 1 | 16 | 54 |
| 4880 | 2 | 53 | 54 |
| 4881 | 1 | 107 | 20 |
| 4882 | 2 | 34 | 58 |
| 4883 | 1 | 28 | 54 |
| 4884 | 2 | 65 | 54 |
| 4885 | 1 | 8 | 56 |
| 4886 | 2 | 110 | 24 |
| 4887 | 1 | 75 | 52 |
| 4888 | 2 | 3 | 52 |
| 4889 | 1 | 20 | 56 |
| 4890 | 2 | 122 | 24 |
| 4891 | 1 | 87 | 52 |
| 4892 | 2 | 15 | 52 |
| 4893 | 1 | 32 | 56 |
| 4894 | 2 | 134 | 24 |
| 4895 | 1 | 99 | 52 |
| 4896 | 2 | 27 | 52 |
| 4897 | 1 | 38 | 62 |
| 4898 | 2 | 74 | 64 |
| 4899 | 1 | 5 | 62 |
| 4900 | 2 | 2 | 65 |
| 4901 | 1 | 50 | 62 |
| 4902 | 2 | 86 | 64 |
| 4903 | 1 | 17 | 62 |
| 4904 | 2 | 14 | 65 |
| 4905 | 1 | 62 | 62 |
| 4906 | 2 | 98 | 64 |
| 4907 | 1 | 29 | 62 |
| 4908 | 2 | 26 | 65 |
| 4909 | 1 | 7 | 63 |
| 4910 | 2 | 75 | 29 |
| 4911 | 1 | 75 | 65 |
| 4912 | 2 | 38 | 31 |
| 4913 | 1 | 19 | 63 |
| 4914 | 2 | 87 | 29 |
| 4915 | 1 | 87 | 65 |
| 4916 | 2 | 50 | 31 |
| 4917 | 1 | 31 | 63 |
| 4918 | 2 | 99 | 29 |
| 4919 | 1 | 99 | 65 |
| 4920 | 2 | 62 | 31 |
| 4921 | 1 | 43 | 58 |
| 4922 | 2 | 118 | 63 |
| 4923 | 1 | 120 | 30 |
| 4924 | 2 | 74 | 34 |
| 4925 | 1 | 55 | 58 |
| 4926 | 2 | 130 | 63 |
| 4927 | 1 | 132 | 30 |
| 4928 | 2 | 86 | 34 |
| 4929 | 1 | 67 | 58 |
| 4930 | 2 | 142 | 63 |
| 4931 | 1 | 144 | 30 |
| 4932 | 2 | 98 | 34 |
| 4933 | 1 | 44 | 26 |
| 4934 | 2 | 2 | 33 |
| 4935 | 1 | 110 | 31 |
| 4936 | 2 | 10 | 35 |
| 4937 | 1 | 56 | 26 |
| 4938 | 2 | 14 | 33 |
| 4939 | 1 | 122 | 31 |
| 4940 | 2 | 22 | 35 |
| 4941 | 1 | 68 | 26 |
| 4942 | 2 | 26 | 33 |
| 4943 | 1 | 134 | 31 |
| 4944 | 2 | 34 | 35 |
| 4945 | 1 | 80 | 31 |
| 4946 | 2 | 38 | 32 |
| 4947 | 1 | 113 | 60 |
| 4948 | 2 | 39 | 25 |
| 4949 | 1 | 92 | 31 |
| 4950 | 2 | 50 | 32 |
| 4951 | 1 | 125 | 60 |
| 4952 | 2 | 51 | 25 |
| 4953 | 1 | 104 | 31 |
| 4954 | 2 | 62 | 32 |
| 4955 | 1 | 137 | 60 |
| 4956 | 2 | 63 | 25 |
| 4957 | 1 | 80 | 64 |
| 4958 | 2 | 74 | 35 |
| 4959 | 1 | 4 | 67 |
| 4960 | 2 | 111 | 25 |
| 4961 | 1 | 92 | 64 |
| 4962 | 2 | 86 | 35 |
| 4963 | 1 | 16 | 67 |
| 4964 | 2 | 123 | 25 |
| 4965 | 1 | 104 | 64 |
| 4966 | 2 | 98 | 35 |
| 4967 | 1 | 28 | 67 |
| 4968 | 2 | 135 | 25 |
| 4969 | 1 | 83 | 28 |
| 4970 | 2 | 10 | 66 |
| 4971 | 1 | 4 | 62 |
| 4972 | 2 | 41 | 62 |
| 4973 | 1 | 95 | 28 |
| 4974 | 2 | 22 | 66 |
| 4975 | 1 | 16 | 62 |
| 4976 | 2 | 53 | 62 |
| 4977 | 1 | 107 | 28 |
| 4978 | 2 | 34 | 66 |
| 4979 | 1 | 28 | 62 |
| 4980 | 2 | 65 | 62 |
| 4981 | 1 | 8 | 64 |
| 4982 | 2 | 110 | 32 |
| 4983 | 1 | 75 | 60 |
| 4984 | 2 | 3 | 60 |
| 4985 | 1 | 20 | 64 |
| 4986 | 2 | 122 | 32 |
| 4987 | 1 | 87 | 60 |
| 4988 | 2 | 15 | 60 |
| 4989 | 1 | 32 | 64 |
| 4990 | 2 | 134 | 32 |
| 4991 | 1 | 99 | 60 |
| 4992 | 2 | 27 | 60 |
| 4993 | 1 | 114 | 33 |
| 4994 | 2 | 82 | 38 |
| 4995 | 1 | 46 | 5 |
| 4996 | 2 | 38 | 39 |
| 4997 | 1 | 126 | 33 |
| 4998 | 2 | 94 | 38 |
| 4999 | 1 | 58 | 5 |
| 5000 | 2 | 50 | 39 |
| 5001 | 1 | 138 | 33 |
| 5002 | 2 | 106 | 38 |
| 5003 | 1 | 70 | 5 |
| 5004 | 2 | 62 | 39 |
| 5005 | 1 | 9 | 41 |
| 5006 | 2 | 38 | 6 |
| 5007 | 1 | 8 | 6 |
| 5008 | 2 | 114 | 1 |
| 5009 | 1 | 21 | 41 |
| 5010 | 2 | 50 | 6 |
| 5011 | 1 | 20 | 6 |
| 5012 | 2 | 126 | 1 |
| 5013 | 1 | 33 | 41 |
| 5014 | 2 | 62 | 6 |
| 5015 | 1 | 32 | 6 |
| 5016 | 2 | 138 | 1 |
| 5017 | 1 | 44 | 1 |
| 5018 | 2 | 74 | 9 |
| 5019 | 1 | 45 | 7 |
| 5020 | 2 | 74 | 42 |
| 5021 | 1 | 56 | 1 |
| 5022 | 2 | 86 | 9 |
| 5023 | 1 | 57 | 7 |
| 5024 | 2 | 86 | 42 |
| 5025 | 1 | 68 | 1 |
| 5026 | 2 | 98 | 9 |
| 5027 | 1 | 69 | 7 |
| 5028 | 2 | 98 | 42 |
| 5029 | 1 | 76 | 10 |
| 5030 | 2 | 114 | 6 |
| 5031 | 1 | 2 | 11 |
| 5032 | 2 | 3 | 40 |
| 5033 | 1 | 88 | 10 |
| 5034 | 2 | 126 | 6 |
| 5035 | 1 | 14 | 11 |
| 5036 | 2 | 15 | 40 |
| 5037 | 1 | 100 | 10 |
| 5038 | 2 | 138 | 6 |
| 5039 | 1 | 26 | 11 |
| 5040 | 2 | 27 | 40 |
| 5041 | 1 | 45 | 38 |
| 5042 | 2 | 2 | 43 |
| 5043 | 1 | 84 | 4 |
| 5044 | 2 | 39 | 33 |
| 5045 | 1 | 57 | 38 |
| 5046 | 2 | 14 | 43 |
| 5047 | 1 | 96 | 4 |
| 5048 | 2 | 51 | 33 |
| 5049 | 1 | 69 | 38 |
| 5050 | 2 | 26 | 43 |
| 5051 | 1 | 108 | 4 |
| 5052 | 2 | 63 | 33 |
| 5053 | 1 | 6 | 8 |
| 5054 | 2 | 119 | 5 |
| 5055 | 1 | 81 | 42 |
| 5056 | 2 | 39 | 2 |
| 5057 | 1 | 18 | 8 |
| 5058 | 2 | 131 | 5 |
| 5059 | 1 | 93 | 42 |
| 5060 | 2 | 51 | 2 |
| 5061 | 1 | 30 | 8 |
| 5062 | 2 | 143 | 5 |
| 5063 | 1 | 105 | 42 |
| 5064 | 2 | 63 | 2 |
| 5065 | 1 | 39 | 4 |
| 5066 | 2 | 74 | 43 |
| 5067 | 1 | 12 | 38 |
| 5068 | 2 | 39 | 39 |
| 5069 | 1 | 51 | 4 |
| 5070 | 2 | 86 | 43 |
| 5071 | 1 | 24 | 38 |
| 5072 | 2 | 51 | 39 |
| 5073 | 1 | 63 | 4 |
| 5074 | 2 | 98 | 43 |
| 5075 | 1 | 36 | 38 |
| 5076 | 2 | 63 | 39 |
| 5077 | 1 | 5 | 37 |
| 5078 | 2 | 117 | 33 |
| 5079 | 1 | 111 | 34 |
| 5080 | 2 | 111 | 2 |
| 5081 | 1 | 17 | 37 |
| 5082 | 2 | 129 | 33 |
| 5083 | 1 | 123 | 34 |
| 5084 | 2 | 123 | 2 |
| 5085 | 1 | 29 | 37 |
| 5086 | 2 | 141 | 33 |
| 5087 | 1 | 135 | 34 |
| 5088 | 2 | 135 | 2 |
| 5089 | 1 | 114 | 41 |
| 5090 | 2 | 82 | 46 |
| 5091 | 1 | 46 | 13 |
| 5092 | 2 | 38 | 47 |
| 5093 | 1 | 126 | 41 |
| 5094 | 2 | 94 | 46 |
| 5095 | 1 | 58 | 13 |
| 5096 | 2 | 50 | 47 |
| 5097 | 1 | 138 | 41 |
| 5098 | 2 | 106 | 46 |
| 5099 | 1 | 70 | 13 |
| 5100 | 2 | 62 | 47 |
| 5101 | 1 | 9 | 49 |
| 5102 | 2 | 38 | 14 |
| 5103 | 1 | 8 | 14 |
| 5104 | 2 | 114 | 9 |
| 5105 | 1 | 21 | 49 |
| 5106 | 2 | 50 | 14 |
| 5107 | 1 | 20 | 14 |
| 5108 | 2 | 126 | 9 |
| 5109 | 1 | 33 | 49 |
| 5110 | 2 | 62 | 14 |
| 5111 | 1 | 32 | 14 |
| 5112 | 2 | 138 | 9 |
| 5113 | 1 | 44 | 9 |
| 5114 | 2 | 74 | 17 |
| 5115 | 1 | 45 | 15 |
| 5116 | 2 | 74 | 50 |
| 5117 | 1 | 56 | 9 |
| 5118 | 2 | 86 | 17 |
| 5119 | 1 | 57 | 15 |
| 5120 | 2 | 86 | 50 |
| 5121 | 1 | 68 | 9 |
| 5122 | 2 | 98 | 17 |
| 5123 | 1 | 69 | 15 |
| 5124 | 2 | 98 | 50 |
| 5125 | 1 | 76 | 18 |
| 5126 | 2 | 114 | 14 |
| 5127 | 1 | 2 | 19 |
| 5128 | 2 | 3 | 48 |
| 5129 | 1 | 88 | 18 |
| 5130 | 2 | 126 | 14 |
| 5131 | 1 | 14 | 19 |
| 5132 | 2 | 15 | 48 |
| 5133 | 1 | 100 | 18 |
| 5134 | 2 | 138 | 14 |
| 5135 | 1 | 26 | 19 |
| 5136 | 2 | 27 | 48 |
| 5137 | 1 | 45 | 46 |
| 5138 | 2 | 2 | 51 |
| 5139 | 1 | 84 | 12 |
| 5140 | 2 | 39 | 41 |
| 5141 | 1 | 57 | 46 |
| 5142 | 2 | 14 | 51 |
| 5143 | 1 | 96 | 12 |
| 5144 | 2 | 51 | 41 |
| 5145 | 1 | 69 | 46 |
| 5146 | 2 | 26 | 51 |
| 5147 | 1 | 108 | 12 |
| 5148 | 2 | 63 | 41 |
| 5149 | 1 | 6 | 16 |
| 5150 | 2 | 119 | 13 |
| 5151 | 1 | 81 | 50 |
| 5152 | 2 | 39 | 10 |
| 5153 | 1 | 18 | 16 |
| 5154 | 2 | 131 | 13 |
| 5155 | 1 | 93 | 50 |
| 5156 | 2 | 51 | 10 |
| 5157 | 1 | 30 | 16 |
| 5158 | 2 | 143 | 13 |
| 5159 | 1 | 105 | 50 |
| 5160 | 2 | 63 | 10 |
| 5161 | 1 | 39 | 12 |
| 5162 | 2 | 74 | 51 |
| 5163 | 1 | 12 | 46 |
| 5164 | 2 | 39 | 47 |
| 5165 | 1 | 51 | 12 |
| 5166 | 2 | 86 | 51 |
| 5167 | 1 | 24 | 46 |
| 5168 | 2 | 51 | 47 |
| 5169 | 1 | 63 | 12 |
| 5170 | 2 | 98 | 51 |
| 5171 | 1 | 36 | 46 |
| 5172 | 2 | 63 | 47 |
| 5173 | 1 | 5 | 45 |
| 5174 | 2 | 117 | 41 |
| 5175 | 1 | 111 | 42 |
| 5176 | 2 | 111 | 10 |
| 5177 | 1 | 17 | 45 |
| 5178 | 2 | 129 | 41 |
| 5179 | 1 | 123 | 42 |
| 5180 | 2 | 123 | 10 |
| 5181 | 1 | 29 | 45 |
| 5182 | 2 | 141 | 41 |
| 5183 | 1 | 135 | 42 |
| 5184 | 2 | 135 | 10 |
| 5185 | 1 | 114 | 49 |
| 5186 | 2 | 82 | 54 |
| 5187 | 1 | 46 | 21 |
| 5188 | 2 | 38 | 55 |
| 5189 | 1 | 126 | 49 |
| 5190 | 2 | 94 | 54 |
| 5191 | 1 | 58 | 21 |
| 5192 | 2 | 50 | 55 |
| 5193 | 1 | 138 | 49 |
| 5194 | 2 | 106 | 54 |
| 5195 | 1 | 70 | 21 |
| 5196 | 2 | 62 | 55 |
| 5197 | 1 | 9 | 57 |
| 5198 | 2 | 38 | 22 |
| 5199 | 1 | 8 | 22 |
| 5200 | 2 | 114 | 17 |
| 5201 | 1 | 21 | 57 |
| 5202 | 2 | 50 | 22 |
| 5203 | 1 | 20 | 22 |
| 5204 | 2 | 126 | 17 |
| 5205 | 1 | 33 | 57 |
| 5206 | 2 | 62 | 22 |
| 5207 | 1 | 32 | 22 |
| 5208 | 2 | 138 | 17 |
| 5209 | 1 | 44 | 17 |
| 5210 | 2 | 74 | 25 |
| 5211 | 1 | 45 | 23 |
| 5212 | 2 | 74 | 58 |
| 5213 | 1 | 56 | 17 |
| 5214 | 2 | 86 | 25 |
| 5215 | 1 | 57 | 23 |
| 5216 | 2 | 86 | 58 |
| 5217 | 1 | 68 | 17 |
| 5218 | 2 | 98 | 25 |
| 5219 | 1 | 69 | 23 |
| 5220 | 2 | 98 | 58 |
| 5221 | 1 | 76 | 26 |
| 5222 | 2 | 114 | 22 |
| 5223 | 1 | 2 | 27 |
| 5224 | 2 | 3 | 56 |
| 5225 | 1 | 88 | 26 |
| 5226 | 2 | 126 | 22 |
| 5227 | 1 | 14 | 27 |
| 5228 | 2 | 15 | 56 |
| 5229 | 1 | 100 | 26 |
| 5230 | 2 | 138 | 22 |
| 5231 | 1 | 26 | 27 |
| 5232 | 2 | 27 | 56 |
| 5233 | 1 | 45 | 54 |
| 5234 | 2 | 2 | 59 |
| 5235 | 1 | 84 | 20 |
| 5236 | 2 | 39 | 49 |
| 5237 | 1 | 57 | 54 |
| 5238 | 2 | 14 | 59 |
| 5239 | 1 | 96 | 20 |
| 5240 | 2 | 51 | 49 |
| 5241 | 1 | 69 | 54 |
| 5242 | 2 | 26 | 59 |
| 5243 | 1 | 108 | 20 |
| 5244 | 2 | 63 | 49 |
| 5245 | 1 | 6 | 24 |
| 5246 | 2 | 119 | 21 |
| 5247 | 1 | 81 | 58 |
| 5248 | 2 | 39 | 18 |
| 5249 | 1 | 18 | 24 |
| 5250 | 2 | 131 | 21 |
| 5251 | 1 | 93 | 58 |
| 5252 | 2 | 51 | 18 |
| 5253 | 1 | 30 | 24 |
| 5254 | 2 | 143 | 21 |
| 5255 | 1 | 105 | 58 |
| 5256 | 2 | 63 | 18 |
| 5257 | 1 | 39 | 20 |
| 5258 | 2 | 74 | 59 |
| 5259 | 1 | 12 | 54 |
| 5260 | 2 | 39 | 55 |
| 5261 | 1 | 51 | 20 |
| 5262 | 2 | 86 | 59 |
| 5263 | 1 | 24 | 54 |
| 5264 | 2 | 51 | 55 |
| 5265 | 1 | 63 | 20 |
| 5266 | 2 | 98 | 59 |
| 5267 | 1 | 36 | 54 |
| 5268 | 2 | 63 | 55 |
| 5269 | 1 | 5 | 53 |
| 5270 | 2 | 117 | 49 |
| 5271 | 1 | 111 | 50 |
| 5272 | 2 | 111 | 18 |
| 5273 | 1 | 17 | 53 |
| 5274 | 2 | 129 | 49 |
| 5275 | 1 | 123 | 50 |
| 5276 | 2 | 123 | 18 |
| 5277 | 1 | 29 | 53 |
| 5278 | 2 | 141 | 49 |
| 5279 | 1 | 135 | 50 |
| 5280 | 2 | 135 | 18 |
| 5281 | 1 | 114 | 57 |
| 5282 | 2 | 82 | 62 |
| 5283 | 1 | 46 | 29 |
| 5284 | 2 | 38 | 63 |
| 5285 | 1 | 126 | 57 |
| 5286 | 2 | 94 | 62 |
| 5287 | 1 | 58 | 29 |
| 5288 | 2 | 50 | 63 |
| 5289 | 1 | 138 | 57 |
| 5290 | 2 | 106 | 62 |
| 5291 | 1 | 70 | 29 |
| 5292 | 2 | 62 | 63 |
| 5293 | 1 | 9 | 65 |
| 5294 | 2 | 38 | 30 |
| 5295 | 1 | 8 | 30 |
| 5296 | 2 | 114 | 25 |
| 5297 | 1 | 21 | 65 |
| 5298 | 2 | 50 | 30 |
| 5299 | 1 | 20 | 30 |
| 5300 | 2 | 126 | 25 |
| 5301 | 1 | 33 | 65 |
| 5302 | 2 | 62 | 30 |
| 5303 | 1 | 32 | 30 |
| 5304 | 2 | 138 | 25 |
| 5305 | 1 | 44 | 25 |
| 5306 | 2 | 74 | 33 |
| 5307 | 1 | 45 | 31 |
| 5308 | 2 | 74 | 66 |
| 5309 | 1 | 56 | 25 |
| 5310 | 2 | 86 | 33 |
| 5311 | 1 | 57 | 31 |
| 5312 | 2 | 86 | 66 |
| 5313 | 1 | 68 | 25 |
| 5314 | 2 | 98 | 33 |
| 5315 | 1 | 69 | 31 |
| 5316 | 2 | 98 | 66 |
| 5317 | 1 | 76 | 34 |
| 5318 | 2 | 114 | 30 |
| 5319 | 1 | 2 | 35 |
| 5320 | 2 | 3 | 64 |
| 5321 | 1 | 88 | 34 |
| 5322 | 2 | 126 | 30 |
| 5323 | 1 | 14 | 35 |
| 5324 | 2 | 15 | 64 |
| 5325 | 1 | 100 | 34 |
| 5326 | 2 | 138 | 30 |
| 5327 | 1 | 26 | 35 |
| 5328 | 2 | 27 | 64 |
| 5329 | 1 | 45 | 62 |
| 5330 | 2 | 2 | 67 |
| 5331 | 1 | 84 | 28 |
| 5332 | 2 | 39 | 57 |
| 5333 | 1 | 57 | 62 |
| 5334 | 2 | 14 | 67 |
| 5335 | 1 | 96 | 28 |
| 5336 | 2 | 51 | 57 |
| 5337 | 1 | 69 | 62 |
| 5338 | 2 | 26 | 67 |
| 5339 | 1 | 108 | 28 |
| 5340 | 2 | 63 | 57 |
| 5341 | 1 | 6 | 32 |
| 5342 | 2 | 119 | 29 |
| 5343 | 1 | 81 | 66 |
| 5344 | 2 | 39 | 26 |
| 5345 | 1 | 18 | 32 |
| 5346 | 2 | 131 | 29 |
| 5347 | 1 | 93 | 66 |
| 5348 | 2 | 51 | 26 |
| 5349 | 1 | 30 | 32 |
| 5350 | 2 | 143 | 29 |
| 5351 | 1 | 105 | 66 |
| 5352 | 2 | 63 | 26 |
| 5353 | 1 | 39 | 28 |
| 5354 | 2 | 74 | 67 |
| 5355 | 1 | 12 | 62 |
| 5356 | 2 | 39 | 63 |
| 5357 | 1 | 51 | 28 |
| 5358 | 2 | 86 | 67 |
| 5359 | 1 | 24 | 62 |
| 5360 | 2 | 51 | 63 |
| 5361 | 1 | 63 | 28 |
| 5362 | 2 | 98 | 67 |
| 5363 | 1 | 36 | 62 |
| 5364 | 2 | 63 | 63 |
| 5365 | 1 | 5 | 61 |
| 5366 | 2 | 117 | 57 |
| 5367 | 1 | 111 | 58 |
| 5368 | 2 | 111 | 26 |
| 5369 | 1 | 17 | 61 |
| 5370 | 2 | 129 | 57 |
| 5371 | 1 | 123 | 58 |
| 5372 | 2 | 123 | 26 |
| 5373 | 1 | 29 | 61 |
| 5374 | 2 | 141 | 57 |
| 5375 | 1 | 135 | 58 |
| 5376 | 2 | 135 | 26 |
| 5377 | 1 | 42 | 3 |
| 5378 | 2 | 6 | 42 |
| 5379 | 1 | 75 | 7 |
| 5380 | 2 | 10 | 8 |
| 5381 | 1 | 54 | 3 |
| 5382 | 2 | 18 | 42 |
| 5383 | 1 | 87 | 7 |
| 5384 | 2 | 22 | 8 |
| 5385 | 1 | 66 | 3 |
| 5386 | 2 | 30 | 42 |
| 5387 | 1 | 99 | 7 |
| 5388 | 2 | 34 | 8 |
| 5389 | 1 | 82 | 7 |
| 5390 | 2 | 3 | 37 |
| 5391 | 1 | 3 | 39 |
| 5392 | 2 | 117 | 6 |
| 5393 | 1 | 94 | 7 |
| 5394 | 2 | 15 | 37 |
| 5395 | 1 | 15 | 39 |
| 5396 | 2 | 129 | 6 |
| 5397 | 1 | 106 | 7 |
| 5398 | 2 | 27 | 37 |
| 5399 | 1 | 27 | 39 |
| 5400 | 2 | 141 | 6 |
| 5401 | 1 | 5 | 9 |
| 5402 | 2 | 116 | 38 |
| 5403 | 1 | 80 | 42 |
| 5404 | 2 | 39 | 36 |
| 5405 | 1 | 17 | 9 |
| 5406 | 2 | 128 | 38 |
| 5407 | 1 | 92 | 42 |
| 5408 | 2 | 51 | 36 |
| 5409 | 1 | 29 | 9 |
| 5410 | 2 | 140 | 38 |
| 5411 | 1 | 104 | 42 |
| 5412 | 2 | 63 | 36 |
| 5413 | 1 | 3 | 6 |
| 5414 | 2 | 39 | 3 |
| 5415 | 1 | 7 | 36 |
| 5416 | 2 | 8 | 39 |
| 5417 | 1 | 15 | 6 |
| 5418 | 2 | 51 | 3 |
| 5419 | 1 | 19 | 36 |
| 5420 | 2 | 20 | 39 |
| 5421 | 1 | 27 | 6 |
| 5422 | 2 | 63 | 3 |
| 5423 | 1 | 31 | 36 |
| 5424 | 2 | 32 | 39 |
| 5425 | 1 | 48 | 38 |
| 5426 | 2 | 113 | 35 |
| 5427 | 1 | 116 | 1 |
| 5428 | 2 | 111 | 38 |
| 5429 | 1 | 60 | 38 |
| 5430 | 2 | 125 | 35 |
| 5431 | 1 | 128 | 1 |
| 5432 | 2 | 123 | 38 |
| 5433 | 1 | 72 | 38 |
| 5434 | 2 | 137 | 35 |
| 5435 | 1 | 140 | 1 |
| 5436 | 2 | 135 | 38 |
| 5437 | 1 | 9 | 8 |
| 5438 | 2 | 84 | 6 |
| 5439 | 1 | 42 | 35 |
| 5440 | 2 | 39 | 7 |
| 5441 | 1 | 21 | 8 |
| 5442 | 2 | 96 | 6 |
| 5443 | 1 | 54 | 35 |
| 5444 | 2 | 51 | 7 |
| 5445 | 1 | 33 | 8 |
| 5446 | 2 | 108 | 6 |
| 5447 | 1 | 66 | 35 |
| 5448 | 2 | 63 | 7 |
| 5449 | 1 | 83 | 10 |
| 5450 | 2 | 76 | 38 |
| 5451 | 1 | 10 | 4 |
| 5452 | 2 | 78 | 42 |
| 5453 | 1 | 95 | 10 |
| 5454 | 2 | 88 | 38 |
| 5455 | 1 | 22 | 4 |
| 5456 | 2 | 90 | 42 |
| 5457 | 1 | 107 | 10 |
| 5458 | 2 | 100 | 38 |
| 5459 | 1 | 34 | 4 |
| 5460 | 2 | 102 | 42 |
| 5461 | 1 | 120 | 4 |
| 5462 | 2 | 111 | 5 |
| 5463 | 1 | 111 | 7 |
| 5464 | 2 | 117 | 2 |
| 5465 | 1 | 132 | 4 |
| 5466 | 2 | 123 | 5 |
| 5467 | 1 | 123 | 7 |
| 5468 | 2 | 129 | 2 |
| 5469 | 1 | 144 | 4 |
| 5470 | 2 | 135 | 5 |
| 5471 | 1 | 135 | 7 |
| 5472 | 2 | 141 | 2 |
| 5473 | 1 | 42 | 11 |
| 5474 | 2 | 6 | 50 |
| 5475 | 1 | 75 | 15 |
| 5476 | 2 | 10 | 16 |
| 5477 | 1 | 54 | 11 |
| 5478 | 2 | 18 | 50 |
| 5479 | 1 | 87 | 15 |
| 5480 | 2 | 22 | 16 |
| 5481 | 1 | 66 | 11 |
| 5482 | 2 | 30 | 50 |
| 5483 | 1 | 99 | 15 |
| 5484 | 2 | 34 | 16 |
| 5485 | 1 | 82 | 15 |
| 5486 | 2 | 3 | 45 |
| 5487 | 1 | 3 | 47 |
| 5488 | 2 | 117 | 14 |
| 5489 | 1 | 94 | 15 |
| 5490 | 2 | 15 | 45 |
| 5491 | 1 | 15 | 47 |
| 5492 | 2 | 129 | 14 |
| 5493 | 1 | 106 | 15 |
| 5494 | 2 | 27 | 45 |
| 5495 | 1 | 27 | 47 |
| 5496 | 2 | 141 | 14 |
| 5497 | 1 | 5 | 17 |
| 5498 | 2 | 116 | 46 |
| 5499 | 1 | 80 | 50 |
| 5500 | 2 | 39 | 44 |
| 5501 | 1 | 17 | 17 |
| 5502 | 2 | 128 | 46 |
| 5503 | 1 | 92 | 50 |
| 5504 | 2 | 51 | 44 |
| 5505 | 1 | 29 | 17 |
| 5506 | 2 | 140 | 46 |
| 5507 | 1 | 104 | 50 |
| 5508 | 2 | 63 | 44 |
| 5509 | 1 | 3 | 14 |
| 5510 | 2 | 39 | 11 |
| 5511 | 1 | 7 | 44 |
| 5512 | 2 | 8 | 47 |
| 5513 | 1 | 15 | 14 |
| 5514 | 2 | 51 | 11 |
| 5515 | 1 | 19 | 44 |
| 5516 | 2 | 20 | 47 |
| 5517 | 1 | 27 | 14 |
| 5518 | 2 | 63 | 11 |
| 5519 | 1 | 31 | 44 |
| 5520 | 2 | 32 | 47 |
| 5521 | 1 | 48 | 46 |
| 5522 | 2 | 113 | 43 |
| 5523 | 1 | 116 | 9 |
| 5524 | 2 | 111 | 46 |
| 5525 | 1 | 60 | 46 |
| 5526 | 2 | 125 | 43 |
| 5527 | 1 | 128 | 9 |
| 5528 | 2 | 123 | 46 |
| 5529 | 1 | 72 | 46 |
| 5530 | 2 | 137 | 43 |
| 5531 | 1 | 140 | 9 |
| 5532 | 2 | 135 | 46 |
| 5533 | 1 | 9 | 16 |
| 5534 | 2 | 84 | 14 |
| 5535 | 1 | 42 | 43 |
| 5536 | 2 | 39 | 15 |
| 5537 | 1 | 21 | 16 |
| 5538 | 2 | 96 | 14 |
| 5539 | 1 | 54 | 43 |
| 5540 | 2 | 51 | 15 |
| 5541 | 1 | 33 | 16 |
| 5542 | 2 | 108 | 14 |
| 5543 | 1 | 66 | 43 |
| 5544 | 2 | 63 | 15 |
| 5545 | 1 | 83 | 18 |
| 5546 | 2 | 76 | 46 |
| 5547 | 1 | 10 | 12 |
| 5548 | 2 | 78 | 50 |
| 5549 | 1 | 95 | 18 |
| 5550 | 2 | 88 | 46 |
| 5551 | 1 | 22 | 12 |
| 5552 | 2 | 90 | 50 |
| 5553 | 1 | 107 | 18 |
| 5554 | 2 | 100 | 46 |
| 5555 | 1 | 34 | 12 |
| 5556 | 2 | 102 | 50 |
| 5557 | 1 | 120 | 12 |
| 5558 | 2 | 111 | 13 |
| 5559 | 1 | 111 | 15 |
| 5560 | 2 | 117 | 10 |
| 5561 | 1 | 132 | 12 |
| 5562 | 2 | 123 | 13 |
| 5563 | 1 | 123 | 15 |
| 5564 | 2 | 129 | 10 |
| 5565 | 1 | 144 | 12 |
| 5566 | 2 | 135 | 13 |
| 5567 | 1 | 135 | 15 |
| 5568 | 2 | 141 | 10 |
| 5569 | 1 | 42 | 19 |
| 5570 | 2 | 6 | 58 |
| 5571 | 1 | 75 | 23 |
| 5572 | 2 | 10 | 24 |
| 5573 | 1 | 54 | 19 |
| 5574 | 2 | 18 | 58 |
| 5575 | 1 | 87 | 23 |
| 5576 | 2 | 22 | 24 |
| 5577 | 1 | 66 | 19 |
| 5578 | 2 | 30 | 58 |
| 5579 | 1 | 99 | 23 |
| 5580 | 2 | 34 | 24 |
| 5581 | 1 | 82 | 23 |
| 5582 | 2 | 3 | 53 |
| 5583 | 1 | 3 | 55 |
| 5584 | 2 | 117 | 22 |
| 5585 | 1 | 94 | 23 |
| 5586 | 2 | 15 | 53 |
| 5587 | 1 | 15 | 55 |
| 5588 | 2 | 129 | 22 |
| 5589 | 1 | 106 | 23 |
| 5590 | 2 | 27 | 53 |
| 5591 | 1 | 27 | 55 |
| 5592 | 2 | 141 | 22 |
| 5593 | 1 | 5 | 25 |
| 5594 | 2 | 116 | 54 |
| 5595 | 1 | 80 | 58 |
| 5596 | 2 | 39 | 52 |
| 5597 | 1 | 17 | 25 |
| 5598 | 2 | 128 | 54 |
| 5599 | 1 | 92 | 58 |
| 5600 | 2 | 51 | 52 |
| 5601 | 1 | 29 | 25 |
| 5602 | 2 | 140 | 54 |
| 5603 | 1 | 104 | 58 |
| 5604 | 2 | 63 | 52 |
| 5605 | 1 | 3 | 22 |
| 5606 | 2 | 39 | 19 |
| 5607 | 1 | 7 | 52 |
| 5608 | 2 | 8 | 55 |
| 5609 | 1 | 15 | 22 |
| 5610 | 2 | 51 | 19 |
| 5611 | 1 | 19 | 52 |
| 5612 | 2 | 20 | 55 |
| 5613 | 1 | 27 | 22 |
| 5614 | 2 | 63 | 19 |
| 5615 | 1 | 31 | 52 |
| 5616 | 2 | 32 | 55 |
| 5617 | 1 | 48 | 54 |
| 5618 | 2 | 113 | 51 |
| 5619 | 1 | 116 | 17 |
| 5620 | 2 | 111 | 54 |
| 5621 | 1 | 60 | 54 |
| 5622 | 2 | 125 | 51 |
| 5623 | 1 | 128 | 17 |
| 5624 | 2 | 123 | 54 |
| 5625 | 1 | 72 | 54 |
| 5626 | 2 | 137 | 51 |
| 5627 | 1 | 140 | 17 |
| 5628 | 2 | 135 | 54 |
| 5629 | 1 | 9 | 24 |
| 5630 | 2 | 84 | 22 |
| 5631 | 1 | 42 | 51 |
| 5632 | 2 | 39 | 23 |
| 5633 | 1 | 21 | 24 |
| 5634 | 2 | 96 | 22 |
| 5635 | 1 | 54 | 51 |
| 5636 | 2 | 51 | 23 |
| 5637 | 1 | 33 | 24 |
| 5638 | 2 | 108 | 22 |
| 5639 | 1 | 66 | 51 |
| 5640 | 2 | 63 | 23 |
| 5641 | 1 | 83 | 26 |
| 5642 | 2 | 76 | 54 |
| 5643 | 1 | 10 | 20 |
| 5644 | 2 | 78 | 58 |
| 5645 | 1 | 95 | 26 |
| 5646 | 2 | 88 | 54 |
| 5647 | 1 | 22 | 20 |
| 5648 | 2 | 90 | 58 |
| 5649 | 1 | 107 | 26 |
| 5650 | 2 | 100 | 54 |
| 5651 | 1 | 34 | 20 |
| 5652 | 2 | 102 | 58 |
| 5653 | 1 | 120 | 20 |
| 5654 | 2 | 111 | 21 |
| 5655 | 1 | 111 | 23 |
| 5656 | 2 | 117 | 18 |
| 5657 | 1 | 132 | 20 |
| 5658 | 2 | 123 | 21 |
| 5659 | 1 | 123 | 23 |
| 5660 | 2 | 129 | 18 |
| 5661 | 1 | 144 | 20 |
| 5662 | 2 | 135 | 21 |
| 5663 | 1 | 135 | 23 |
| 5664 | 2 | 141 | 18 |
| 5665 | 1 | 42 | 27 |
| 5666 | 2 | 6 | 66 |
| 5667 | 1 | 75 | 31 |
| 5668 | 2 | 10 | 32 |
| 5669 | 1 | 54 | 27 |
| 5670 | 2 | 18 | 66 |
| 5671 | 1 | 87 | 31 |
| 5672 | 2 | 22 | 32 |
| 5673 | 1 | 66 | 27 |
| 5674 | 2 | 30 | 66 |
| 5675 | 1 | 99 | 31 |
| 5676 | 2 | 34 | 32 |
| 5677 | 1 | 82 | 31 |
| 5678 | 2 | 3 | 61 |
| 5679 | 1 | 3 | 63 |
| 5680 | 2 | 117 | 30 |
| 5681 | 1 | 94 | 31 |
| 5682 | 2 | 15 | 61 |
| 5683 | 1 | 15 | 63 |
| 5684 | 2 | 129 | 30 |
| 5685 | 1 | 106 | 31 |
| 5686 | 2 | 27 | 61 |
| 5687 | 1 | 27 | 63 |
| 5688 | 2 | 141 | 30 |
| 5689 | 1 | 5 | 33 |
| 5690 | 2 | 116 | 62 |
| 5691 | 1 | 80 | 66 |
| 5692 | 2 | 39 | 60 |
| 5693 | 1 | 17 | 33 |
| 5694 | 2 | 128 | 62 |
| 5695 | 1 | 92 | 66 |
| 5696 | 2 | 51 | 60 |
| 5697 | 1 | 29 | 33 |
| 5698 | 2 | 140 | 62 |
| 5699 | 1 | 104 | 66 |
| 5700 | 2 | 63 | 60 |
| 5701 | 1 | 3 | 30 |
| 5702 | 2 | 39 | 27 |
| 5703 | 1 | 7 | 60 |
| 5704 | 2 | 8 | 63 |
| 5705 | 1 | 15 | 30 |
| 5706 | 2 | 51 | 27 |
| 5707 | 1 | 19 | 60 |
| 5708 | 2 | 20 | 63 |
| 5709 | 1 | 27 | 30 |
| 5710 | 2 | 63 | 27 |
| 5711 | 1 | 31 | 60 |
| 5712 | 2 | 32 | 63 |
| 5713 | 1 | 48 | 62 |
| 5714 | 2 | 113 | 59 |
| 5715 | 1 | 116 | 25 |
| 5716 | 2 | 111 | 62 |
| 5717 | 1 | 60 | 62 |
| 5718 | 2 | 125 | 59 |
| 5719 | 1 | 128 | 25 |
| 5720 | 2 | 123 | 62 |
| 5721 | 1 | 72 | 62 |
| 5722 | 2 | 137 | 59 |
| 5723 | 1 | 140 | 25 |
| 5724 | 2 | 135 | 62 |
| 5725 | 1 | 9 | 32 |
| 5726 | 2 | 84 | 30 |
| 5727 | 1 | 42 | 59 |
| 5728 | 2 | 39 | 31 |
| 5729 | 1 | 21 | 32 |
| 5730 | 2 | 96 | 30 |
| 5731 | 1 | 54 | 59 |
| 5732 | 2 | 51 | 31 |
| 5733 | 1 | 33 | 32 |
| 5734 | 2 | 108 | 30 |
| 5735 | 1 | 66 | 59 |
| 5736 | 2 | 63 | 31 |
| 5737 | 1 | 83 | 34 |
| 5738 | 2 | 76 | 62 |
| 5739 | 1 | 10 | 28 |
| 5740 | 2 | 78 | 66 |
| 5741 | 1 | 95 | 34 |
| 5742 | 2 | 88 | 62 |
| 5743 | 1 | 22 | 28 |
| 5744 | 2 | 90 | 66 |
| 5745 | 1 | 107 | 34 |
| 5746 | 2 | 100 | 62 |
| 5747 | 1 | 34 | 28 |
| 5748 | 2 | 102 | 66 |
| 5749 | 1 | 120 | 28 |
| 5750 | 2 | 111 | 29 |
| 5751 | 1 | 111 | 31 |
| 5752 | 2 | 117 | 26 |
| 5753 | 1 | 132 | 28 |
| 5754 | 2 | 123 | 29 |
| 5755 | 1 | 123 | 31 |
| 5756 | 2 | 129 | 26 |
| 5757 | 1 | 144 | 28 |
| 5758 | 2 | 135 | 29 |
| 5759 | 1 | 135 | 31 |
| 5760 | 2 | 141 | 26 |
| 5761 | 1 | 120 | 7 |
| 5762 | 2 | 47 | 38 |
| 5763 | 1 | 79 | 37 |
| 5764 | 2 | 75 | 39 |
| 5765 | 1 | 132 | 7 |
| 5766 | 2 | 59 | 38 |
| 5767 | 1 | 91 | 37 |
| 5768 | 2 | 87 | 39 |
| 5769 | 1 | 144 | 7 |
| 5770 | 2 | 71 | 38 |
| 5771 | 1 | 103 | 37 |
| 5772 | 2 | 99 | 39 |
| 5773 | 1 | 5 | 10 |
| 5774 | 2 | 80 | 38 |
| 5775 | 1 | 75 | 8 |
| 5776 | 2 | 111 | 36 |
| 5777 | 1 | 17 | 10 |
| 5778 | 2 | 92 | 38 |
| 5779 | 1 | 87 | 8 |
| 5780 | 2 | 123 | 36 |
| 5781 | 1 | 29 | 10 |
| 5782 | 2 | 104 | 38 |
| 5783 | 1 | 99 | 8 |
| 5784 | 2 | 135 | 36 |
| 5785 | 1 | 48 | 6 |
| 5786 | 2 | 39 | 35 |
| 5787 | 1 | 48 | 34 |
| 5788 | 2 | 3 | 8 |
| 5789 | 1 | 60 | 6 |
| 5790 | 2 | 51 | 35 |
| 5791 | 1 | 60 | 34 |
| 5792 | 2 | 15 | 8 |
| 5793 | 1 | 72 | 6 |
| 5794 | 2 | 63 | 35 |
| 5795 | 1 | 72 | 34 |
| 5796 | 2 | 27 | 8 |
| 5797 | 1 | 47 | 5 |
| 5798 | 2 | 119 | 39 |
| 5799 | 1 | 114 | 36 |
| 5800 | 2 | 39 | 5 |
| 5801 | 1 | 59 | 5 |
| 5802 | 2 | 131 | 39 |
| 5803 | 1 | 126 | 36 |
| 5804 | 2 | 51 | 5 |
| 5805 | 1 | 71 | 5 |
| 5806 | 2 | 143 | 39 |
| 5807 | 1 | 138 | 36 |
| 5808 | 2 | 63 | 5 |
| 5809 | 1 | 3 | 9 |
| 5810 | 2 | 111 | 37 |
| 5811 | 1 | 48 | 3 |
| 5812 | 2 | 75 | 42 |
| 5813 | 1 | 15 | 9 |
| 5814 | 2 | 123 | 37 |
| 5815 | 1 | 60 | 3 |
| 5816 | 2 | 87 | 42 |
| 5817 | 1 | 27 | 9 |
| 5818 | 2 | 135 | 37 |
| 5819 | 1 | 72 | 3 |
| 5820 | 2 | 99 | 42 |
| 5821 | 1 | 3 | 41 |
| 5822 | 2 | 75 | 9 |
| 5823 | 1 | 4 | 36 |
| 5824 | 2 | 111 | 39 |
| 5825 | 1 | 15 | 41 |
| 5826 | 2 | 87 | 9 |
| 5827 | 1 | 16 | 36 |
| 5828 | 2 | 123 | 39 |
| 5829 | 1 | 27 | 41 |
| 5830 | 2 | 99 | 9 |
| 5831 | 1 | 28 | 36 |
| 5832 | 2 | 135 | 39 |
| 5833 | 1 | 77 | 43 |
| 5834 | 2 | 120 | 2 |
| 5835 | 1 | 118 | 5 |
| 5836 | 2 | 114 | 8 |
| 5837 | 1 | 89 | 43 |
| 5838 | 2 | 132 | 2 |
| 5839 | 1 | 130 | 5 |
| 5840 | 2 | 126 | 8 |
| 5841 | 1 | 101 | 43 |
| 5842 | 2 | 144 | 2 |
| 5843 | 1 | 142 | 5 |
| 5844 | 2 | 138 | 8 |
| 5845 | 1 | 12 | 7 |
| 5846 | 2 | 111 | 6 |
| 5847 | 1 | 79 | 9 |
| 5848 | 2 | 3 | 11 |
| 5849 | 1 | 24 | 7 |
| 5850 | 2 | 123 | 6 |
| 5851 | 1 | 91 | 9 |
| 5852 | 2 | 15 | 11 |
| 5853 | 1 | 36 | 7 |
| 5854 | 2 | 135 | 6 |
| 5855 | 1 | 103 | 9 |
| 5856 | 2 | 27 | 11 |
| 5857 | 1 | 120 | 15 |
| 5858 | 2 | 47 | 46 |
| 5859 | 1 | 79 | 45 |
| 5860 | 2 | 75 | 47 |
| 5861 | 1 | 132 | 15 |
| 5862 | 2 | 59 | 46 |
| 5863 | 1 | 91 | 45 |
| 5864 | 2 | 87 | 47 |
| 5865 | 1 | 144 | 15 |
| 5866 | 2 | 71 | 46 |
| 5867 | 1 | 103 | 45 |
| 5868 | 2 | 99 | 47 |
| 5869 | 1 | 5 | 18 |
| 5870 | 2 | 80 | 46 |
| 5871 | 1 | 75 | 16 |
| 5872 | 2 | 111 | 44 |
| 5873 | 1 | 17 | 18 |
| 5874 | 2 | 92 | 46 |
| 5875 | 1 | 87 | 16 |
| 5876 | 2 | 123 | 44 |
| 5877 | 1 | 29 | 18 |
| 5878 | 2 | 104 | 46 |
| 5879 | 1 | 99 | 16 |
| 5880 | 2 | 135 | 44 |
| 5881 | 1 | 48 | 14 |
| 5882 | 2 | 39 | 43 |
| 5883 | 1 | 48 | 42 |
| 5884 | 2 | 3 | 16 |
| 5885 | 1 | 60 | 14 |
| 5886 | 2 | 51 | 43 |
| 5887 | 1 | 60 | 42 |
| 5888 | 2 | 15 | 16 |
| 5889 | 1 | 72 | 14 |
| 5890 | 2 | 63 | 43 |
| 5891 | 1 | 72 | 42 |
| 5892 | 2 | 27 | 16 |
| 5893 | 1 | 47 | 13 |
| 5894 | 2 | 119 | 47 |
| 5895 | 1 | 114 | 44 |
| 5896 | 2 | 39 | 13 |
| 5897 | 1 | 59 | 13 |
| 5898 | 2 | 131 | 47 |
| 5899 | 1 | 126 | 44 |
| 5900 | 2 | 51 | 13 |
| 5901 | 1 | 71 | 13 |
| 5902 | 2 | 143 | 47 |
| 5903 | 1 | 138 | 44 |
| 5904 | 2 | 63 | 13 |
| 5905 | 1 | 3 | 17 |
| 5906 | 2 | 111 | 45 |
| 5907 | 1 | 48 | 11 |
| 5908 | 2 | 75 | 50 |
| 5909 | 1 | 15 | 17 |
| 5910 | 2 | 123 | 45 |
| 5911 | 1 | 60 | 11 |
| 5912 | 2 | 87 | 50 |
| 5913 | 1 | 27 | 17 |
| 5914 | 2 | 135 | 45 |
| 5915 | 1 | 72 | 11 |
| 5916 | 2 | 99 | 50 |
| 5917 | 1 | 3 | 49 |
| 5918 | 2 | 75 | 17 |
| 5919 | 1 | 4 | 44 |
| 5920 | 2 | 111 | 47 |
| 5921 | 1 | 15 | 49 |
| 5922 | 2 | 87 | 17 |
| 5923 | 1 | 16 | 44 |
| 5924 | 2 | 123 | 47 |
| 5925 | 1 | 27 | 49 |
| 5926 | 2 | 99 | 17 |
| 5927 | 1 | 28 | 44 |
| 5928 | 2 | 135 | 47 |
| 5929 | 1 | 77 | 51 |
| 5930 | 2 | 120 | 10 |
| 5931 | 1 | 118 | 13 |
| 5932 | 2 | 114 | 16 |
| 5933 | 1 | 89 | 51 |
| 5934 | 2 | 132 | 10 |
| 5935 | 1 | 130 | 13 |
| 5936 | 2 | 126 | 16 |
| 5937 | 1 | 101 | 51 |
| 5938 | 2 | 144 | 10 |
| 5939 | 1 | 142 | 13 |
| 5940 | 2 | 138 | 16 |
| 5941 | 1 | 12 | 15 |
| 5942 | 2 | 111 | 14 |
| 5943 | 1 | 79 | 17 |
| 5944 | 2 | 3 | 19 |
| 5945 | 1 | 24 | 15 |
| 5946 | 2 | 123 | 14 |
| 5947 | 1 | 91 | 17 |
| 5948 | 2 | 15 | 19 |
| 5949 | 1 | 36 | 15 |
| 5950 | 2 | 135 | 14 |
| 5951 | 1 | 103 | 17 |
| 5952 | 2 | 27 | 19 |
| 5953 | 1 | 120 | 23 |
| 5954 | 2 | 47 | 54 |
| 5955 | 1 | 79 | 53 |
| 5956 | 2 | 75 | 55 |
| 5957 | 1 | 132 | 23 |
| 5958 | 2 | 59 | 54 |
| 5959 | 1 | 91 | 53 |
| 5960 | 2 | 87 | 55 |
| 5961 | 1 | 144 | 23 |
| 5962 | 2 | 71 | 54 |
| 5963 | 1 | 103 | 53 |
| 5964 | 2 | 99 | 55 |
| 5965 | 1 | 5 | 26 |
| 5966 | 2 | 80 | 54 |
| 5967 | 1 | 75 | 24 |
| 5968 | 2 | 111 | 52 |
| 5969 | 1 | 17 | 26 |
| 5970 | 2 | 92 | 54 |
| 5971 | 1 | 87 | 24 |
| 5972 | 2 | 123 | 52 |
| 5973 | 1 | 29 | 26 |
| 5974 | 2 | 104 | 54 |
| 5975 | 1 | 99 | 24 |
| 5976 | 2 | 135 | 52 |
| 5977 | 1 | 48 | 22 |
| 5978 | 2 | 39 | 51 |
| 5979 | 1 | 48 | 50 |
| 5980 | 2 | 3 | 24 |
| 5981 | 1 | 60 | 22 |
| 5982 | 2 | 51 | 51 |
| 5983 | 1 | 60 | 50 |
| 5984 | 2 | 15 | 24 |
| 5985 | 1 | 72 | 22 |
| 5986 | 2 | 63 | 51 |
| 5987 | 1 | 72 | 50 |
| 5988 | 2 | 27 | 24 |
| 5989 | 1 | 47 | 21 |
| 5990 | 2 | 119 | 55 |
| 5991 | 1 | 114 | 52 |
| 5992 | 2 | 39 | 21 |
| 5993 | 1 | 59 | 21 |
| 5994 | 2 | 131 | 55 |
| 5995 | 1 | 126 | 52 |
| 5996 | 2 | 51 | 21 |
| 5997 | 1 | 71 | 21 |
| 5998 | 2 | 143 | 55 |
| 5999 | 1 | 138 | 52 |
| 6000 | 2 | 63 | 21 |
| 6001 | 1 | 3 | 25 |
| 6002 | 2 | 111 | 53 |
| 6003 | 1 | 48 | 19 |
| 6004 | 2 | 75 | 58 |
| 6005 | 1 | 15 | 25 |
| 6006 | 2 | 123 | 53 |
| 6007 | 1 | 60 | 19 |
| 6008 | 2 | 87 | 58 |
| 6009 | 1 | 27 | 25 |
| 6010 | 2 | 135 | 53 |
| 6011 | 1 | 72 | 19 |
| 6012 | 2 | 99 | 58 |
| 6013 | 1 | 3 | 57 |
| 6014 | 2 | 75 | 25 |
| 6015 | 1 | 4 | 52 |
| 6016 | 2 | 111 | 55 |
| 6017 | 1 | 15 | 57 |
| 6018 | 2 | 87 | 25 |
| 6019 | 1 | 16 | 52 |
| 6020 | 2 | 123 | 55 |
| 6021 | 1 | 27 | 57 |
| 6022 | 2 | 99 | 25 |
| 6023 | 1 | 28 | 52 |
| 6024 | 2 | 135 | 55 |
| 6025 | 1 | 77 | 59 |
| 6026 | 2 | 120 | 18 |
| 6027 | 1 | 118 | 21 |
| 6028 | 2 | 114 | 24 |
| 6029 | 1 | 89 | 59 |
| 6030 | 2 | 132 | 18 |
| 6031 | 1 | 130 | 21 |
| 6032 | 2 | 126 | 24 |
| 6033 | 1 | 101 | 59 |
| 6034 | 2 | 144 | 18 |
| 6035 | 1 | 142 | 21 |
| 6036 | 2 | 138 | 24 |
| 6037 | 1 | 12 | 23 |
| 6038 | 2 | 111 | 22 |
| 6039 | 1 | 79 | 25 |
| 6040 | 2 | 3 | 27 |
| 6041 | 1 | 24 | 23 |
| 6042 | 2 | 123 | 22 |
| 6043 | 1 | 91 | 25 |
| 6044 | 2 | 15 | 27 |
| 6045 | 1 | 36 | 23 |
| 6046 | 2 | 135 | 22 |
| 6047 | 1 | 103 | 25 |
| 6048 | 2 | 27 | 27 |
| 6049 | 1 | 120 | 31 |
| 6050 | 2 | 47 | 62 |
| 6051 | 1 | 79 | 61 |
| 6052 | 2 | 75 | 63 |
| 6053 | 1 | 132 | 31 |
| 6054 | 2 | 59 | 62 |
| 6055 | 1 | 91 | 61 |
| 6056 | 2 | 87 | 63 |
| 6057 | 1 | 144 | 31 |
| 6058 | 2 | 71 | 62 |
| 6059 | 1 | 103 | 61 |
| 6060 | 2 | 99 | 63 |
| 6061 | 1 | 5 | 34 |
| 6062 | 2 | 80 | 62 |
| 6063 | 1 | 75 | 32 |
| 6064 | 2 | 111 | 60 |
| 6065 | 1 | 17 | 34 |
| 6066 | 2 | 92 | 62 |
| 6067 | 1 | 87 | 32 |
| 6068 | 2 | 123 | 60 |
| 6069 | 1 | 29 | 34 |
| 6070 | 2 | 104 | 62 |
| 6071 | 1 | 99 | 32 |
| 6072 | 2 | 135 | 60 |
| 6073 | 1 | 48 | 30 |
| 6074 | 2 | 39 | 59 |
| 6075 | 1 | 48 | 58 |
| 6076 | 2 | 3 | 32 |
| 6077 | 1 | 60 | 30 |
| 6078 | 2 | 51 | 59 |
| 6079 | 1 | 60 | 58 |
| 6080 | 2 | 15 | 32 |
| 6081 | 1 | 72 | 30 |
| 6082 | 2 | 63 | 59 |
| 6083 | 1 | 72 | 58 |
| 6084 | 2 | 27 | 32 |
| 6085 | 1 | 47 | 29 |
| 6086 | 2 | 119 | 63 |
| 6087 | 1 | 114 | 60 |
| 6088 | 2 | 39 | 29 |
| 6089 | 1 | 59 | 29 |
| 6090 | 2 | 131 | 63 |
| 6091 | 1 | 126 | 60 |
| 6092 | 2 | 51 | 29 |
| 6093 | 1 | 71 | 29 |
| 6094 | 2 | 143 | 63 |
| 6095 | 1 | 138 | 60 |
| 6096 | 2 | 63 | 29 |
| 6097 | 1 | 3 | 33 |
| 6098 | 2 | 111 | 61 |
| 6099 | 1 | 48 | 27 |
| 6100 | 2 | 75 | 66 |
| 6101 | 1 | 15 | 33 |
| 6102 | 2 | 123 | 61 |
| 6103 | 1 | 60 | 27 |
| 6104 | 2 | 87 | 66 |
| 6105 | 1 | 27 | 33 |
| 6106 | 2 | 135 | 61 |
| 6107 | 1 | 72 | 27 |
| 6108 | 2 | 99 | 66 |
| 6109 | 1 | 3 | 65 |
| 6110 | 2 | 75 | 33 |
| 6111 | 1 | 4 | 60 |
| 6112 | 2 | 111 | 63 |
| 6113 | 1 | 15 | 65 |
| 6114 | 2 | 87 | 33 |
| 6115 | 1 | 16 | 60 |
| 6116 | 2 | 123 | 63 |
| 6117 | 1 | 27 | 65 |
| 6118 | 2 | 99 | 33 |
| 6119 | 1 | 28 | 60 |
| 6120 | 2 | 135 | 63 |
| 6121 | 1 | 77 | 67 |
| 6122 | 2 | 120 | 26 |
| 6123 | 1 | 118 | 29 |
| 6124 | 2 | 114 | 32 |
| 6125 | 1 | 89 | 67 |
| 6126 | 2 | 132 | 26 |
| 6127 | 1 | 130 | 29 |
| 6128 | 2 | 126 | 32 |
| 6129 | 1 | 101 | 67 |
| 6130 | 2 | 144 | 26 |
| 6131 | 1 | 142 | 29 |
| 6132 | 2 | 138 | 32 |
| 6133 | 1 | 12 | 31 |
| 6134 | 2 | 111 | 30 |
| 6135 | 1 | 79 | 33 |
| 6136 | 2 | 3 | 35 |
| 6137 | 1 | 24 | 31 |
| 6138 | 2 | 123 | 30 |
| 6139 | 1 | 91 | 33 |
| 6140 | 2 | 15 | 35 |
| 6141 | 1 | 36 | 31 |
| 6142 | 2 | 135 | 30 |
| 6143 | 1 | 103 | 33 |
| 6144 | 2 | 27 | 35 |
| 6145 | 1 | 47 | 39 |
| 6146 | 2 | 39 | 8 |
| 6147 | 1 | 120 | 38 |
| 6148 | 2 | 48 | 1 |
| 6149 | 1 | 59 | 39 |
| 6150 | 2 | 51 | 8 |
| 6151 | 1 | 132 | 38 |
| 6152 | 2 | 60 | 1 |
| 6153 | 1 | 71 | 39 |
| 6154 | 2 | 63 | 8 |
| 6155 | 1 | 144 | 38 |
| 6156 | 2 | 72 | 1 |
| 6157 | 1 | 81 | 4 |
| 6158 | 2 | 75 | 11 |
| 6159 | 1 | 80 | 36 |
| 6160 | 2 | 76 | 5 |
| 6161 | 1 | 93 | 4 |
| 6162 | 2 | 87 | 11 |
| 6163 | 1 | 92 | 36 |
| 6164 | 2 | 88 | 5 |
| 6165 | 1 | 105 | 4 |
| 6166 | 2 | 99 | 11 |
| 6167 | 1 | 104 | 36 |
| 6168 | 2 | 100 | 5 |
| 6169 | 1 | 111 | 40 |
| 6170 | 2 | 76 | 36 |
| 6171 | 1 | 4 | 8 |
| 6172 | 2 | 4 | 37 |
| 6173 | 1 | 123 | 40 |
| 6174 | 2 | 88 | 36 |
| 6175 | 1 | 16 | 8 |
| 6176 | 2 | 16 | 37 |
| 6177 | 1 | 135 | 40 |
| 6178 | 2 | 100 | 36 |
| 6179 | 1 | 28 | 8 |
| 6180 | 2 | 28 | 37 |
| 6181 | 1 | 112 | 39 |
| 6182 | 2 | 47 | 36 |
| 6183 | 1 | 76 | 8 |
| 6184 | 2 | 40 | 34 |
| 6185 | 1 | 124 | 39 |
| 6186 | 2 | 59 | 36 |
| 6187 | 1 | 88 | 8 |
| 6188 | 2 | 52 | 34 |
| 6189 | 1 | 136 | 39 |
| 6190 | 2 | 71 | 36 |
| 6191 | 1 | 100 | 8 |
| 6192 | 2 | 64 | 34 |
| 6193 | 1 | 112 | 40 |
| 6194 | 2 | 11 | 36 |
| 6195 | 1 | 112 | 6 |
| 6196 | 2 | 40 | 5 |
| 6197 | 1 | 124 | 40 |
| 6198 | 2 | 23 | 36 |
| 6199 | 1 | 124 | 6 |
| 6200 | 2 | 52 | 5 |
| 6201 | 1 | 136 | 40 |
| 6202 | 2 | 35 | 36 |
| 6203 | 1 | 136 | 6 |
| 6204 | 2 | 64 | 5 |
| 6205 | 1 | 76 | 39 |
| 6206 | 2 | 113 | 8 |
| 6207 | 1 | 112 | 7 |
| 6208 | 2 | 40 | 37 |
| 6209 | 1 | 88 | 39 |
| 6210 | 2 | 125 | 8 |
| 6211 | 1 | 124 | 7 |
| 6212 | 2 | 52 | 37 |
| 6213 | 1 | 100 | 39 |
| 6214 | 2 | 137 | 8 |
| 6215 | 1 | 136 | 7 |
| 6216 | 2 | 64 | 37 |
| 6217 | 1 | 5 | 43 |
| 6218 | 2 | 79 | 41 |
| 6219 | 1 | 42 | 5 |
| 6220 | 2 | 4 | 9 |
| 6221 | 1 | 17 | 43 |
| 6222 | 2 | 91 | 41 |
| 6223 | 1 | 54 | 5 |
| 6224 | 2 | 16 | 9 |
| 6225 | 1 | 29 | 43 |
| 6226 | 2 | 103 | 41 |
| 6227 | 1 | 66 | 5 |
| 6228 | 2 | 28 | 9 |
| 6229 | 1 | 47 | 6 |
| 6230 | 2 | 40 | 3 |
| 6231 | 1 | 48 | 8 |
| 6232 | 2 | 5 | 6 |
| 6233 | 1 | 59 | 6 |
| 6234 | 2 | 52 | 3 |
| 6235 | 1 | 60 | 8 |
| 6236 | 2 | 17 | 6 |
| 6237 | 1 | 71 | 6 |
| 6238 | 2 | 64 | 3 |
| 6239 | 1 | 72 | 8 |
| 6240 | 2 | 29 | 6 |
| 6241 | 1 | 47 | 47 |
| 6242 | 2 | 39 | 16 |
| 6243 | 1 | 120 | 46 |
| 6244 | 2 | 48 | 9 |
| 6245 | 1 | 59 | 47 |
| 6246 | 2 | 51 | 16 |
| 6247 | 1 | 132 | 46 |
| 6248 | 2 | 60 | 9 |
| 6249 | 1 | 71 | 47 |
| 6250 | 2 | 63 | 16 |
| 6251 | 1 | 144 | 46 |
| 6252 | 2 | 72 | 9 |
| 6253 | 1 | 81 | 12 |
| 6254 | 2 | 75 | 19 |
| 6255 | 1 | 80 | 44 |
| 6256 | 2 | 76 | 13 |
| 6257 | 1 | 93 | 12 |
| 6258 | 2 | 87 | 19 |
| 6259 | 1 | 92 | 44 |
| 6260 | 2 | 88 | 13 |
| 6261 | 1 | 105 | 12 |
| 6262 | 2 | 99 | 19 |
| 6263 | 1 | 104 | 44 |
| 6264 | 2 | 100 | 13 |
| 6265 | 1 | 111 | 48 |
| 6266 | 2 | 76 | 44 |
| 6267 | 1 | 4 | 16 |
| 6268 | 2 | 4 | 45 |
| 6269 | 1 | 123 | 48 |
| 6270 | 2 | 88 | 44 |
| 6271 | 1 | 16 | 16 |
| 6272 | 2 | 16 | 45 |
| 6273 | 1 | 135 | 48 |
| 6274 | 2 | 100 | 44 |
| 6275 | 1 | 28 | 16 |
| 6276 | 2 | 28 | 45 |
| 6277 | 1 | 112 | 47 |
| 6278 | 2 | 47 | 44 |
| 6279 | 1 | 76 | 16 |
| 6280 | 2 | 40 | 42 |
| 6281 | 1 | 124 | 47 |
| 6282 | 2 | 59 | 44 |
| 6283 | 1 | 88 | 16 |
| 6284 | 2 | 52 | 42 |
| 6285 | 1 | 136 | 47 |
| 6286 | 2 | 71 | 44 |
| 6287 | 1 | 100 | 16 |
| 6288 | 2 | 64 | 42 |
| 6289 | 1 | 112 | 48 |
| 6290 | 2 | 11 | 44 |
| 6291 | 1 | 112 | 14 |
| 6292 | 2 | 40 | 13 |
| 6293 | 1 | 124 | 48 |
| 6294 | 2 | 23 | 44 |
| 6295 | 1 | 124 | 14 |
| 6296 | 2 | 52 | 13 |
| 6297 | 1 | 136 | 48 |
| 6298 | 2 | 35 | 44 |
| 6299 | 1 | 136 | 14 |
| 6300 | 2 | 64 | 13 |
| 6301 | 1 | 76 | 47 |
| 6302 | 2 | 113 | 16 |
| 6303 | 1 | 112 | 15 |
| 6304 | 2 | 40 | 45 |
| 6305 | 1 | 88 | 47 |
| 6306 | 2 | 125 | 16 |
| 6307 | 1 | 124 | 15 |
| 6308 | 2 | 52 | 45 |
| 6309 | 1 | 100 | 47 |
| 6310 | 2 | 137 | 16 |
| 6311 | 1 | 136 | 15 |
| 6312 | 2 | 64 | 45 |
| 6313 | 1 | 5 | 51 |
| 6314 | 2 | 79 | 49 |
| 6315 | 1 | 42 | 13 |
| 6316 | 2 | 4 | 17 |
| 6317 | 1 | 17 | 51 |
| 6318 | 2 | 91 | 49 |
| 6319 | 1 | 54 | 13 |
| 6320 | 2 | 16 | 17 |
| 6321 | 1 | 29 | 51 |
| 6322 | 2 | 103 | 49 |
| 6323 | 1 | 66 | 13 |
| 6324 | 2 | 28 | 17 |
| 6325 | 1 | 47 | 14 |
| 6326 | 2 | 40 | 11 |
| 6327 | 1 | 48 | 16 |
| 6328 | 2 | 5 | 14 |
| 6329 | 1 | 59 | 14 |
| 6330 | 2 | 52 | 11 |
| 6331 | 1 | 60 | 16 |
| 6332 | 2 | 17 | 14 |
| 6333 | 1 | 71 | 14 |
| 6334 | 2 | 64 | 11 |
| 6335 | 1 | 72 | 16 |
| 6336 | 2 | 29 | 14 |
| 6337 | 1 | 47 | 55 |
| 6338 | 2 | 39 | 24 |
| 6339 | 1 | 120 | 54 |
| 6340 | 2 | 48 | 17 |
| 6341 | 1 | 59 | 55 |
| 6342 | 2 | 51 | 24 |
| 6343 | 1 | 132 | 54 |
| 6344 | 2 | 60 | 17 |
| 6345 | 1 | 71 | 55 |
| 6346 | 2 | 63 | 24 |
| 6347 | 1 | 144 | 54 |
| 6348 | 2 | 72 | 17 |
| 6349 | 1 | 81 | 20 |
| 6350 | 2 | 75 | 27 |
| 6351 | 1 | 80 | 52 |
| 6352 | 2 | 76 | 21 |
| 6353 | 1 | 93 | 20 |
| 6354 | 2 | 87 | 27 |
| 6355 | 1 | 92 | 52 |
| 6356 | 2 | 88 | 21 |
| 6357 | 1 | 105 | 20 |
| 6358 | 2 | 99 | 27 |
| 6359 | 1 | 104 | 52 |
| 6360 | 2 | 100 | 21 |
| 6361 | 1 | 111 | 56 |
| 6362 | 2 | 76 | 52 |
| 6363 | 1 | 4 | 24 |
| 6364 | 2 | 4 | 53 |
| 6365 | 1 | 123 | 56 |
| 6366 | 2 | 88 | 52 |
| 6367 | 1 | 16 | 24 |
| 6368 | 2 | 16 | 53 |
| 6369 | 1 | 135 | 56 |
| 6370 | 2 | 100 | 52 |
| 6371 | 1 | 28 | 24 |
| 6372 | 2 | 28 | 53 |
| 6373 | 1 | 112 | 55 |
| 6374 | 2 | 47 | 52 |
| 6375 | 1 | 76 | 24 |
| 6376 | 2 | 40 | 50 |
| 6377 | 1 | 124 | 55 |
| 6378 | 2 | 59 | 52 |
| 6379 | 1 | 88 | 24 |
| 6380 | 2 | 52 | 50 |
| 6381 | 1 | 136 | 55 |
| 6382 | 2 | 71 | 52 |
| 6383 | 1 | 100 | 24 |
| 6384 | 2 | 64 | 50 |
| 6385 | 1 | 112 | 56 |
| 6386 | 2 | 11 | 52 |
| 6387 | 1 | 112 | 22 |
| 6388 | 2 | 40 | 21 |
| 6389 | 1 | 124 | 56 |
| 6390 | 2 | 23 | 52 |
| 6391 | 1 | 124 | 22 |
| 6392 | 2 | 52 | 21 |
| 6393 | 1 | 136 | 56 |
| 6394 | 2 | 35 | 52 |
| 6395 | 1 | 136 | 22 |
| 6396 | 2 | 64 | 21 |
| 6397 | 1 | 76 | 55 |
| 6398 | 2 | 113 | 24 |
| 6399 | 1 | 112 | 23 |
| 6400 | 2 | 40 | 53 |
| 6401 | 1 | 88 | 55 |
| 6402 | 2 | 125 | 24 |
| 6403 | 1 | 124 | 23 |
| 6404 | 2 | 52 | 53 |
| 6405 | 1 | 100 | 55 |
| 6406 | 2 | 137 | 24 |
| 6407 | 1 | 136 | 23 |
| 6408 | 2 | 64 | 53 |
| 6409 | 1 | 5 | 59 |
| 6410 | 2 | 79 | 57 |
| 6411 | 1 | 42 | 21 |
| 6412 | 2 | 4 | 25 |
| 6413 | 1 | 17 | 59 |
| 6414 | 2 | 91 | 57 |
| 6415 | 1 | 54 | 21 |
| 6416 | 2 | 16 | 25 |
| 6417 | 1 | 29 | 59 |
| 6418 | 2 | 103 | 57 |
| 6419 | 1 | 66 | 21 |
| 6420 | 2 | 28 | 25 |
| 6421 | 1 | 47 | 22 |
| 6422 | 2 | 40 | 19 |
| 6423 | 1 | 48 | 24 |
| 6424 | 2 | 5 | 22 |
| 6425 | 1 | 59 | 22 |
| 6426 | 2 | 52 | 19 |
| 6427 | 1 | 60 | 24 |
| 6428 | 2 | 17 | 22 |
| 6429 | 1 | 71 | 22 |
| 6430 | 2 | 64 | 19 |
| 6431 | 1 | 72 | 24 |
| 6432 | 2 | 29 | 22 |
| 6433 | 1 | 47 | 63 |
| 6434 | 2 | 39 | 32 |
| 6435 | 1 | 120 | 62 |
| 6436 | 2 | 48 | 25 |
| 6437 | 1 | 59 | 63 |
| 6438 | 2 | 51 | 32 |
| 6439 | 1 | 132 | 62 |
| 6440 | 2 | 60 | 25 |
| 6441 | 1 | 71 | 63 |
| 6442 | 2 | 63 | 32 |
| 6443 | 1 | 144 | 62 |
| 6444 | 2 | 72 | 25 |
| 6445 | 1 | 81 | 28 |
| 6446 | 2 | 75 | 35 |
| 6447 | 1 | 80 | 60 |
| 6448 | 2 | 76 | 29 |
| 6449 | 1 | 93 | 28 |
| 6450 | 2 | 87 | 35 |
| 6451 | 1 | 92 | 60 |
| 6452 | 2 | 88 | 29 |
| 6453 | 1 | 105 | 28 |
| 6454 | 2 | 99 | 35 |
| 6455 | 1 | 104 | 60 |
| 6456 | 2 | 100 | 29 |
| 6457 | 1 | 111 | 64 |
| 6458 | 2 | 76 | 60 |
| 6459 | 1 | 4 | 32 |
| 6460 | 2 | 4 | 61 |
| 6461 | 1 | 123 | 64 |
| 6462 | 2 | 88 | 60 |
| 6463 | 1 | 16 | 32 |
| 6464 | 2 | 16 | 61 |
| 6465 | 1 | 135 | 64 |
| 6466 | 2 | 100 | 60 |
| 6467 | 1 | 28 | 32 |
| 6468 | 2 | 28 | 61 |
| 6469 | 1 | 112 | 63 |
| 6470 | 2 | 47 | 60 |
| 6471 | 1 | 76 | 32 |
| 6472 | 2 | 40 | 58 |
| 6473 | 1 | 124 | 63 |
| 6474 | 2 | 59 | 60 |
| 6475 | 1 | 88 | 32 |
| 6476 | 2 | 52 | 58 |
| 6477 | 1 | 136 | 63 |
| 6478 | 2 | 71 | 60 |
| 6479 | 1 | 100 | 32 |
| 6480 | 2 | 64 | 58 |
| 6481 | 1 | 112 | 64 |
| 6482 | 2 | 11 | 60 |
| 6483 | 1 | 112 | 30 |
| 6484 | 2 | 40 | 29 |
| 6485 | 1 | 124 | 64 |
| 6486 | 2 | 23 | 60 |
| 6487 | 1 | 124 | 30 |
| 6488 | 2 | 52 | 29 |
| 6489 | 1 | 136 | 64 |
| 6490 | 2 | 35 | 60 |
| 6491 | 1 | 136 | 30 |
| 6492 | 2 | 64 | 29 |
| 6493 | 1 | 76 | 63 |
| 6494 | 2 | 113 | 32 |
| 6495 | 1 | 112 | 31 |
| 6496 | 2 | 40 | 61 |
| 6497 | 1 | 88 | 63 |
| 6498 | 2 | 125 | 32 |
| 6499 | 1 | 124 | 31 |
| 6500 | 2 | 52 | 61 |
| 6501 | 1 | 100 | 63 |
| 6502 | 2 | 137 | 32 |
| 6503 | 1 | 136 | 31 |
| 6504 | 2 | 64 | 61 |
| 6505 | 1 | 5 | 67 |
| 6506 | 2 | 79 | 65 |
| 6507 | 1 | 42 | 29 |
| 6508 | 2 | 4 | 33 |
| 6509 | 1 | 17 | 67 |
| 6510 | 2 | 91 | 65 |
| 6511 | 1 | 54 | 29 |
| 6512 | 2 | 16 | 33 |
| 6513 | 1 | 29 | 67 |
| 6514 | 2 | 103 | 65 |
| 6515 | 1 | 66 | 29 |
| 6516 | 2 | 28 | 33 |
| 6517 | 1 | 47 | 30 |
| 6518 | 2 | 40 | 27 |
| 6519 | 1 | 48 | 32 |
| 6520 | 2 | 5 | 30 |
| 6521 | 1 | 59 | 30 |
| 6522 | 2 | 52 | 27 |
| 6523 | 1 | 60 | 32 |
| 6524 | 2 | 17 | 30 |
| 6525 | 1 | 71 | 30 |
| 6526 | 2 | 64 | 27 |
| 6527 | 1 | 72 | 32 |
| 6528 | 2 | 29 | 30 |
| 6529 | 1 | 3 | 43 |
| 6530 | 2 | 111 | 8 |
| 6531 | 1 | 40 | 35 |
| 6532 | 2 | 76 | 37 |
| 6533 | 1 | 15 | 43 |
| 6534 | 2 | 123 | 8 |
| 6535 | 1 | 52 | 35 |
| 6536 | 2 | 88 | 37 |
| 6537 | 1 | 27 | 43 |
| 6538 | 2 | 135 | 8 |
| 6539 | 1 | 64 | 35 |
| 6540 | 2 | 100 | 37 |
| 6541 | 1 | 43 | 38 |
| 6542 | 2 | 4 | 5 |
| 6543 | 1 | 40 | 38 |
| 6544 | 2 | 112 | 34 |
| 6545 | 1 | 55 | 38 |
| 6546 | 2 | 16 | 5 |
| 6547 | 1 | 52 | 38 |
| 6548 | 2 | 124 | 34 |
| 6549 | 1 | 67 | 38 |
| 6550 | 2 | 28 | 5 |
| 6551 | 1 | 64 | 38 |
| 6552 | 2 | 136 | 34 |
| 6553 | 1 | 116 | 7 |
| 6554 | 2 | 5 | 8 |
| 6555 | 1 | 4 | 10 |
| 6556 | 2 | 4 | 6 |
| 6557 | 1 | 128 | 7 |
| 6558 | 2 | 17 | 8 |
| 6559 | 1 | 16 | 10 |
| 6560 | 2 | 16 | 6 |
| 6561 | 1 | 140 | 7 |
| 6562 | 2 | 29 | 8 |
| 6563 | 1 | 28 | 10 |
| 6564 | 2 | 28 | 6 |
| 6565 | 1 | 45 | 37 |
| 6566 | 2 | 40 | 2 |
| 6567 | 1 | 40 | 4 |
| 6568 | 2 | 76 | 6 |
| 6569 | 1 | 57 | 37 |
| 6570 | 2 | 52 | 2 |
| 6571 | 1 | 52 | 4 |
| 6572 | 2 | 88 | 6 |
| 6573 | 1 | 69 | 37 |
| 6574 | 2 | 64 | 2 |
| 6575 | 1 | 64 | 4 |
| 6576 | 2 | 100 | 6 |
| 6577 | 1 | 113 | 2 |
| 6578 | 2 | 119 | 37 |
| 6579 | 1 | 42 | 39 |
| 6580 | 2 | 40 | 6 |
| 6581 | 1 | 125 | 2 |
| 6582 | 2 | 131 | 37 |
| 6583 | 1 | 54 | 39 |
| 6584 | 2 | 52 | 6 |
| 6585 | 1 | 137 | 2 |
| 6586 | 2 | 143 | 37 |
| 6587 | 1 | 66 | 39 |
| 6588 | 2 | 64 | 6 |
| 6589 | 1 | 114 | 39 |
| 6590 | 2 | 112 | 35 |
| 6591 | 1 | 9 | 5 |
| 6592 | 2 | 82 | 40 |
| 6593 | 1 | 126 | 39 |
| 6594 | 2 | 124 | 35 |
| 6595 | 1 | 21 | 5 |
| 6596 | 2 | 94 | 40 |
| 6597 | 1 | 138 | 39 |
| 6598 | 2 | 136 | 35 |
| 6599 | 1 | 33 | 5 |
| 6600 | 2 | 106 | 40 |
| 6601 | 1 | 76 | 40 |
| 6602 | 2 | 4 | 7 |
| 6603 | 1 | 76 | 42 |
| 6604 | 2 | 112 | 38 |
| 6605 | 1 | 88 | 40 |
| 6606 | 2 | 16 | 7 |
| 6607 | 1 | 88 | 42 |
| 6608 | 2 | 124 | 38 |
| 6609 | 1 | 100 | 40 |
| 6610 | 2 | 28 | 7 |
| 6611 | 1 | 100 | 42 |
| 6612 | 2 | 136 | 38 |
| 6613 | 1 | 112 | 4 |
| 6614 | 2 | 83 | 38 |
| 6615 | 1 | 42 | 4 |
| 6616 | 2 | 40 | 7 |
| 6617 | 1 | 124 | 4 |
| 6618 | 2 | 95 | 38 |
| 6619 | 1 | 54 | 4 |
| 6620 | 2 | 52 | 7 |
| 6621 | 1 | 136 | 4 |
| 6622 | 2 | 107 | 38 |
| 6623 | 1 | 66 | 4 |
| 6624 | 2 | 64 | 7 |
| 6625 | 1 | 3 | 51 |
| 6626 | 2 | 111 | 16 |
| 6627 | 1 | 40 | 43 |
| 6628 | 2 | 76 | 45 |
| 6629 | 1 | 15 | 51 |
| 6630 | 2 | 123 | 16 |
| 6631 | 1 | 52 | 43 |
| 6632 | 2 | 88 | 45 |
| 6633 | 1 | 27 | 51 |
| 6634 | 2 | 135 | 16 |
| 6635 | 1 | 64 | 43 |
| 6636 | 2 | 100 | 45 |
| 6637 | 1 | 43 | 46 |
| 6638 | 2 | 4 | 13 |
| 6639 | 1 | 40 | 46 |
| 6640 | 2 | 112 | 42 |
| 6641 | 1 | 55 | 46 |
| 6642 | 2 | 16 | 13 |
| 6643 | 1 | 52 | 46 |
| 6644 | 2 | 124 | 42 |
| 6645 | 1 | 67 | 46 |
| 6646 | 2 | 28 | 13 |
| 6647 | 1 | 64 | 46 |
| 6648 | 2 | 136 | 42 |
| 6649 | 1 | 116 | 15 |
| 6650 | 2 | 5 | 16 |
| 6651 | 1 | 4 | 18 |
| 6652 | 2 | 4 | 14 |
| 6653 | 1 | 128 | 15 |
| 6654 | 2 | 17 | 16 |
| 6655 | 1 | 16 | 18 |
| 6656 | 2 | 16 | 14 |
| 6657 | 1 | 140 | 15 |
| 6658 | 2 | 29 | 16 |
| 6659 | 1 | 28 | 18 |
| 6660 | 2 | 28 | 14 |
| 6661 | 1 | 45 | 45 |
| 6662 | 2 | 40 | 10 |
| 6663 | 1 | 40 | 12 |
| 6664 | 2 | 76 | 14 |
| 6665 | 1 | 57 | 45 |
| 6666 | 2 | 52 | 10 |
| 6667 | 1 | 52 | 12 |
| 6668 | 2 | 88 | 14 |
| 6669 | 1 | 69 | 45 |
| 6670 | 2 | 64 | 10 |
| 6671 | 1 | 64 | 12 |
| 6672 | 2 | 100 | 14 |
| 6673 | 1 | 113 | 10 |
| 6674 | 2 | 119 | 45 |
| 6675 | 1 | 42 | 47 |
| 6676 | 2 | 40 | 14 |
| 6677 | 1 | 125 | 10 |
| 6678 | 2 | 131 | 45 |
| 6679 | 1 | 54 | 47 |
| 6680 | 2 | 52 | 14 |
| 6681 | 1 | 137 | 10 |
| 6682 | 2 | 143 | 45 |
| 6683 | 1 | 66 | 47 |
| 6684 | 2 | 64 | 14 |
| 6685 | 1 | 114 | 47 |
| 6686 | 2 | 112 | 43 |
| 6687 | 1 | 9 | 13 |
| 6688 | 2 | 82 | 48 |
| 6689 | 1 | 126 | 47 |
| 6690 | 2 | 124 | 43 |
| 6691 | 1 | 21 | 13 |
| 6692 | 2 | 94 | 48 |
| 6693 | 1 | 138 | 47 |
| 6694 | 2 | 136 | 43 |
| 6695 | 1 | 33 | 13 |
| 6696 | 2 | 106 | 48 |
| 6697 | 1 | 76 | 48 |
| 6698 | 2 | 4 | 15 |
| 6699 | 1 | 76 | 50 |
| 6700 | 2 | 112 | 46 |
| 6701 | 1 | 88 | 48 |
| 6702 | 2 | 16 | 15 |
| 6703 | 1 | 88 | 50 |
| 6704 | 2 | 124 | 46 |
| 6705 | 1 | 100 | 48 |
| 6706 | 2 | 28 | 15 |
| 6707 | 1 | 100 | 50 |
| 6708 | 2 | 136 | 46 |
| 6709 | 1 | 112 | 12 |
| 6710 | 2 | 83 | 46 |
| 6711 | 1 | 42 | 12 |
| 6712 | 2 | 40 | 15 |
| 6713 | 1 | 124 | 12 |
| 6714 | 2 | 95 | 46 |
| 6715 | 1 | 54 | 12 |
| 6716 | 2 | 52 | 15 |
| 6717 | 1 | 136 | 12 |
| 6718 | 2 | 107 | 46 |
| 6719 | 1 | 66 | 12 |
| 6720 | 2 | 64 | 15 |
| 6721 | 1 | 3 | 59 |
| 6722 | 2 | 111 | 24 |
| 6723 | 1 | 40 | 51 |
| 6724 | 2 | 76 | 53 |
| 6725 | 1 | 15 | 59 |
| 6726 | 2 | 123 | 24 |
| 6727 | 1 | 52 | 51 |
| 6728 | 2 | 88 | 53 |
| 6729 | 1 | 27 | 59 |
| 6730 | 2 | 135 | 24 |
| 6731 | 1 | 64 | 51 |
| 6732 | 2 | 100 | 53 |
| 6733 | 1 | 43 | 54 |
| 6734 | 2 | 4 | 21 |
| 6735 | 1 | 40 | 54 |
| 6736 | 2 | 112 | 50 |
| 6737 | 1 | 55 | 54 |
| 6738 | 2 | 16 | 21 |
| 6739 | 1 | 52 | 54 |
| 6740 | 2 | 124 | 50 |
| 6741 | 1 | 67 | 54 |
| 6742 | 2 | 28 | 21 |
| 6743 | 1 | 64 | 54 |
| 6744 | 2 | 136 | 50 |
| 6745 | 1 | 116 | 23 |
| 6746 | 2 | 5 | 24 |
| 6747 | 1 | 4 | 26 |
| 6748 | 2 | 4 | 22 |
| 6749 | 1 | 128 | 23 |
| 6750 | 2 | 17 | 24 |
| 6751 | 1 | 16 | 26 |
| 6752 | 2 | 16 | 22 |
| 6753 | 1 | 140 | 23 |
| 6754 | 2 | 29 | 24 |
| 6755 | 1 | 28 | 26 |
| 6756 | 2 | 28 | 22 |
| 6757 | 1 | 45 | 53 |
| 6758 | 2 | 40 | 18 |
| 6759 | 1 | 40 | 20 |
| 6760 | 2 | 76 | 22 |
| 6761 | 1 | 57 | 53 |
| 6762 | 2 | 52 | 18 |
| 6763 | 1 | 52 | 20 |
| 6764 | 2 | 88 | 22 |
| 6765 | 1 | 69 | 53 |
| 6766 | 2 | 64 | 18 |
| 6767 | 1 | 64 | 20 |
| 6768 | 2 | 100 | 22 |
| 6769 | 1 | 113 | 18 |
| 6770 | 2 | 119 | 53 |
| 6771 | 1 | 42 | 55 |
| 6772 | 2 | 40 | 22 |
| 6773 | 1 | 125 | 18 |
| 6774 | 2 | 131 | 53 |
| 6775 | 1 | 54 | 55 |
| 6776 | 2 | 52 | 22 |
| 6777 | 1 | 137 | 18 |
| 6778 | 2 | 143 | 53 |
| 6779 | 1 | 66 | 55 |
| 6780 | 2 | 64 | 22 |
| 6781 | 1 | 114 | 55 |
| 6782 | 2 | 112 | 51 |
| 6783 | 1 | 9 | 21 |
| 6784 | 2 | 82 | 56 |
| 6785 | 1 | 126 | 55 |
| 6786 | 2 | 124 | 51 |
| 6787 | 1 | 21 | 21 |
| 6788 | 2 | 94 | 56 |
| 6789 | 1 | 138 | 55 |
| 6790 | 2 | 136 | 51 |
| 6791 | 1 | 33 | 21 |
| 6792 | 2 | 106 | 56 |
| 6793 | 1 | 76 | 56 |
| 6794 | 2 | 4 | 23 |
| 6795 | 1 | 76 | 58 |
| 6796 | 2 | 112 | 54 |
| 6797 | 1 | 88 | 56 |
| 6798 | 2 | 16 | 23 |
| 6799 | 1 | 88 | 58 |
| 6800 | 2 | 124 | 54 |
| 6801 | 1 | 100 | 56 |
| 6802 | 2 | 28 | 23 |
| 6803 | 1 | 100 | 58 |
| 6804 | 2 | 136 | 54 |
| 6805 | 1 | 112 | 20 |
| 6806 | 2 | 83 | 54 |
| 6807 | 1 | 42 | 20 |
| 6808 | 2 | 40 | 23 |
| 6809 | 1 | 124 | 20 |
| 6810 | 2 | 95 | 54 |
| 6811 | 1 | 54 | 20 |
| 6812 | 2 | 52 | 23 |
| 6813 | 1 | 136 | 20 |
| 6814 | 2 | 107 | 54 |
| 6815 | 1 | 66 | 20 |
| 6816 | 2 | 64 | 23 |
| 6817 | 1 | 3 | 67 |
| 6818 | 2 | 111 | 32 |
| 6819 | 1 | 40 | 59 |
| 6820 | 2 | 76 | 61 |
| 6821 | 1 | 15 | 67 |
| 6822 | 2 | 123 | 32 |
| 6823 | 1 | 52 | 59 |
| 6824 | 2 | 88 | 61 |
| 6825 | 1 | 27 | 67 |
| 6826 | 2 | 135 | 32 |
| 6827 | 1 | 64 | 59 |
| 6828 | 2 | 100 | 61 |
| 6829 | 1 | 43 | 62 |
| 6830 | 2 | 4 | 29 |
| 6831 | 1 | 40 | 62 |
| 6832 | 2 | 112 | 58 |
| 6833 | 1 | 55 | 62 |
| 6834 | 2 | 16 | 29 |
| 6835 | 1 | 52 | 62 |
| 6836 | 2 | 124 | 58 |
| 6837 | 1 | 67 | 62 |
| 6838 | 2 | 28 | 29 |
| 6839 | 1 | 64 | 62 |
| 6840 | 2 | 136 | 58 |
| 6841 | 1 | 116 | 31 |
| 6842 | 2 | 5 | 32 |
| 6843 | 1 | 4 | 34 |
| 6844 | 2 | 4 | 30 |
| 6845 | 1 | 128 | 31 |
| 6846 | 2 | 17 | 32 |
| 6847 | 1 | 16 | 34 |
| 6848 | 2 | 16 | 30 |
| 6849 | 1 | 140 | 31 |
| 6850 | 2 | 29 | 32 |
| 6851 | 1 | 28 | 34 |
| 6852 | 2 | 28 | 30 |
| 6853 | 1 | 45 | 61 |
| 6854 | 2 | 40 | 26 |
| 6855 | 1 | 40 | 28 |
| 6856 | 2 | 76 | 30 |
| 6857 | 1 | 57 | 61 |
| 6858 | 2 | 52 | 26 |
| 6859 | 1 | 52 | 28 |
| 6860 | 2 | 88 | 30 |
| 6861 | 1 | 69 | 61 |
| 6862 | 2 | 64 | 26 |
| 6863 | 1 | 64 | 28 |
| 6864 | 2 | 100 | 30 |
| 6865 | 1 | 113 | 26 |
| 6866 | 2 | 119 | 61 |
| 6867 | 1 | 42 | 63 |
| 6868 | 2 | 40 | 30 |
| 6869 | 1 | 125 | 26 |
| 6870 | 2 | 131 | 61 |
| 6871 | 1 | 54 | 63 |
| 6872 | 2 | 52 | 30 |
| 6873 | 1 | 137 | 26 |
| 6874 | 2 | 143 | 61 |
| 6875 | 1 | 66 | 63 |
| 6876 | 2 | 64 | 30 |
| 6877 | 1 | 114 | 63 |
| 6878 | 2 | 112 | 59 |
| 6879 | 1 | 9 | 29 |
| 6880 | 2 | 82 | 64 |
| 6881 | 1 | 126 | 63 |
| 6882 | 2 | 124 | 59 |
| 6883 | 1 | 21 | 29 |
| 6884 | 2 | 94 | 64 |
| 6885 | 1 | 138 | 63 |
| 6886 | 2 | 136 | 59 |
| 6887 | 1 | 33 | 29 |
| 6888 | 2 | 106 | 64 |
| 6889 | 1 | 76 | 64 |
| 6890 | 2 | 4 | 31 |
| 6891 | 1 | 76 | 66 |
| 6892 | 2 | 112 | 62 |
| 6893 | 1 | 88 | 64 |
| 6894 | 2 | 16 | 31 |
| 6895 | 1 | 88 | 66 |
| 6896 | 2 | 124 | 62 |
| 6897 | 1 | 100 | 64 |
| 6898 | 2 | 28 | 31 |
| 6899 | 1 | 100 | 66 |
| 6900 | 2 | 136 | 62 |
| 6901 | 1 | 112 | 28 |
| 6902 | 2 | 83 | 62 |
| 6903 | 1 | 42 | 28 |
| 6904 | 2 | 40 | 31 |
| 6905 | 1 | 124 | 28 |
| 6906 | 2 | 95 | 62 |
| 6907 | 1 | 54 | 28 |
| 6908 | 2 | 52 | 31 |
| 6909 | 1 | 136 | 28 |
| 6910 | 2 | 107 | 62 |
| 6911 | 1 | 66 | 28 |
| 6912 | 2 | 64 | 31 |
| 6913 | 1 | 12 | 6 |
| 6914 | 2 | 4 | 42 |
| 6915 | 1 | 41 | 3 |
| 6916 | 2 | 12 | 39 |
| 6917 | 1 | 24 | 6 |
| 6918 | 2 | 16 | 42 |
| 6919 | 1 | 53 | 3 |
| 6920 | 2 | 24 | 39 |
| 6921 | 1 | 36 | 6 |
| 6922 | 2 | 28 | 42 |
| 6923 | 1 | 65 | 3 |
| 6924 | 2 | 36 | 39 |
| 6925 | 1 | 114 | 5 |
| 6926 | 2 | 80 | 41 |
| 6927 | 1 | 42 | 1 |
| 6928 | 2 | 113 | 4 |
| 6929 | 1 | 126 | 5 |
| 6930 | 2 | 92 | 41 |
| 6931 | 1 | 54 | 1 |
| 6932 | 2 | 125 | 4 |
| 6933 | 1 | 138 | 5 |
| 6934 | 2 | 104 | 41 |
| 6935 | 1 | 66 | 1 |
| 6936 | 2 | 137 | 4 |
| 6937 | 1 | 11 | 43 |
| 6938 | 2 | 76 | 11 |
| 6939 | 1 | 114 | 40 |
| 6940 | 2 | 41 | 4 |
| 6941 | 1 | 23 | 43 |
| 6942 | 2 | 88 | 11 |
| 6943 | 1 | 126 | 40 |
| 6944 | 2 | 53 | 4 |
| 6945 | 1 | 35 | 43 |
| 6946 | 2 | 100 | 11 |
| 6947 | 1 | 138 | 40 |
| 6948 | 2 | 65 | 4 |
| 6949 | 1 | 41 | 1 |
| 6950 | 2 | 112 | 8 |
| 6951 | 1 | 79 | 42 |
| 6952 | 2 | 5 | 39 |
| 6953 | 1 | 53 | 1 |
| 6954 | 2 | 124 | 8 |
| 6955 | 1 | 91 | 42 |
| 6956 | 2 | 17 | 39 |
| 6957 | 1 | 65 | 1 |
| 6958 | 2 | 136 | 8 |
| 6959 | 1 | 103 | 42 |
| 6960 | 2 | 29 | 39 |
| 6961 | 1 | 114 | 38 |
| 6962 | 2 | 41 | 37 |
| 6963 | 1 | 114 | 4 |
| 6964 | 2 | 41 | 39 |
| 6965 | 1 | 126 | 38 |
| 6966 | 2 | 53 | 37 |
| 6967 | 1 | 126 | 4 |
| 6968 | 2 | 53 | 39 |
| 6969 | 1 | 138 | 38 |
| 6970 | 2 | 65 | 37 |
| 6971 | 1 | 138 | 4 |
| 6972 | 2 | 65 | 39 |
| 6973 | 1 | 42 | 8 |
| 6974 | 2 | 113 | 6 |
| 6975 | 1 | 84 | 11 |
| 6976 | 2 | 7 | 5 |
| 6977 | 1 | 54 | 8 |
| 6978 | 2 | 125 | 6 |
| 6979 | 1 | 96 | 11 |
| 6980 | 2 | 19 | 5 |
| 6981 | 1 | 66 | 8 |
| 6982 | 2 | 137 | 6 |
| 6983 | 1 | 108 | 11 |
| 6984 | 2 | 31 | 5 |
| 6985 | 1 | 79 | 4 |
| 6986 | 2 | 41 | 6 |
| 6987 | 1 | 116 | 33 |
| 6988 | 2 | 113 | 7 |
| 6989 | 1 | 91 | 4 |
| 6990 | 2 | 53 | 6 |
| 6991 | 1 | 128 | 33 |
| 6992 | 2 | 125 | 7 |
| 6993 | 1 | 103 | 4 |
| 6994 | 2 | 65 | 6 |
| 6995 | 1 | 140 | 33 |
| 6996 | 2 | 137 | 7 |
| 6997 | 1 | 8 | 9 |
| 6998 | 2 | 5 | 41 |
| 6999 | 1 | 44 | 38 |
| 7000 | 2 | 77 | 42 |
| 7001 | 1 | 20 | 9 |
| 7002 | 2 | 17 | 41 |
| 7003 | 1 | 56 | 38 |
| 7004 | 2 | 89 | 42 |
| 7005 | 1 | 32 | 9 |
| 7006 | 2 | 29 | 41 |
| 7007 | 1 | 68 | 38 |
| 7008 | 2 | 101 | 42 |
| 7009 | 1 | 12 | 14 |
| 7010 | 2 | 4 | 50 |
| 7011 | 1 | 41 | 11 |
| 7012 | 2 | 12 | 47 |
| 7013 | 1 | 24 | 14 |
| 7014 | 2 | 16 | 50 |
| 7015 | 1 | 53 | 11 |
| 7016 | 2 | 24 | 47 |
| 7017 | 1 | 36 | 14 |
| 7018 | 2 | 28 | 50 |
| 7019 | 1 | 65 | 11 |
| 7020 | 2 | 36 | 47 |
| 7021 | 1 | 114 | 13 |
| 7022 | 2 | 80 | 49 |
| 7023 | 1 | 42 | 9 |
| 7024 | 2 | 113 | 12 |
| 7025 | 1 | 126 | 13 |
| 7026 | 2 | 92 | 49 |
| 7027 | 1 | 54 | 9 |
| 7028 | 2 | 125 | 12 |
| 7029 | 1 | 138 | 13 |
| 7030 | 2 | 104 | 49 |
| 7031 | 1 | 66 | 9 |
| 7032 | 2 | 137 | 12 |
| 7033 | 1 | 11 | 51 |
| 7034 | 2 | 76 | 19 |
| 7035 | 1 | 114 | 48 |
| 7036 | 2 | 41 | 12 |
| 7037 | 1 | 23 | 51 |
| 7038 | 2 | 88 | 19 |
| 7039 | 1 | 126 | 48 |
| 7040 | 2 | 53 | 12 |
| 7041 | 1 | 35 | 51 |
| 7042 | 2 | 100 | 19 |
| 7043 | 1 | 138 | 48 |
| 7044 | 2 | 65 | 12 |
| 7045 | 1 | 41 | 9 |
| 7046 | 2 | 112 | 16 |
| 7047 | 1 | 79 | 50 |
| 7048 | 2 | 5 | 47 |
| 7049 | 1 | 53 | 9 |
| 7050 | 2 | 124 | 16 |
| 7051 | 1 | 91 | 50 |
| 7052 | 2 | 17 | 47 |
| 7053 | 1 | 65 | 9 |
| 7054 | 2 | 136 | 16 |
| 7055 | 1 | 103 | 50 |
| 7056 | 2 | 29 | 47 |
| 7057 | 1 | 114 | 46 |
| 7058 | 2 | 41 | 45 |
| 7059 | 1 | 114 | 12 |
| 7060 | 2 | 41 | 47 |
| 7061 | 1 | 126 | 46 |
| 7062 | 2 | 53 | 45 |
| 7063 | 1 | 126 | 12 |
| 7064 | 2 | 53 | 47 |
| 7065 | 1 | 138 | 46 |
| 7066 | 2 | 65 | 45 |
| 7067 | 1 | 138 | 12 |
| 7068 | 2 | 65 | 47 |
| 7069 | 1 | 42 | 16 |
| 7070 | 2 | 113 | 14 |
| 7071 | 1 | 84 | 19 |
| 7072 | 2 | 7 | 13 |
| 7073 | 1 | 54 | 16 |
| 7074 | 2 | 125 | 14 |
| 7075 | 1 | 96 | 19 |
| 7076 | 2 | 19 | 13 |
| 7077 | 1 | 66 | 16 |
| 7078 | 2 | 137 | 14 |
| 7079 | 1 | 108 | 19 |
| 7080 | 2 | 31 | 13 |
| 7081 | 1 | 79 | 12 |
| 7082 | 2 | 41 | 14 |
| 7083 | 1 | 116 | 41 |
| 7084 | 2 | 113 | 15 |
| 7085 | 1 | 91 | 12 |
| 7086 | 2 | 53 | 14 |
| 7087 | 1 | 128 | 41 |
| 7088 | 2 | 125 | 15 |
| 7089 | 1 | 103 | 12 |
| 7090 | 2 | 65 | 14 |
| 7091 | 1 | 140 | 41 |
| 7092 | 2 | 137 | 15 |
| 7093 | 1 | 8 | 17 |
| 7094 | 2 | 5 | 49 |
| 7095 | 1 | 44 | 46 |
| 7096 | 2 | 77 | 50 |
| 7097 | 1 | 20 | 17 |
| 7098 | 2 | 17 | 49 |
| 7099 | 1 | 56 | 46 |
| 7100 | 2 | 89 | 50 |
| 7101 | 1 | 32 | 17 |
| 7102 | 2 | 29 | 49 |
| 7103 | 1 | 68 | 46 |
| 7104 | 2 | 101 | 50 |
| 7105 | 1 | 12 | 22 |
| 7106 | 2 | 4 | 58 |
| 7107 | 1 | 41 | 19 |
| 7108 | 2 | 12 | 55 |
| 7109 | 1 | 24 | 22 |
| 7110 | 2 | 16 | 58 |
| 7111 | 1 | 53 | 19 |
| 7112 | 2 | 24 | 55 |
| 7113 | 1 | 36 | 22 |
| 7114 | 2 | 28 | 58 |
| 7115 | 1 | 65 | 19 |
| 7116 | 2 | 36 | 55 |
| 7117 | 1 | 114 | 21 |
| 7118 | 2 | 80 | 57 |
| 7119 | 1 | 42 | 17 |
| 7120 | 2 | 113 | 20 |
| 7121 | 1 | 126 | 21 |
| 7122 | 2 | 92 | 57 |
| 7123 | 1 | 54 | 17 |
| 7124 | 2 | 125 | 20 |
| 7125 | 1 | 138 | 21 |
| 7126 | 2 | 104 | 57 |
| 7127 | 1 | 66 | 17 |
| 7128 | 2 | 137 | 20 |
| 7129 | 1 | 11 | 59 |
| 7130 | 2 | 76 | 27 |
| 7131 | 1 | 114 | 56 |
| 7132 | 2 | 41 | 20 |
| 7133 | 1 | 23 | 59 |
| 7134 | 2 | 88 | 27 |
| 7135 | 1 | 126 | 56 |
| 7136 | 2 | 53 | 20 |
| 7137 | 1 | 35 | 59 |
| 7138 | 2 | 100 | 27 |
| 7139 | 1 | 138 | 56 |
| 7140 | 2 | 65 | 20 |
| 7141 | 1 | 41 | 17 |
| 7142 | 2 | 112 | 24 |
| 7143 | 1 | 79 | 58 |
| 7144 | 2 | 5 | 55 |
| 7145 | 1 | 53 | 17 |
| 7146 | 2 | 124 | 24 |
| 7147 | 1 | 91 | 58 |
| 7148 | 2 | 17 | 55 |
| 7149 | 1 | 65 | 17 |
| 7150 | 2 | 136 | 24 |
| 7151 | 1 | 103 | 58 |
| 7152 | 2 | 29 | 55 |
| 7153 | 1 | 114 | 54 |
| 7154 | 2 | 41 | 53 |
| 7155 | 1 | 114 | 20 |
| 7156 | 2 | 41 | 55 |
| 7157 | 1 | 126 | 54 |
| 7158 | 2 | 53 | 53 |
| 7159 | 1 | 126 | 20 |
| 7160 | 2 | 53 | 55 |
| 7161 | 1 | 138 | 54 |
| 7162 | 2 | 65 | 53 |
| 7163 | 1 | 138 | 20 |
| 7164 | 2 | 65 | 55 |
| 7165 | 1 | 42 | 24 |
| 7166 | 2 | 113 | 22 |
| 7167 | 1 | 84 | 27 |
| 7168 | 2 | 7 | 21 |
| 7169 | 1 | 54 | 24 |
| 7170 | 2 | 125 | 22 |
| 7171 | 1 | 96 | 27 |
| 7172 | 2 | 19 | 21 |
| 7173 | 1 | 66 | 24 |
| 7174 | 2 | 137 | 22 |
| 7175 | 1 | 108 | 27 |
| 7176 | 2 | 31 | 21 |
| 7177 | 1 | 79 | 20 |
| 7178 | 2 | 41 | 22 |
| 7179 | 1 | 116 | 49 |
| 7180 | 2 | 113 | 23 |
| 7181 | 1 | 91 | 20 |
| 7182 | 2 | 53 | 22 |
| 7183 | 1 | 128 | 49 |
| 7184 | 2 | 125 | 23 |
| 7185 | 1 | 103 | 20 |
| 7186 | 2 | 65 | 22 |
| 7187 | 1 | 140 | 49 |
| 7188 | 2 | 137 | 23 |
| 7189 | 1 | 8 | 25 |
| 7190 | 2 | 5 | 57 |
| 7191 | 1 | 44 | 54 |
| 7192 | 2 | 77 | 58 |
| 7193 | 1 | 20 | 25 |
| 7194 | 2 | 17 | 57 |
| 7195 | 1 | 56 | 54 |
| 7196 | 2 | 89 | 58 |
| 7197 | 1 | 32 | 25 |
| 7198 | 2 | 29 | 57 |
| 7199 | 1 | 68 | 54 |
| 7200 | 2 | 101 | 58 |
| 7201 | 1 | 12 | 30 |
| 7202 | 2 | 4 | 66 |
| 7203 | 1 | 41 | 27 |
| 7204 | 2 | 12 | 63 |
| 7205 | 1 | 24 | 30 |
| 7206 | 2 | 16 | 66 |
| 7207 | 1 | 53 | 27 |
| 7208 | 2 | 24 | 63 |
| 7209 | 1 | 36 | 30 |
| 7210 | 2 | 28 | 66 |
| 7211 | 1 | 65 | 27 |
| 7212 | 2 | 36 | 63 |
| 7213 | 1 | 114 | 29 |
| 7214 | 2 | 80 | 65 |
| 7215 | 1 | 42 | 25 |
| 7216 | 2 | 113 | 28 |
| 7217 | 1 | 126 | 29 |
| 7218 | 2 | 92 | 65 |
| 7219 | 1 | 54 | 25 |
| 7220 | 2 | 125 | 28 |
| 7221 | 1 | 138 | 29 |
| 7222 | 2 | 104 | 65 |
| 7223 | 1 | 66 | 25 |
| 7224 | 2 | 137 | 28 |
| 7225 | 1 | 11 | 67 |
| 7226 | 2 | 76 | 35 |
| 7227 | 1 | 114 | 64 |
| 7228 | 2 | 41 | 28 |
| 7229 | 1 | 23 | 67 |
| 7230 | 2 | 88 | 35 |
| 7231 | 1 | 126 | 64 |
| 7232 | 2 | 53 | 28 |
| 7233 | 1 | 35 | 67 |
| 7234 | 2 | 100 | 35 |
| 7235 | 1 | 138 | 64 |
| 7236 | 2 | 65 | 28 |
| 7237 | 1 | 41 | 25 |
| 7238 | 2 | 112 | 32 |
| 7239 | 1 | 79 | 66 |
| 7240 | 2 | 5 | 63 |
| 7241 | 1 | 53 | 25 |
| 7242 | 2 | 124 | 32 |
| 7243 | 1 | 91 | 66 |
| 7244 | 2 | 17 | 63 |
| 7245 | 1 | 65 | 25 |
| 7246 | 2 | 136 | 32 |
| 7247 | 1 | 103 | 66 |
| 7248 | 2 | 29 | 63 |
| 7249 | 1 | 114 | 62 |
| 7250 | 2 | 41 | 61 |
| 7251 | 1 | 114 | 28 |
| 7252 | 2 | 41 | 63 |
| 7253 | 1 | 126 | 62 |
| 7254 | 2 | 53 | 61 |
| 7255 | 1 | 126 | 28 |
| 7256 | 2 | 53 | 63 |
| 7257 | 1 | 138 | 62 |
| 7258 | 2 | 65 | 61 |
| 7259 | 1 | 138 | 28 |
| 7260 | 2 | 65 | 63 |
| 7261 | 1 | 42 | 32 |
| 7262 | 2 | 113 | 30 |
| 7263 | 1 | 84 | 35 |
| 7264 | 2 | 7 | 29 |
| 7265 | 1 | 54 | 32 |
| 7266 | 2 | 125 | 30 |
| 7267 | 1 | 96 | 35 |
| 7268 | 2 | 19 | 29 |
| 7269 | 1 | 66 | 32 |
| 7270 | 2 | 137 | 30 |
| 7271 | 1 | 108 | 35 |
| 7272 | 2 | 31 | 29 |
| 7273 | 1 | 79 | 28 |
| 7274 | 2 | 41 | 30 |
| 7275 | 1 | 116 | 57 |
| 7276 | 2 | 113 | 31 |
| 7277 | 1 | 91 | 28 |
| 7278 | 2 | 53 | 30 |
| 7279 | 1 | 128 | 57 |
| 7280 | 2 | 125 | 31 |
| 7281 | 1 | 103 | 28 |
| 7282 | 2 | 65 | 30 |
| 7283 | 1 | 140 | 57 |
| 7284 | 2 | 137 | 31 |
| 7285 | 1 | 8 | 33 |
| 7286 | 2 | 5 | 65 |
| 7287 | 1 | 44 | 62 |
| 7288 | 2 | 77 | 66 |
| 7289 | 1 | 20 | 33 |
| 7290 | 2 | 17 | 65 |
| 7291 | 1 | 56 | 62 |
| 7292 | 2 | 89 | 66 |
| 7293 | 1 | 32 | 33 |
| 7294 | 2 | 29 | 65 |
| 7295 | 1 | 68 | 62 |
| 7296 | 2 | 101 | 66 |
| 7297 | 1 | 117 | 39 |
| 7298 | 2 | 76 | 43 |
| 7299 | 1 | 43 | 33 |
| 7300 | 2 | 41 | 36 |
| 7301 | 1 | 129 | 39 |
| 7302 | 2 | 88 | 43 |
| 7303 | 1 | 55 | 33 |
| 7304 | 2 | 53 | 36 |
| 7305 | 1 | 141 | 39 |
| 7306 | 2 | 100 | 43 |
| 7307 | 1 | 67 | 33 |
| 7308 | 2 | 65 | 36 |
| 7309 | 1 | 116 | 3 |
| 7310 | 2 | 5 | 36 |
| 7311 | 1 | 6 | 38 |
| 7312 | 2 | 77 | 8 |
| 7313 | 1 | 128 | 3 |
| 7314 | 2 | 17 | 36 |
| 7315 | 1 | 18 | 38 |
| 7316 | 2 | 89 | 8 |
| 7317 | 1 | 140 | 3 |
| 7318 | 2 | 29 | 36 |
| 7319 | 1 | 30 | 38 |
| 7320 | 2 | 101 | 8 |
| 7321 | 1 | 120 | 40 |
| 7322 | 2 | 6 | 9 |
| 7323 | 1 | 42 | 33 |
| 7324 | 2 | 113 | 5 |
| 7325 | 1 | 132 | 40 |
| 7326 | 2 | 18 | 9 |
| 7327 | 1 | 54 | 33 |
| 7328 | 2 | 125 | 5 |
| 7329 | 1 | 144 | 40 |
| 7330 | 2 | 30 | 9 |
| 7331 | 1 | 66 | 33 |
| 7332 | 2 | 137 | 5 |
| 7333 | 1 | 41 | 2 |
| 7334 | 2 | 41 | 34 |
| 7335 | 1 | 47 | 33 |
| 7336 | 2 | 5 | 40 |
| 7337 | 1 | 53 | 2 |
| 7338 | 2 | 53 | 34 |
| 7339 | 1 | 59 | 33 |
| 7340 | 2 | 17 | 40 |
| 7341 | 1 | 65 | 2 |
| 7342 | 2 | 65 | 34 |
| 7343 | 1 | 71 | 33 |
| 7344 | 2 | 29 | 40 |
| 7345 | 1 | 11 | 8 |
| 7346 | 2 | 77 | 41 |
| 7347 | 1 | 114 | 34 |
| 7348 | 2 | 78 | 4 |
| 7349 | 1 | 23 | 8 |
| 7350 | 2 | 89 | 41 |
| 7351 | 1 | 126 | 34 |
| 7352 | 2 | 90 | 4 |
| 7353 | 1 | 35 | 8 |
| 7354 | 2 | 101 | 41 |
| 7355 | 1 | 138 | 34 |
| 7356 | 2 | 102 | 4 |
| 7357 | 1 | 82 | 37 |
| 7358 | 2 | 42 | 40 |
| 7359 | 1 | 78 | 36 |
| 7360 | 2 | 6 | 5 |
| 7361 | 1 | 94 | 37 |
| 7362 | 2 | 54 | 40 |
| 7363 | 1 | 90 | 36 |
| 7364 | 2 | 18 | 5 |
| 7365 | 1 | 106 | 37 |
| 7366 | 2 | 66 | 40 |
| 7367 | 1 | 102 | 36 |
| 7368 | 2 | 30 | 5 |
| 7369 | 1 | 115 | 2 |
| 7370 | 2 | 41 | 7 |
| 7371 | 1 | 78 | 9 |
| 7372 | 2 | 78 | 37 |
| 7373 | 1 | 127 | 2 |
| 7374 | 2 | 53 | 7 |
| 7375 | 1 | 90 | 9 |
| 7376 | 2 | 90 | 37 |
| 7377 | 1 | 139 | 2 |
| 7378 | 2 | 65 | 7 |
| 7379 | 1 | 102 | 9 |
| 7380 | 2 | 102 | 37 |
| 7381 | 1 | 83 | 5 |
| 7382 | 2 | 48 | 37 |
| 7383 | 1 | 78 | 10 |
| 7384 | 2 | 6 | 6 |
| 7385 | 1 | 95 | 5 |
| 7386 | 2 | 60 | 37 |
| 7387 | 1 | 90 | 10 |
| 7388 | 2 | 18 | 6 |
| 7389 | 1 | 107 | 5 |
| 7390 | 2 | 72 | 37 |
| 7391 | 1 | 102 | 10 |
| 7392 | 2 | 30 | 6 |
| 7393 | 1 | 117 | 47 |
| 7394 | 2 | 76 | 51 |
| 7395 | 1 | 43 | 41 |
| 7396 | 2 | 41 | 44 |
| 7397 | 1 | 129 | 47 |
| 7398 | 2 | 88 | 51 |
| 7399 | 1 | 55 | 41 |
| 7400 | 2 | 53 | 44 |
| 7401 | 1 | 141 | 47 |
| 7402 | 2 | 100 | 51 |
| 7403 | 1 | 67 | 41 |
| 7404 | 2 | 65 | 44 |
| 7405 | 1 | 116 | 11 |
| 7406 | 2 | 5 | 44 |
| 7407 | 1 | 6 | 46 |
| 7408 | 2 | 77 | 16 |
| 7409 | 1 | 128 | 11 |
| 7410 | 2 | 17 | 44 |
| 7411 | 1 | 18 | 46 |
| 7412 | 2 | 89 | 16 |
| 7413 | 1 | 140 | 11 |
| 7414 | 2 | 29 | 44 |
| 7415 | 1 | 30 | 46 |
| 7416 | 2 | 101 | 16 |
| 7417 | 1 | 120 | 48 |
| 7418 | 2 | 6 | 17 |
| 7419 | 1 | 42 | 41 |
| 7420 | 2 | 113 | 13 |
| 7421 | 1 | 132 | 48 |
| 7422 | 2 | 18 | 17 |
| 7423 | 1 | 54 | 41 |
| 7424 | 2 | 125 | 13 |
| 7425 | 1 | 144 | 48 |
| 7426 | 2 | 30 | 17 |
| 7427 | 1 | 66 | 41 |
| 7428 | 2 | 137 | 13 |
| 7429 | 1 | 41 | 10 |
| 7430 | 2 | 41 | 42 |
| 7431 | 1 | 47 | 41 |
| 7432 | 2 | 5 | 48 |
| 7433 | 1 | 53 | 10 |
| 7434 | 2 | 53 | 42 |
| 7435 | 1 | 59 | 41 |
| 7436 | 2 | 17 | 48 |
| 7437 | 1 | 65 | 10 |
| 7438 | 2 | 65 | 42 |
| 7439 | 1 | 71 | 41 |
| 7440 | 2 | 29 | 48 |
| 7441 | 1 | 11 | 16 |
| 7442 | 2 | 77 | 49 |
| 7443 | 1 | 114 | 42 |
| 7444 | 2 | 78 | 12 |
| 7445 | 1 | 23 | 16 |
| 7446 | 2 | 89 | 49 |
| 7447 | 1 | 126 | 42 |
| 7448 | 2 | 90 | 12 |
| 7449 | 1 | 35 | 16 |
| 7450 | 2 | 101 | 49 |
| 7451 | 1 | 138 | 42 |
| 7452 | 2 | 102 | 12 |
| 7453 | 1 | 82 | 45 |
| 7454 | 2 | 42 | 48 |
| 7455 | 1 | 78 | 44 |
| 7456 | 2 | 6 | 13 |
| 7457 | 1 | 94 | 45 |
| 7458 | 2 | 54 | 48 |
| 7459 | 1 | 90 | 44 |
| 7460 | 2 | 18 | 13 |
| 7461 | 1 | 106 | 45 |
| 7462 | 2 | 66 | 48 |
| 7463 | 1 | 102 | 44 |
| 7464 | 2 | 30 | 13 |
| 7465 | 1 | 115 | 10 |
| 7466 | 2 | 41 | 15 |
| 7467 | 1 | 78 | 17 |
| 7468 | 2 | 78 | 45 |
| 7469 | 1 | 127 | 10 |
| 7470 | 2 | 53 | 15 |
| 7471 | 1 | 90 | 17 |
| 7472 | 2 | 90 | 45 |
| 7473 | 1 | 139 | 10 |
| 7474 | 2 | 65 | 15 |
| 7475 | 1 | 102 | 17 |
| 7476 | 2 | 102 | 45 |
| 7477 | 1 | 83 | 13 |
| 7478 | 2 | 48 | 45 |
| 7479 | 1 | 78 | 18 |
| 7480 | 2 | 6 | 14 |
| 7481 | 1 | 95 | 13 |
| 7482 | 2 | 60 | 45 |
| 7483 | 1 | 90 | 18 |
| 7484 | 2 | 18 | 14 |
| 7485 | 1 | 107 | 13 |
| 7486 | 2 | 72 | 45 |
| 7487 | 1 | 102 | 18 |
| 7488 | 2 | 30 | 14 |
| 7489 | 1 | 117 | 55 |
| 7490 | 2 | 76 | 59 |
| 7491 | 1 | 43 | 49 |
| 7492 | 2 | 41 | 52 |
| 7493 | 1 | 129 | 55 |
| 7494 | 2 | 88 | 59 |
| 7495 | 1 | 55 | 49 |
| 7496 | 2 | 53 | 52 |
| 7497 | 1 | 141 | 55 |
| 7498 | 2 | 100 | 59 |
| 7499 | 1 | 67 | 49 |
| 7500 | 2 | 65 | 52 |
| 7501 | 1 | 116 | 19 |
| 7502 | 2 | 5 | 52 |
| 7503 | 1 | 6 | 54 |
| 7504 | 2 | 77 | 24 |
| 7505 | 1 | 128 | 19 |
| 7506 | 2 | 17 | 52 |
| 7507 | 1 | 18 | 54 |
| 7508 | 2 | 89 | 24 |
| 7509 | 1 | 140 | 19 |
| 7510 | 2 | 29 | 52 |
| 7511 | 1 | 30 | 54 |
| 7512 | 2 | 101 | 24 |
| 7513 | 1 | 120 | 56 |
| 7514 | 2 | 6 | 25 |
| 7515 | 1 | 42 | 49 |
| 7516 | 2 | 113 | 21 |
| 7517 | 1 | 132 | 56 |
| 7518 | 2 | 18 | 25 |
| 7519 | 1 | 54 | 49 |
| 7520 | 2 | 125 | 21 |
| 7521 | 1 | 144 | 56 |
| 7522 | 2 | 30 | 25 |
| 7523 | 1 | 66 | 49 |
| 7524 | 2 | 137 | 21 |
| 7525 | 1 | 41 | 18 |
| 7526 | 2 | 41 | 50 |
| 7527 | 1 | 47 | 49 |
| 7528 | 2 | 5 | 56 |
| 7529 | 1 | 53 | 18 |
| 7530 | 2 | 53 | 50 |
| 7531 | 1 | 59 | 49 |
| 7532 | 2 | 17 | 56 |
| 7533 | 1 | 65 | 18 |
| 7534 | 2 | 65 | 50 |
| 7535 | 1 | 71 | 49 |
| 7536 | 2 | 29 | 56 |
| 7537 | 1 | 11 | 24 |
| 7538 | 2 | 77 | 57 |
| 7539 | 1 | 114 | 50 |
| 7540 | 2 | 78 | 20 |
| 7541 | 1 | 23 | 24 |
| 7542 | 2 | 89 | 57 |
| 7543 | 1 | 126 | 50 |
| 7544 | 2 | 90 | 20 |
| 7545 | 1 | 35 | 24 |
| 7546 | 2 | 101 | 57 |
| 7547 | 1 | 138 | 50 |
| 7548 | 2 | 102 | 20 |
| 7549 | 1 | 82 | 53 |
| 7550 | 2 | 42 | 56 |
| 7551 | 1 | 78 | 52 |
| 7552 | 2 | 6 | 21 |
| 7553 | 1 | 94 | 53 |
| 7554 | 2 | 54 | 56 |
| 7555 | 1 | 90 | 52 |
| 7556 | 2 | 18 | 21 |
| 7557 | 1 | 106 | 53 |
| 7558 | 2 | 66 | 56 |
| 7559 | 1 | 102 | 52 |
| 7560 | 2 | 30 | 21 |
| 7561 | 1 | 115 | 18 |
| 7562 | 2 | 41 | 23 |
| 7563 | 1 | 78 | 25 |
| 7564 | 2 | 78 | 53 |
| 7565 | 1 | 127 | 18 |
| 7566 | 2 | 53 | 23 |
| 7567 | 1 | 90 | 25 |
| 7568 | 2 | 90 | 53 |
| 7569 | 1 | 139 | 18 |
| 7570 | 2 | 65 | 23 |
| 7571 | 1 | 102 | 25 |
| 7572 | 2 | 102 | 53 |
| 7573 | 1 | 83 | 21 |
| 7574 | 2 | 48 | 53 |
| 7575 | 1 | 78 | 26 |
| 7576 | 2 | 6 | 22 |
| 7577 | 1 | 95 | 21 |
| 7578 | 2 | 60 | 53 |
| 7579 | 1 | 90 | 26 |
| 7580 | 2 | 18 | 22 |
| 7581 | 1 | 107 | 21 |
| 7582 | 2 | 72 | 53 |
| 7583 | 1 | 102 | 26 |
| 7584 | 2 | 30 | 22 |
| 7585 | 1 | 117 | 63 |
| 7586 | 2 | 76 | 67 |
| 7587 | 1 | 43 | 57 |
| 7588 | 2 | 41 | 60 |
| 7589 | 1 | 129 | 63 |
| 7590 | 2 | 88 | 67 |
| 7591 | 1 | 55 | 57 |
| 7592 | 2 | 53 | 60 |
| 7593 | 1 | 141 | 63 |
| 7594 | 2 | 100 | 67 |
| 7595 | 1 | 67 | 57 |
| 7596 | 2 | 65 | 60 |
| 7597 | 1 | 116 | 27 |
| 7598 | 2 | 5 | 60 |
| 7599 | 1 | 6 | 62 |
| 7600 | 2 | 77 | 32 |
| 7601 | 1 | 128 | 27 |
| 7602 | 2 | 17 | 60 |
| 7603 | 1 | 18 | 62 |
| 7604 | 2 | 89 | 32 |
| 7605 | 1 | 140 | 27 |
| 7606 | 2 | 29 | 60 |
| 7607 | 1 | 30 | 62 |
| 7608 | 2 | 101 | 32 |
| 7609 | 1 | 120 | 64 |
| 7610 | 2 | 6 | 33 |
| 7611 | 1 | 42 | 57 |
| 7612 | 2 | 113 | 29 |
| 7613 | 1 | 132 | 64 |
| 7614 | 2 | 18 | 33 |
| 7615 | 1 | 54 | 57 |
| 7616 | 2 | 125 | 29 |
| 7617 | 1 | 144 | 64 |
| 7618 | 2 | 30 | 33 |
| 7619 | 1 | 66 | 57 |
| 7620 | 2 | 137 | 29 |
| 7621 | 1 | 41 | 26 |
| 7622 | 2 | 41 | 58 |
| 7623 | 1 | 47 | 57 |
| 7624 | 2 | 5 | 64 |
| 7625 | 1 | 53 | 26 |
| 7626 | 2 | 53 | 58 |
| 7627 | 1 | 59 | 57 |
| 7628 | 2 | 17 | 64 |
| 7629 | 1 | 65 | 26 |
| 7630 | 2 | 65 | 58 |
| 7631 | 1 | 71 | 57 |
| 7632 | 2 | 29 | 64 |
| 7633 | 1 | 11 | 32 |
| 7634 | 2 | 77 | 65 |
| 7635 | 1 | 114 | 58 |
| 7636 | 2 | 78 | 28 |
| 7637 | 1 | 23 | 32 |
| 7638 | 2 | 89 | 65 |
| 7639 | 1 | 126 | 58 |
| 7640 | 2 | 90 | 28 |
| 7641 | 1 | 35 | 32 |
| 7642 | 2 | 101 | 65 |
| 7643 | 1 | 138 | 58 |
| 7644 | 2 | 102 | 28 |
| 7645 | 1 | 82 | 61 |
| 7646 | 2 | 42 | 64 |
| 7647 | 1 | 78 | 60 |
| 7648 | 2 | 6 | 29 |
| 7649 | 1 | 94 | 61 |
| 7650 | 2 | 54 | 64 |
| 7651 | 1 | 90 | 60 |
| 7652 | 2 | 18 | 29 |
| 7653 | 1 | 106 | 61 |
| 7654 | 2 | 66 | 64 |
| 7655 | 1 | 102 | 60 |
| 7656 | 2 | 30 | 29 |
| 7657 | 1 | 115 | 26 |
| 7658 | 2 | 41 | 31 |
| 7659 | 1 | 78 | 33 |
| 7660 | 2 | 78 | 61 |
| 7661 | 1 | 127 | 26 |
| 7662 | 2 | 53 | 31 |
| 7663 | 1 | 90 | 33 |
| 7664 | 2 | 90 | 61 |
| 7665 | 1 | 139 | 26 |
| 7666 | 2 | 65 | 31 |
| 7667 | 1 | 102 | 33 |
| 7668 | 2 | 102 | 61 |
| 7669 | 1 | 83 | 29 |
| 7670 | 2 | 48 | 61 |
| 7671 | 1 | 78 | 34 |
| 7672 | 2 | 6 | 30 |
| 7673 | 1 | 95 | 29 |
| 7674 | 2 | 60 | 61 |
| 7675 | 1 | 90 | 34 |
| 7676 | 2 | 18 | 30 |
| 7677 | 1 | 107 | 29 |
| 7678 | 2 | 72 | 61 |
| 7679 | 1 | 102 | 34 |
| 7680 | 2 | 30 | 30 |
| 7681 | 1 | 78 | 43 |
| 7682 | 2 | 6 | 7 |
| 7683 | 1 | 117 | 34 |
| 7684 | 2 | 6 | 10 |
| 7685 | 1 | 90 | 43 |
| 7686 | 2 | 18 | 7 |
| 7687 | 1 | 129 | 34 |
| 7688 | 2 | 18 | 10 |
| 7689 | 1 | 102 | 43 |
| 7690 | 2 | 30 | 7 |
| 7691 | 1 | 141 | 34 |
| 7692 | 2 | 30 | 10 |
| 7693 | 1 | 80 | 10 |
| 7694 | 2 | 6 | 39 |
| 7695 | 1 | 8 | 7 |
| 7696 | 2 | 79 | 5 |
| 7697 | 1 | 92 | 10 |
| 7698 | 2 | 18 | 39 |
| 7699 | 1 | 20 | 7 |
| 7700 | 2 | 91 | 5 |
| 7701 | 1 | 104 | 10 |
| 7702 | 2 | 30 | 39 |
| 7703 | 1 | 32 | 7 |
| 7704 | 2 | 103 | 5 |
| 7705 | 1 | 78 | 39 |
| 7706 | 2 | 80 | 5 |
| 7707 | 1 | 118 | 34 |
| 7708 | 2 | 43 | 3 |
| 7709 | 1 | 90 | 39 |
| 7710 | 2 | 92 | 5 |
| 7711 | 1 | 130 | 34 |
| 7712 | 2 | 55 | 3 |
| 7713 | 1 | 102 | 39 |
| 7714 | 2 | 104 | 5 |
| 7715 | 1 | 142 | 34 |
| 7716 | 2 | 67 | 3 |
| 7717 | 1 | 43 | 39 |
| 7718 | 2 | 6 | 41 |
| 7719 | 1 | 117 | 4 |
| 7720 | 2 | 115 | 35 |
| 7721 | 1 | 55 | 39 |
| 7722 | 2 | 18 | 41 |
| 7723 | 1 | 129 | 4 |
| 7724 | 2 | 127 | 35 |
| 7725 | 1 | 67 | 39 |
| 7726 | 2 | 30 | 41 |
| 7727 | 1 | 141 | 4 |
| 7728 | 2 | 139 | 35 |
| 7729 | 1 | 9 | 42 |
| 7730 | 2 | 115 | 36 |
| 7731 | 1 | 116 | 40 |
| 7732 | 2 | 116 | 4 |
| 7733 | 1 | 21 | 42 |
| 7734 | 2 | 127 | 36 |
| 7735 | 1 | 128 | 40 |
| 7736 | 2 | 128 | 4 |
| 7737 | 1 | 33 | 42 |
| 7738 | 2 | 139 | 36 |
| 7739 | 1 | 140 | 40 |
| 7740 | 2 | 140 | 4 |
| 7741 | 1 | 120 | 5 |
| 7742 | 2 | 43 | 5 |
| 7743 | 1 | 81 | 36 |
| 7744 | 2 | 116 | 36 |
| 7745 | 1 | 132 | 5 |
| 7746 | 2 | 55 | 5 |
| 7747 | 1 | 93 | 36 |
| 7748 | 2 | 128 | 36 |
| 7749 | 1 | 144 | 5 |
| 7750 | 2 | 67 | 5 |
| 7751 | 1 | 105 | 36 |
| 7752 | 2 | 140 | 36 |
| 7753 | 1 | 8 | 43 |
| 7754 | 2 | 7 | 40 |
| 7755 | 1 | 45 | 2 |
| 7756 | 2 | 44 | 5 |
| 7757 | 1 | 20 | 43 |
| 7758 | 2 | 19 | 40 |
| 7759 | 1 | 57 | 2 |
| 7760 | 2 | 56 | 5 |
| 7761 | 1 | 32 | 43 |
| 7762 | 2 | 31 | 40 |
| 7763 | 1 | 69 | 2 |
| 7764 | 2 | 68 | 5 |
| 7765 | 1 | 116 | 35 |
| 7766 | 2 | 79 | 10 |
| 7767 | 1 | 8 | 10 |
| 7768 | 2 | 80 | 8 |
| 7769 | 1 | 128 | 35 |
| 7770 | 2 | 91 | 10 |
| 7771 | 1 | 20 | 10 |
| 7772 | 2 | 92 | 8 |
| 7773 | 1 | 140 | 35 |
| 7774 | 2 | 103 | 10 |
| 7775 | 1 | 32 | 10 |
| 7776 | 2 | 104 | 8 |
| 7777 | 1 | 78 | 51 |
| 7778 | 2 | 6 | 15 |
| 7779 | 1 | 117 | 42 |
| 7780 | 2 | 6 | 18 |
| 7781 | 1 | 90 | 51 |
| 7782 | 2 | 18 | 15 |
| 7783 | 1 | 129 | 42 |
| 7784 | 2 | 18 | 18 |
| 7785 | 1 | 102 | 51 |
| 7786 | 2 | 30 | 15 |
| 7787 | 1 | 141 | 42 |
| 7788 | 2 | 30 | 18 |
| 7789 | 1 | 80 | 18 |
| 7790 | 2 | 6 | 47 |
| 7791 | 1 | 8 | 15 |
| 7792 | 2 | 79 | 13 |
| 7793 | 1 | 92 | 18 |
| 7794 | 2 | 18 | 47 |
| 7795 | 1 | 20 | 15 |
| 7796 | 2 | 91 | 13 |
| 7797 | 1 | 104 | 18 |
| 7798 | 2 | 30 | 47 |
| 7799 | 1 | 32 | 15 |
| 7800 | 2 | 103 | 13 |
| 7801 | 1 | 78 | 47 |
| 7802 | 2 | 80 | 13 |
| 7803 | 1 | 118 | 42 |
| 7804 | 2 | 43 | 11 |
| 7805 | 1 | 90 | 47 |
| 7806 | 2 | 92 | 13 |
| 7807 | 1 | 130 | 42 |
| 7808 | 2 | 55 | 11 |
| 7809 | 1 | 102 | 47 |
| 7810 | 2 | 104 | 13 |
| 7811 | 1 | 142 | 42 |
| 7812 | 2 | 67 | 11 |
| 7813 | 1 | 43 | 47 |
| 7814 | 2 | 6 | 49 |
| 7815 | 1 | 117 | 12 |
| 7816 | 2 | 115 | 43 |
| 7817 | 1 | 55 | 47 |
| 7818 | 2 | 18 | 49 |
| 7819 | 1 | 129 | 12 |
| 7820 | 2 | 127 | 43 |
| 7821 | 1 | 67 | 47 |
| 7822 | 2 | 30 | 49 |
| 7823 | 1 | 141 | 12 |
| 7824 | 2 | 139 | 43 |
| 7825 | 1 | 9 | 50 |
| 7826 | 2 | 115 | 44 |
| 7827 | 1 | 116 | 48 |
| 7828 | 2 | 116 | 12 |
| 7829 | 1 | 21 | 50 |
| 7830 | 2 | 127 | 44 |
| 7831 | 1 | 128 | 48 |
| 7832 | 2 | 128 | 12 |
| 7833 | 1 | 33 | 50 |
| 7834 | 2 | 139 | 44 |
| 7835 | 1 | 140 | 48 |
| 7836 | 2 | 140 | 12 |
| 7837 | 1 | 120 | 13 |
| 7838 | 2 | 43 | 13 |
| 7839 | 1 | 81 | 44 |
| 7840 | 2 | 116 | 44 |
| 7841 | 1 | 132 | 13 |
| 7842 | 2 | 55 | 13 |
| 7843 | 1 | 93 | 44 |
| 7844 | 2 | 128 | 44 |
| 7845 | 1 | 144 | 13 |
| 7846 | 2 | 67 | 13 |
| 7847 | 1 | 105 | 44 |
| 7848 | 2 | 140 | 44 |
| 7849 | 1 | 8 | 51 |
| 7850 | 2 | 7 | 48 |
| 7851 | 1 | 45 | 10 |
| 7852 | 2 | 44 | 13 |
| 7853 | 1 | 20 | 51 |
| 7854 | 2 | 19 | 48 |
| 7855 | 1 | 57 | 10 |
| 7856 | 2 | 56 | 13 |
| 7857 | 1 | 32 | 51 |
| 7858 | 2 | 31 | 48 |
| 7859 | 1 | 69 | 10 |
| 7860 | 2 | 68 | 13 |
| 7861 | 1 | 116 | 43 |
| 7862 | 2 | 79 | 18 |
| 7863 | 1 | 8 | 18 |
| 7864 | 2 | 80 | 16 |
| 7865 | 1 | 128 | 43 |
| 7866 | 2 | 91 | 18 |
| 7867 | 1 | 20 | 18 |
| 7868 | 2 | 92 | 16 |
| 7869 | 1 | 140 | 43 |
| 7870 | 2 | 103 | 18 |
| 7871 | 1 | 32 | 18 |
| 7872 | 2 | 104 | 16 |
| 7873 | 1 | 78 | 59 |
| 7874 | 2 | 6 | 23 |
| 7875 | 1 | 117 | 50 |
| 7876 | 2 | 6 | 26 |
| 7877 | 1 | 90 | 59 |
| 7878 | 2 | 18 | 23 |
| 7879 | 1 | 129 | 50 |
| 7880 | 2 | 18 | 26 |
| 7881 | 1 | 102 | 59 |
| 7882 | 2 | 30 | 23 |
| 7883 | 1 | 141 | 50 |
| 7884 | 2 | 30 | 26 |
| 7885 | 1 | 80 | 26 |
| 7886 | 2 | 6 | 55 |
| 7887 | 1 | 8 | 23 |
| 7888 | 2 | 79 | 21 |
| 7889 | 1 | 92 | 26 |
| 7890 | 2 | 18 | 55 |
| 7891 | 1 | 20 | 23 |
| 7892 | 2 | 91 | 21 |
| 7893 | 1 | 104 | 26 |
| 7894 | 2 | 30 | 55 |
| 7895 | 1 | 32 | 23 |
| 7896 | 2 | 103 | 21 |
| 7897 | 1 | 78 | 55 |
| 7898 | 2 | 80 | 21 |
| 7899 | 1 | 118 | 50 |
| 7900 | 2 | 43 | 19 |
| 7901 | 1 | 90 | 55 |
| 7902 | 2 | 92 | 21 |
| 7903 | 1 | 130 | 50 |
| 7904 | 2 | 55 | 19 |
| 7905 | 1 | 102 | 55 |
| 7906 | 2 | 104 | 21 |
| 7907 | 1 | 142 | 50 |
| 7908 | 2 | 67 | 19 |
| 7909 | 1 | 43 | 55 |
| 7910 | 2 | 6 | 57 |
| 7911 | 1 | 117 | 20 |
| 7912 | 2 | 115 | 51 |
| 7913 | 1 | 55 | 55 |
| 7914 | 2 | 18 | 57 |
| 7915 | 1 | 129 | 20 |
| 7916 | 2 | 127 | 51 |
| 7917 | 1 | 67 | 55 |
| 7918 | 2 | 30 | 57 |
| 7919 | 1 | 141 | 20 |
| 7920 | 2 | 139 | 51 |
| 7921 | 1 | 9 | 58 |
| 7922 | 2 | 115 | 52 |
| 7923 | 1 | 116 | 56 |
| 7924 | 2 | 116 | 20 |
| 7925 | 1 | 21 | 58 |
| 7926 | 2 | 127 | 52 |
| 7927 | 1 | 128 | 56 |
| 7928 | 2 | 128 | 20 |
| 7929 | 1 | 33 | 58 |
| 7930 | 2 | 139 | 52 |
| 7931 | 1 | 140 | 56 |
| 7932 | 2 | 140 | 20 |
| 7933 | 1 | 120 | 21 |
| 7934 | 2 | 43 | 21 |
| 7935 | 1 | 81 | 52 |
| 7936 | 2 | 116 | 52 |
| 7937 | 1 | 132 | 21 |
| 7938 | 2 | 55 | 21 |
| 7939 | 1 | 93 | 52 |
| 7940 | 2 | 128 | 52 |
| 7941 | 1 | 144 | 21 |
| 7942 | 2 | 67 | 21 |
| 7943 | 1 | 105 | 52 |
| 7944 | 2 | 140 | 52 |
| 7945 | 1 | 8 | 59 |
| 7946 | 2 | 7 | 56 |
| 7947 | 1 | 45 | 18 |
| 7948 | 2 | 44 | 21 |
| 7949 | 1 | 20 | 59 |
| 7950 | 2 | 19 | 56 |
| 7951 | 1 | 57 | 18 |
| 7952 | 2 | 56 | 21 |
| 7953 | 1 | 32 | 59 |
| 7954 | 2 | 31 | 56 |
| 7955 | 1 | 69 | 18 |
| 7956 | 2 | 68 | 21 |
| 7957 | 1 | 116 | 51 |
| 7958 | 2 | 79 | 26 |
| 7959 | 1 | 8 | 26 |
| 7960 | 2 | 80 | 24 |
| 7961 | 1 | 128 | 51 |
| 7962 | 2 | 91 | 26 |
| 7963 | 1 | 20 | 26 |
| 7964 | 2 | 92 | 24 |
| 7965 | 1 | 140 | 51 |
| 7966 | 2 | 103 | 26 |
| 7967 | 1 | 32 | 26 |
| 7968 | 2 | 104 | 24 |
| 7969 | 1 | 78 | 67 |
| 7970 | 2 | 6 | 31 |
| 7971 | 1 | 117 | 58 |
| 7972 | 2 | 6 | 34 |
| 7973 | 1 | 90 | 67 |
| 7974 | 2 | 18 | 31 |
| 7975 | 1 | 129 | 58 |
| 7976 | 2 | 18 | 34 |
| 7977 | 1 | 102 | 67 |
| 7978 | 2 | 30 | 31 |
| 7979 | 1 | 141 | 58 |
| 7980 | 2 | 30 | 34 |
| 7981 | 1 | 80 | 34 |
| 7982 | 2 | 6 | 63 |
| 7983 | 1 | 8 | 31 |
| 7984 | 2 | 79 | 29 |
| 7985 | 1 | 92 | 34 |
| 7986 | 2 | 18 | 63 |
| 7987 | 1 | 20 | 31 |
| 7988 | 2 | 91 | 29 |
| 7989 | 1 | 104 | 34 |
| 7990 | 2 | 30 | 63 |
| 7991 | 1 | 32 | 31 |
| 7992 | 2 | 103 | 29 |
| 7993 | 1 | 78 | 63 |
| 7994 | 2 | 80 | 29 |
| 7995 | 1 | 118 | 58 |
| 7996 | 2 | 43 | 27 |
| 7997 | 1 | 90 | 63 |
| 7998 | 2 | 92 | 29 |
| 7999 | 1 | 130 | 58 |
| 8000 | 2 | 55 | 27 |
| 8001 | 1 | 102 | 63 |
| 8002 | 2 | 104 | 29 |
| 8003 | 1 | 142 | 58 |
| 8004 | 2 | 67 | 27 |
| 8005 | 1 | 43 | 63 |
| 8006 | 2 | 6 | 65 |
| 8007 | 1 | 117 | 28 |
| 8008 | 2 | 115 | 59 |
| 8009 | 1 | 55 | 63 |
| 8010 | 2 | 18 | 65 |
| 8011 | 1 | 129 | 28 |
| 8012 | 2 | 127 | 59 |
| 8013 | 1 | 67 | 63 |
| 8014 | 2 | 30 | 65 |
| 8015 | 1 | 141 | 28 |
| 8016 | 2 | 139 | 59 |
| 8017 | 1 | 9 | 66 |
| 8018 | 2 | 115 | 60 |
| 8019 | 1 | 116 | 64 |
| 8020 | 2 | 116 | 28 |
| 8021 | 1 | 21 | 66 |
| 8022 | 2 | 127 | 60 |
| 8023 | 1 | 128 | 64 |
| 8024 | 2 | 128 | 28 |
| 8025 | 1 | 33 | 66 |
| 8026 | 2 | 139 | 60 |
| 8027 | 1 | 140 | 64 |
| 8028 | 2 | 140 | 28 |
| 8029 | 1 | 120 | 29 |
| 8030 | 2 | 43 | 29 |
| 8031 | 1 | 81 | 60 |
| 8032 | 2 | 116 | 60 |
| 8033 | 1 | 132 | 29 |
| 8034 | 2 | 55 | 29 |
| 8035 | 1 | 93 | 60 |
| 8036 | 2 | 128 | 60 |
| 8037 | 1 | 144 | 29 |
| 8038 | 2 | 67 | 29 |
| 8039 | 1 | 105 | 60 |
| 8040 | 2 | 140 | 60 |
| 8041 | 1 | 8 | 67 |
| 8042 | 2 | 7 | 64 |
| 8043 | 1 | 45 | 26 |
| 8044 | 2 | 44 | 29 |
| 8045 | 1 | 20 | 67 |
| 8046 | 2 | 19 | 64 |
| 8047 | 1 | 57 | 26 |
| 8048 | 2 | 56 | 29 |
| 8049 | 1 | 32 | 67 |
| 8050 | 2 | 31 | 64 |
| 8051 | 1 | 69 | 26 |
| 8052 | 2 | 68 | 29 |
| 8053 | 1 | 116 | 59 |
| 8054 | 2 | 79 | 34 |
| 8055 | 1 | 8 | 34 |
| 8056 | 2 | 80 | 32 |
| 8057 | 1 | 128 | 59 |
| 8058 | 2 | 91 | 34 |
| 8059 | 1 | 20 | 34 |
| 8060 | 2 | 92 | 32 |
| 8061 | 1 | 140 | 59 |
| 8062 | 2 | 103 | 34 |
| 8063 | 1 | 32 | 34 |
| 8064 | 2 | 104 | 32 |
| 8065 | 1 | 44 | 4 |
| 8066 | 2 | 42 | 37 |
| 8067 | 1 | 80 | 39 |
| 8068 | 2 | 43 | 4 |
| 8069 | 1 | 56 | 4 |
| 8070 | 2 | 54 | 37 |
| 8071 | 1 | 92 | 39 |
| 8072 | 2 | 55 | 4 |
| 8073 | 1 | 68 | 4 |
| 8074 | 2 | 66 | 37 |
| 8075 | 1 | 104 | 39 |
| 8076 | 2 | 67 | 4 |
| 8077 | 1 | 79 | 36 |
| 8078 | 2 | 78 | 41 |
| 8079 | 1 | 45 | 1 |
| 8080 | 2 | 79 | 7 |
| 8081 | 1 | 91 | 36 |
| 8082 | 2 | 90 | 41 |
| 8083 | 1 | 57 | 1 |
| 8084 | 2 | 91 | 7 |
| 8085 | 1 | 103 | 36 |
| 8086 | 2 | 102 | 41 |
| 8087 | 1 | 69 | 1 |
| 8088 | 2 | 103 | 7 |
| 8089 | 1 | 10 | 10 |
| 8090 | 2 | 42 | 7 |
| 8091 | 1 | 84 | 42 |
| 8092 | 2 | 45 | 4 |
| 8093 | 1 | 22 | 10 |
| 8094 | 2 | 54 | 7 |
| 8095 | 1 | 96 | 42 |
| 8096 | 2 | 57 | 4 |
| 8097 | 1 | 34 | 10 |
| 8098 | 2 | 66 | 7 |
| 8099 | 1 | 108 | 42 |
| 8100 | 2 | 69 | 4 |
| 8101 | 1 | 7 | 10 |
| 8102 | 2 | 43 | 2 |
| 8103 | 1 | 115 | 39 |
| 8104 | 2 | 79 | 39 |
| 8105 | 1 | 19 | 10 |
| 8106 | 2 | 55 | 2 |
| 8107 | 1 | 127 | 39 |
| 8108 | 2 | 91 | 39 |
| 8109 | 1 | 31 | 10 |
| 8110 | 2 | 67 | 2 |
| 8111 | 1 | 139 | 39 |
| 8112 | 2 | 103 | 39 |
| 8113 | 1 | 44 | 7 |
| 8114 | 2 | 7 | 42 |
| 8115 | 1 | 117 | 35 |
| 8116 | 2 | 116 | 39 |
| 8117 | 1 | 56 | 7 |
| 8118 | 2 | 19 | 42 |
| 8119 | 1 | 129 | 35 |
| 8120 | 2 | 128 | 39 |
| 8121 | 1 | 68 | 7 |
| 8122 | 2 | 31 | 42 |
| 8123 | 1 | 141 | 35 |
| 8124 | 2 | 140 | 39 |
| 8125 | 1 | 45 | 5 |
| 8126 | 2 | 8 | 37 |
| 8127 | 1 | 10 | 6 |
| 8128 | 2 | 116 | 37 |
| 8129 | 1 | 57 | 5 |
| 8130 | 2 | 20 | 37 |
| 8131 | 1 | 22 | 6 |
| 8132 | 2 | 128 | 37 |
| 8133 | 1 | 69 | 5 |
| 8134 | 2 | 32 | 37 |
| 8135 | 1 | 34 | 6 |
| 8136 | 2 | 140 | 37 |
| 8137 | 1 | 46 | 1 |
| 8138 | 2 | 44 | 34 |
| 8139 | 1 | 82 | 36 |
| 8140 | 2 | 45 | 3 |
| 8141 | 1 | 58 | 1 |
| 8142 | 2 | 56 | 34 |
| 8143 | 1 | 94 | 36 |
| 8144 | 2 | 57 | 3 |
| 8145 | 1 | 70 | 1 |
| 8146 | 2 | 68 | 34 |
| 8147 | 1 | 106 | 36 |
| 8148 | 2 | 69 | 3 |
| 8149 | 1 | 10 | 36 |
| 8150 | 2 | 80 | 6 |
| 8151 | 1 | 82 | 6 |
| 8152 | 2 | 81 | 37 |
| 8153 | 1 | 22 | 36 |
| 8154 | 2 | 92 | 6 |
| 8155 | 1 | 94 | 6 |
| 8156 | 2 | 93 | 37 |
| 8157 | 1 | 34 | 36 |
| 8158 | 2 | 104 | 6 |
| 8159 | 1 | 106 | 6 |
| 8160 | 2 | 105 | 37 |
| 8161 | 1 | 44 | 12 |
| 8162 | 2 | 42 | 45 |
| 8163 | 1 | 80 | 47 |
| 8164 | 2 | 43 | 12 |
| 8165 | 1 | 56 | 12 |
| 8166 | 2 | 54 | 45 |
| 8167 | 1 | 92 | 47 |
| 8168 | 2 | 55 | 12 |
| 8169 | 1 | 68 | 12 |
| 8170 | 2 | 66 | 45 |
| 8171 | 1 | 104 | 47 |
| 8172 | 2 | 67 | 12 |
| 8173 | 1 | 79 | 44 |
| 8174 | 2 | 78 | 49 |
| 8175 | 1 | 45 | 9 |
| 8176 | 2 | 79 | 15 |
| 8177 | 1 | 91 | 44 |
| 8178 | 2 | 90 | 49 |
| 8179 | 1 | 57 | 9 |
| 8180 | 2 | 91 | 15 |
| 8181 | 1 | 103 | 44 |
| 8182 | 2 | 102 | 49 |
| 8183 | 1 | 69 | 9 |
| 8184 | 2 | 103 | 15 |
| 8185 | 1 | 10 | 18 |
| 8186 | 2 | 42 | 15 |
| 8187 | 1 | 84 | 50 |
| 8188 | 2 | 45 | 12 |
| 8189 | 1 | 22 | 18 |
| 8190 | 2 | 54 | 15 |
| 8191 | 1 | 96 | 50 |
| 8192 | 2 | 57 | 12 |
| 8193 | 1 | 34 | 18 |
| 8194 | 2 | 66 | 15 |
| 8195 | 1 | 108 | 50 |
| 8196 | 2 | 69 | 12 |
| 8197 | 1 | 7 | 18 |
| 8198 | 2 | 43 | 10 |
| 8199 | 1 | 115 | 47 |
| 8200 | 2 | 79 | 47 |
| 8201 | 1 | 19 | 18 |
| 8202 | 2 | 55 | 10 |
| 8203 | 1 | 127 | 47 |
| 8204 | 2 | 91 | 47 |
| 8205 | 1 | 31 | 18 |
| 8206 | 2 | 67 | 10 |
| 8207 | 1 | 139 | 47 |
| 8208 | 2 | 103 | 47 |
| 8209 | 1 | 44 | 15 |
| 8210 | 2 | 7 | 50 |
| 8211 | 1 | 117 | 43 |
| 8212 | 2 | 116 | 47 |
| 8213 | 1 | 56 | 15 |
| 8214 | 2 | 19 | 50 |
| 8215 | 1 | 129 | 43 |
| 8216 | 2 | 128 | 47 |
| 8217 | 1 | 68 | 15 |
| 8218 | 2 | 31 | 50 |
| 8219 | 1 | 141 | 43 |
| 8220 | 2 | 140 | 47 |
| 8221 | 1 | 45 | 13 |
| 8222 | 2 | 8 | 45 |
| 8223 | 1 | 10 | 14 |
| 8224 | 2 | 116 | 45 |
| 8225 | 1 | 57 | 13 |
| 8226 | 2 | 20 | 45 |
| 8227 | 1 | 22 | 14 |
| 8228 | 2 | 128 | 45 |
| 8229 | 1 | 69 | 13 |
| 8230 | 2 | 32 | 45 |
| 8231 | 1 | 34 | 14 |
| 8232 | 2 | 140 | 45 |
| 8233 | 1 | 46 | 9 |
| 8234 | 2 | 44 | 42 |
| 8235 | 1 | 82 | 44 |
| 8236 | 2 | 45 | 11 |
| 8237 | 1 | 58 | 9 |
| 8238 | 2 | 56 | 42 |
| 8239 | 1 | 94 | 44 |
| 8240 | 2 | 57 | 11 |
| 8241 | 1 | 70 | 9 |
| 8242 | 2 | 68 | 42 |
| 8243 | 1 | 106 | 44 |
| 8244 | 2 | 69 | 11 |
| 8245 | 1 | 10 | 44 |
| 8246 | 2 | 80 | 14 |
| 8247 | 1 | 82 | 14 |
| 8248 | 2 | 81 | 45 |
| 8249 | 1 | 22 | 44 |
| 8250 | 2 | 92 | 14 |
| 8251 | 1 | 94 | 14 |
| 8252 | 2 | 93 | 45 |
| 8253 | 1 | 34 | 44 |
| 8254 | 2 | 104 | 14 |
| 8255 | 1 | 106 | 14 |
| 8256 | 2 | 105 | 45 |
| 8257 | 1 | 44 | 20 |
| 8258 | 2 | 42 | 53 |
| 8259 | 1 | 80 | 55 |
| 8260 | 2 | 43 | 20 |
| 8261 | 1 | 56 | 20 |
| 8262 | 2 | 54 | 53 |
| 8263 | 1 | 92 | 55 |
| 8264 | 2 | 55 | 20 |
| 8265 | 1 | 68 | 20 |
| 8266 | 2 | 66 | 53 |
| 8267 | 1 | 104 | 55 |
| 8268 | 2 | 67 | 20 |
| 8269 | 1 | 79 | 52 |
| 8270 | 2 | 78 | 57 |
| 8271 | 1 | 45 | 17 |
| 8272 | 2 | 79 | 23 |
| 8273 | 1 | 91 | 52 |
| 8274 | 2 | 90 | 57 |
| 8275 | 1 | 57 | 17 |
| 8276 | 2 | 91 | 23 |
| 8277 | 1 | 103 | 52 |
| 8278 | 2 | 102 | 57 |
| 8279 | 1 | 69 | 17 |
| 8280 | 2 | 103 | 23 |
| 8281 | 1 | 10 | 26 |
| 8282 | 2 | 42 | 23 |
| 8283 | 1 | 84 | 58 |
| 8284 | 2 | 45 | 20 |
| 8285 | 1 | 22 | 26 |
| 8286 | 2 | 54 | 23 |
| 8287 | 1 | 96 | 58 |
| 8288 | 2 | 57 | 20 |
| 8289 | 1 | 34 | 26 |
| 8290 | 2 | 66 | 23 |
| 8291 | 1 | 108 | 58 |
| 8292 | 2 | 69 | 20 |
| 8293 | 1 | 7 | 26 |
| 8294 | 2 | 43 | 18 |
| 8295 | 1 | 115 | 55 |
| 8296 | 2 | 79 | 55 |
| 8297 | 1 | 19 | 26 |
| 8298 | 2 | 55 | 18 |
| 8299 | 1 | 127 | 55 |
| 8300 | 2 | 91 | 55 |
| 8301 | 1 | 31 | 26 |
| 8302 | 2 | 67 | 18 |
| 8303 | 1 | 139 | 55 |
| 8304 | 2 | 103 | 55 |
| 8305 | 1 | 44 | 23 |
| 8306 | 2 | 7 | 58 |
| 8307 | 1 | 117 | 51 |
| 8308 | 2 | 116 | 55 |
| 8309 | 1 | 56 | 23 |
| 8310 | 2 | 19 | 58 |
| 8311 | 1 | 129 | 51 |
| 8312 | 2 | 128 | 55 |
| 8313 | 1 | 68 | 23 |
| 8314 | 2 | 31 | 58 |
| 8315 | 1 | 141 | 51 |
| 8316 | 2 | 140 | 55 |
| 8317 | 1 | 45 | 21 |
| 8318 | 2 | 8 | 53 |
| 8319 | 1 | 10 | 22 |
| 8320 | 2 | 116 | 53 |
| 8321 | 1 | 57 | 21 |
| 8322 | 2 | 20 | 53 |
| 8323 | 1 | 22 | 22 |
| 8324 | 2 | 128 | 53 |
| 8325 | 1 | 69 | 21 |
| 8326 | 2 | 32 | 53 |
| 8327 | 1 | 34 | 22 |
| 8328 | 2 | 140 | 53 |
| 8329 | 1 | 46 | 17 |
| 8330 | 2 | 44 | 50 |
| 8331 | 1 | 82 | 52 |
| 8332 | 2 | 45 | 19 |
| 8333 | 1 | 58 | 17 |
| 8334 | 2 | 56 | 50 |
| 8335 | 1 | 94 | 52 |
| 8336 | 2 | 57 | 19 |
| 8337 | 1 | 70 | 17 |
| 8338 | 2 | 68 | 50 |
| 8339 | 1 | 106 | 52 |
| 8340 | 2 | 69 | 19 |
| 8341 | 1 | 10 | 52 |
| 8342 | 2 | 80 | 22 |
| 8343 | 1 | 82 | 22 |
| 8344 | 2 | 81 | 53 |
| 8345 | 1 | 22 | 52 |
| 8346 | 2 | 92 | 22 |
| 8347 | 1 | 94 | 22 |
| 8348 | 2 | 93 | 53 |
| 8349 | 1 | 34 | 52 |
| 8350 | 2 | 104 | 22 |
| 8351 | 1 | 106 | 22 |
| 8352 | 2 | 105 | 53 |
| 8353 | 1 | 44 | 28 |
| 8354 | 2 | 42 | 61 |
| 8355 | 1 | 80 | 63 |
| 8356 | 2 | 43 | 28 |
| 8357 | 1 | 56 | 28 |
| 8358 | 2 | 54 | 61 |
| 8359 | 1 | 92 | 63 |
| 8360 | 2 | 55 | 28 |
| 8361 | 1 | 68 | 28 |
| 8362 | 2 | 66 | 61 |
| 8363 | 1 | 104 | 63 |
| 8364 | 2 | 67 | 28 |
| 8365 | 1 | 79 | 60 |
| 8366 | 2 | 78 | 65 |
| 8367 | 1 | 45 | 25 |
| 8368 | 2 | 79 | 31 |
| 8369 | 1 | 91 | 60 |
| 8370 | 2 | 90 | 65 |
| 8371 | 1 | 57 | 25 |
| 8372 | 2 | 91 | 31 |
| 8373 | 1 | 103 | 60 |
| 8374 | 2 | 102 | 65 |
| 8375 | 1 | 69 | 25 |
| 8376 | 2 | 103 | 31 |
| 8377 | 1 | 10 | 34 |
| 8378 | 2 | 42 | 31 |
| 8379 | 1 | 84 | 66 |
| 8380 | 2 | 45 | 28 |
| 8381 | 1 | 22 | 34 |
| 8382 | 2 | 54 | 31 |
| 8383 | 1 | 96 | 66 |
| 8384 | 2 | 57 | 28 |
| 8385 | 1 | 34 | 34 |
| 8386 | 2 | 66 | 31 |
| 8387 | 1 | 108 | 66 |
| 8388 | 2 | 69 | 28 |
| 8389 | 1 | 7 | 34 |
| 8390 | 2 | 43 | 26 |
| 8391 | 1 | 115 | 63 |
| 8392 | 2 | 79 | 63 |
| 8393 | 1 | 19 | 34 |
| 8394 | 2 | 55 | 26 |
| 8395 | 1 | 127 | 63 |
| 8396 | 2 | 91 | 63 |
| 8397 | 1 | 31 | 34 |
| 8398 | 2 | 67 | 26 |
| 8399 | 1 | 139 | 63 |
| 8400 | 2 | 103 | 63 |
| 8401 | 1 | 44 | 31 |
| 8402 | 2 | 7 | 66 |
| 8403 | 1 | 117 | 59 |
| 8404 | 2 | 116 | 63 |
| 8405 | 1 | 56 | 31 |
| 8406 | 2 | 19 | 66 |
| 8407 | 1 | 129 | 59 |
| 8408 | 2 | 128 | 63 |
| 8409 | 1 | 68 | 31 |
| 8410 | 2 | 31 | 66 |
| 8411 | 1 | 141 | 59 |
| 8412 | 2 | 140 | 63 |
| 8413 | 1 | 45 | 29 |
| 8414 | 2 | 8 | 61 |
| 8415 | 1 | 10 | 30 |
| 8416 | 2 | 116 | 61 |
| 8417 | 1 | 57 | 29 |
| 8418 | 2 | 20 | 61 |
| 8419 | 1 | 22 | 30 |
| 8420 | 2 | 128 | 61 |
| 8421 | 1 | 69 | 29 |
| 8422 | 2 | 32 | 61 |
| 8423 | 1 | 34 | 30 |
| 8424 | 2 | 140 | 61 |
| 8425 | 1 | 46 | 25 |
| 8426 | 2 | 44 | 58 |
| 8427 | 1 | 82 | 60 |
| 8428 | 2 | 45 | 27 |
| 8429 | 1 | 58 | 25 |
| 8430 | 2 | 56 | 58 |
| 8431 | 1 | 94 | 60 |
| 8432 | 2 | 57 | 27 |
| 8433 | 1 | 70 | 25 |
| 8434 | 2 | 68 | 58 |
| 8435 | 1 | 106 | 60 |
| 8436 | 2 | 69 | 27 |
| 8437 | 1 | 10 | 60 |
| 8438 | 2 | 80 | 30 |
| 8439 | 1 | 82 | 30 |
| 8440 | 2 | 81 | 61 |
| 8441 | 1 | 22 | 60 |
| 8442 | 2 | 92 | 30 |
| 8443 | 1 | 94 | 30 |
| 8444 | 2 | 93 | 61 |
| 8445 | 1 | 34 | 60 |
| 8446 | 2 | 104 | 30 |
| 8447 | 1 | 106 | 30 |
| 8448 | 2 | 105 | 61 |
| 8449 | 1 | 82 | 8 |
| 8450 | 2 | 117 | 3 |
| 8451 | 1 | 11 | 37 |
| 8452 | 2 | 9 | 10 |
| 8453 | 1 | 94 | 8 |
| 8454 | 2 | 129 | 3 |
| 8455 | 1 | 23 | 37 |
| 8456 | 2 | 21 | 10 |
| 8457 | 1 | 106 | 8 |
| 8458 | 2 | 141 | 3 |
| 8459 | 1 | 35 | 37 |
| 8460 | 2 | 33 | 10 |
| 8461 | 1 | 120 | 39 |
| 8462 | 2 | 9 | 39 |
| 8463 | 1 | 46 | 4 |
| 8464 | 2 | 9 | 11 |
| 8465 | 1 | 132 | 39 |
| 8466 | 2 | 21 | 39 |
| 8467 | 1 | 58 | 4 |
| 8468 | 2 | 21 | 11 |
| 8469 | 1 | 144 | 39 |
| 8470 | 2 | 33 | 39 |
| 8471 | 1 | 70 | 4 |
| 8472 | 2 | 33 | 11 |
| 8473 | 1 | 11 | 11 |
| 8474 | 2 | 81 | 39 |
| 8475 | 1 | 47 | 37 |
| 8476 | 2 | 117 | 40 |
| 8477 | 1 | 23 | 11 |
| 8478 | 2 | 93 | 39 |
| 8479 | 1 | 59 | 37 |
| 8480 | 2 | 129 | 40 |
| 8481 | 1 | 35 | 11 |
| 8482 | 2 | 105 | 39 |
| 8483 | 1 | 71 | 37 |
| 8484 | 2 | 141 | 40 |
| 8485 | 1 | 118 | 36 |
| 8486 | 2 | 81 | 40 |
| 8487 | 1 | 46 | 38 |
| 8488 | 2 | 118 | 33 |
| 8489 | 1 | 130 | 36 |
| 8490 | 2 | 93 | 40 |
| 8491 | 1 | 58 | 38 |
| 8492 | 2 | 130 | 33 |
| 8493 | 1 | 142 | 36 |
| 8494 | 2 | 105 | 40 |
| 8495 | 1 | 70 | 38 |
| 8496 | 2 | 142 | 33 |
| 8497 | 1 | 47 | 4 |
| 8498 | 2 | 10 | 41 |
| 8499 | 1 | 84 | 39 |
| 8500 | 2 | 83 | 9 |
| 8501 | 1 | 59 | 4 |
| 8502 | 2 | 22 | 41 |
| 8503 | 1 | 96 | 39 |
| 8504 | 2 | 95 | 9 |
| 8505 | 1 | 71 | 4 |
| 8506 | 2 | 34 | 41 |
| 8507 | 1 | 108 | 39 |
| 8508 | 2 | 107 | 9 |
| 8509 | 1 | 11 | 5 |
| 8510 | 2 | 46 | 39 |
| 8511 | 1 | 120 | 3 |
| 8512 | 2 | 119 | 38 |
| 8513 | 1 | 23 | 5 |
| 8514 | 2 | 58 | 39 |
| 8515 | 1 | 132 | 3 |
| 8516 | 2 | 131 | 38 |
| 8517 | 1 | 35 | 5 |
| 8518 | 2 | 70 | 39 |
| 8519 | 1 | 144 | 3 |
| 8520 | 2 | 143 | 38 |
| 8521 | 1 | 83 | 41 |
| 8522 | 2 | 118 | 40 |
| 8523 | 1 | 48 | 4 |
| 8524 | 2 | 119 | 6 |
| 8525 | 1 | 95 | 41 |
| 8526 | 2 | 130 | 40 |
| 8527 | 1 | 60 | 4 |
| 8528 | 2 | 131 | 6 |
| 8529 | 1 | 107 | 41 |
| 8530 | 2 | 142 | 40 |
| 8531 | 1 | 72 | 4 |
| 8532 | 2 | 143 | 6 |
| 8533 | 1 | 11 | 7 |
| 8534 | 2 | 47 | 34 |
| 8535 | 1 | 84 | 5 |
| 8536 | 2 | 83 | 42 |
| 8537 | 1 | 23 | 7 |
| 8538 | 2 | 59 | 34 |
| 8539 | 1 | 96 | 5 |
| 8540 | 2 | 95 | 42 |
| 8541 | 1 | 35 | 7 |
| 8542 | 2 | 71 | 34 |
| 8543 | 1 | 108 | 5 |
| 8544 | 2 | 107 | 42 |
| 8545 | 1 | 82 | 16 |
| 8546 | 2 | 117 | 11 |
| 8547 | 1 | 11 | 45 |
| 8548 | 2 | 9 | 18 |
| 8549 | 1 | 94 | 16 |
| 8550 | 2 | 129 | 11 |
| 8551 | 1 | 23 | 45 |
| 8552 | 2 | 21 | 18 |
| 8553 | 1 | 106 | 16 |
| 8554 | 2 | 141 | 11 |
| 8555 | 1 | 35 | 45 |
| 8556 | 2 | 33 | 18 |
| 8557 | 1 | 120 | 47 |
| 8558 | 2 | 9 | 47 |
| 8559 | 1 | 46 | 12 |
| 8560 | 2 | 9 | 19 |
| 8561 | 1 | 132 | 47 |
| 8562 | 2 | 21 | 47 |
| 8563 | 1 | 58 | 12 |
| 8564 | 2 | 21 | 19 |
| 8565 | 1 | 144 | 47 |
| 8566 | 2 | 33 | 47 |
| 8567 | 1 | 70 | 12 |
| 8568 | 2 | 33 | 19 |
| 8569 | 1 | 11 | 19 |
| 8570 | 2 | 81 | 47 |
| 8571 | 1 | 47 | 45 |
| 8572 | 2 | 117 | 48 |
| 8573 | 1 | 23 | 19 |
| 8574 | 2 | 93 | 47 |
| 8575 | 1 | 59 | 45 |
| 8576 | 2 | 129 | 48 |
| 8577 | 1 | 35 | 19 |
| 8578 | 2 | 105 | 47 |
| 8579 | 1 | 71 | 45 |
| 8580 | 2 | 141 | 48 |
| 8581 | 1 | 118 | 44 |
| 8582 | 2 | 81 | 48 |
| 8583 | 1 | 46 | 46 |
| 8584 | 2 | 118 | 41 |
| 8585 | 1 | 130 | 44 |
| 8586 | 2 | 93 | 48 |
| 8587 | 1 | 58 | 46 |
| 8588 | 2 | 130 | 41 |
| 8589 | 1 | 142 | 44 |
| 8590 | 2 | 105 | 48 |
| 8591 | 1 | 70 | 46 |
| 8592 | 2 | 142 | 41 |
| 8593 | 1 | 47 | 12 |
| 8594 | 2 | 10 | 49 |
| 8595 | 1 | 84 | 47 |
| 8596 | 2 | 83 | 17 |
| 8597 | 1 | 59 | 12 |
| 8598 | 2 | 22 | 49 |
| 8599 | 1 | 96 | 47 |
| 8600 | 2 | 95 | 17 |
| 8601 | 1 | 71 | 12 |
| 8602 | 2 | 34 | 49 |
| 8603 | 1 | 108 | 47 |
| 8604 | 2 | 107 | 17 |
| 8605 | 1 | 11 | 13 |
| 8606 | 2 | 46 | 47 |
| 8607 | 1 | 120 | 11 |
| 8608 | 2 | 119 | 46 |
| 8609 | 1 | 23 | 13 |
| 8610 | 2 | 58 | 47 |
| 8611 | 1 | 132 | 11 |
| 8612 | 2 | 131 | 46 |
| 8613 | 1 | 35 | 13 |
| 8614 | 2 | 70 | 47 |
| 8615 | 1 | 144 | 11 |
| 8616 | 2 | 143 | 46 |
| 8617 | 1 | 83 | 49 |
| 8618 | 2 | 118 | 48 |
| 8619 | 1 | 48 | 12 |
| 8620 | 2 | 119 | 14 |
| 8621 | 1 | 95 | 49 |
| 8622 | 2 | 130 | 48 |
| 8623 | 1 | 60 | 12 |
| 8624 | 2 | 131 | 14 |
| 8625 | 1 | 107 | 49 |
| 8626 | 2 | 142 | 48 |
| 8627 | 1 | 72 | 12 |
| 8628 | 2 | 143 | 14 |
| 8629 | 1 | 11 | 15 |
| 8630 | 2 | 47 | 42 |
| 8631 | 1 | 84 | 13 |
| 8632 | 2 | 83 | 50 |
| 8633 | 1 | 23 | 15 |
| 8634 | 2 | 59 | 42 |
| 8635 | 1 | 96 | 13 |
| 8636 | 2 | 95 | 50 |
| 8637 | 1 | 35 | 15 |
| 8638 | 2 | 71 | 42 |
| 8639 | 1 | 108 | 13 |
| 8640 | 2 | 107 | 50 |
| 8641 | 1 | 82 | 24 |
| 8642 | 2 | 117 | 19 |
| 8643 | 1 | 11 | 53 |
| 8644 | 2 | 9 | 26 |
| 8645 | 1 | 94 | 24 |
| 8646 | 2 | 129 | 19 |
| 8647 | 1 | 23 | 53 |
| 8648 | 2 | 21 | 26 |
| 8649 | 1 | 106 | 24 |
| 8650 | 2 | 141 | 19 |
| 8651 | 1 | 35 | 53 |
| 8652 | 2 | 33 | 26 |
| 8653 | 1 | 120 | 55 |
| 8654 | 2 | 9 | 55 |
| 8655 | 1 | 46 | 20 |
| 8656 | 2 | 9 | 27 |
| 8657 | 1 | 132 | 55 |
| 8658 | 2 | 21 | 55 |
| 8659 | 1 | 58 | 20 |
| 8660 | 2 | 21 | 27 |
| 8661 | 1 | 144 | 55 |
| 8662 | 2 | 33 | 55 |
| 8663 | 1 | 70 | 20 |
| 8664 | 2 | 33 | 27 |
| 8665 | 1 | 11 | 27 |
| 8666 | 2 | 81 | 55 |
| 8667 | 1 | 47 | 53 |
| 8668 | 2 | 117 | 56 |
| 8669 | 1 | 23 | 27 |
| 8670 | 2 | 93 | 55 |
| 8671 | 1 | 59 | 53 |
| 8672 | 2 | 129 | 56 |
| 8673 | 1 | 35 | 27 |
| 8674 | 2 | 105 | 55 |
| 8675 | 1 | 71 | 53 |
| 8676 | 2 | 141 | 56 |
| 8677 | 1 | 118 | 52 |
| 8678 | 2 | 81 | 56 |
| 8679 | 1 | 46 | 54 |
| 8680 | 2 | 118 | 49 |
| 8681 | 1 | 130 | 52 |
| 8682 | 2 | 93 | 56 |
| 8683 | 1 | 58 | 54 |
| 8684 | 2 | 130 | 49 |
| 8685 | 1 | 142 | 52 |
| 8686 | 2 | 105 | 56 |
| 8687 | 1 | 70 | 54 |
| 8688 | 2 | 142 | 49 |
| 8689 | 1 | 47 | 20 |
| 8690 | 2 | 10 | 57 |
| 8691 | 1 | 84 | 55 |
| 8692 | 2 | 83 | 25 |
| 8693 | 1 | 59 | 20 |
| 8694 | 2 | 22 | 57 |
| 8695 | 1 | 96 | 55 |
| 8696 | 2 | 95 | 25 |
| 8697 | 1 | 71 | 20 |
| 8698 | 2 | 34 | 57 |
| 8699 | 1 | 108 | 55 |
| 8700 | 2 | 107 | 25 |
| 8701 | 1 | 11 | 21 |
| 8702 | 2 | 46 | 55 |
| 8703 | 1 | 120 | 19 |
| 8704 | 2 | 119 | 54 |
| 8705 | 1 | 23 | 21 |
| 8706 | 2 | 58 | 55 |
| 8707 | 1 | 132 | 19 |
| 8708 | 2 | 131 | 54 |
| 8709 | 1 | 35 | 21 |
| 8710 | 2 | 70 | 55 |
| 8711 | 1 | 144 | 19 |
| 8712 | 2 | 143 | 54 |
| 8713 | 1 | 83 | 57 |
| 8714 | 2 | 118 | 56 |
| 8715 | 1 | 48 | 20 |
| 8716 | 2 | 119 | 22 |
| 8717 | 1 | 95 | 57 |
| 8718 | 2 | 130 | 56 |
| 8719 | 1 | 60 | 20 |
| 8720 | 2 | 131 | 22 |
| 8721 | 1 | 107 | 57 |
| 8722 | 2 | 142 | 56 |
| 8723 | 1 | 72 | 20 |
| 8724 | 2 | 143 | 22 |
| 8725 | 1 | 11 | 23 |
| 8726 | 2 | 47 | 50 |
| 8727 | 1 | 84 | 21 |
| 8728 | 2 | 83 | 58 |
| 8729 | 1 | 23 | 23 |
| 8730 | 2 | 59 | 50 |
| 8731 | 1 | 96 | 21 |
| 8732 | 2 | 95 | 58 |
| 8733 | 1 | 35 | 23 |
| 8734 | 2 | 71 | 50 |
| 8735 | 1 | 108 | 21 |
| 8736 | 2 | 107 | 58 |
| 8737 | 1 | 82 | 32 |
| 8738 | 2 | 117 | 27 |
| 8739 | 1 | 11 | 61 |
| 8740 | 2 | 9 | 34 |
| 8741 | 1 | 94 | 32 |
| 8742 | 2 | 129 | 27 |
| 8743 | 1 | 23 | 61 |
| 8744 | 2 | 21 | 34 |
| 8745 | 1 | 106 | 32 |
| 8746 | 2 | 141 | 27 |
| 8747 | 1 | 35 | 61 |
| 8748 | 2 | 33 | 34 |
| 8749 | 1 | 120 | 63 |
| 8750 | 2 | 9 | 63 |
| 8751 | 1 | 46 | 28 |
| 8752 | 2 | 9 | 35 |
| 8753 | 1 | 132 | 63 |
| 8754 | 2 | 21 | 63 |
| 8755 | 1 | 58 | 28 |
| 8756 | 2 | 21 | 35 |
| 8757 | 1 | 144 | 63 |
| 8758 | 2 | 33 | 63 |
| 8759 | 1 | 70 | 28 |
| 8760 | 2 | 33 | 35 |
| 8761 | 1 | 11 | 35 |
| 8762 | 2 | 81 | 63 |
| 8763 | 1 | 47 | 61 |
| 8764 | 2 | 117 | 64 |
| 8765 | 1 | 23 | 35 |
| 8766 | 2 | 93 | 63 |
| 8767 | 1 | 59 | 61 |
| 8768 | 2 | 129 | 64 |
| 8769 | 1 | 35 | 35 |
| 8770 | 2 | 105 | 63 |
| 8771 | 1 | 71 | 61 |
| 8772 | 2 | 141 | 64 |
| 8773 | 1 | 118 | 60 |
| 8774 | 2 | 81 | 64 |
| 8775 | 1 | 46 | 62 |
| 8776 | 2 | 118 | 57 |
| 8777 | 1 | 130 | 60 |
| 8778 | 2 | 93 | 64 |
| 8779 | 1 | 58 | 62 |
| 8780 | 2 | 130 | 57 |
| 8781 | 1 | 142 | 60 |
| 8782 | 2 | 105 | 64 |
| 8783 | 1 | 70 | 62 |
| 8784 | 2 | 142 | 57 |
| 8785 | 1 | 47 | 28 |
| 8786 | 2 | 10 | 65 |
| 8787 | 1 | 84 | 63 |
| 8788 | 2 | 83 | 33 |
| 8789 | 1 | 59 | 28 |
| 8790 | 2 | 22 | 65 |
| 8791 | 1 | 96 | 63 |
| 8792 | 2 | 95 | 33 |
| 8793 | 1 | 71 | 28 |
| 8794 | 2 | 34 | 65 |
| 8795 | 1 | 108 | 63 |
| 8796 | 2 | 107 | 33 |
| 8797 | 1 | 11 | 29 |
| 8798 | 2 | 46 | 63 |
| 8799 | 1 | 120 | 27 |
| 8800 | 2 | 119 | 62 |
| 8801 | 1 | 23 | 29 |
| 8802 | 2 | 58 | 63 |
| 8803 | 1 | 132 | 27 |
| 8804 | 2 | 131 | 62 |
| 8805 | 1 | 35 | 29 |
| 8806 | 2 | 70 | 63 |
| 8807 | 1 | 144 | 27 |
| 8808 | 2 | 143 | 62 |
| 8809 | 1 | 83 | 65 |
| 8810 | 2 | 118 | 64 |
| 8811 | 1 | 48 | 28 |
| 8812 | 2 | 119 | 30 |
| 8813 | 1 | 95 | 65 |
| 8814 | 2 | 130 | 64 |
| 8815 | 1 | 60 | 28 |
| 8816 | 2 | 131 | 30 |
| 8817 | 1 | 107 | 65 |
| 8818 | 2 | 142 | 64 |
| 8819 | 1 | 72 | 28 |
| 8820 | 2 | 143 | 30 |
| 8821 | 1 | 11 | 31 |
| 8822 | 2 | 47 | 58 |
| 8823 | 1 | 84 | 29 |
| 8824 | 2 | 83 | 66 |
| 8825 | 1 | 23 | 31 |
| 8826 | 2 | 59 | 58 |
| 8827 | 1 | 96 | 29 |
| 8828 | 2 | 95 | 66 |
| 8829 | 1 | 35 | 31 |
| 8830 | 2 | 71 | 58 |
| 8831 | 1 | 108 | 29 |
| 8832 | 2 | 107 | 66 |
| 8833 | 1 | 45 | 40 |
| 8834 | 2 | 9 | 9 |
| 8835 | 1 | 118 | 6 |
| 8836 | 2 | 118 | 2 |
| 8837 | 1 | 57 | 40 |
| 8838 | 2 | 21 | 9 |
| 8839 | 1 | 130 | 6 |
| 8840 | 2 | 130 | 2 |
| 8841 | 1 | 69 | 40 |
| 8842 | 2 | 33 | 9 |
| 8843 | 1 | 142 | 6 |
| 8844 | 2 | 142 | 2 |
| 8845 | 1 | 46 | 34 |
| 8846 | 2 | 45 | 6 |
| 8847 | 1 | 46 | 35 |
| 8848 | 2 | 10 | 39 |
| 8849 | 1 | 58 | 34 |
| 8850 | 2 | 57 | 6 |
| 8851 | 1 | 58 | 35 |
| 8852 | 2 | 22 | 39 |
| 8853 | 1 | 70 | 34 |
| 8854 | 2 | 69 | 6 |
| 8855 | 1 | 70 | 35 |
| 8856 | 2 | 34 | 39 |
| 8857 | 1 | 10 | 40 |
| 8858 | 2 | 81 | 41 |
| 8859 | 1 | 84 | 36 |
| 8860 | 2 | 10 | 9 |
| 8861 | 1 | 22 | 40 |
| 8862 | 2 | 93 | 41 |
| 8863 | 1 | 96 | 36 |
| 8864 | 2 | 22 | 9 |
| 8865 | 1 | 34 | 40 |
| 8866 | 2 | 105 | 41 |
| 8867 | 1 | 108 | 36 |
| 8868 | 2 | 34 | 9 |
| 8869 | 1 | 83 | 8 |
| 8870 | 2 | 117 | 38 |
| 8871 | 1 | 119 | 33 |
| 8872 | 2 | 82 | 9 |
| 8873 | 1 | 95 | 8 |
| 8874 | 2 | 129 | 38 |
| 8875 | 1 | 131 | 33 |
| 8876 | 2 | 94 | 9 |
| 8877 | 1 | 107 | 8 |
| 8878 | 2 | 141 | 38 |
| 8879 | 1 | 143 | 33 |
| 8880 | 2 | 106 | 9 |
| 8881 | 1 | 11 | 9 |
| 8882 | 2 | 119 | 34 |
| 8883 | 1 | 84 | 9 |
| 8884 | 2 | 12 | 36 |
| 8885 | 1 | 23 | 9 |
| 8886 | 2 | 131 | 34 |
| 8887 | 1 | 96 | 9 |
| 8888 | 2 | 24 | 36 |
| 8889 | 1 | 35 | 9 |
| 8890 | 2 | 143 | 34 |
| 8891 | 1 | 108 | 9 |
| 8892 | 2 | 36 | 36 |
| 8893 | 1 | 12 | 8 |
| 8894 | 2 | 119 | 4 |
| 8895 | 1 | 48 | 36 |
| 8896 | 2 | 120 | 37 |
| 8897 | 1 | 24 | 8 |
| 8898 | 2 | 131 | 4 |
| 8899 | 1 | 60 | 36 |
| 8900 | 2 | 132 | 37 |
| 8901 | 1 | 36 | 8 |
| 8902 | 2 | 143 | 4 |
| 8903 | 1 | 72 | 36 |
| 8904 | 2 | 144 | 37 |
| 8905 | 1 | 120 | 8 |
| 8906 | 2 | 119 | 36 |
| 8907 | 1 | 48 | 2 |
| 8908 | 2 | 48 | 7 |
| 8909 | 1 | 132 | 8 |
| 8910 | 2 | 131 | 36 |
| 8911 | 1 | 60 | 2 |
| 8912 | 2 | 60 | 7 |
| 8913 | 1 | 144 | 8 |
| 8914 | 2 | 143 | 36 |
| 8915 | 1 | 72 | 2 |
| 8916 | 2 | 72 | 7 |
| 8917 | 1 | 84 | 37 |
| 8918 | 2 | 11 | 40 |
| 8919 | 1 | 84 | 10 |
| 8920 | 2 | 12 | 43 |
| 8921 | 1 | 96 | 37 |
| 8922 | 2 | 23 | 40 |
| 8923 | 1 | 96 | 10 |
| 8924 | 2 | 24 | 43 |
| 8925 | 1 | 108 | 37 |
| 8926 | 2 | 35 | 40 |
| 8927 | 1 | 108 | 10 |
| 8928 | 2 | 36 | 43 |
| 8929 | 1 | 45 | 48 |
| 8930 | 2 | 9 | 17 |
| 8931 | 1 | 118 | 14 |
| 8932 | 2 | 118 | 10 |
| 8933 | 1 | 57 | 48 |
| 8934 | 2 | 21 | 17 |
| 8935 | 1 | 130 | 14 |
| 8936 | 2 | 130 | 10 |
| 8937 | 1 | 69 | 48 |
| 8938 | 2 | 33 | 17 |
| 8939 | 1 | 142 | 14 |
| 8940 | 2 | 142 | 10 |
| 8941 | 1 | 46 | 42 |
| 8942 | 2 | 45 | 14 |
| 8943 | 1 | 46 | 43 |
| 8944 | 2 | 10 | 47 |
| 8945 | 1 | 58 | 42 |
| 8946 | 2 | 57 | 14 |
| 8947 | 1 | 58 | 43 |
| 8948 | 2 | 22 | 47 |
| 8949 | 1 | 70 | 42 |
| 8950 | 2 | 69 | 14 |
| 8951 | 1 | 70 | 43 |
| 8952 | 2 | 34 | 47 |
| 8953 | 1 | 10 | 48 |
| 8954 | 2 | 81 | 49 |
| 8955 | 1 | 84 | 44 |
| 8956 | 2 | 10 | 17 |
| 8957 | 1 | 22 | 48 |
| 8958 | 2 | 93 | 49 |
| 8959 | 1 | 96 | 44 |
| 8960 | 2 | 22 | 17 |
| 8961 | 1 | 34 | 48 |
| 8962 | 2 | 105 | 49 |
| 8963 | 1 | 108 | 44 |
| 8964 | 2 | 34 | 17 |
| 8965 | 1 | 83 | 16 |
| 8966 | 2 | 117 | 46 |
| 8967 | 1 | 119 | 41 |
| 8968 | 2 | 82 | 17 |
| 8969 | 1 | 95 | 16 |
| 8970 | 2 | 129 | 46 |
| 8971 | 1 | 131 | 41 |
| 8972 | 2 | 94 | 17 |
| 8973 | 1 | 107 | 16 |
| 8974 | 2 | 141 | 46 |
| 8975 | 1 | 143 | 41 |
| 8976 | 2 | 106 | 17 |
| 8977 | 1 | 11 | 17 |
| 8978 | 2 | 119 | 42 |
| 8979 | 1 | 84 | 17 |
| 8980 | 2 | 12 | 44 |
| 8981 | 1 | 23 | 17 |
| 8982 | 2 | 131 | 42 |
| 8983 | 1 | 96 | 17 |
| 8984 | 2 | 24 | 44 |
| 8985 | 1 | 35 | 17 |
| 8986 | 2 | 143 | 42 |
| 8987 | 1 | 108 | 17 |
| 8988 | 2 | 36 | 44 |
| 8989 | 1 | 12 | 16 |
| 8990 | 2 | 119 | 12 |
| 8991 | 1 | 48 | 44 |
| 8992 | 2 | 120 | 45 |
| 8993 | 1 | 24 | 16 |
| 8994 | 2 | 131 | 12 |
| 8995 | 1 | 60 | 44 |
| 8996 | 2 | 132 | 45 |
| 8997 | 1 | 36 | 16 |
| 8998 | 2 | 143 | 12 |
| 8999 | 1 | 72 | 44 |
| 9000 | 2 | 144 | 45 |
| 9001 | 1 | 120 | 16 |
| 9002 | 2 | 119 | 44 |
| 9003 | 1 | 48 | 10 |
| 9004 | 2 | 48 | 15 |
| 9005 | 1 | 132 | 16 |
| 9006 | 2 | 131 | 44 |
| 9007 | 1 | 60 | 10 |
| 9008 | 2 | 60 | 15 |
| 9009 | 1 | 144 | 16 |
| 9010 | 2 | 143 | 44 |
| 9011 | 1 | 72 | 10 |
| 9012 | 2 | 72 | 15 |
| 9013 | 1 | 84 | 45 |
| 9014 | 2 | 11 | 48 |
| 9015 | 1 | 84 | 18 |
| 9016 | 2 | 12 | 51 |
| 9017 | 1 | 96 | 45 |
| 9018 | 2 | 23 | 48 |
| 9019 | 1 | 96 | 18 |
| 9020 | 2 | 24 | 51 |
| 9021 | 1 | 108 | 45 |
| 9022 | 2 | 35 | 48 |
| 9023 | 1 | 108 | 18 |
| 9024 | 2 | 36 | 51 |
| 9025 | 1 | 45 | 56 |
| 9026 | 2 | 9 | 25 |
| 9027 | 1 | 118 | 22 |
| 9028 | 2 | 118 | 18 |
| 9029 | 1 | 57 | 56 |
| 9030 | 2 | 21 | 25 |
| 9031 | 1 | 130 | 22 |
| 9032 | 2 | 130 | 18 |
| 9033 | 1 | 69 | 56 |
| 9034 | 2 | 33 | 25 |
| 9035 | 1 | 142 | 22 |
| 9036 | 2 | 142 | 18 |
| 9037 | 1 | 46 | 50 |
| 9038 | 2 | 45 | 22 |
| 9039 | 1 | 46 | 51 |
| 9040 | 2 | 10 | 55 |
| 9041 | 1 | 58 | 50 |
| 9042 | 2 | 57 | 22 |
| 9043 | 1 | 58 | 51 |
| 9044 | 2 | 22 | 55 |
| 9045 | 1 | 70 | 50 |
| 9046 | 2 | 69 | 22 |
| 9047 | 1 | 70 | 51 |
| 9048 | 2 | 34 | 55 |
| 9049 | 1 | 10 | 56 |
| 9050 | 2 | 81 | 57 |
| 9051 | 1 | 84 | 52 |
| 9052 | 2 | 10 | 25 |
| 9053 | 1 | 22 | 56 |
| 9054 | 2 | 93 | 57 |
| 9055 | 1 | 96 | 52 |
| 9056 | 2 | 22 | 25 |
| 9057 | 1 | 34 | 56 |
| 9058 | 2 | 105 | 57 |
| 9059 | 1 | 108 | 52 |
| 9060 | 2 | 34 | 25 |
| 9061 | 1 | 83 | 24 |
| 9062 | 2 | 117 | 54 |
| 9063 | 1 | 119 | 49 |
| 9064 | 2 | 82 | 25 |
| 9065 | 1 | 95 | 24 |
| 9066 | 2 | 129 | 54 |
| 9067 | 1 | 131 | 49 |
| 9068 | 2 | 94 | 25 |
| 9069 | 1 | 107 | 24 |
| 9070 | 2 | 141 | 54 |
| 9071 | 1 | 143 | 49 |
| 9072 | 2 | 106 | 25 |
| 9073 | 1 | 11 | 25 |
| 9074 | 2 | 119 | 50 |
| 9075 | 1 | 84 | 25 |
| 9076 | 2 | 12 | 52 |
| 9077 | 1 | 23 | 25 |
| 9078 | 2 | 131 | 50 |
| 9079 | 1 | 96 | 25 |
| 9080 | 2 | 24 | 52 |
| 9081 | 1 | 35 | 25 |
| 9082 | 2 | 143 | 50 |
| 9083 | 1 | 108 | 25 |
| 9084 | 2 | 36 | 52 |
| 9085 | 1 | 12 | 24 |
| 9086 | 2 | 119 | 20 |
| 9087 | 1 | 48 | 52 |
| 9088 | 2 | 120 | 53 |
| 9089 | 1 | 24 | 24 |
| 9090 | 2 | 131 | 20 |
| 9091 | 1 | 60 | 52 |
| 9092 | 2 | 132 | 53 |
| 9093 | 1 | 36 | 24 |
| 9094 | 2 | 143 | 20 |
| 9095 | 1 | 72 | 52 |
| 9096 | 2 | 144 | 53 |
| 9097 | 1 | 120 | 24 |
| 9098 | 2 | 119 | 52 |
| 9099 | 1 | 48 | 18 |
| 9100 | 2 | 48 | 23 |
| 9101 | 1 | 132 | 24 |
| 9102 | 2 | 131 | 52 |
| 9103 | 1 | 60 | 18 |
| 9104 | 2 | 60 | 23 |
| 9105 | 1 | 144 | 24 |
| 9106 | 2 | 143 | 52 |
| 9107 | 1 | 72 | 18 |
| 9108 | 2 | 72 | 23 |
| 9109 | 1 | 84 | 53 |
| 9110 | 2 | 11 | 56 |
| 9111 | 1 | 84 | 26 |
| 9112 | 2 | 12 | 59 |
| 9113 | 1 | 96 | 53 |
| 9114 | 2 | 23 | 56 |
| 9115 | 1 | 96 | 26 |
| 9116 | 2 | 24 | 59 |
| 9117 | 1 | 108 | 53 |
| 9118 | 2 | 35 | 56 |
| 9119 | 1 | 108 | 26 |
| 9120 | 2 | 36 | 59 |
| 9121 | 1 | 45 | 64 |
| 9122 | 2 | 9 | 33 |
| 9123 | 1 | 118 | 30 |
| 9124 | 2 | 118 | 26 |
| 9125 | 1 | 57 | 64 |
| 9126 | 2 | 21 | 33 |
| 9127 | 1 | 130 | 30 |
| 9128 | 2 | 130 | 26 |
| 9129 | 1 | 69 | 64 |
| 9130 | 2 | 33 | 33 |
| 9131 | 1 | 142 | 30 |
| 9132 | 2 | 142 | 26 |
| 9133 | 1 | 46 | 58 |
| 9134 | 2 | 45 | 30 |
| 9135 | 1 | 46 | 59 |
| 9136 | 2 | 10 | 63 |
| 9137 | 1 | 58 | 58 |
| 9138 | 2 | 57 | 30 |
| 9139 | 1 | 58 | 59 |
| 9140 | 2 | 22 | 63 |
| 9141 | 1 | 70 | 58 |
| 9142 | 2 | 69 | 30 |
| 9143 | 1 | 70 | 59 |
| 9144 | 2 | 34 | 63 |
| 9145 | 1 | 10 | 64 |
| 9146 | 2 | 81 | 65 |
| 9147 | 1 | 84 | 60 |
| 9148 | 2 | 10 | 33 |
| 9149 | 1 | 22 | 64 |
| 9150 | 2 | 93 | 65 |
| 9151 | 1 | 96 | 60 |
| 9152 | 2 | 22 | 33 |
| 9153 | 1 | 34 | 64 |
| 9154 | 2 | 105 | 65 |
| 9155 | 1 | 108 | 60 |
| 9156 | 2 | 34 | 33 |
| 9157 | 1 | 83 | 32 |
| 9158 | 2 | 117 | 62 |
| 9159 | 1 | 119 | 57 |
| 9160 | 2 | 82 | 33 |
| 9161 | 1 | 95 | 32 |
| 9162 | 2 | 129 | 62 |
| 9163 | 1 | 131 | 57 |
| 9164 | 2 | 94 | 33 |
| 9165 | 1 | 107 | 32 |
| 9166 | 2 | 141 | 62 |
| 9167 | 1 | 143 | 57 |
| 9168 | 2 | 106 | 33 |
| 9169 | 1 | 11 | 33 |
| 9170 | 2 | 119 | 58 |
| 9171 | 1 | 84 | 33 |
| 9172 | 2 | 12 | 60 |
| 9173 | 1 | 23 | 33 |
| 9174 | 2 | 131 | 58 |
| 9175 | 1 | 96 | 33 |
| 9176 | 2 | 24 | 60 |
| 9177 | 1 | 35 | 33 |
| 9178 | 2 | 143 | 58 |
| 9179 | 1 | 108 | 33 |
| 9180 | 2 | 36 | 60 |
| 9181 | 1 | 12 | 32 |
| 9182 | 2 | 119 | 28 |
| 9183 | 1 | 48 | 60 |
| 9184 | 2 | 120 | 61 |
| 9185 | 1 | 24 | 32 |
| 9186 | 2 | 131 | 28 |
| 9187 | 1 | 60 | 60 |
| 9188 | 2 | 132 | 61 |
| 9189 | 1 | 36 | 32 |
| 9190 | 2 | 143 | 28 |
| 9191 | 1 | 72 | 60 |
| 9192 | 2 | 144 | 61 |
| 9193 | 1 | 120 | 32 |
| 9194 | 2 | 119 | 60 |
| 9195 | 1 | 48 | 26 |
| 9196 | 2 | 48 | 31 |
| 9197 | 1 | 132 | 32 |
| 9198 | 2 | 131 | 60 |
| 9199 | 1 | 60 | 26 |
| 9200 | 2 | 60 | 31 |
| 9201 | 1 | 144 | 32 |
| 9202 | 2 | 143 | 60 |
| 9203 | 1 | 72 | 26 |
| 9204 | 2 | 72 | 31 |
| 9205 | 1 | 84 | 61 |
| 9206 | 2 | 11 | 64 |
| 9207 | 1 | 84 | 34 |
| 9208 | 2 | 12 | 67 |
| 9209 | 1 | 96 | 61 |
| 9210 | 2 | 23 | 64 |
| 9211 | 1 | 96 | 34 |
| 9212 | 2 | 24 | 67 |
| 9213 | 1 | 108 | 61 |
| 9214 | 2 | 35 | 64 |
| 9215 | 1 | 108 | 34 |
| 9216 | 2 | 36 | 67 |

In another embodiment, the relative positions of the 9216 dots, in X and Y coordinates range from (0,0) to (64+VO, 144). In this exemplary embodiment, the number of rows has increased by a vertical offset (VO), where different compound placement zones have an equal number of rows, but include a different subset of the 64+VO rows. For example, FIGS. 3 illustrates four adjacent zones 330, 340, 350, and 360. The vertically offset zones allow the placement of alignment dots 320 on opposite sides of the ChemCard, efficient spacing between the dots 130, without requiring the size of the ChemCard to increase. In the exemplary embodiment of FIG. 3, adjacent zones have the same number of rows (64), but they are offset by VO=5 rows. Thus, the rows in the second and fourth zones 340 and 360 are numbered from 1 to 64, while the rows in the first and third zones are numbered from 6 to 69. One of skill in the art will recognize that the vertical offset in FIG. 3 (e.g. 5) is exemplary, and any vertical offset, e.g. V=1, 2, 3, 4, 6, 8, 10, 12, 15, 20 may be implement according to the same concepts.

In one embodiment, the method of FIGS. 4 and 5 is executed using software running on a computer. One objective of the algorithm (from which the software is based) is to determine a Sequence Array SA having elements representative of dispense positions on a ChemCard, such that each of the compounds placed in each of the dispenses satisfy the neighboring compound constraints, as discussed above. More specifically, the constraints for a particular compound are satisfied when each occurrence of a particular compound has different neighbors within their respective neighbor distances. Likewise, constraints for a particular dispense position are satisfied when each of the compounds in the dispense satisfy the constraints. For example, in an embodiment using a 9,216 compound carrying ChemCard and a 12 pipette dispense mechanism (where all 12 pipettes are fired simultaneously), there are 768 possible dispense positions. In this case, A=768 and S_{A} contains non-repeating values from 1 to 768 which represent dispense locations on the ChemCard. An array C of size A (C_{A}) contains, at each location A, a plurality J of compounds to be dispensed at the dispense position stored in S_{A}. Thus, in an embodiment that dispenses 2 occurrences of each compound, each compound will be in two different elements of C_{A}. For example, C₁ may contain compounds 1-12 and C₂ may contain compounds 1-12, while C₃ and C₄ each contain compounds 13-24. Thus, if S₁=324, S₂=531, C₁=compounds 1-12, and C₂=compounds 1-12 then compounds 1-12 will be dispensed at dispense positions 324 and 531. When the algorithm is determining the array S_{A}, each of the 12 compounds in each of the dispenses S_{A} are tested against the constraints to determine if other occurrences of each of the compounds has the same neighbors. If any of the 12 compounds in the dispense violate the constraint check then the dispense position S_{A} violates the constraints and the value of S_{A} (representing a dispense position) must be changed in order to produce a complete array S_{A} that satisfies the constraints.

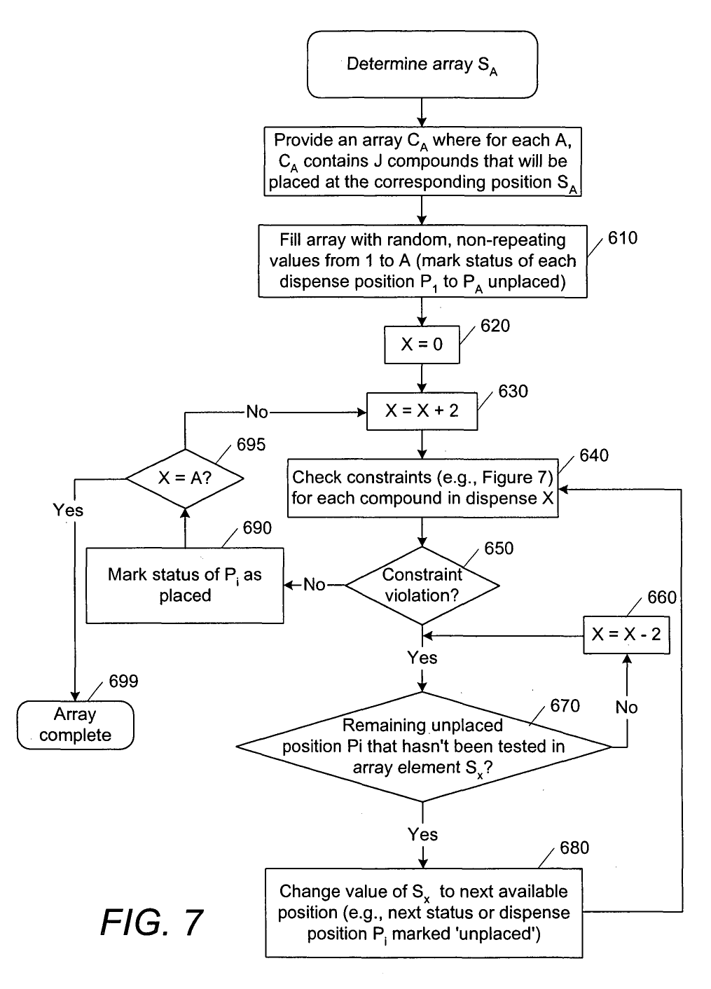

FIG. 7 is a flow chart illustrating a method of determining an array S_{A} such that each of the compounds in each of the A dispense positions satisfy the neighbor constraints. In advantageous embodiments, each of the dispense positions dispenses a plurality J of compounds. As discussed above, this may be accomplished by firing all of the pipettes in a dispense mechanism simultaneously (e.g., J=number of pipettes), or, alternatively, firing only a portion of the pipettes in a dispense mechanism over a particular location (e.g., J=number of pipettes fired over each ChemCard location). The plurality of dispense positions are provided in an array P_{A}, where a first element contains the X,Y coordinates of the location of the dispense on the ChemCard and a second element contains a status indicator representative of whether each particular position P_{A} has been placed in the array S_{A}. Thus, as an element of array S_{A} is filled with a dispense position P_{A}, the status indicator of the particular dispense position in P_{A} is changed to indicate the position has been placed in the array S_{A}. For example, if S₁ is filled with dispense position 425, the status indicator of P₄₂₅ would be set to placed. In an advantageous embodiment, each of the status indicators for the dispense positions P are set to unplaced before the algorithm begins filling the array S_{A}.

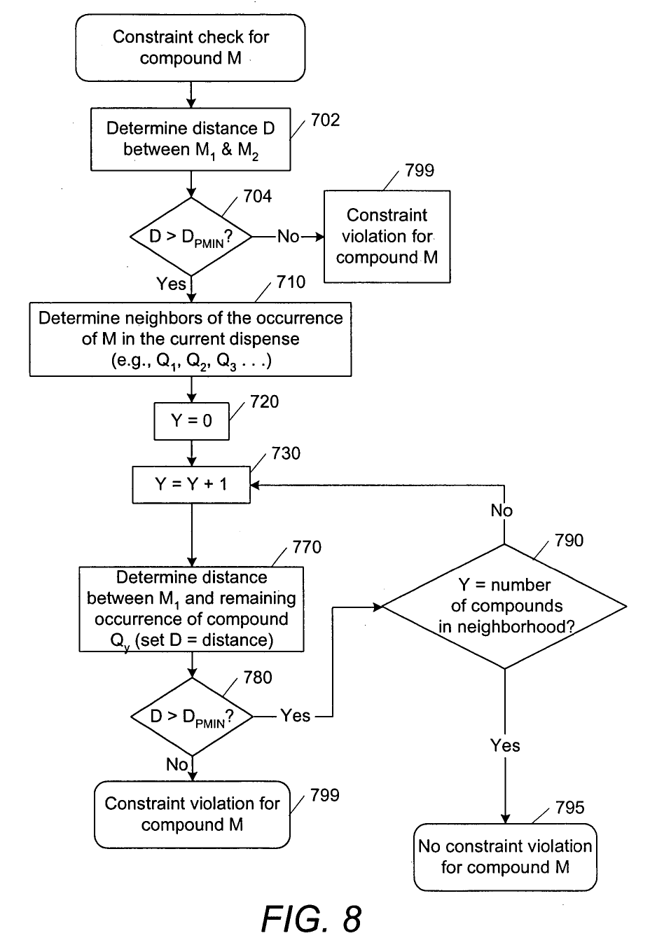

In block 610, the array S_{A} is filled with random, non repeating values from 1 to A. Thus, in an embodiment having 768 dispense positions (i.e. A=768), the values of each S_{A} is a random value between 1 and 768, where each of the values of different elements of S_{A} are unique. For example, S₁=324, S₂=531, S₃=13, S₄=230. . . S₇₆₈=654, where each of the values of S_{A} represent dispense positions on a ChemCard (and corresponds with a dispense position P_{A}). Although each of the A elements of the array S_{A} are filled with dispense positions, the corresponding status indicator P for the dispense positions is not set to placed as the dispense positions in S_{A} at this point are preliminary (e.g., constraint checks have not yet been performed). The status indicator for a particular dispense position P is set to placed only after the constraint checks (e.g., FIG. 8) have been satisfied for the particular dispense position P.

In another embodiment, the array S_{A} may be initialized and filled with any value. For example, each element of S_{A} may be set to zero (0).

In block 620 a counter X is set to zero. The counter X is used to step through each element of the array S_{A}, setting the value of each S_{A} to a dispense position after each of the J compounds within each of the dispense positions is checked against the neighbor constraints. In the 768 dispense position example, X will increment from 0 to 768, checking for constraint violations at each dispense position, before completing the array S_{A}. In alternative embodiments, the counter X may begin at any location within the array S_{A} and reset to zero after checking the last member of the array. For example, in a 768 dispense position system, X may be set to 300, iteratively incremented to 768 after checking for constraint violations at each dispense position, reset to zero (0), and iteratively incremented to 298 after checking for constraint violations at each dispense position. Alternatively, each dispense position may be tested against the constraints in any other order, so long as each of the dispense positions (and more particularly, each of the compounds within a dispense) are tested against the neighbor constraints.

In block 630, counter X is incremented to the next dispense position to be tested. In the example of FIG. 7, each of the compounds is placed on the ChemCard twice, in sequential dispenses (e.g., the same J compounds are dispensed at position S₁ and S₂). Thus, the constraints only need to be checked after each pair of compounds is assigned to a particular dispense position. Thus, X is set to increment by 2 after each round of constraint checks. As will be discussed in more detail below, any number of each of the compounds may be placed on the ChemCard, and, thus, X may be incremented by varying amounts.

In block 640, each of the J compounds in C_{X} is checked against the neighbor constraints (as described with reference to FIG. 8, for example). For example, if the number of compounds in each dispense J=6 then the constraints check is performed for each of the 6 compounds in the dispense C_{X} at the dispense position in S_{X}. In general, the neighbor constraint check determines whether a particular compound (e.g., at dispense location S_{X}) has different neighbors (within the defined neighbor distance) than other occurrences of the particular compound (e.g., at other dispense locations). One method of performing a neighbor constraint check is described in detail with reference to FIG. 8. However, other methods of performing the neighbor constraint check are contemplated.

Block 650 is a decision block that determines whether or not there is a constraint violation for any of the J compounds in C_{X} at the dispense position S_{X}. As stated above, if any of the J compounds in C_{X} violate the neighbor constraints at dispense position S_{X} then the value of S_{X} must be changed to another dispense position. If there are no constraint violations for any of the compounds in C_{X} then the method continues to Block 690 where the status of the dispense position used in S_{X} is set to placed. However, if there are constraint violations for any of the compounds in C_{X} then the method continues to Blocks 670, and then to block 660 or 680 where adjustments to the position of at least the dispense position in S_{X} are made.

Block 670 is a decision block that determines if there are remaining unplaced dispense positions that have not been tested at the dispense location S_{X}. In one embodiment, the status of the positions P are polled in order to determined which dispense positions remain unplaced. As discussed above, in order to ensure that each compound in the array satisfies the neighbor constraints, when a constraint violation is found for any one of the J compounds C_{X} at a dispense position S_{X}, the dispense position S_{X} is changed to another available dispense position and the constraint for each of the J compounds at the new dispense position S_{X} are tested. For example, if S₂₃₂=342 and one of the J compounds in C₂₃₂ violates the neighbor constraints, S₂₃₂ may be changed to another dispense position. In one embodiment, S_{X} is set to the value of the next dispense positions that has a status indicator set to unplaced. Thus, if the status indicators in P₃₄₃ and P₃₄₄ have already been set to placed (e.g., S₅₃=343 and S₂₁₁=344 and the neighbor constraints were satisfied for the compounds C₅₃ at dispense position 343 and for the compounds C₂₁₁, at dispense position 344), but the status indicator for P₃₄₅ is set to unplaced, Block 680 will set the value of S_{X} to 345. However, if there are no remaining dispense positions that have not been tested against the neighbor constraints at the position of S_{X} (e.g., every P with a status indicator set to unplaced has already been tested at dispense position S_{X}), then the method continues to Block 660 where the value of X is decremented.

If Block 670 determines there are no remaining dispense positions that have not been tested against the neighbor constraints at the position of S_{X}, at Block 660 the value of X is decremented by 2. As discussed above, the method of FIG. 7 is exemplary of a system that places two occurrences of each compound on the ChemCard, and, thus, the constraints only need be checked for every other element of the array. Thus, in a system that places 4 occurrences of each compound on a single ChemCard, the value of X may be decremented by 4, for example. After X has been decremented, the method returns to Block 670 to determine if there are remaining dispense positions that have not been tested at the dispense location S_{X}. Note that the current value of S_{X} (e.g., after S_{X} has been decremented in Block 660) will have satisfied the neighbor constraints in a previous iteration of the constraint checks of Block 640. However, because no acceptable dispense position could be found for the next array element S_{X+2}, the value of S_{X} may be changed in an attempt to adjust at least some of the neighbors of S_{X+2} and allow S_{X+2} to pass the neighbor constraint test. Thus, the status indicator for the dispense position P that had already been set to placed at position S_{X} is set to unplaced to ensure that the position is available for another position in array S_{A}. The method then returns to Block 640 where the constraints are checked for the new dispense position in S_{X} .

If Block 650 determines that there is no constraint violation for the J compounds C_{X} at dispense position S_{X} then the method continues to Block 690 where the status indicator for the dispense position tested in S_{X} is set to placed. For example, if S_{X}=562, then the status indicator of P₅₆₂ is set to placed. In one embodiment, the status indicator may be set to either a 1 or a 0, where 1 indicates a placed dispense position and a 0 indicates an unplaced dispense position. In this embodiment, the array may be initialized with all zeros at the beginning of the method of FIG. 7. Likewise, when a dispense position S_{X} satisfies the constraints, the status indicator corresponding with the dispense position of S_{X} may be changed to a one, indicating that the dispense position has been filled.

Block 695 is a decision block that determines whether each of the array elements in S_{X} has been tested against the neighbor constraints by determining if counter X is equal to A. For example, in a system where the total dispense positions A=768, when X=766 at Block 695, the value of X will increment to 768 at Block 630 and the last element of S_{A} will be checked against the constraints in Block 640. After determining that the constraints for the dispense position in S₇₆₈ are satisfied, the array S_{A} is complete. Therefore, at Block 695 the method determines that X=A (e.g., 768=768) and the method continues to Block 699 which indicates that the array S_{A} is complete.

FIG. 8 is a flowchart illustrating one method of performing the neighbor constraint checks for an exemplary compound M. In one embodiment, an algorithm searches for a pattern where one dot of each compound is at least a minimum distance (e.g., the neighbor distance) from every other compound in the array by checking the distance between each pair of dots in the array. However, such an algorithm would perform many unnecessary calculations as constraints are checked with respect to dots that are not within the neighbor distance Dₚₘᵢₙ (and therefore do not violate a constraint for the particular compound) of any of the multiple dots of a particular compound. Therefore, in an advantageous embodiment, rather than testing each occurrence of a particular dot with every other dot in the array, only those dots within the neighbor distance Dₚₘᵢₙ are checked.

As discussed above, for each dispense position a plurality of J compounds may be placed. Thus, for each dispense position, the flowchart of FIG. 8 may need to be executed for each of the plurality of J compounds before the neighbor constraints are satisfied for the particular dispense position. The exemplary constraint check method of FIG. 8 is specific to a system that places pairs of compounds in the array. However, a similar method may easily be derived, based on the same nearest neighbor principles, for a system that places any number of dots of each compound in the array. The method of FIG. 8 makes reference to M₁ and M₂, which are indicative of two occurrences of the compound M. As indicated above, the method of FIG. 8 may be implemented in the array development method of FIG. 7, as well as any other method that requires a neighbor constraint check.

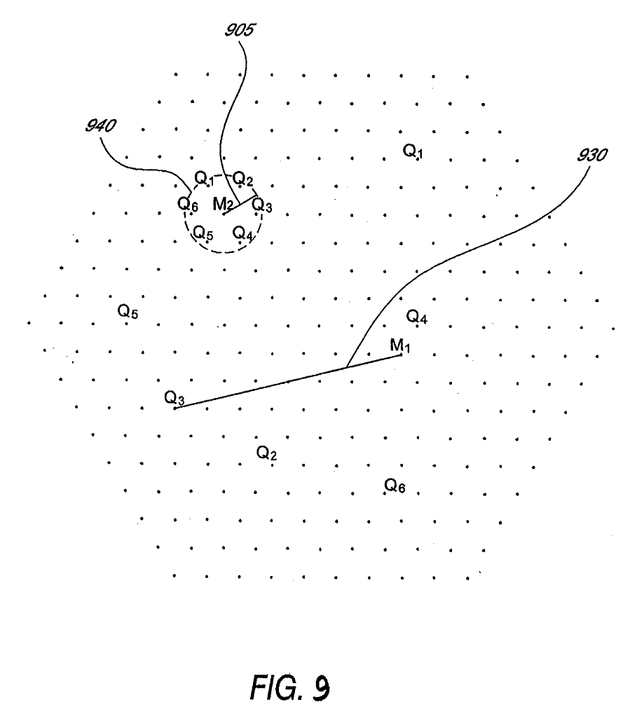

In order to provide a graphic example of the operation of the neighbor constraint check of FIG. 8, FIG. 9 will now be described and referred to with reference to the constraint check method of FIG. 8. FIG. 9 illustrates a portion of a placement array geographically arranged as the compounds would be placed on a ChemCard. The exemplary embodiment of FIG. 9 utilizes a honeycomb placement pattern. The compound M, including M₁ and M₂, represents a dot for which constraints are being checked, wherein M₁ is to be dispensed in a first dispense (e.g., for a particular dispense T, M₁ is one of the compounds J in C_{T} that will be dispensed at location S_{T}) and M₂ is to be dispensed in a second dispense (e.g., M₂ is one of the compounds J in C_{T+}that will be dispensed at location S_{T+1}). The pairs of dots labeled Q₁- Q₆ represent the pairs of neighbors of M₂ at the current location within the array S. Each of the unlabeled dots (representative of dot placement locations) may have a compound already assigned to them, or, alternatively, may be positions that have not been assigned compounds yet.

In Block 702 the variable D is set to equal the distance between M₁ and M₂. In an embodiment that places multiple compounds in each dispense position (e.g., J>1), so long as the relative positions of each of the compounds in the dispense mechanism remains constant, D will remain constant for each of the compounds in the dispense.

In Block 704 the variable D is compared to the neighbor distance Dₚₘᵢₙ. If the distance D is less than Dₚₘᵢₙ there is a constraint violation and the method ends. More particularly, if D is less than Dₚₘᵢₙ then both occurrence of M (i.e. M₁ and M₂) are close enough to one another so that an active spot pattern diffusing from compound M may not be definitively attributable to the compound M. This may occur as a result of the diffusion spots from M₁ and M₂ overlapping so that a spot detection machine recognizes only a single spot pattern. If the distance D is not less than Dₚₘᵢₙ there is no constraint violation and the method continues to Block 710.

In Block 710, the method determines the neighbors of the occurrence of M at the current dispense position (designated as M₂). Thus, when used as part of Block 640 (FIG. 7), Block 710 determines the neighbors of the compound M₂ at dispense position S_{X} (where compound M₁ is placed at dispense position S_{X-1}). In the example of FIG. 9, the neighbors are those compounds within the radius of the circle 940 having a radius of Dₚₘᵢₙ, where the line 905 is set to the distance Dₚₘᵢₙ. The neighbors of compound M₂ are referred to herein as Q, wherein Q includes each neighbor Q₁, Q₂, Q₃. . . Q_{Y}, where Y is the total number of neighbors of M₂. The number of dots in a neighborhood, called the size of the neighborhood, depends on the value of Dₚₘᵢₙ, the dot grid spacing, and the location of the central dot (e.g., edge dots have smaller sets). A neighborhood's size is always much smaller than the total number of dots on a card. Thus, in the example of FIG. 9, the neighbors of M₂ include Q₁- Q₆. In other embodiments, the number of neighbors of compound M may vary greatly depending on the particular assay process and the method of determining the neighbors.

In Block 720 the variable Y is initialized to the value of zero. The variable Y is incremented as neighbor constraints are checked for each neighbor Q_{Y} of M₂.

In Block 730 the variable Y is incremented by 1 to move the constraint check to the next neighbor. For example, if the method had just completed checking the neighbor constraints with respect to M and Q₃ then Y is incremented in Block 730 so that the neighbor constraints may now be checked with respect to M and Q₄.

In Block 770 the variable D is set equal to the distance between Ml and the occurrence of compound Q_{Y} that is not a neighbor to M₂. With reference to exemplary FIG. 9, D is illustrated by the line 930 for the situation where Y=3 (i.e. constraint are being checked for M with respect to neighbor Q₃).

Block 780 is a decisions block that determines whether the distance D between M₁ and the occurrence of compound Q_{Y} that is not a neighbor to M₂ is greater than the neighbor distance Dₚₘᵢₙ. As described above, the neighbor distance Dₚₘᵢₙ may be determined based on many factors. If the distance D is greater than the neighbor distance Dₚₘᵢₙ, the constraints for compound M have been met and the method continues to Block 790. However, if the distance D is less than Dₚₘᵢₙ the method continues to Block 799 indicating a constraint violation.

By halting the process as soon as the constraint is violated, the method rejects not only the current sub-pattern, but all patterns of which it is a part. For example, consider an array where the first 49 dispense positions have satisfied the constraints S_{A} (1 to 49) and then the algorithm discovers that dispense position 50 violates the constraint check. Thus, all patterns that start with this sub-pattern of 50 dispense positions, S_{A} (1 to 50), would violate the constraint. This set of all patterns having the same sub-pattern consists of (768-50)?=718! patterns. Therefore, with the discovery of one constraint violation a large number of unsuitable patterns can be eliminated from consideration.

If the method determines that a particular neighbor Q_{Y} satisfies the neighbor constraint check in block 780 the method continues to Block 790. In decision Block 790, if the variable Y is equal to the number of compounds in the neighborhood of M₂ the method continues to Block 795 and an indication that no constraint violations for compound M at the dispense position S_{X} is returned. However, if Y is less than the number of neighbors in the neighborhood of M₂, the method returns to block 730 where Y is incremented and the neighbor constraints are checked for another neighbor of M₂.

Specific parts, shapes, materials, functions and modules have been set forth, herein. However, a skilled technologist will realize that there are many ways to fabricate the system of the present invention, and that there are many parts, components, modules or functions that may be substituted for those listed above. While the above detailed description has shown, described, and pointed out the fundamental novel features of the invention as applied to various embodiments, it will be understood that various omissions and substitutions and changes in the form and details of the components illustrated may be made by those skilled in the art, without departing from the spirit or essential characteristics of the invention.

What is claimed is:

1. An array of n unique materials, each of which appears in the array at least two times and which has a plurality of neighboring materials, wherein for each of the n materials in the array, the neighboring materials in one occurrence of the material are different from the neighboring materials of all other occurrences of the material, wherein n is greater than 25:

2. The array of claim 1 wherein said materials are considered neighboring materials of a particular material if they are within a predetermined radius around said particular material.

3. The array of claim 2 wherein said predetermined radius is in the range of about 1 mm to 1 cm.

4. The array of claim 2 wherein said predetermined radius is about 3 mm.

5. The array of claim 1 wherein said materials are considered neighboring materials of a particular material if they are within a predetermined dot-pitch distance from said particular material.

6. The array of claim 4, wherein said predetermined dot-pitch distance is 1 dot pitch.

7. The array of claim 4, wherein said predetermined dot-pitch distance is between 1 and 5 dot pitch distances.

8. The array of claim 4, wherein said predetermined dot-pitch distance is greater than 5 dot pitch distances.

9. The array of claim 1, wherein n is greater than 300.

10. The array of claim 1, wherein n is greater than 4000.

11. The array of claim 1, wherein n is greater than 9000.

12. The array of claim 1, further in combination with a planar porous assay matrix, such that a surface of the porous assay matrix is in contact with each of the n materials of the array in such a manner that the materials can diffuse into the porous assay matrix.

13. The array of claim 12 further comprising a substantially non porous assay matrix in contact with said porous assay matrix and containing at least one assay reagent capable of interacting with any of the materials on the porous assay matrix.

14. The array of claim 12, in which the porous assay matrix contains at least one assay reagent capable of interacting with any of the materials in the array that is active in a test assay, wherein the test assay displays positive results (if any) within a time period t, during which time t the materials diffuse within the porous assay matrix to form a spot of diameter d, and wherein “neighboring materials” are within a predetermined distance of the center of the spot.

15. The array of claim 12 further comprising a second porous assay matrix in contact with said porous assay matrix and containing at least one assay reagent capable of interacting with any of the materials in the array that is active in a test assay, wherein the test assay displays positive results (if any) within a time period t, during which time t the materials diffuse within the porous assay matrix and the second porous assay matrix to form a spot of diameter d on the second porous assay matrix and wherein “neighboring materials” are within a predetermined distance of the center of the spot.

16. The array of claim 12, wherein “neighboring materials” include materials within a radius of about 3.5 mm.

17. A method of testing a plurality of samples of different substances for their ability to enhance or inhibit a biological process, the method comprising:
providing an array of at least two dots of each of said samples on a planar matrix such that each of said at least two dots is centered at its own distinct site, wherein at least one of said at least two dots of each of said samples is at least a predetermined distance from at least one of said dots of each of said plurality of samples;
transferring the array from the planar matrix into a uniformly dispersed assay reagent; and
observing the interaction of each of said substances with said assay reagent and correlating said interaction with an ability of each of said substances to enhance or inhibit said biological process.

18. The method of claim 17, further comprising:
providing a second array of at least two dots of each of said samples on a second planar matrix such that each of said at least two dots is centered at its own distinct site, wherein at least one of said at least two dots of each of said samples is at least a predetermined distance from at least one of said dots of each of said plurality of samples, and a pattern of placement of said dots on said second array is different than a pattern of placement of said dots on said first array;
placing said uniformly dispersed assay reagent on said second planar matrix; and
observing the interaction of each of said substances with said assay reagent and correlating said interaction with an ability of each of said substances to enhance or inhibit said biological process.

19. The method of claim 17, further comprising placing a second uniformly dispersed assay reagent on said uniformly dispersed assay reagent, observing a second interaction of each of said substances with said second uniformly dispersed assay reagent, and correlating said second interaction with an ability of each of said substances to enhance or inhibit said biological process.

20. The method of claim 19, further comprising placing a third uniformly dispersed assay reagent on said second uniformly dispersed assay reagent, observing a third interaction of each of said substances with said third uniformly dispersed assay reagent, and correlating said third interaction with an ability of each of said substances to enhance or inhibit said biological process.

21. The method of claim 17, wherein said planar matrix comprises a plurality of alignment dots configured to diffuse into said uniformly dispersed assay reagent; said alignment dots on said uniformly dispersed assay reagent providing reference points for aligning said uniformly dispersed assay reagent with said planar matrix so that each particular interaction is correlated with each particular substance from which each interaction resulted.

22. The method of claim 20, wherein said planar matrix comprises a plurality of alignment dots configured to diffuse into said uniformly dispersed assay reagent, said second uniformly dispersed assay reagent, and said third uniformly dispersed assay reagent; said alignment dots on said third uniformly dispersed assay reagent providing reference points for aligning said third uniformly dispersed assay reagent with said planar matrix so that each particular third interaction is correlated with each particular substance from which each third interaction resulted.

23. A method of testing a plurality of samples of different substances for their ability to enhance or inhibit a biological process, the method comprising:
depositing in an array at least one dot of each of said samples onto a plurality of planar matrixes such that each of said at least one dot is centered at its own distinct site, wherein at least one of said dots of each of said samples is at least a predetermined distance from at least one of said dots of each of said plurality of samples;
transferring the array of samples from the plurality of planar matrixes into a uniformly dispersed assay reagent; and
observing the interaction of each of said substances with said assay reagent and correlating said interaction with an ability of each of said substances to enhance or inhibit said biological process.

24. A system for creating an array of sample compounds comprising:
a relatively flat carrier configured to support a plurality of samples; and
a dispensing mechanism configured to dispense at least two dots of each of said plurality of samples of different compounds on said flat carrier such that each of said at least two dots of each of said plurality of samples has a different set of neighboring compounds within a predetermined distance.

25. The system of claim 24, wherein said samples are chemically bonded to said relatively flat carrier.

26. The system of claim 24, wherein said samples are free to diffuse from said relatively flat carrier.

27. The system of claim 24, wherein said dispensing mechanism simultaneously dispenses a portion of said plurality of samples in a first relative orientation on at least two locations on said flat container.

28. The system of claim 27, wherein none of said samples in said portion of said plurality of samples are neighboring compounds.

29. The system of claim 27, wherein said portion of said plurality of samples comprises twelve samples.

30. The system of claim 24, wherein said flat carrier comprises a plurality of alignment dots configured to diffuse into a material brought into contact with the flat carrier and provide reference points for aligning said material brought into contact with the flat carrier with said flat carrier so that each particular diffused dot is correlated with each particular dot on said flat carrier from which said diffused dot resulted.

31. An array of samples in which each sample appears at least twice and there are at least 768 dispenses of groups of 12 samples.

32. An array of samples in which each sample appears at least twice and there are at least 4608 samples, wherein the array comprises 144 columns and 64 rows and the samples are arranged substantially according to the relative coordinates shown in Table 1.

33. A method for performing an assay, comprising:
providing a substantially planar substrate having an array of at least two dots of each of a plurality of test materials, wherein each of said dots has a plurality of neighboring test materials and is placed such that the neighboring materials in one occurrence of the material are different from the neighboring materials of all other occurrences of the material; and
transferring the array of test materials into a uniformly-dispersed assay reagent that participates in indicating which of the test materials are active in the assay while maintaining the relative positioning of the test materials vis-a-vis each other.

34. The method of claim 33, wherein transferring the array of test materials comprises contacting the array with the assay reagent and allowing the materials to diffuse into the assay reagent.

35. The method of claim 33, wherein transferring the array of test materials comprises contacting the array with a non-porous surface carrying said uniformly dispersed assay reagent and allowing the materials to have surface reactions with said assay reagent.

36. The method of claim 33, wherein transferring the array comprises transferring the materials into a first matrix, and then contacting the first matrix with the assay reagent.

37. The method of claim 33, wherein transferring the array comprises contacting the array with a gel into which the materials can diffuse.

38. The method of claim 33, wherein the uniformly-dispersed assay reagent is surface bound on a non-porous surface so that transferring the array comprises contacting the array with said non-porous surface.

39. An algorithm for formulating an array S having A elements (S_{A}), wherein the value of each element S_{A} represents a dispense position on a card, wherein a dispensing mechanism dispenses a plurality of compounds at each dispense position, the algorithm comprising:
(a) setting each of the A elements of the array S to a random, non-repeating value from 1- A, wherein each S_{A} is set to a preliminary dispense position;
(b) providing an array C having A elements (C_{A}), wherein each of said A elements of the array C comprises J representations of compounds;
(c) setting a counter x to zero;
(d) incrementing x by 2;
(e) determining whether each of said J compounds in C_{X} satisfy a placement constraint with respect to certain other compounds in the array C;
(f) if step (e) determines that there in a constraint violation with one or more of said J compounds in Cₓ;
(1) decrementing x by 2;
(2) determining if there are remaining dispense positions that have not been tested in S_{X} against the constraints of step (e);
(3) if step (2) determines that there are no remaining dispense positions that have not been tested in S_{X} against the constraints of step (e), repeating steps (1) and (2);
(4) changing the value of S_{X} to a remaining dispense position that has not been tested in S_{X} against the constraints of step (e);
(5) return to step (e);
(g) if step (e) determines that there is not a constraint violation with any of said J compounds in C_{X}, return to step (d).

40. The array of claim 39, wherein Cₓ and Cₓ₊₁ are equal and wherein x=any odd integer<A.

41. A method for creating an array of a plurality of materials, each material occurring at least twice within the array and having different neighboring materials in each occurrence, the method comprising:
(a) creating a candidate arrangement of materials containing pairs of n said materials in a spatial relationship;
(b) testing whether a first occurrence and a second occurrence of each of said materials have different neighbors;
(c) if the criteria of (b) is not satisfied, changing said spatial relationship of at least some of n said materials; and
(d) repeating steps (b) and (c) until the criteria of (b) is satisfied.

42. The method of claim 41, wherein said candidate arrangement created in step (a) comprises each of said plurality of materials such that when the criteria of (b) is satisfied, the array is complete.

43. The method of claim 41, wherein said candidate arrangement created in step (a) comprises a portion of said plurality of materials, the method further comprising:
(e) expanding said candidate arrangement of materials by increasing n by a predetermined number;
(f) repeating steps (b)-(e) until a final candidate arrangement is created containing each of said plurality of materials.

44. The method of claim 43, wherein said changing said spatial relationship of at least some of n said materials comprises:
determining whether a placement of a nth material in every open location within said candidate arrangement fails to satisfy the criteria of (b);
decrementing n if said nth material fails to satisfy the criteria of (b) in every open location within said candidate arrangement; and
changing said spatial relationship of at least some of n said materials.

45. A method for formulating an array S having A elements (S_{A}), wherein the value of each element of S_{A} represents a dispense position for one or more materials on a substrate, wherein each material occurs at least two times (C₁ and C₂) in S_{A}, with the constraint that when the distance from one of C₁ and C₂ to any occurrence of any other material is less than or equal to a minimum value D, then the distance from the other of C₁ and C₂ to any occurrence of such other material is greater than D, comprising:
tentatively assigning a plurality of elements to S_{A};
for each first occurrence of a material, determining a set of Q materials that are within a distance of D;
for each second occurrence of a material, ascertaining whether each of the Q materials are within a distance of D and, if so, changing the location of at least one material.

46. A method for formulating an array containing pairs of materials, wherein each member of the pair has different neighboring materials than the other member of the pair, comprising:
(a) tentatively assigning a plurality of materials to array locations;
(b) testing to ascertain whether a first member of a pair has different neighboring materials than a second member of a pair, and if not, then
(c) sequentially repeating step (a) with at least one altered material location and repeating step (b), until each member of each pair has different neighboring materials.

47. The method of claim 46, wherein the testing step comprises measuring the distance from the first member of the pair to the tentative array locations of some or all of the other materials in the array, measuring the distance from the second member of the pair to the tentative array locations of some or all of the other materials in the array, and performing a comparison of data resulting from the measurements.

48. The method of claim 47, wherein the data resulting from the measurements comprises a first set of materials located within a predetermined distance of the first member of the pair.

49. The method of claim 48, wherein the data resulting from the measurements further comprises a second set of materials located within a predetermined distance of the first member.

50. A method of creating an array of sample compounds comprising:
providing a relatively flat carrier configured to support a plurality of samples; and
dispensing at least two dots of each of said plurality of samples of different compounds on said flat carrier such that each of said at least two dots of each of said plurality of samples has a different set of neighboring compounds within a predetermined distance.
